(19) United States
(12) Patent Application Publication
(10) Pub. No.: US 2023/0348450 A1
(43) Pub. Date: Nov. 2, 2023

(54) CARBOXYLIC ACID-CONTAINING COMPOUNDS AS MODULATORS OF BIS-PHOSPHOGLYCERATE MUTASE FOR THE TREATMENT OF SICKLE CELL DISEASE

(71) Applicant: Genzyme Corporation, Cambridge, MA (US)

(72) Inventors: Kunal Desai, Waltham, MA (US); Zhong Fang, Waltham, MA (US); Kevin Guertin, Waltham, MA (US); Vu Hong, Cambridge, MA (US); John Ziqi Jiang, Waltham, MA (US); Sungtaek Lim, Waltham, MA (US); Mark Munson, Natick, MA (US)

(21) Appl. No.: 18/025,847

(22) PCT Filed: Sep. 14, 2021
(86) PCT No.: PCT/US2021/050210

§ 371 Date: Mar. 10, 2023

Related U.S. Application Data
(60) Provisional application No. 63/077,969, filed on Sep. 14, 2020.

(51) Int. Cl. C07D 417/12; C07D 417/14; C07D 417/04; C07D 401/04; C07D 277/28; C07D 277/24; C07D 277/48

(52) U.S. Cl. CPC C07D 417/12; C07D 417/14; C07D 417/04; C07D 401/04; C07D 277/28; C07D 277/24; C07D 277/48

(57) ABSTRACT

Provided herein are compounds and compositions thereof for modulating bis-phosphoglycerate mutase (BPGM) for treating sickle cell disease.

### CROSS-REFERENCE TO RELATED APPLICATIONS

This application claims the benefit under 35 U.S.C. § 119(e) of U.S. Provisional Application No. 63/077,969, filed Sep. 14, 2020, which is hereby incorporated by reference in its entirety.

### FIELD

The present disclosure relates generally to compounds, compositions, and methods for treating sickle cell disease.

### BACKGROUND

Bis-phosphoglycerate mutase (BPGM) is an enzyme in the glucose metabolism pathway that regulates the levels of 2,3-bis-phosphoglycerate (2,3-BPG) inside the red blood cell (RBC). 2,3-BPG is a known allosteric modulator of hemoglobin that stabilizes the de-oxy or “T-State” of hemoglobin that has a tendency to polymerize, resulting in the sickle cell morphology. There is currently a large, unmet medical need for safe and effective oral therapies for the treatment of sickle cell disease. BPGM modulators that lower 2,3-BPG levels offer a novel mechanism of action from existing therapies and may significantly reduce sickling in sickle cell disease patients.

Accordingly, in one aspect, provided herein are compounds which are modulators of BPGM for use in treating sickle cell disease.

### SUMMARY

Described herein, in certain embodiments, are compounds and compositions thereof for modulating bis-phosphoglycerate mutase (BPGM) for treating sickle cell disease.

The following embodiments are encompassed.

Embodiment 1 is a compound of Formula (I):

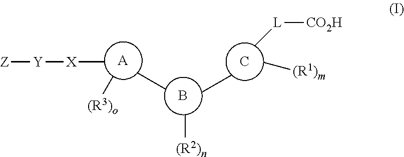

or a pharmaceutically acceptable salt thereof, wherein:
Ring A is phenylene, a 5- to 6-membered heteroarylene, or a 4- to 6-membered heterocyclylene, wherein the heteroarylene and heterocyclylene contain 1-3 heteroatoms selected from N and S;
Ring B is phenylene, a 5- to 6-membered heteroarylene, or a 4- to 6-membered heterocyclylene, wherein the heteroarylene and heterocyclylene contain 1-3 heteroatoms selected from N, O, and S;
Ring C is phenylene or a 5- to 6-membered heteroarylene, wherein the heteroarylene contains 1-3 heteroatoms selected from N, O, and S;
each R¹ is independently —OH, halo, C₁-C₆ alkyl, C₁-C₆ haloalkyl, —CN, —CO₂H, —NR⁵R⁶, or —N(H)CO₂(C₁-C₆ alkyl);
each R² is independently C₁-C₆ alkyl, C₁-C₆ haloalkyl, —CN, halo, —OH, oxo, or phenyl optionally substituted with 1-3 halo or —OH groups;
each R³ is independently C₁-C₆ alkyl or C₁-C₆ haloalkyl;
m is 0-4;
n is 0-4;
o is 0-4;
L is a bond, —OCR⁵R⁶—, —CR⁵R⁶—, —C(O)N(H)CR⁵R⁶CH₂—, —C(O)N(H)CR⁵R⁶—, —(C(O)N(H)CH₂)_{q}—, —C(O)N(H)SO₂(C₆H₄)OCH₂—, —C(O)-(5- to 6-membered heterocyclylene)-OCR⁵CR⁶—, —C(O)N(H)CR⁵R⁶C(O)-(5- to 6-membered heterocyclylene)-, or —S(O)₂CR⁵R⁶—, wherein the heterocyclylene contains 1-3 heteroatoms selected from N and 0;
each R⁵ and R⁶ is independently H, C₁-C₆ alkyl, C₁-C₆ haloalkyl, or phenyl;
q is 1 or 2;
X is —CR⁷R⁸—, —C(O)—, —N(H)—, or a bond; Y is —O—, —N(H)—, —CR⁷R⁸—, —OCR⁷R⁸—, or a bond;
each R⁷ and R⁸ is independently H or C₁-C₆ alkyl;
Z is Z¹ or Z²;
Z¹ is H, halo, C₁-C₆ alkyl, C₁-C₆ haloalkyl, —C(O)(C₁-C₆ alkyl), —C(O)NR⁵R⁶, —CH₂C(O)NR⁵R⁶, —CO₂(C₁-C₆ alkyl), —CO₂(C₁-C₆ haloalkyl), —C(O)(C₁-C₆ alkyl), —C(O)(C₁-C₆ haloalkyl), —SO₂(C₁-C₆ alkyl), —C(O)(C₁-C₆ alkylene)-NR⁵R⁶, —CO₂(C₁-C₆ alkylene)-NR⁵R⁶, —N(H)C(O)(C₁-C₆ alkyl), —C(O)NR⁹(C₁-C₆ alkylene)-NR⁵R⁶, —(C₁-C₆ alkylene)-OR⁹, —C(O)C(O)O(C₁-C₆ alkyl), —C(O)C(O)—NR⁵R⁶, —(CH₂CH₂O)ᵣ(C₁-C₆ alkyl), or —C(O)CH₂(OCH₂CH₂)ᵣO(C₁-C₆ alkyl), wherein C₁-C₆ alkylene is optionally substituted with 1-6 halo, C₁-C₆ alkyl, or C₁-C₆ haloalkyl;
R⁹ is H or C₁-C₆ alkyl;
r is 1-4;
Z² is phenyl, —C(O)(phenyl), 5- to 6-membered heteroaryl, —C(O)-(5- to 6-membered heteroaryl), —CR⁵R⁶-(5- to 6-membered heteroaryl), 4- to 6-membered heterocyclyl, —CR⁵R⁶-(4- to 6-membered heterocyclyl), —C(O)-(4- to 6-membered heterocyclyl), C₃-C₆ cycloalkyl, —C(O)(C₃-C₆ cycloalkyl), —CO₂(C₃-C₆ cycloalkyl), or —CR⁵R⁶—(C₃-C₆ cycloalkyl),
   wherein the heteroaryl and heterocyclyl contain 1-3 heteroatoms selected from N and O, and
   wherein the phenyl, heteroaryl, and heterocyclyl are optionally substituted by 1-5 R¹⁰; and
each R¹⁰ is independently halo, —OH, C₁-C₆ alkyl, C₁-C₆ haloalkyl, —CN, oxo, —NR⁵R⁶, or —N(H)C(O)(C₁-C₆ alkyl).

Embodiment 2 is a compound of embodiment 1, or a pharmaceutically acceptable salt thereof, wherein:
Ring C is phenylene, pyrazolylene, furanylene, thienylene, pyridinylene, pyrrolylene, pyrimidinylene, or thiazolylene.

Embodiment 3 is the compound of embodiment 1 or 2, or a pharmaceutically acceptable salt thereof, wherein:
L is a bond.

Embodiment 4 is the compound of embodiment 1 or 2, or a pharmaceutically acceptable salt thereof, wherein:
L is —OCR⁵R⁶—, —CR⁵R⁶—, —C(O)N(H)CR⁵R⁶CH₂—, —C(O)N(H)CR⁵R⁶—, —C(O)N(H)SO₂(C₆H₄)OCH₂—, —SO₂CR⁵R⁶—, —C(O)-(5- to 6-membered heterocyclylene)-OCR⁵CR⁶—, —C(O)N(H)CR⁵R⁶C(O)-(5- to 6-membered heterocyclylene)-, or —(C(O)N(H)CH₂)_{q}—.

Embodiment 5 is the compound of embodiment 4, or a pharmaceutically acceptable salt thereof, wherein:
R⁵ and R⁶ are each H.

Embodiment 6 is the compound of embodiment 4, or a pharmaceutically acceptable salt thereof, wherein:
R⁵ is H; and
R⁶ is phenyl.

Embodiment 7 is the compound of any one of embodiments 1-6, or a pharmaceutically acceptable salt thereof, wherein:
-L-CO₂H is —CO₂H, —CH₂CO₂H, —OCH₂CO₂H, —C(O)N(H)CH(C₆H₅)CH₂CO₂H, —C(O)N(H)CH(C₆H₅)CO₂H, —SO₂CH₂CO₂H, —C(O)N(H)CH₂C(O)N(H)CH₂CO₂H, —C(O)N(H)CH₂CO₂H, —C(O)N(H)SO₂(C₆H₄)OCH₂CO₂H,

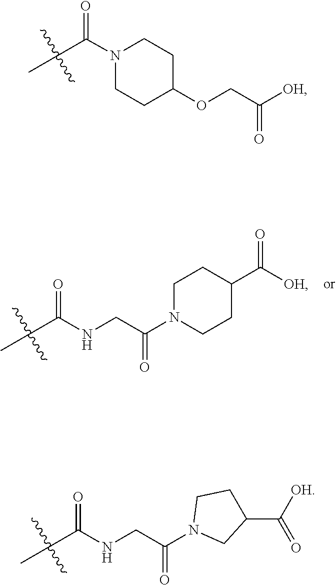 [SMILES: *C(=O)N1CCC(OCC(=O)O)CC1 ; *C(=O)NCC(=O)N1CCC(C(=O)O)CC1 ; *C(=O)NCC(=O)N1CCC(C(=O)O)C1]

Embodiment 8 is the compound of any one of embodiments 1-7, or a pharmaceutically acceptable salt thereof, wherein:
each R¹ is independently halo, —OH, C₁-C₃ haloalkyl, C₁-C₃ alkyl, —CO₂H, —CN, —NH₂, or —N(H)CO₂(C₁-C₃ alkyl).

Embodiment 9 is the compound of embodiment 8, or a pharmaceutically acceptable salt thereof, wherein:
each R¹ is independently F, Cl, —OH, —CHF₂, —CF₃, isopropyl, —CH₃, —CO₂H, —CN, —NH₂, or —N(H)CO₂CH₃.

Embodiment 10 is the compound of any one of embodiments 1-9, or a pharmaceutically acceptable salt thereof, wherein:
m is 0, 1, 2, or 3.

Embodiment 11 is the compound of any one of embodiments 1-10, or a pharmaceutically acceptable salt thereof, wherein:
Ring B is pyrazolylene, thienylene, phenylene, pyridinylene, triazolylene, pyrimidinylene, thiazolylene, piperidinylene, thiadiazolylene, isothiazolylene, oxadiazolylene, oxazolylene, or 2H-pyridinylene.

Embodiment 12 is the compound of any one of embodiments 1-11, or a pharmaceutically acceptable salt thereof, wherein:
each R² is independently C₁-C₃ alkyl, halo, phenyl substituted with 1-2 OH groups, —CN, —OH, or oxo.

Embodiment 13 is the compound of embodiment 12, or a pharmaceutically acceptable salt thereof, wherein:
each R² is independently —CH₃, 2-hydroxyphenyl, —CN, F, —OH, or oxo.

Embodiment 14 is the compound of any one of embodiments 1-13, or a pharmaceutically acceptable salt thereof, wherein:
n is 0 or 1.

Embodiment 15 is the compound of any one of embodiments 1-14, or a pharmaceutically acceptable salt thereof, wherein:
Ring A is thiazolylene, piperidinylene, pyridinylene, pyrazolylene, phenylene, azetidinylene, or pyrrolidinylene.

Embodiment 16 is the compound of any one of embodiments 1-15, or a pharmaceutically acceptable salt thereof, wherein:
each R³ is independently C₁-C₃ alkyl.

Embodiment 17 is the compound of embodiment 16, or a pharmaceutically acceptable salt thereof, wherein:
each R³ is independently —CH₃ or isopropyl.

Embodiment 18 is the compound of any one of embodiments 1-17, or a pharmaceutically acceptable salt thereof, wherein:
X is —CR⁷R⁸—.

Embodiment 19 is the compound of embodiment 18, or a pharmaceutically acceptable salt thereof, wherein:
R⁷ and R⁸ are independently H or —CH₃.

Embodiment 20 is the compound of embodiment 19, or a pharmaceutically acceptable salt thereof, wherein:
R⁷ and R⁸ are each H.

Embodiment 21 is the compound of embodiment 19, or a pharmaceutically acceptable salt thereof, wherein:
R⁷ is H; and
R⁸ is —CH₃.

Embodiment 22 is the compound of any one of embodiments 1-17, or a pharmaceutically acceptable salt thereof, wherein:
X is —C(O)—, —N(H)—, or a bond.

Embodiment 23 is the compound of any one of embodiments 1-22, or a pharmaceutically acceptable salt thereof, wherein:
Y is —O—.

Embodiment 24 is the compound of any one of embodiments 1-22, or a pharmaceutically acceptable salt thereof, wherein:
Y is a bond, —N(H)—, —OCR⁷R⁸—, or —CR⁷R⁸—.

Embodiment 25 is the compound of embodiment 24, or a pharmaceutically acceptable salt thereof, wherein:
R⁷ and R⁸ are each H.

Embodiment 26 is the compound of any one of embodiments 1-25, or a pharmaceutically acceptable salt thereof, wherein:
Z is Z¹.

Embodiment 27 is the compound of embodiment 26, or a pharmaceutically acceptable salt thereof, wherein:
Z¹ is H, halo, C₁-C₃ alkyl, C₁-C₃ haloalkyl, —C(O)(C₁-C₃ alkyl), —C(O)NR⁵R⁶, —CH₂C(O)NR⁵R⁶, —CO₂(C₁-C₃ alkyl), —CO₂(C₁-C₃ haloalkyl), —C(O)(C₁-C₃ alkyl), —C(O)(C₁-C₃ haloalkyl), —SO₂(C₁-C₃ alkyl), —C(O)(C₁-C₃ alkylene)-NR⁵R⁶, —CO₂(C₁-C₃ alkylene)-NR⁵R⁶, —N(H)C(O)(C₁-C₃ alkyl), —(C₁-C₃ alkylene)-OR⁹, —C(O)NR⁹(C₁-C₃ alkylene)-NR⁵R⁶, —C(O)C(O)O(C₁-C₆ alkyl), —C(O)C(O)—NR⁵R⁶, —(CH₂CH₂O)ᵣ(C₁-C₃ alkyl), or —C(O)CH₂(OCH₂CH₂)ᵣO(C₁-C₃ alkyl), wherein C₁-C₃ alkylene is optionally substituted with 1-2 halo, C₁-C₃ alkyl, or C₁-C₃ haloalkyl;
R⁵ and R⁶ are independently H or C₁-C₃ alkyl; and
R⁹ is H or C₁-C₃ alkyl.

Embodiment 28 is the compound of embodiment 26 or 27, or a pharmaceutically acceptable salt thereof, wherein:
Z¹ is —C(O)N(CH₃)₂, —C(O)N(H)CH₃, —C(O)NH₂, H, —CH₂C(CH₃)₃, —C(O)C(CH₃)₃, —C(O)N(CH₃)CH₂CH₂N(CH₃)₂, —C(O)OCH₂CH₂N(CH₃)₂, —C(O)CH₂CH₂N(CH₃)₂, —C(O)OCH₃, —C(O)CH(CH₃)CH₂N(CH₃)₂, —C(O)CH₂N(CH₃)₂, —C(O)CH₂(OCH₂CH₂)₄OCH₃, —C(O)C(CH₃)₂N(CH₃)₂, —C(O)OCH₂CF₃, —CH₂C(O)NH₂, —CH₂C(O)N(CH₃)₂, —CH₂CH₂OCH₃, isopropyl, —C(O)CH₃, —C(O)CO₂CH₃, —C(O)C(O)NH₂, —C(O)C(O)N(CH₃)₂, —SO₂CH₃, —CO₂CH₂CH₃, —CO(isopropyl), —CO₂(isopropyl), F, —N(H)C(O)CH₃, —(CH₂CH₂O)₃CH₃, or —CF₃.

Embodiment 29 is the compound of any one of embodiments 1-25, or a pharmaceutically acceptable salt thereof, wherein:
is Z².

Embodiment 30 is the compound of embodiment 29, or a pharmaceutically acceptable salt thereof, wherein:
Z² is phenyl, —C(O)(phenyl), 5- to 6-membered heteroaryl, —C(O)-(5- to 6-membered heteroaryl), —CR⁵R⁶-(5- to 6-membered heteroaryl), 4- to 6-membered heterocyclyl, —CR⁵R⁶-(4- to 6-membered heterocyclyl), —C(O)-(4- to 6-membered heterocyclyl), C₃-C₆ cycloalkyl, —C(O)(C₃-C₆ cycloalkyl), —CO₂(C₃-C₆ cycloalkyl), or —CR⁵R⁶—(C₃-C₆ cycloalkyl),
   wherein the heteroaryl and heterocyclyl contain 1-3 heteroatoms selected from N and O, and
   wherein the phenyl, heteroaryl, and heterocyclyl are optionally substituted by 1-5 R¹⁰; and
R⁵ and R⁶ are independently H or C₁-C₃ alkyl.

Embodiment 31 is the compound of embodiment 29 or 30, or a pharmaceutically acceptable salt thereof, wherein:
Z² is pyridinyl, pyrimidinyl, pyrrolidinyl, tetrahydropyranyl, tetrahydrofuranyl, pyridazinyl, —C(O)(pyradazinyl), pyrazolyl, —C(O)(cyclopropyl), —CO₂(cyclopropyl), dihydropyridinyl, dihydropyrimidinyl, phenyl, —C(O)(phenyl), —C(O)(piperazinyl), —C(O)(piperidinyl), —C(O)(pyrrolidinyl), —CH₂(pyridinyl), —C(O)(isoxazolyl), —CH(CH₃)(pyridinyl), —C(O)(pyrazolyl), cyclohexyl, cyclobutyl, —C(O)(pyridinyl), —C(O)(pyrimidinyl), —C(O)(cyclopentyl), —C(O)(oxetanyl), morpholinyl, oxazolidinyl, or piperidinyl,
   wherein the phenyl, heteroaryl, and heterocyclyl are optionally substituted by 1-3 R¹⁰.

Embodiment 32 is the compound of any one of embodiments 1-25 and 29-31, or a pharmaceutically acceptable salt thereof, wherein:
each R¹⁰ is independently halo, —OH, C₁-C₃ haloalkyl, C₁-C₃ alkyl, —CN, oxo, —NH₂, or —N(H)C(O)(C₁-C₃ alkyl).

Embodiment 33 is the compound of embodiment 32, or a pharmaceutically acceptable salt thereof, wherein:
each R¹⁰ is independently F, Cl, —OH, —CF₃, isopropyl, —CH₃, —CN, oxo, —NH₂, or —NHC(O)CH₃.

Embodiment 34 is the compound of any one of embodiments 1-25 and 29-33, or a pharmaceutically acceptable salt thereof, wherein:
Z—Y—X— is

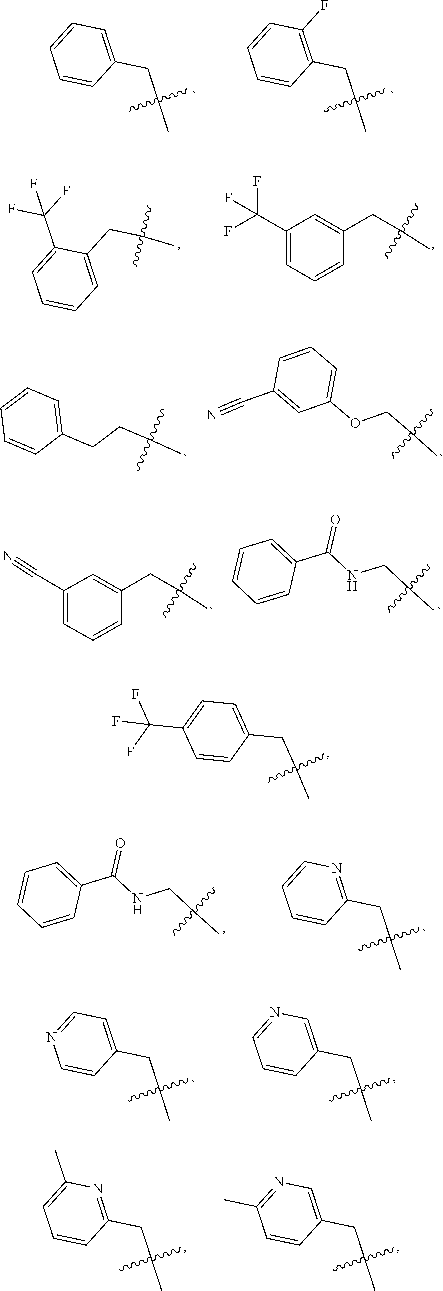

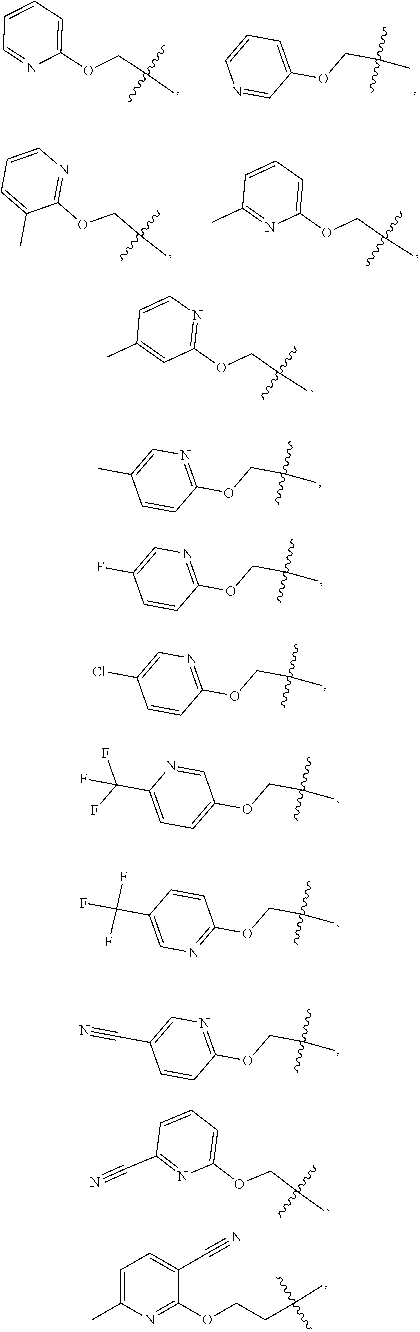

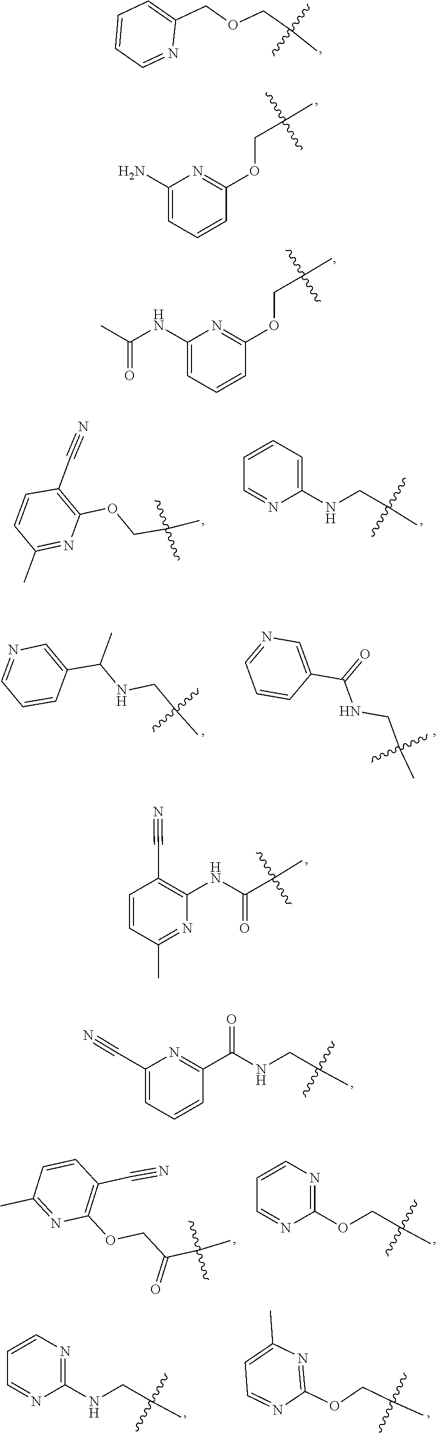

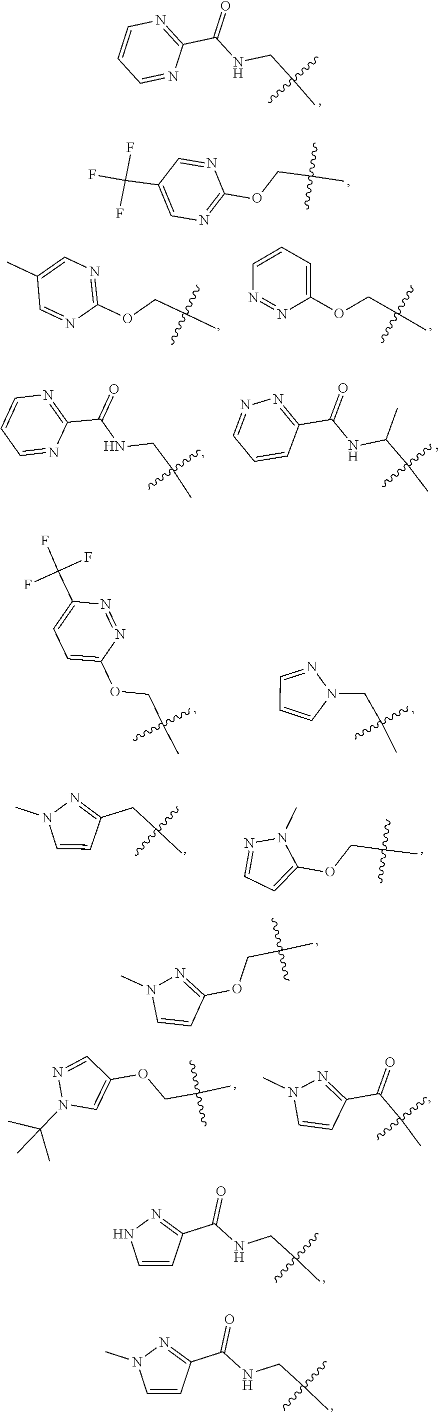

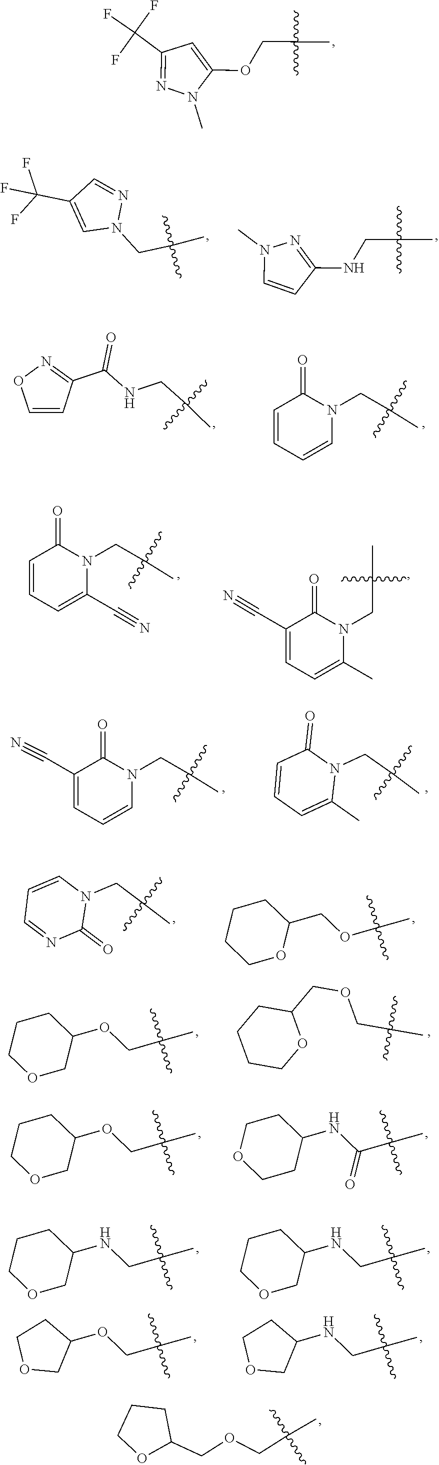

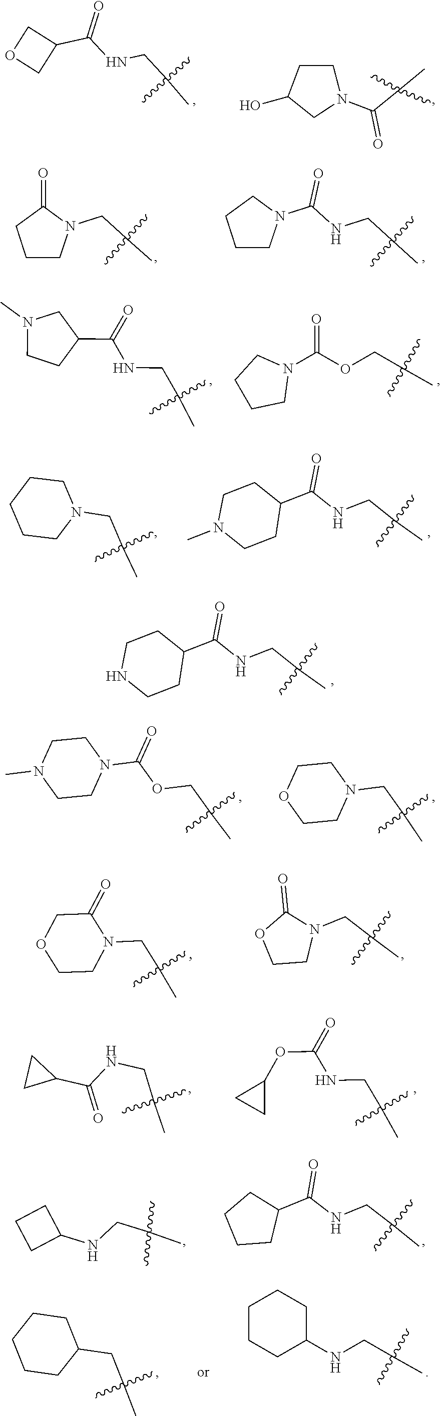

Embodiment 35 is the compound of any one of embodiments 1-28, or a pharmaceutically acceptable salt thereof, wherein:
Z—Y—X— is

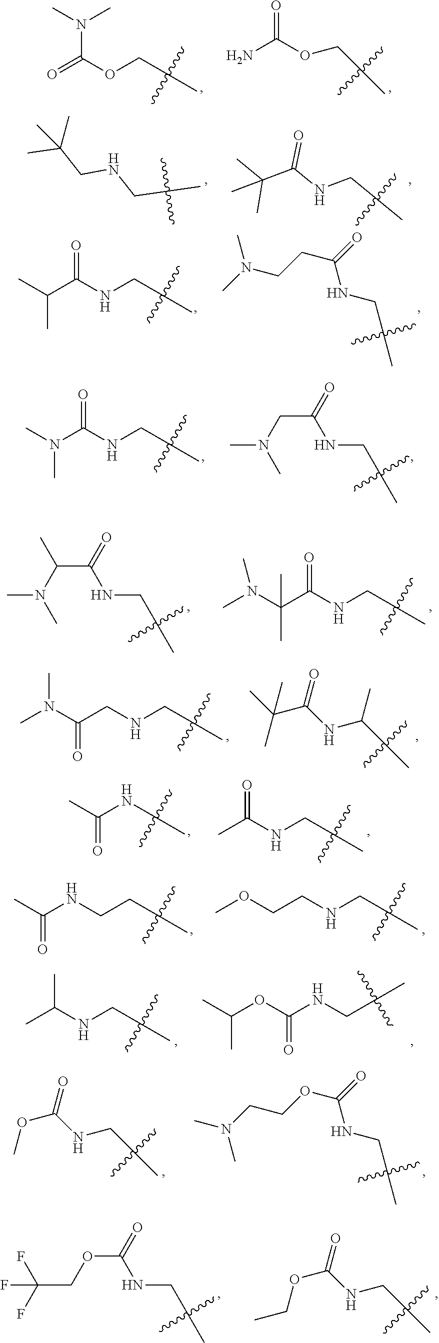

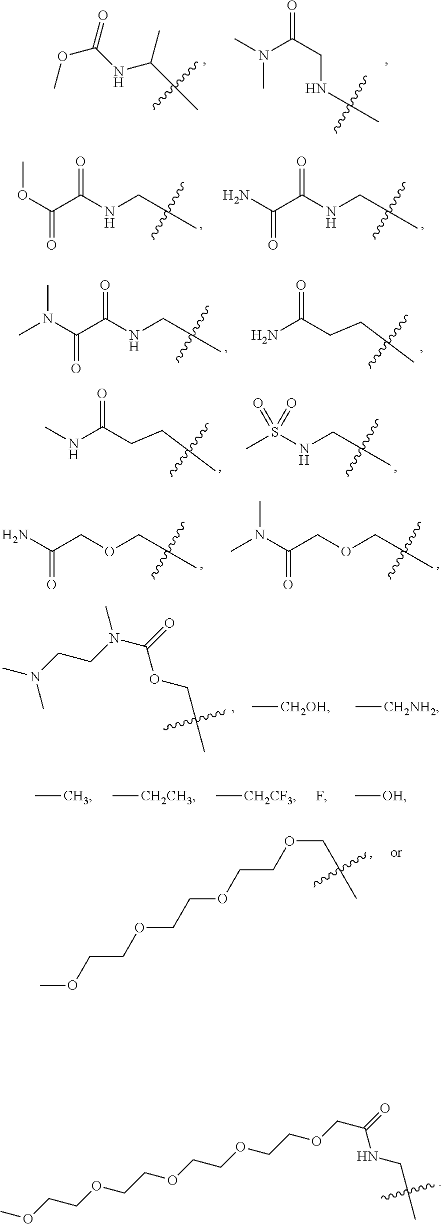

Embodiment 36 is a compound selected from the compounds in Table 1, or a pharmaceutically acceptable salt thereof.

Embodiment 37 is a compound selected from the compounds in Table 2, or a pharmaceutically acceptable salt thereof.

Embodiment 38 is a pharmaceutical composition comprising the compound of any one of embodiments 1-37, or a pharmaceutically acceptable salt thereof, and at least one pharmaceutically acceptable excipient.

Embodiment 39 is a method of modulating bis-phosphoglycerate mutase (BPGM) comprising contacting an effective amount of the compound of any one of embodiments 1-37, or a pharmaceutically acceptable salt thereof, or the pharmaceutical composition of embodiment 38, with the BPGM.

Embodiment 40 is a method of treating sickle cell disease in a subject in need thereof, comprising administering to the subject a therapeutically effective amount of the compound of any one of embodiments 1-37, or a pharmaceutically acceptable salt thereof, or the pharmaceutical composition of embodiment 38.

### DETAILED DESCRIPTION

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the disclosure.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

“Alkyl” refers to an unbranched or branched saturated hydrocarbon chain. As used herein, alkyl has 1 to 20 carbon atoms (i.e., C₁-C₂₀ alkyl), 1 to 10 carbon atoms (i.e., C₁-C₁₀ alkyl), 1 to 6 carbon atoms (i.e., C₁-C₆ alkyl) or 1 to 3 carbon atoms (i.e., C₁-C₃ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), isobutyl (i.e., —CH₂CH(CH₃)₂), sec-butyl (i.e., —CH(CH₃)CH₂CH₃), and tert-butyl (i.e., —C(CH₃)₃); and “propyl” includes n-propyl (i.e., —(CH₂)₂CH₃) and isopropyl (i.e., —CH(CH₃)₂).

“Haloalkyl” refers to an unbranched or branched alkyl group as defined above, wherein one or more hydrogen atoms are replaced by a halogen. For example, “C₁-C₆ haloalkyl” refers to a C₁-C₆ alkyl which is substituted by one or more halogen atoms. A C₁ haloalkyl refers to a methyl group that may be substituted by 1-3 halo groups, a C₂ haloalkyl refers to an ethyl group that may be substituted by 1-5 halo groups, a C₃ haloalkyl refers to a propyl group that may be substituted by 1-7 halo groups, etc. Examples of haloalkyl include trifluoromethyl, difluoromethyl, trichloromethyl, 2,2,2-trifluoroethyl, 1,2-difluoroethyl, 3-bromo-2-fluoropropyl, 1,2-dibromoethyl, and the like. A haloalkyl may contain one or more halo atoms that are the same (i.e., all fluoro) or a mixture of halo atoms (i.e., chloro and fluoro).

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond). As used herein, cycloalkyl has from 3 to 20 ring carbon atoms (i.e., C₃-C₂₀ cycloalkyl), 3 to 10 ring carbon atoms (i.e., C₃-C₁₀ cycloalkyl), or 3 to 6 ring carbon atoms (i.e., C₃-C₆ cycloalkyl). Cycloalkyl also includes “spiro cycloalkyl” when there are two positions for substitution on the same carbon atom. Monocyclic radicals include, for example, cyclopropyl, cyclobutyl, cyclopentyl, cyclohexyl, cycloheptyl and cyclooctyl. Polycyclic radicals include, for example, adamantyl, norbornyl, decalinyl, 7,7-dimethyl-bicyclo[2.2.1]heptanyl and the like. Further, the term cycloalkyl is intended to encompass any non-aromatic ring which may be fused to an aryl ring, regardless of the attachment to the remainder of the molecule.

“Halogen” or “halo” includes fluoro, chloro, bromo, and iodo.

“Oxo” refers to the atom (═O) or (O).

“Heteroaryl” refers to an aromatic group (e.g., a 5-14 membered ring system) having a single ring, multiple rings, or multiple fused rings, with one or more ring heteroatoms independently selected from nitrogen, oxygen and sulfur. As used herein, heteroaryl includes 1 to 10 ring carbon atoms and 1 to 4 heteroatoms independently selected from nitrogen, oxygen and sulfur within the ring. Examples of heteroaryl groups include pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl and thiophenyl (i.e., thienyl).

“Heterocyclyl” refers to a saturated or unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more oxo (C═O) or N-oxide (N—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 1 to 10 ring carbon atoms, 1 to 8 carbon atoms, 1 to 6 carbon atoms, or 1 to 4 carbon atoms, and 1 to 5 ring heteroatoms, 1 to 4 heteroatoms, 1 to 3 heteroatoms, or 1 to 2 heteroatoms independently selected from nitrogen, sulfur and oxygen. Examples of heterocyclyl groups include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, 1-oxo-thiomorpholinyl, and 1,1-dioxo-thiomorpholinyl.

Certain commonly used alternative chemical names may be used. For example, a divalent group such as a divalent “alkyl” group, a divalent “phenyl” group, a divalent “heteroaryl” group, a divalent “heterocyclyl” group etc., may also be referred to as an “alkylene” group, an “phenylene” group, a “heteroarylene” group, or a “heterocyclylene” group, respectively.

The terms “optional” or “optionally” means that the subsequently described event or circumstance may or may not occur and that the description includes instances where said event or circumstance occurs and instances in which it does not. Also, the term “optionally substituted” refers to any one or more hydrogen atoms on the designated atom or group may or may not be replaced by a moiety other than hydrogen.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan.

Any compound or Formula described herein is intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. Various isotopically labeled compounds of the present disclosure, for example those into which radioactive isotopes such as 3H and ¹⁴C are incorporated, are included in this disclosure. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds described herein in which from 1 to n hydrogens attached to a carbon atom is/are replaced by deuterium, in which n is the number of hydrogens in the molecule. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

“Pharmaceutically acceptable” refers to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The compounds disclosed herein, or their pharmaceutically acceptable salts, may include an asymmetric center and may thus give rise to enantiomers, diastereomers, and other stereoisomeric forms that may be defined, in terms of absolute stereochemistry, as (R)- or (S)- or, as (D)- or (L)- for amino acids. The disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

“Tautomer” refers to alternate forms of a compound that differ in the position of a proton, such as enol-keto and imine-enamine tautomers, or the tautomeric forms of heteroaryl groups containing a ring atom attached to both a ring —NH-moiety and a ring=N moiety such as pyrazoles, imidazoles, benzimidazoles, triazoles, and tetrazoles. All tautomeric forms of the compounds described herein are intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

“Diastereoisomers” are stereoisomers that have at least two asymmetric atoms, but which are not mirror-images of each other.

As used herein, “pharmaceutically acceptable carrier” or “pharmaceutically acceptable excipient” or “excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Effective amount” or dose of a compound or a composition refers to that amount of the compound or the composition that results in an intended result as desired based on the disclosure herein. Effective amounts can be determined by standard pharmaceutical procedures in cell cultures or experimental animals including, without limitation, by determining the LD₅₀ (the dose lethal to 50% of the population) and the ED₅₀ (the dose therapeutically effective in 50% of the population).

“Therapeutically effective amount” or dose of a compound or a compositions refers to that amount of the compound or the composition that results in reduction or inhibition of symptoms or a prolongation of survival in a subject (i.e., a human patient). The results may require multiple doses of the compound or the composition.

“Treating” or “treatment” of a disease in a subject refers to 1) preventing the disease from occurring in a patient that is predisposed or does not yet display symptoms of the disease; 2) inhibiting the disease or arresting its development; or 3) ameliorating or causing regression of the disease. As used herein, “treatment” or “treating” is an approach for obtaining beneficial or desired results including clinical results. For the purposes of this disclosures, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a subject. Also encompassed by “treatment” is a reduction of pathological consequence of the disease or disorder. The methods of the disclosure contemplate any one or more of these aspects of treatment.

As used herein, the terms “individual(s),” “subject(s),” and “patient(s)” mean any mammal. Examples include, but are not limited to, mice, rats, hamsters, guinea pigs, pigs, rabbits, cats, dogs, goats, sheep, cows, and humans. In some embodiments, the mammal is a human.

Although various features of the disclosure may be described in the context of a single embodiment, the features may also be provided separately or in any suitable combination. Conversely, although the disclosure may be described herein in the context of separate embodiments for clarity, the disclosure may also be implemented in a single embodiment.

Compounds

In one aspect, provided herein is a compound of Formula (I):

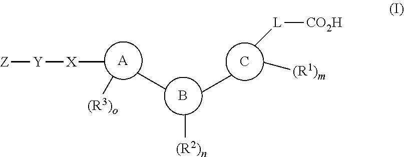

or a pharmaceutically acceptable salt thereof, wherein:
Ring A is phenylene, a 5- to 6-membered heteroarylene, or a 4- to 6-membered heterocyclylene, wherein the heteroarylene and heterocyclylene contain 1-3 heteroatoms selected from N and S;
Ring B is phenylene, a 5- to 6-membered heteroarylene, or a 4- to 6-membered heterocyclylene, wherein the heteroarylene and heterocyclylene contain 1-3 heteroatoms selected from N, O, and S;
Ring C is phenylene or a 5- to 6-membered heteroarylene, wherein the heteroarylene contains 1-3 heteroatoms selected from N, O, and S;
each R¹ is independently —OH, halo, C₁-C₆ alkyl, C₁-C₆ haloalkyl, —CN, —CO₂H, —NR⁵R⁶, or —N(H)CO₂(C₁-C₆ alkyl);
each R² is independently C₁-C₆ alkyl, C₁-C₆ haloalkyl, —CN, halo, —OH, oxo, or phenyl optionally substituted with 1-3 halo or —OH groups;
each R³ is independently C₁-C₆ alkyl or C₁-C₆ haloalkyl;
m is 0-4;
n is 0-4;
o is 0-4;
L is a bond, —OCR⁵R⁶—, —CR⁵R⁶—, —C(O)N(H)CR⁵R⁶CH₂—, —C(O)N(H)CR⁵R⁶—, —C(O)N(H)SO₂(C₆H₄)OCH₂—, —(C(O)N(H)CH₂)_{q}—, —C(O)-(5- to 6-membered heterocyclylene)-OCR⁵CR⁶—, —C(O)N(H)CR⁵R⁶C(O)-(5- to 6-membered heterocyclylene)-, or —S(O)₂CR⁵R⁶—, wherein the heterocyclylene contains 1-3 heteroatoms selected from N and 0;
each R⁵ and R⁶ is independently H, C₁-C₆ alkyl, C₁-C₆ haloalkyl, or phenyl;
q is 1 or 2;
X is —CR⁷R⁸—, —C(O)—, —N(H)—, or a bond; Y is —O—, —N(H)—, —CR⁷R⁸—, —OCR⁷R⁸—, or a bond;
each R⁷ and R⁸ is independently H or C₁-C₆ alkyl;
Z is Z¹ or Z²; Z¹ is H, halo, C₁-C₆ alkyl, C₁-C₆ haloalkyl, —C(O)(C₁-C₆ alkyl), —C(O)NR⁵R⁶, —CH₂C(O)NR⁵R⁶, —CO₂(C₁-C₆ alkyl), —CO₂(C₁-C₆ haloalkyl), —C(O)(C₁-C₆ alkyl), —C(O)(C₁-C₆ haloalkyl), —SO₂(C₁-C₆ alkyl), —C(O)(C₁-C₆ alkylene)-NR⁵R⁶, —CO₂(C₁-C₆ alkylene)-NR⁵R⁶, —N(H)C(O)(C₁-C₆ alkyl), —C(O)NR⁹(C₁-C₆ alkylene)-NR⁵R⁶, —(C₁-C₆ alkylene)-OR⁹, —C(O)C(O)O(C₁-C₆ alkyl), —C(O)C(O)—NR⁵R⁶, —(CH₂CH₂O)ᵣ(C₁-C₆ alkyl), or —C(O)CH₂(OCH₂CH₂)ᵣO(C₁-C₆ alkyl),
   wherein C₁-C₆ alkylene is optionally substituted with 1-6 halo, C₁-C₆ alkyl, or C₁-C₆ haloalkyl;
R⁹ is H or C₁-C₆ alkyl;
r is 1-4;
Z² is phenyl, —C(O)(phenyl), 5- to 6-membered heteroaryl, —C(O)-(5- to 6-membered heteroaryl), —CR⁵R⁶-(5- to 6-membered heteroaryl), 4- to 6-membered heterocyclyl, —CR⁵R⁶-(4- to 6-membered heterocyclyl), —C(O)-(4- to 6-membered heterocyclyl), C₃-C₆ cycloalkyl, —C(O)(C₃-C₆ cycloalkyl), —CO₂(C₃-C₆ cycloalkyl), or —CR⁵R⁶—(C₃-C₆ cycloalkyl),
   wherein the heteroaryl and heterocyclyl contain 1-3 heteroatoms selected from N and O, and
   wherein the phenyl, heteroaryl, and heterocyclyl are optionally substituted by 1-5 R¹⁰; and
each R¹⁰ is independently halo, —OH, C₁-C₆ alkyl, C₁-C₆ haloalkyl, —CN, oxo, —NR⁵R⁶, or —N(H)C(O)(C₁-C₆ alkyl).

Some embodiments provide for a compound of Formula (I-a):

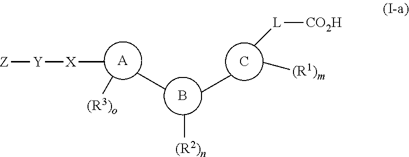

or a pharmaceutically acceptable salt thereof, wherein:
Ring A is thiazolylene;
Ring B is phenylene, a 5- to 6-membered heteroarylene, or a 4- to 6-membered heterocyclylene, wherein the heteroarylene and heterocyclylene contain 1-3 heteroatoms selected from N, O, and S;
Ring C is phenylene or a 5- to 6-membered heteroarylene, wherein the heteroarylene contains 1-3 heteroatoms selected from N, O, and S;
each R¹ is independently —OH, halo, C₁-C₆ alkyl, C₁-C₆ haloalkyl, —CN, —CO₂H, —NR⁵R⁶, or —N(H)CO₂(C₁-C₆ alkyl);
each R² is independently C₁-C₆ alkyl, C₁-C₆ haloalkyl, —CN, halo, —OH, oxo, or phenyl optionally substituted with 1-3 halo or —OH groups;
each R³ is independently C₁-C₆ alkyl or C₁-C₆ haloalkyl;
m is 0-4;
n is 0-4;
o is 0-4;
L is a bond, —OCR⁵R⁶—, —CR⁵R⁶—, —C(O)N(H)CR⁵R⁶CH₂—, —C(O)N(H)CR⁵R⁶—, —C(O)N(H)SO₂(C₆H₄)OCH₂—, —(C(O)N(H)CH₂)_{q}—, —C(O)-(5- to 6-membered heterocyclylene)-OCR⁵CR⁶—, —C(O)N(H)CR⁵R⁶C(O)-(5- to 6-membered heterocyclylene)-, or —S(O)₂CR⁵R⁶—,
   wherein the heterocyclylene contains 1-3 heteroatoms selected from N and 0;
each R⁵ and R⁶ is independently H, C₁-C₆ alkyl, C₁-C₆ haloalkyl, or phenyl;
q is 1 or 2;
X is —CR⁷R⁸—;
Y is —O—, —N(H)—, —CR⁷R⁸—, —OCR⁷R⁸—, or a bond;
each R⁷ and R⁸ is independently H or C₁-C₆ alkyl;
Z is Z¹ or Z²;
Z¹ is H, halo, C₁-C₆ alkyl, C₁-C₆ haloalkyl, —C(O)(C₁-C₆ alkyl), —C(O)NR⁵R⁶, —CH₂C(O)NR⁵R⁶, —CO₂(C₁-C₆ alkyl), —CO₂(C₁-C₆ haloalkyl), —C(O)(C₁-C₆ alkyl), —C(O)(C₁-C₆ haloalkyl), —SO₂(C₁-C₆ alkyl), —C(O)(C₁-C₆ alkylene)-NR⁵R⁶, —CO₂(C₁-C₆ alkylene)-NR⁵R⁶, —N(H)C(O)(C₁-C₆ alkyl), —C(O)NR⁹(C₁-C₆ alkylene)-NR⁵R⁶, —(C₁-C₆ alkylene)-OR⁹, —C(O)C(O)O(C₁-C₆ alkyl), —C(O)C(O)—NR⁵R⁶, —(CH₂CH₂O)ᵣ(C₁-C₆ alkyl), or —C(O)CH₂(OCH₂CH₂)ᵣO(C₁-C₆ alkyl),
   wherein C₁-C₆ alkylene is optionally substituted with 1-6 halo, C₁-C₆ alkyl, or C₁-C₆ haloalkyl;
R⁹ is H or C₁-C₆ alkyl;
r is 1-4;
Z² is phenyl, —C(O)(phenyl), 5- to 6-membered heteroaryl, —C(O)-(5- to 6-membered heteroaryl), —CR⁵R⁶-(5- to 6-membered heteroaryl), 4- to 6-membered heterocyclyl, —CR⁵R⁶-(4- to 6-membered heterocyclyl), —C(O)-(4- to 6-membered heterocyclyl), C₃-C₆ cycloalkyl, —C(O)(C₃-C₆ cycloalkyl), —CO₂(C₃-C₆ cycloalkyl), or —CR⁵R⁶—(C₃-C₆ cycloalkyl),
   wherein the heteroaryl and heterocyclyl contain 1-3 heteroatoms selected from N and O, and
   wherein the phenyl, heteroaryl, and heterocyclyl are optionally substituted by 1-5 R¹⁰; and
each R¹⁰ is independently halo, —OH, C₁-C₆ alkyl, C₁-C₆ haloalkyl, —CN, oxo, —NR⁵R⁶, or —N(H)C(O)(C₁-C₆ alkyl).

Some embodiments provide for a compound of Formula (I-al):

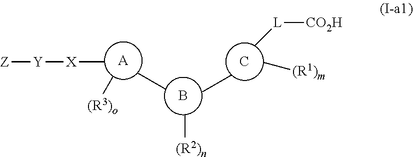

or a pharmaceutically acceptable salt thereof, wherein:
Ring A is thiazolylene;
Ring B is phenylene, a 5- to 6-membered heteroarylene, or a 4- to 6-membered heterocyclylene,
   wherein the heteroarylene and heterocyclylene contain 1-3 heteroatoms selected from N, O, and S;
Ring C is phenylene or a 5- to 6-membered heteroarylene, wherein the heteroarylene contains 1-3 heteroatoms selected from N, O, and S;
each R¹ is independently —OH, halo, C₁-C₆ alkyl, C₁-C₆ haloalkyl, —CN, —CO₂H, —NR⁵R⁶, or —N(H)CO₂(C₁-C₆ alkyl);
each R² is independently C₁-C₆ alkyl, C₁-C₆ haloalkyl, —CN, halo, —OH, oxo, or phenyl optionally substituted with 1-3 halo or —OH groups;
each R³ is independently C₁-C₆ alkyl or C₁-C₆ haloalkyl;
m is 0-4;
n is 0-4;
o is 0-4;
L is a bond, —OCR⁵R⁶—, —CR⁵R⁶—, —C(O)N(H)CR⁵R⁶CH₂—, —C(O)N(H)CR⁵R⁶—, —C(O)N(H)SO₂(C₆H₄)OCH₂—, —(C(O)N(H)CH₂)_{q}—, —C(O)-(5- to 6-membered heterocyclylene)-OCR⁵CR⁶—, —C(O)N(H)CR⁵R⁶C(O)-(5- to 6-membered heterocyclylene)-, or —S(O)₂CR⁵R⁶—, wherein the heterocyclylene contains 1-3 heteroatoms selected from N and 0;
each R⁵ and R⁶ is independently H, C₁-C₆ alkyl, C₁-C₆ haloalkyl, or phenyl;
q is 1 or 2;
X is —CR⁷R⁸—;
Y is —O—, —N(H)—, —CR⁷R⁸—, or —OCR⁷R⁸—;
each R⁷ and R⁸ is independently H or C₁-C₆ alkyl;
Z is Z²;
Z² is phenyl, —C(O)(phenyl), 5- to 6-membered heteroaryl, —C(O)-(5- to 6-membered heteroaryl), —CR⁵R⁶-(5- to 6-membered heteroaryl), 4- to 6-membered heterocyclyl, —CR⁵R⁶-(4- to 6-membered heterocyclyl), —C(O)-(4- to 6-membered heterocyclyl), C₃-C₆ cycloalkyl, —C(O)(C₃-C₆ cycloalkyl), —CO₂(C₃-C₆ cycloalkyl), or —CR⁵R⁶—(C₃-C₆ cycloalkyl),
   wherein the heteroaryl and heterocyclyl contain 1-3 heteroatoms selected from N and O, and
   wherein the phenyl, heteroaryl, and heterocyclyl are optionally substituted by 1-5 R¹⁰; and
each R¹⁰ is independently halo, —OH, C₁-C₆ alkyl, C₁-C₆ haloalkyl, —CN, oxo, —NR⁵R⁶, or —N(H)C(O)(C₁-C₆ alkyl).

In some embodiments, Ring C is phenylene. In some embodiments, Ring C is a 5- to 6-membered heteroarylene containing 1-3 heteroatoms selected from N, O, and S. In some embodiments, Ring C is a 5-membered heteroarylene containing 1-3 heteroatoms selected from N, O, and S. In some embodiments, Ring C is a 6-membered heteroarylene containing 1-3 nitrogen heteroatoms. In some embodiments, Ring C is phenylene, pyrazolylene, furanylene, thienylene, pyridinylene, pyrrolylene, pyrimidinylene, or thiazolylene.

In some embodiments, L is a bond. In some embodiments, L is —OCR⁵R⁶—, —CR⁵R⁶—, —C(O)N(H)CR⁵R⁶CH₂—, —C(O)N(H)CR⁵R⁶—, —(C(O)N(H)CH₂)_{q}—, or —S(O)₂CR⁵R⁶—. In some embodiments, L is —OCR⁵R⁶—. In some embodiments, L is —CR⁵R⁶—. In some embodiments, L is —C(O)N(H)CR⁵R⁶CH₂—. In some embodiments, L is —C(O)N(H)CR⁵R⁶—. In some embodiments, L is —(C(O)N(H)CH₂)_{q}—, wherein q is 1 or 2. In some embodiments, q is 1. In other embodiments, q is 2. In some embodiments, L is —S(O)₂CR⁵R⁶—. In some embodiments, L is —C(O)N(H)CR⁵R⁶C(O)-(5- to 6-membered heterocyclylene)-, wherein the heterocyclylene contains 1-3 heteroatoms selected from N and O. In some embodiments, L is —C(O)N(H)CR⁵R⁶C(O)-(5- to 6-membered heterocyclylene)-, wherein the heterocyclylene contains 1 nitrogen heteroatom. In some embodiments, L is —C(O)-(5- to 6-membered heterocyclylene)-OCR⁵CR⁶, wherein the heterocyclylene contains 1-3 heteroatoms selected from N and O. In some embodiments, L is —C(O)-(5- to 6-membered heterocyclylene)-OCR⁵CR⁶, wherein the heterocyclylene contains 1 nitrogen heteroatom. In some embodiments, L is

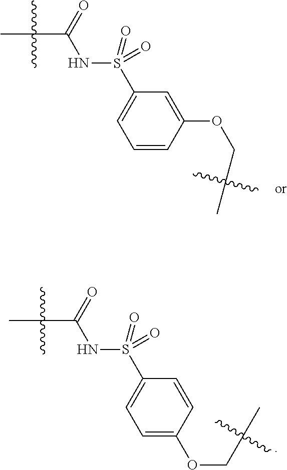 [SMILES: *COc1cccc(S(=O)(=O)NC(*)=O)c1 ; *COc1ccc(S(=O)(=O)NC(*)=O)cc1]

—C(O)N(H)SO₂(C₆H₄)OCH₂—. In some embodiments, L is or O.

In some embodiments, each R⁵ and R⁶ is independently H, C₁-C₆ alkyl, C₁-C₆ haloalkyl, or phenyl. In some embodiments, each R⁵ and R⁶ is independently H, C₁-C₃ alkyl, C₁-C₃ haloalkyl, or phenyl. In some embodiments, R⁵ and R⁶ are each H. In some embodiments, R⁵ is H and R⁶ is phenyl. In some embodiments, R⁵ is H and R⁶ is methyl. In some embodiments, R⁵ and R⁶ are each methyl. In some embodiments, R⁵ and R⁶ are each independently C₁-C₃ alkyl. In some embodiments, R⁵ and R⁶ are each independently C₁-C₃ haloalkyl. In some embodiments, R⁵ is H and R⁶ is C₁-C₆ alkyl, C₁-C₆ haloalkyl, or phenyl. In some embodiments, R⁵ is H and R⁶ is C₁-C₃ alkyl, C₁-C₃ haloalkyl, or phenyl.

In some embodiments, -L-CO₂H is —CO₂H, —CH₂CO₂H, —OCH₂CO₂H, —C(O)N(H)C(C₆H₅)CH₂CO₂H, —C(O)N(H)C(C₆H₅)CO₂H, —SO₂CH₂CO₂H, —C(O)N(H)CH₂C(O)N(H)CH₂CO₂H, or —C(O)N(H)CH₂CO₂H. In some embodiments, -L-CO₂H is —CO₂H. In some embodiments, -L-CO₂H is —CH₂CO₂H. In some embodiments, -L-CO₂H is —OCH₂CO₂H. In some embodiments, -L-CO₂H is —C(O)N(H)C(C₆H₅)CH₂CO₂H. In some embodiments, -L-CO₂H is —C(O)N(H)C(C₆H₅)CO₂H. In some embodiments, -L-CO₂H is —SO₂CH₂CO₂H. In some embodiments, -L-CO₂H is —C(O)N(H)CH₂C(O)N(H)CH₂CO₂H. In some embodiments, -L-CO₂H is —C(O)N(H)CH₂CO₂H.

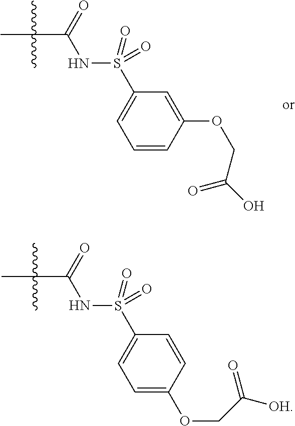 [SMILES: *C(=O)NS(=O)(=O)c1cccc(OCC(=O)O)c1 ; *C(=O)NS(=O)(=O)c1ccc(OCC(=O)O)cc1]

In some embodiments, -L-CO₂H is OH or O. In some embodiments, -L-CO₂H is

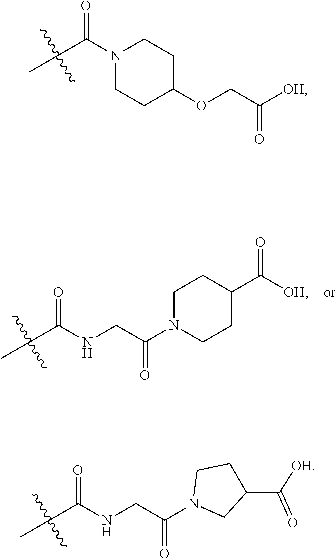 [SMILES: *C(=O)N1CCC(OCC(=O)O)CC1 ; *C(=O)NCC(=O)N1CCC(C(=O)O)CC1 ; *C(=O)NCC(=O)N1CCC(C(=O)O)C1]

In some embodiments, -L-CO₂H is

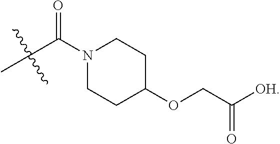 [SMILES: *C(=O)N1CCC(OCC(=O)O)CC1]

In some embodiments, -L-CO₂H is

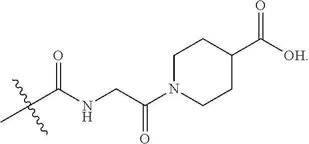 [SMILES: *C(=O)NCC(=O)N1CCC(C(=O)O)CC1]

In some embodiments, -L-CO₂H is

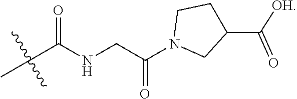 [SMILES: *C(=O)NCC(=O)N1CCC(C(=O)O)C1]

In some embodiments, each R¹ is independently —OH, halo, C₁-C₆ alkyl, C₁-C₆ haloalkyl, —CN, —CO₂H, —NR⁵R⁶, —N(H)CO₂(C₁-C₆ alkyl). In some embodiments, each R¹ is independently halo, —OH, C₁-C₃ haloalkyl, C₁-C₃ alkyl, —CO₂H, —CN, —NH₂, or —N(H)CO₂(C₁-C₃ alkyl). In some embodiments, each R¹ is independently F, Cl, —OH, —CHF₂, —CF₃, isopropyl, —CH₃, —CO₂H, —CN, —NH₂, or —N(H)CO₂CH₃.

In some embodiments, m is 0. In some embodiments, m is 1-4. In some embodiments, m is 1-3. In some embodiments, m is 0, 1, 2, or 3. In some embodiments, m is 0. In some embodiments, m is 1. In some embodiments, m is 2. In some embodiments, m is 3. In some embodiments, m is 4.

In some embodiments, Ring B is phenylene. In some embodiments, Ring B is a 5- to 6-membered heteroarylene or a 4- to 6-membered heterocyclylene, wherein the heteroarylene and heterocyclylene contain 1-3 heteroatoms selected from N, O, and S. In some embodiments, Ring B is a 5- to 6-membered heteroarylene containing 1-3 heteroatoms selected from N, O, and S. In some embodiments, Ring B is a 5-membered heteroarylene containing 1-3 heteroatoms selected from N, O, and S. In some embodiments, Ring B is a 6-membered heteroarylene containing 1-3 nitrogen heteroatoms. In some embodiments, Ring B is a 4- to 6-membered heterocyclylene containing 1-3 heteroatoms selected from N, O, and S. In some embodiments, Ring B is a 6-membered heterocyclylene containing 1-3 nitrogen heteroatoms. In some embodiments, Ring B is pyrazolylene, thienylene, phenylene, pyridinylene, triazolylene, pyrimidinylene, thiazolylene, piperidinylene, thiadiazolylene, isothiazolylene, oxadiazolylene, oxazolylene, or 2H-pyridinylene. In some embodiments, Ring B is pyrazolylene. In some embodiments, Ring B is thienylene. In some embodiments, Ring B is pyridinylene. In some embodiments, Ring B is triazolylene. In some embodiments, Ring B is pyrimidinylene. In some embodiments, Ring B is thiazolylene. In some embodiments, Ring B is piperidinylene. In some embodiments, Ring B is thiadiazolylene. In some embodiments, Ring B is isothiazolylene. In some embodiments, Ring B is oxadiazolylene. In some embodiments, Ring B is oxazolylene. In some embodiments, Ring B is 2H-pyridinylene.

In some embodiments, each R² is independently C₁-C₆ alkyl, C₁-C₆ haloalkyl, —CN, halo, —OH, oxo, or phenyl optionally substituted with 1-3 halo or —OH groups. In some embodiments, each R² is independently C₁-C₃ alkyl, C₁-C₃ haloalkyl, —CN, halo, —OH, oxo, or phenyl optionally substituted with 1-3 halo or —OH groups. In some embodiments, R² is C₁-C₃ alkyl. In some embodiments, R² is C₁-C₃ haloalkyl. In some embodiments, R² is —CN. In some embodiments, R² is halo. In some embodiments, R² is —OH. In some embodiments, R² is oxo. In some embodiments, R² is phenyl optionally substituted with 1-3 halo or —OH groups. In some embodiments, R² is oxo. In some embodiments, R² is phenyl optionally substituted with 1 halo or —OH. In some embodiments, each R² is independently —CH₃, 2-hydroxyphenyl, —CN, F, —OH, or oxo.

In some embodiments, n is 0. In some embodiments, n is 1-4. In some embodiments, n is 1-3. In some embodiments, n is 0 or 1. In some embodiments, n is 1. In some embodiments, n is 2. In some embodiments, n is 3. In some embodiments, n is 4.

In some embodiments, Ring A is phenylene. In some embodiments, Ring A is phenylene, and o is 1. In some embodiments, Ring A is a 5- to 6-membered heteroarylene or a 4- to 6-membered heterocyclylene, wherein the heteroarylene and heterocyclylene contain 1-3 heteroatoms selected from N and S. In some embodiments, Ring A is a 5- to 6-membered heteroarylene containing 1-3 heteroatoms selected from N and S. In some embodiments, Ring A is a 5- to 6-membered heteroarylene containing 1-3 heteroatoms selected from N and S, and o is 1. In some embodiments, Ring A is a 5-membered heteroarylene containing 1-3 nitrogen heteroatoms. In some embodiments, Ring A is a 6-membered heteroarylene containing 1-3 nitrogen heteroatoms. In some embodiments, Ring A is a 4- to 6-membered heterocyclylene containing 1-3 heteroatoms selected from N and S. In some embodiments, Ring A is a 4 to 6-membered heterocyclylene containing 1-3 heteroatoms selected from N and S, and o is 1. In some embodiments, Ring A is a 4-membered heterocyclylene containing 1-2 nitrogen heteroatoms. In some embodiments, Ring A is a 5-membered heterocyclylene containing 1-3 nitrogen heteroatoms. In some embodiments, Ring A is a 6-membered heterocyclylene containing 1-3 nitrogen heteroatoms. In some embodiments, Ring A is thiazolylene, piperidinylene, pyridinylene, pyrazolylene, phenylene, azetidinylene, or pyrrolidinylene. In some embodiments, Ring A is thiazolylene. In some embodiments, Ring A is piperidinylene. In some embodiments, Ring A is pyridinylene. In some embodiments, Ring A is pyrazolylene. In some embodiments, Ring A is azetidinylene. In some embodiments, Ring A is pyrrolidinylene.

In some embodiments, each R³ is independently C₁-C₆ alkyl or C₁-C₆ haloalkyl. In some embodiments, each R³ is independently C₁-C₃ alkyl. In some embodiments, each R³ is independently C₁-C₃ haloalkyl. In some embodiments, each R³ is independently —CH₃ or isopropyl. In some embodiments, R³ is methyl. In some embodiments, o is 1, and R³ is methyl. In some embodiments, R³ is isopropyl.

In some embodiments, o is 0-4. In some embodiments, o is 0. In some embodiments, o is 1. In some embodiments, o is 1-4. In some embodiments, o is 1 or 2. In some embodiments, o is 2. In some embodiments, o is 3. In some embodiments, o is 4.

In some embodiments, X is a bond. In some embodiments, X is —CR⁷R⁸—, —C(O)—, or —N(H)—. In some embodiments, X is —CR⁷R⁸—. In some embodiments, X is —C(O)—, —N(H)—, or a bond. In some embodiments, X is —C(O)—. In some embodiments, X is —N(H)—. In some embodiments, X is —CH₂—. In some embodiments, X is —CH(CH₃)—.

In some embodiments, each R⁷ and R⁸ is independently H or C₁-C₆ alkyl. In some embodiments, R⁷ is H and R⁸ is C₁-C₆ alkyl. In some embodiments, R⁷ is H and R⁸ is C₁-C₃ alkyl. In some embodiments, R⁷ and R⁸ are independently H or —CH₃. In some embodiments, R⁷ and R⁸ are each H. In some embodiments, R⁷ is H, and R⁸ is —CH₃. In some embodiments, R⁷ and R⁸ are each C₁-C₆ alkyl. In some embodiments, R⁷ and R⁸ are each C₁-C₃ alkyl. In some embodiments, R⁷ and R⁸ are each methyl.

In some embodiments, Y is a bond. In some embodiments, Y is —O—, —N(H)—, —CR⁷R⁸—, or —OCR⁷R⁸—. In some embodiments, Y is —O—. In some embodiments, Y is a bond, —N(H)—, —OCR⁷R⁸—, or —CR⁷R⁸—. In some embodiments, Y is —N(H)—. In some embodiments, Y is —OCR⁷R⁸—. In some embodiments, Y is —CR⁷R⁸—. In some embodiments, Y is —CH₂—. In some embodiments, Y is —OCH₂—.

In some embodiments, Z is Z¹.

In some embodiments, Z¹ is H, halo, C₁-C₆ alkyl, C₁-C₆ haloalkyl, —C(O)(C₁-C₆ alkyl) —C(O)NR⁵R⁶, —CH₂C(O)NR⁵R⁶, —CO₂(C₁-C₆ alkyl), —CO₂(C₁-C₆ haloalkyl), —C(O)(C₁-C₆ alkyl), —C(O)(C₁-C₆ haloalkyl), —SO₂(C₁-C₆ alkyl), —C(O)(C₁-C₆ alkylene)-NR⁵R⁶, —CO₂(C₁-C₆ alkylene)-NR⁵R⁶, —N(H)C(O)(C₁-C₆ alkyl), —C(O)NR⁹(C₁-C₆ alkylene)-NR⁵R⁶, —(C₁-C₆ alkylene)-OR⁹, —C(O)C(O)O(C₁-C₆ alkyl), —C(O)C(O)—NR⁵R⁶, —(CH₂CH₂O)ᵣ(C₁-C₆ alkyl), or —C(O)CH₂(OCH₂CH₂)ᵣO(C₁-C₆ alkyl), wherein C₁-C₆ alkylene is optionally substituted with 1-6 halo, C₁-C₆ alkyl, or C₁-C₆ haloalkyl.

In some embodiments, Z¹ is H. In some embodiments, Z¹ is halo. In some embodiments, Z¹ is C₁-C₆ alkyl. In some embodiments, Z¹ is C₁-C₆ haloalkyl. In some embodiments, Z¹ is —C(O)(C₁-C₆ alkyl). In some embodiments, Z¹ is —C(O)NR⁵R⁶. In some embodiments, Z¹ is —CH₂C(O)NR⁵R⁶. In some embodiments, Z¹ is —CO₂(C₁-C₆ alkyl). In some embodiments, Z¹ is —CO₂(C₁-C₆ haloalkyl). In some embodiments, Z¹ is —C(O)(C₁-C₆ alkyl). In some embodiments, Z¹ is —C(O)(C₁-C₆ haloalkyl). In some embodiments, Z¹ is —SO₂(C₁-C₆ alkyl). In some embodiments, Z¹ is —C(O)(C₁-C₆ alkylene)-NR⁵R⁶, wherein C₁-C₆ alkylene is optionally substituted with 1-6 halo, C₁-C₆ alkyl, or C₁-C₆ haloalkyl. In some embodiments, Z¹ is —CO₂(C₁-C₆ alkylene)-NR⁵R⁶, wherein C₁-C₆ alkylene is optionally substituted with 1-6 halo, C₁-C₆ alkyl, or C₁-C₆ haloalkyl. In some embodiments, Z¹ is —N(H)C(O)(C₁-C₆ alkyl). In some embodiments, Z¹ is —C(O)NR⁹(C₁-C₆ alkylene)-NR⁵R⁶, wherein C₁-C₆ alkylene is optionally substituted with 1-6 halo, C₁-C₆ alkyl, or C₁-C₆ haloalkyl. In some embodiments, Z¹ is —(C₁-C₆ alkylene)-OR⁹, wherein C₁-C₆ alkylene is optionally substituted with 1-6 halo, C₁-C₆ alkyl, or C₁-C₆ haloalkyl. In some embodiments, Z¹ is —C(O)C(O)O(C₁-C₆ alkyl). In some embodiments, Z¹ is —C(O)C(O)—NR⁵R⁶. In some embodiments, Z¹ is —(CH₂CH₂O)ᵣ(C₁-C₆ alkyl). In some embodiments, Z¹ is —C(O)CH₂(OCH₂CH₂)ᵣO(C₁-C₆ alkyl).

In some embodiments, Z¹ is H, halo, C₁-C₃ alkyl, C₁-C₃ haloalkyl, —C(O)(C₁-C₃ alkyl), —C(O)NR⁵R⁶, —CH₂C(O)NR⁵R⁶, —CO₂(C₁-C₃ alkyl), —CO₂(C₁-C₃ haloalkyl), —C(O)(C₁-C₃ alkyl), —C(O)(C₁-C₃ haloalkyl), —SO₂(C₁-C₃ alkyl), —C(O)(C₁-C₃ alkylene)-NR⁵R⁶, —CO₂(C₁-C₃ alkylene)-NR⁵R⁶, —N(H)C(O)(C₁-C₃ alkyl), —(C₁-C₃ alkylene)-OR⁹, —C(O)NR⁹(C₁-C₃ alkylene)-NR⁵R⁶, —C(O)C(O)O(C₁-C₆ alkyl), —C(O)C(O)—NR⁵R⁶, —(CH₂CH₂O)ᵣ(C₁-C₃ alkyl), or —C(O)CH₂(OCH₂CH₂)ᵣO(C₁-C₃ alkyl). In any of these embodiments, the C₁-C₃ alkylene is optionally substituted with 1-2 halo, C₁-C₃ alkyl, or C₁-C₃ haloalkyl. In some variations, R⁵ and R⁶ are independently H or C₁-C₃ alkyl, and R⁹ is H or C₁-C₃ alkyl.

In some embodiments, Z¹ is —C(O)N(CH₃)₂, —C(O)N(H)CH₃, —C(O)NH₂, H, —CH₂C(CH₃)₃, —C(O)C(CH₃)₃, —C(O)N(CH₃)CH₂CH₂N(CH₃)₂, —C(O)OCH₂CH₂N(CH₃)₂, —C(O)CH₂CH₂N(CH₃)₂, —C(O)OCH₃, —C(O)CH(CH₃)CH₂N(CH₃)₂, —C(O)CH₂N(CH₃)₂, —C(O)CH₂(OCH₂CH₂)₄OCH₃, —C(O)C(CH₃)₂N(CH₃)₂, —C(O)OCH₂CF₃, —CH₂C(O)NH₂, —CH₂C(O)N(CH₃)₂, —CH₂CH₂OCH₃, isopropyl, —C(O)CH₃, —C(O)CO₂CH₃, —C(O)C(O)NH₂, —C(O)C(O)N(CH₃)₂, —SO₂CH₃, —CO₂CH₂CH₃, —CO(isopropyl), —CO₂(isopropyl), F, —N(H)C(O)CH₃, —(CH₂CH₂O)₃CH₃, or —CF₃.

In some embodiments, r is 1-4. In some embodiments, r is 1 or 2. In some embodiments, r is 1. In some embodiments, r is 2. In some embodiments, r is 3. In some embodiments, r is 4.

In some embodiments, R⁹ is H or C₁-C₆ alkyl. In some embodiments, R⁹ is H or C₁-C₃ alkyl. In some embodiments, R⁹ is H. In some embodiments, R⁹ is C₁-C₆ alkyl. In some embodiments, R⁹ is C₁-C₃ alkyl. In some embodiments, R⁹ is methyl, ethyl, or propyl. In some embodiments, R⁹ is methyl.

In some embodiments, Z is Z².

In some embodiments, Z² is phenyl, —C(O)(phenyl), 5- to 6-membered heteroaryl, —C(O)-(5- to 6-membered heteroaryl), —CR⁵R⁶-(5- to 6-membered heteroaryl), 4- to 6-membered heterocyclyl, —CR⁵R⁶-(4- to 6-membered heterocyclyl), —C(O)-(4- to 6-membered heterocyclyl), C₃-C₆ cycloalkyl, —C(O)(C₃-C₆ cycloalkyl), —CO₂(C₃-C₆ cycloalkyl), or —CR⁵R⁶—(C₃-C₆ cycloalkyl), wherein the heteroaryl and heterocyclyl contain 1-3 heteroatoms selected from N and O, and wherein the phenyl, heteroaryl, and heterocyclyl are optionally substituted by 1-5 R¹⁰.

In some embodiments, Z² is phenyl. In some embodiments, Z² is —C(O)(phenyl). In some embodiments, Z² is 5- to 6-membered heteroaryl. In some embodiments, Z² is —C(O)-(5- to 6-membered heteroaryl). In some embodiments, Z² is —CR⁵R⁶-(5- to 6-membered heteroaryl). In some embodiments, Z² is 4- to 6-membered heterocyclyl. In some embodiments, Z² is —CR⁵R⁶-(4- to 6-membered heterocyclyl). In some embodiments, Z² is —C(O)-(4- to 6-membered heterocyclyl). In some embodiments, Z² is C₃-C₆ cycloalkyl. In some embodiments, Z² is —C(O)(C₃-C₆ cycloalkyl). In some embodiments, Z² is —CO₂(C₃-C₆ cycloalkyl). In some embodiments, Z² is —CR⁵R⁶—(C₃-C₆ cycloalkyl). In any of these variations, the heteroaryl and heterocyclyl contain 1-3 heteroatoms selected from N and O. In any of these variations, and the phenyl, heteroaryl, and heterocyclyl are optionally substituted by 1-5 R¹⁰.

In some embodiments, Z² is phenyl, —C(O)(phenyl), 5- to 6-membered heteroaryl, —C(O)-(5- to 6-membered heteroaryl), —CR⁵R⁶-(5- to 6-membered heteroaryl), 4- to 6-membered heterocyclyl, —CR⁵R⁶-(4- to 6-membered heterocyclyl), —C(O)-(4- to 6-membered heterocyclyl), C₃-C₆ cycloalkyl, —C(O)(C₃-C₆ cycloalkyl), —CO₂(C₃-C₆ cycloalkyl), or —CR⁵R⁶—(C₃-C₆ cycloalkyl). In any of these variations, the heteroaryl and heterocyclyl contain 1-3 heteroatoms selected from N and O; the phenyl, heteroaryl, and heterocyclyl are optionally substituted by 1-5 R^{1′}; and R⁵ and R⁶ are independently H or C₁-C₃ alkyl.

In some embodiments, Z² is pyridinyl, pyrimidinyl, pyrrolidinyl, tetrahydropyranyl, tetrahydrofuranyl, pyridazinyl, —C(O)(pyradazinyl), pyrazolyl, —C(O)(cyclopropyl), —CO₂(cyclopropyl), dihydropyridinyl, dihydropyrimidinyl, phenyl, —C(O)(phenyl), —C(O)(piperazinyl), —C(O)(piperidinyl), —C(O)(pyrrolidinyl), —CH₂(pyridinyl), —C(O)(isoxazolyl), —CH(CH₃)(pyridinyl), —C(O)(pyrazolyl), cyclohexyl, cyclobutyl, —C(O)(pyridinyl), —C(O)(pyrimidinyl), —C(O)(cyclopentyl), —C(O)(oxetanyl), morpholinyl, oxazolidinyl, or piperidinyl. In any of these variations, the phenyl, heteroaryl, and heterocyclyl are optionally substituted by 1-3 R¹⁰.

In some embodiments, each R¹⁰ is independently halo, —OH, C₁-C₆ alkyl, C₁-C₆ haloalkyl, —CN, oxo, —NR⁵R⁶, or —N(H)C(O)(C₁-C₆ alkyl). In some embodiments, R¹⁰ is halo. In some embodiments, R¹⁰ is —OH. In some embodiments, R¹⁰ is C₁-C₆ alkyl. In some embodiments, R¹⁰ is C₁-C₆ haloalkyl. In some embodiments, R¹⁰ is —CN. In some embodiments, R¹⁰ is oxo. In some embodiments, R¹⁰ is —NR⁵R⁶. In some embodiments, R¹⁰ is —N(H)C(O)(C₁-C₆ alkyl). In some embodiments, each R¹⁰ is independently halo, —OH, C₁-C₃ haloalkyl, C₁-C₃ alkyl, —CN, oxo, —NH₂, or —N(H)C(O)(C₁-C₃ alkyl). In some embodiments, each R¹⁰ is independently F, Cl, —OH, —CF₃, isopropyl, —CH₃, —CN, oxo, —NH₂, or —NHC(O)CH₃.

In some embodiments, Z—Y—X— is not an alkyl. In some embodiments, Z—Y—X— is not H.

In some embodiments, Z—Y—X— is

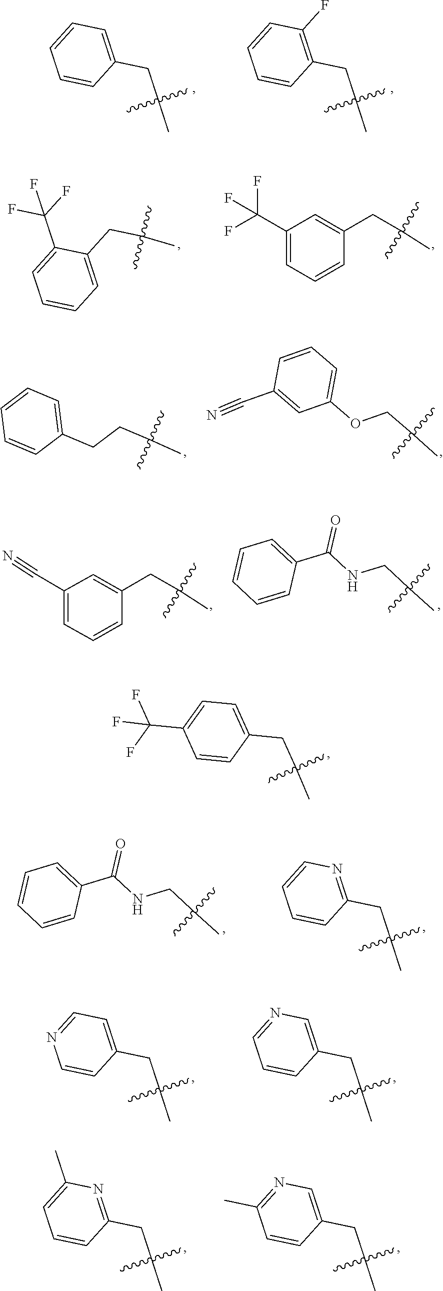

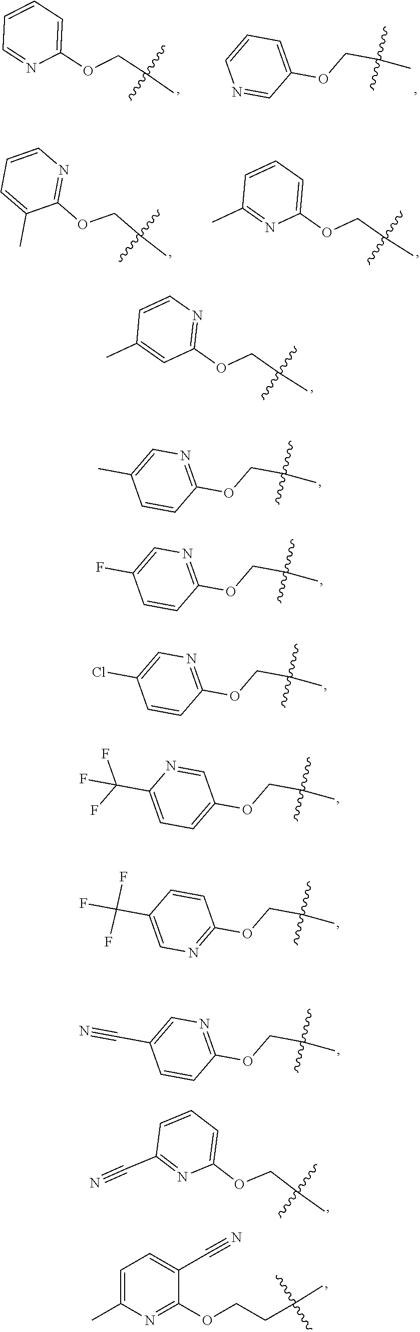

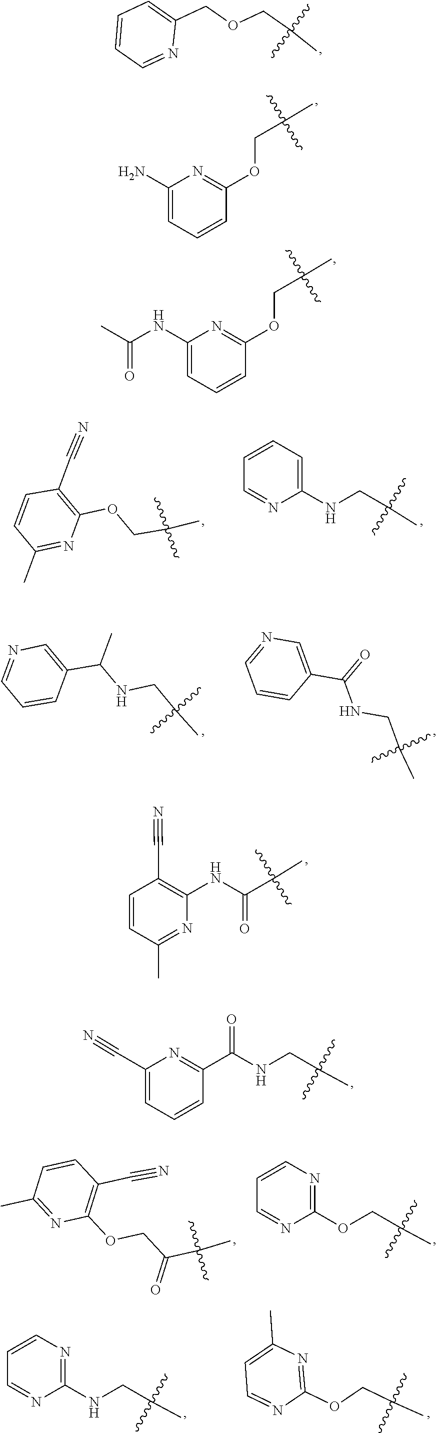

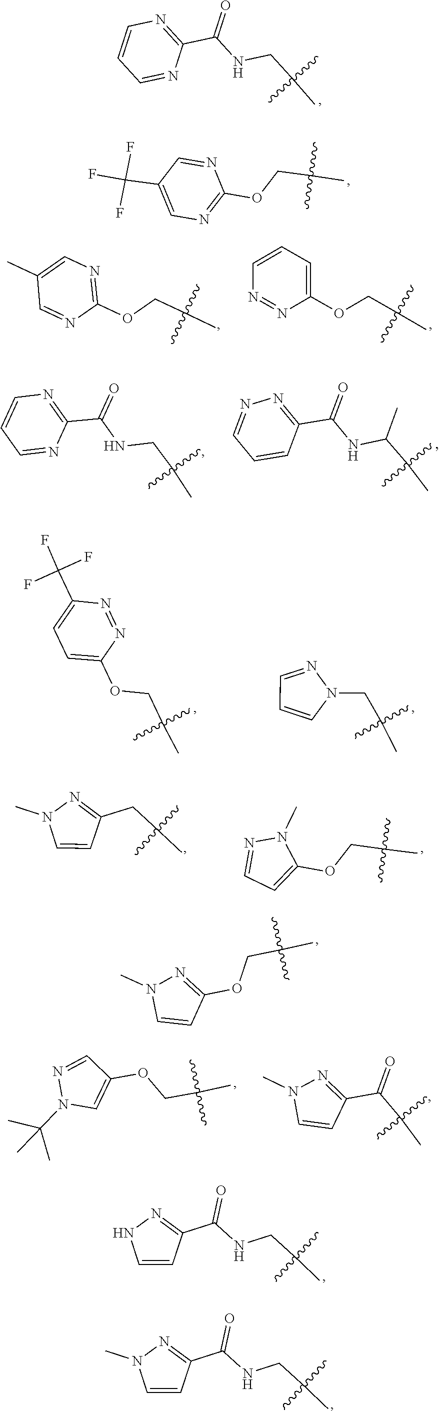

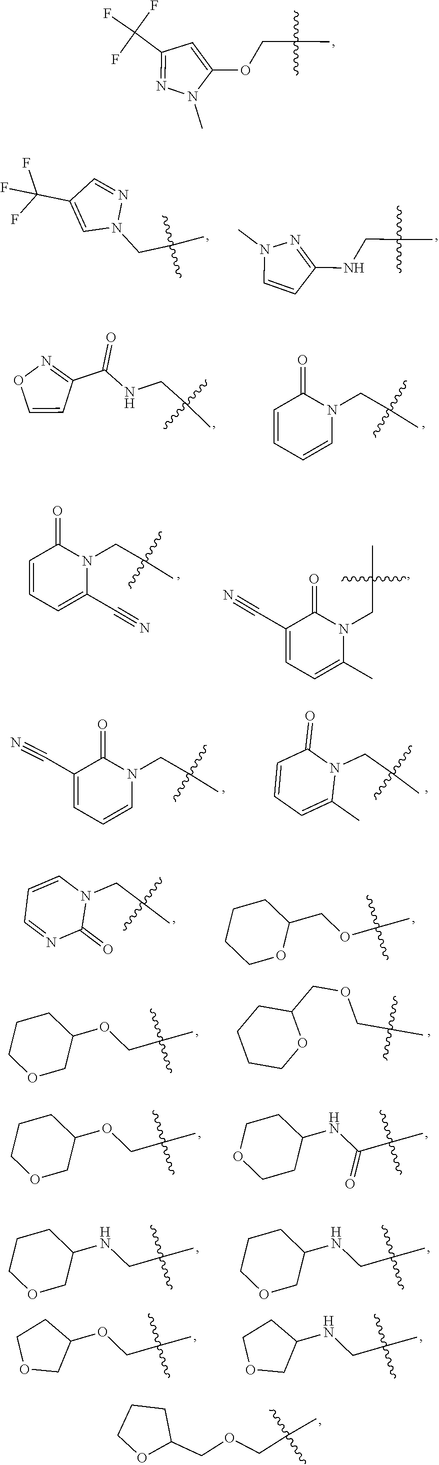

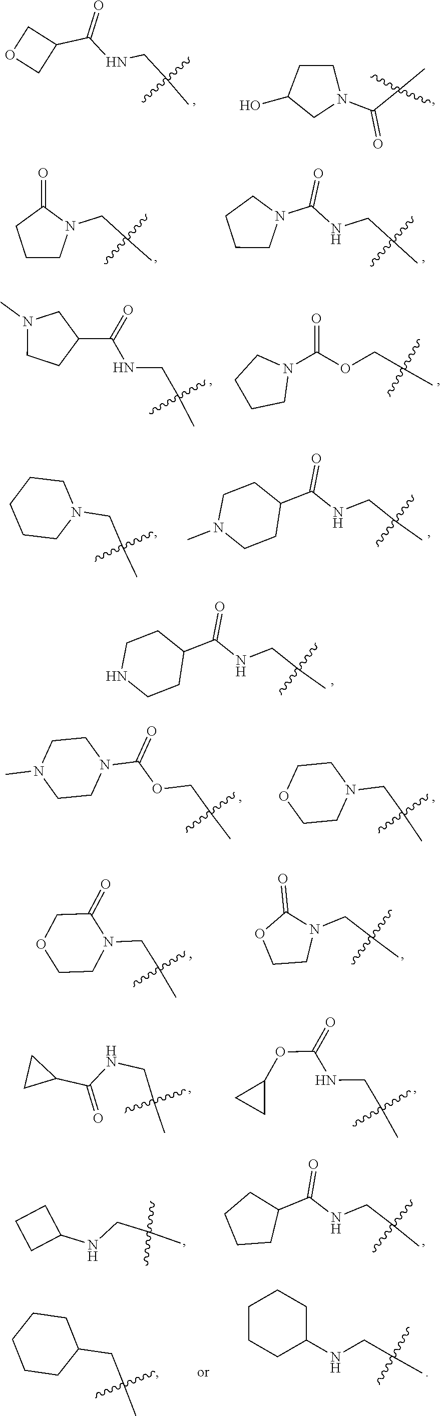

In some embodiments, Z—Y—X— is

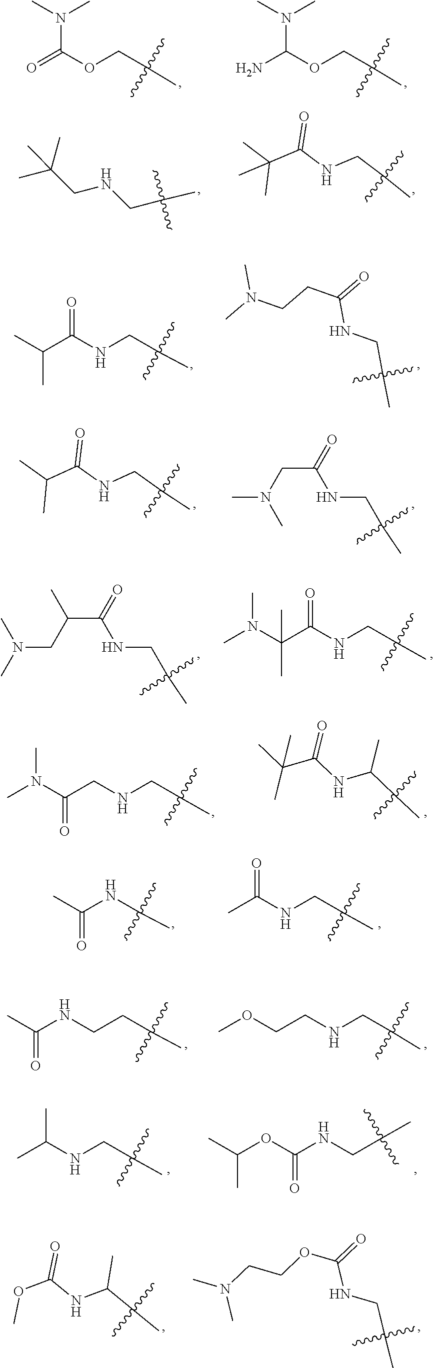

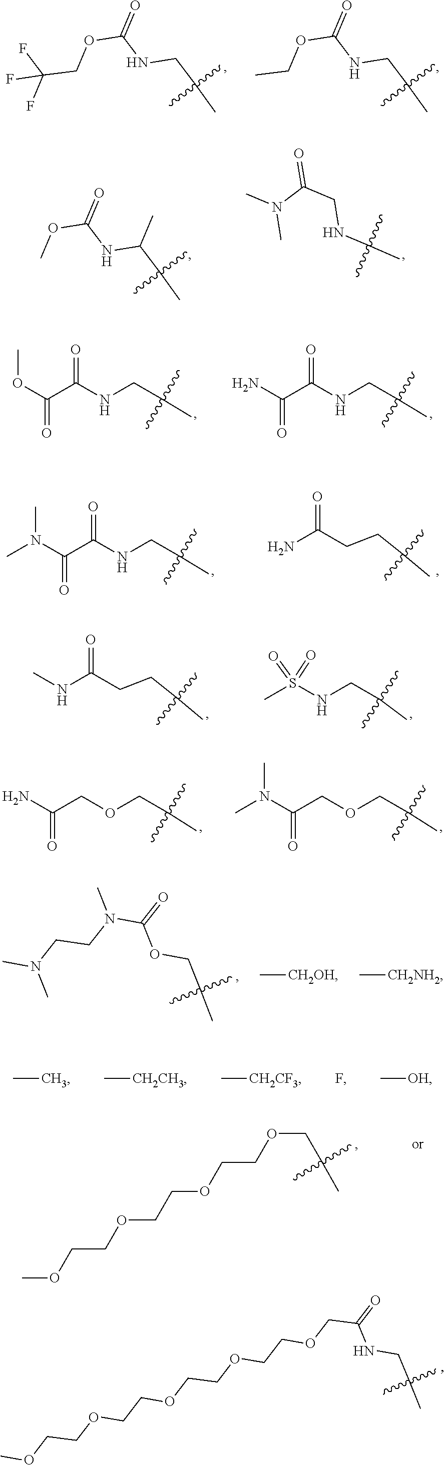

In some embodiments, the compound of Formula (I) is a compound of Formula (II), (III), (IV), (V), (VI), or (VII):

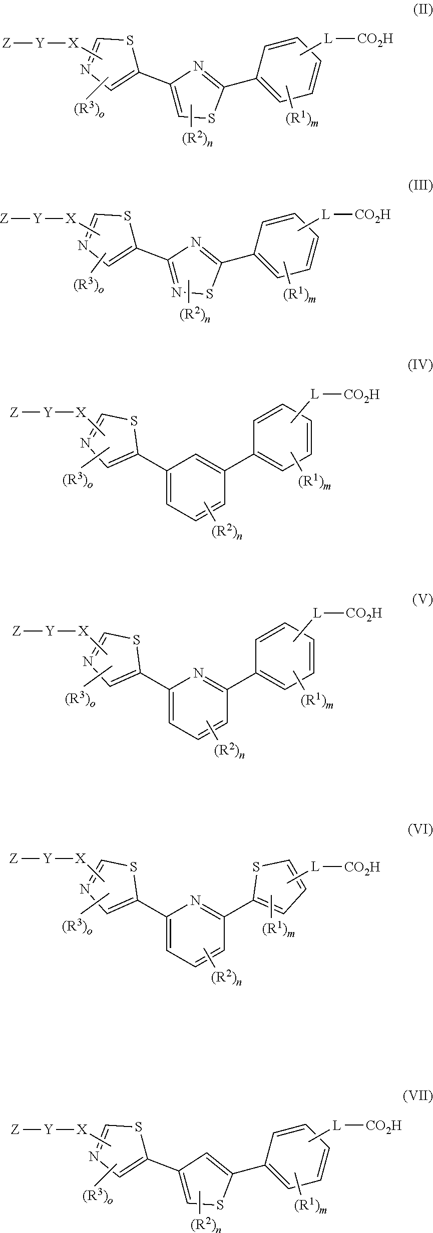

wherein L, R¹, m, R², n, R³, o, X, Y, and Z are as described for Formula (I).

In some embodiments, the compound of Formula (I) is a compound of Formula (II), wherein L, R, m, R², n, R³, o, X, Y, and Z are as described for Formula (I). In some embodiments, the compound of Formula (I) is a compound of Formula (III), wherein L, R¹, m, R², n, R³, o, X, Y, and Z are as described for Formula (I). In some embodiments, the compound of Formula (I) is a compound of Formula (IV), wherein L, R¹, m, R², n, R³, o, X, Y, and Z are as described for Formula (I). In some embodiments, the compound of Formula (I) is a compound of Formula (V), wherein L, R¹, m, R², n, R³, o, X, Y, and Z are as described for Formula (I). In some embodiments, the compound of Formula (I) is a compound of Formula (VI), wherein L, R¹, m, R², n, R³, o, X, Y, and Z are as described for Formula (I). In some embodiments, the compound of Formula (I) is a compound of Formula (VII), wherein L, R¹, m, R², n, R³, o, X, Y, and Z are as described for Formula (I).

In some embodiments, the compound is of Formula (II)-(VII) and L is a bond. In some embodiments, the compound is of Formula (II)-(VII) and L is —S(O)₂CR⁵R⁶— (such as —S(O)₂—CH₂—). In some embodiments, the compound is of Formula (II)-(VII) and X is —CR⁷R⁸— (such as —CH₂—). In some embodiments, the compound is of Formula (II)-(VII) and Y is —O—. In some embodiments, the compound is of Formula (II)-(VII) and Y is —N(H)—. In some embodiments, the compound is of Formula (II)-(VII) and Z is Z², wherein Z² is 6-membered heteroaryl (such as pyrimidinyl or pyridinyl). In some embodiments, the compound is of Formula (II)-(VII) and Z is Z¹, wherein Z¹ is —CO₂(C₁-C₆ alkyl) (such as —CO₂CH₃), —CH₂C(O)NR⁵R⁶(such as —CH₂CON(CH₃)₂), or —C(O)(C₁-C₆ alkyl) (such as —COCH₃). In some embodiments, the compound is of Formula (II)-(VII) and R³ is C₁-C₆ alkyl (such as methyl). In some embodiments, the compound is of Formula (II)-(VII) and R¹ is —OH or halo (such as F).

In some embodiments, the compound is of Formula (II)-(VII), wherein L is a bond; R¹ is —OH or halo; X is —CR⁷R⁸—; Y is —O— or —N(H)—; Z is a 6-membered heteroaryl, —CO₂(C₁-C₆ alkyl), —CH₂C(O)NR⁵R⁶, or —C(O)(C₁-C₆ alkyl); and the remaining variables are as described for Formula (I).

In some embodiments, the compound is of Formula (II), wherein L is a bond; R¹ is —OH or halo; X is —CR⁷R⁸—; Y is —O— or —N(H)—; Z is a 6-membered heteroaryl, —CO₂(C₁-C₆ alkyl), —CH₂C(O)NR⁵R⁶, or —C(O)(C₁-C₆ alkyl); and the remaining variables are as described for Formula (I).

In some embodiments, the compound is of Formula (III), wherein L is a bond; R¹ is —OH or halo; X is —CR⁷R⁸—; Y is —O— or —N(H)—; Z is a 6-membered heteroaryl, —CO₂(C₁-C₆ alkyl), —CH₂C(O)NR⁵R⁶, or —C(O)(C₁-C₆ alkyl); and the remaining variables are as described for Formula (I).

In some embodiments, the compound is of Formula (IV), wherein L is a bond; R¹ is —OH or halo; X is —CR⁷R⁸—; Y is —O— or —N(H)—; Z is a 6-membered heteroaryl, —CO₂(C₁-C₆ alkyl), —CH₂C(O)NR⁵R⁶, or —C(O)(C₁-C₆ alkyl); and the remaining variables are as described for Formula (I).

In some embodiments, the compound is of Formula (V), wherein L is a bond; R¹ is —OH or halo; X is —CR⁷R⁸—; Y is —O— or —N(H)—; Z is a 6-membered heteroaryl, —CO₂(C₁-C₆ alkyl), —CH₂C(O)NR⁵R⁶, or —C(O)(C₁-C₆ alkyl); and the remaining variables are as described for Formula (I).

In some embodiments, the compound is of Formula (VI), wherein L is a bond; R¹ is —OH or halo; X is —CR⁷R⁸—; Y is —O— or —N(H)—; Z is a 6-membered heteroaryl, —CO₂(C₁-C₆ alkyl), —CH₂C(O)NR⁵R⁶, or —C(O)(C₁-C₆ alkyl); and the remaining variables are as described for Formula (I).

In some embodiments, the compound is of Formula (VII), wherein L is a bond; R¹ is —OH or halo; X is —CR⁷R⁸—; Y is —O— or —N(H)—; Z is a 6-membered heteroaryl, —CO₂(C₁-C₆ alkyl), —CH₂C(O)NR⁵R⁶, or —C(O)(C₁-C₆ alkyl); and the remaining variables are as described for Formula (I).

In some embodiments, the compound is of Formula (II), wherein L is a bond; R¹ is —OH or halo; X is —CR⁷R⁸—; Y is —O— or —N(H)—; Z is a 6-membered heteroaryl; and the remaining variables are as described for Formula (I). In some embodiments, the compound is of Formula (III), wherein L is a bond; R¹ is —OH or halo; X is —CR⁷R⁸—; Y is —O— or —N(H)—; Z is a 6-membered heteroaryl; and the remaining variables are as described for Formula (I). In some embodiments, the compound is of Formula (IV), wherein L is a bond; R¹ is —OH or halo; X is —CR⁷R⁸—; Y is —O— or —N(H)—; Z is a 6-membered heteroaryl; and the remaining variables are as described for Formula (I). In some embodiments, the compound is of Formula (V), wherein L is a bond; R¹ is —OH or halo; X is —CR⁷R⁸—; Y is —O— or —N(H)—; Z is a 6-membered heteroaryl; and the remaining variables are as described for Formula (I). In some embodiments, the compound is of Formula (VI), wherein L is a bond; R¹ is —OH or halo; X is —CR⁷R⁸—; Y is —O— or —N(H)—; Z is a 6-membered heteroaryl; and the remaining variables are as described for Formula (I). In some embodiments, the compound is of Formula (VII), wherein L is a bond; R¹ is —OH or halo; X is —CR⁷R⁸—; Y is —O— or —N(H)—; Z is a 6-membered heteroaryl; and the remaining variables are as described for Formula (I).

In some embodiments, the compound is of Formula (II), wherein L is a bond; R¹ is —OH or halo; X is —CR⁷R⁸—; Y is —O— or —N(H)—; Z is —CO₂(C₁-C₆ alkyl), —CH₂C(O)NR⁵R⁶, or —C(O)(C₁-C₆ alkyl); and the remaining variables are as described for Formula (I). In some embodiments, the compound is of Formula (III), wherein L is a bond; R¹ is —OH or halo; X is —CR⁷R⁸—; Y is —O— or —N(H)—; Z is —CO₂(C₁-C₆ alkyl), —CH₂C(O)NR⁵R⁶, or —C(O)(C₁-C₆ alkyl); and the remaining variables are as described for Formula (I). In some embodiments, the compound is of Formula (IV), wherein L is a bond; R¹ is —OH or halo; X is —CR⁷R⁸—; Y is —O— or —N(H)—; Z is —CO₂(C₁-C₆ alkyl), —CH₂C(O)NR⁵R⁶, or —C(O)(C₁-C₆ alkyl); and the remaining variables are as described for Formula (I). In some embodiments, the compound is of Formula (V), wherein L is a bond; R¹ is —OH or halo; X is —CR⁷R⁸—; Y is —O— or —N(H)—; Z is —CO₂(C₁-C₆ alkyl), —CH₂C(O)NR⁵R⁶, or —C(O)(C₁-C₆ alkyl); and the remaining variables are as described for Formula (I). In some embodiments, the compound is of Formula (VI), wherein L is a bond; R¹ is —OH or halo; X is —CR⁷R⁸—; Y is —O— or —N(H)—; Z is —CO₂(C₁-C₆ alkyl), —CH₂C(O)NR⁵R⁶, or —C(O)(C₁-C₆ alkyl); and the remaining variables are as described for Formula (I). In some embodiments, the compound is of Formula (VII), wherein L is a bond; R¹ is —OH or halo; X is —CR⁷R⁸—; Y is —O— or —N(H)—; Z is —CO₂(C₁-C₆ alkyl), —CH₂C(O)NR⁵R⁶, or —C(O)(C₁-C₆ alkyl); and the remaining variables are as described for Formula (I).

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to L of Formula (I) may be combined with every description, variation, embodiment, or aspect of Ring A, Ring B, Ring C, R¹, R², R³, m, n, o, R⁵, R⁶, q, X, Y, R⁷, R⁸, Z, Z¹, R⁹, r, Z², and R¹⁰ the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (II)-(VII), and are equally described, the same as if each and every description, variation, embodiment, or aspect were separately and individually listed for all formulae.

In some embodiments, provided is a compound selected from the compounds in Table 1 or a pharmaceutically acceptable salt thereof. In some embodiments, provided is a compound selected from the compounds in Table 2 or a pharmaceutically acceptable salt thereof. Although certain compounds described in the present disclosure, including in Table 1 and Table 2, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1 and Table 2, are herein described.

TABLE 1

| Ex- | |
|---|---|
| am- | |
| ple | Structure |
| 1 | 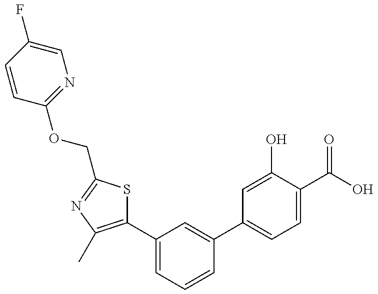 [SMILES: Cc1nc(COc2ccc(F)cn2)sc1-c1cccc(-c2ccc(C(=O)O)c(O)c2)c1] |
| 2 | 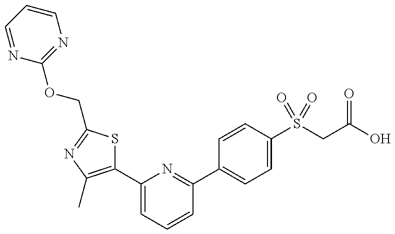 [SMILES: Cc1nc(COc2ncccn2)sc1-c1cccc(-c2ccc(S(=O)(=O)CC(=O)O)cc2)n1] |
| 3 | 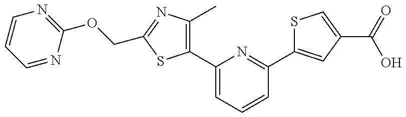 [SMILES: Cc1nc(COc2ncccn2)sc1-c1cccc(-c2cc(C(=O)O)cs2)n1] |
| 4 | 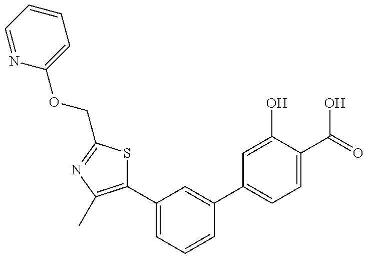 [SMILES: Cc1nc(COc2ccccn2)sc1-c1cccc(-c2ccc(C(=O)O)c(O)c2)c1] |
| 5 | 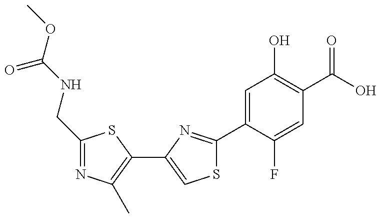 [SMILES: COC(=O)NCc1nc(C)c(-c2csc(-c3cc(O)c(C(=O)O)cc3F)n2)s1] |
| 6 | 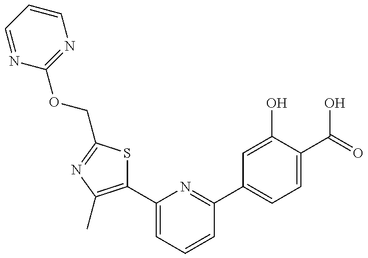 [SMILES: Cc1nc(COc2ncccn2)sc1-c1cccc(-c2ccc(C(=O)O)c(O)c2)n1] |
| 7 | 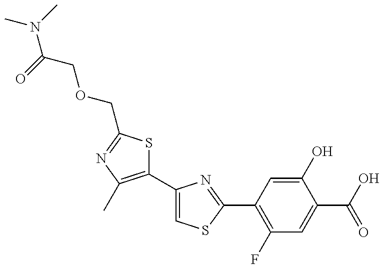 [SMILES: Cc1nc(COCC(=O)N(C)C)sc1-c1csc(-c2cc(O)c(C(=O)O)cc2F)n1] |
| 8 | 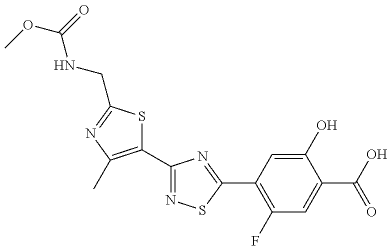 [SMILES: COC(=O)NCc1nc(C)c(-c2nsc(-c3cc(O)c(C(=O)O)cc3F)n2)s1] |
| 9 | 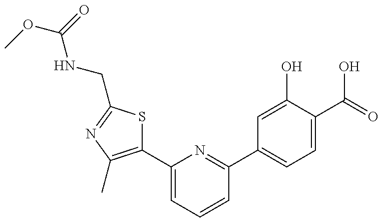 [SMILES: COC(=O)NCc1nc(C)c(-c2cccc(-c3ccc(C(=O)O)c(O)c3)n2)s1] |
| 10 | 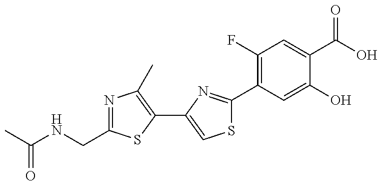 [SMILES: CC(=O)NCc1nc(C)c(-c2csc(-c3cc(O)c(C(=O)O)cc3F)n2)s1] |

or a pharmaceutically acceptable salt thereof.

TABLE 2

| Example | Structure |
|---|---|
| 11 | 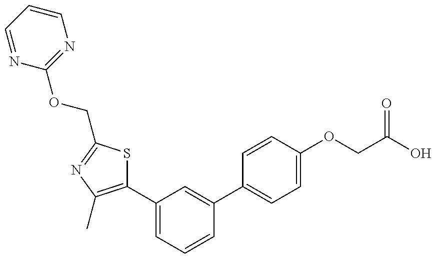 [SMILES: Cc1nc(COc2ncccn2)sc1-c1cccc(-c2ccc(OCC(=O)O)cc2)c1] |
| 12 | 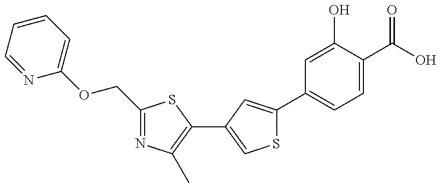 [SMILES: Cc1nc(COc2ccccn2)sc1-c1csc(-c2ccc(C(=O)O)c(O)c2)c1] |
| 13 | 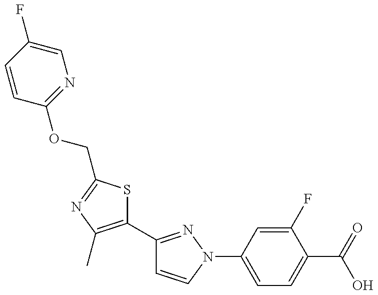 [SMILES: Cc1nc(COc2ccc(F)cn2)sc1-c1ccn(-c2ccc(C(=O)O)c(F)c2)n1] |
| 14 | 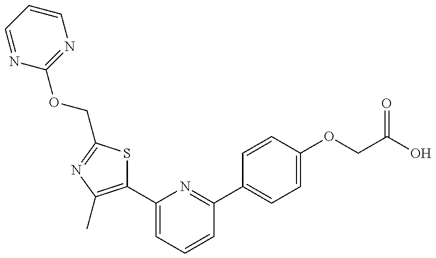 [SMILES: Cc1nc(COc2ncccn2)sc1-c1cccc(-c2ccc(OCC(=O)O)cc2)n1] |
| 15 | 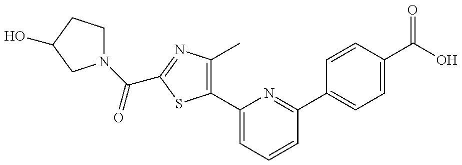 [SMILES: Cc1nc(C(=O)N2CCC(O)C2)sc1-c1cccc(-c2ccc(C(=O)O)cc2)n1] |
| 16 | 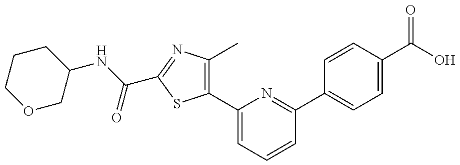 [SMILES: Cc1nc(C(=O)NC2CCCOC2)sc1-c1cccc(-c2ccc(C(=O)O)cc2)n1] |
| 17 | 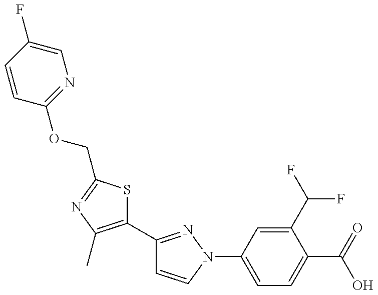 [SMILES: Cc1nc(COc2ccc(F)cn2)sc1-c1ccn(-c2ccc(C(=O)O)c(C(F)F)c2)n1] |
| 18 | 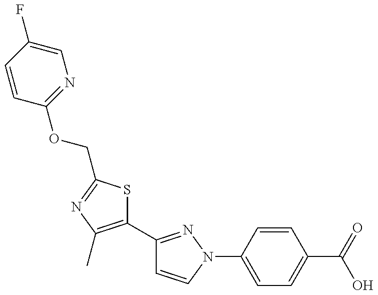 [SMILES: Cc1nc(COc2ccc(F)cn2)sc1-c1ccn(-c2ccc(C(=O)O)cc2)n1] |
| 19 | 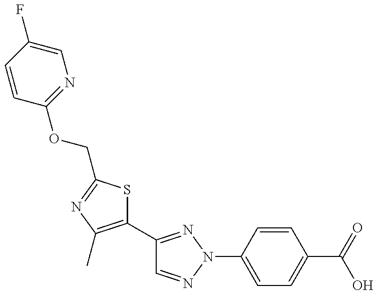 [SMILES: Cc1nc(COc2ccc(F)cn2)sc1-c1cnn(-c2ccc(C(=O)O)cc2)n1] |
| 20 | 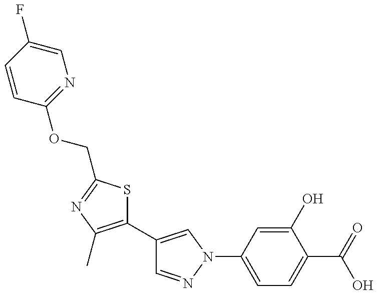 [SMILES: Cc1nc(COc2ccc(F)cn2)sc1-c1cnn(-c2ccc(C(=O)O)c(O)c2)c1] |
| 21 | 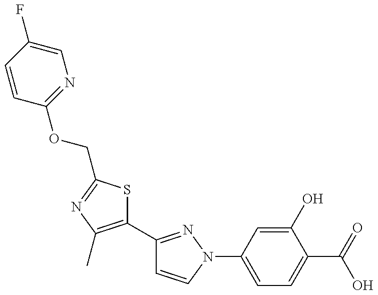 [SMILES: Cc1nc(COc2ccc(F)cn2)sc1-c1ccn(-c2ccc(C(=O)O)c(O)c2)n1] |
| 22 | 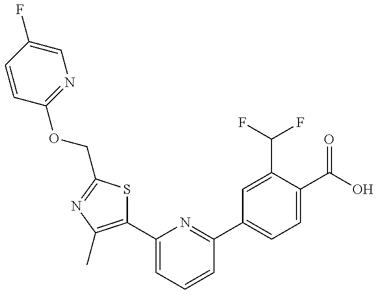 [SMILES: Cc1nc(COc2ccc(F)cn2)sc1-c1cccc(-c2ccc(C(=O)O)c(C(F)F)c2)n1] |
| 23 | 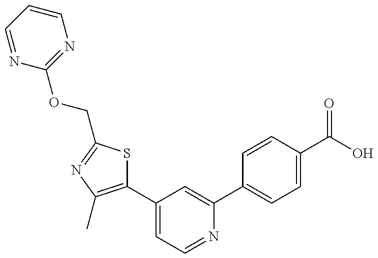 [SMILES: Cc1nc(COc2ncccn2)sc1-c1ccnc(-c2ccc(C(=O)O)cc2)c1] |
| 24 | 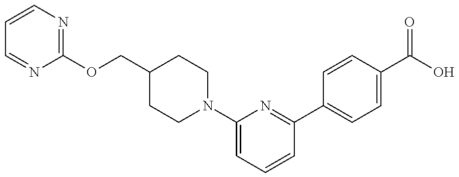 [SMILES: O=C(O)c1ccc(-c2cccc(N3CCC(COc4ncccn4)CC3)n2)cc1] |
| 25 | 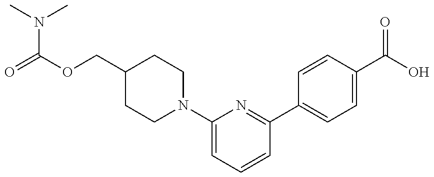 [SMILES: CN(C)C(=O)OCC1CCN(c2cccc(-c3ccc(C(=O)O)cc3)n2)CC1] |
| 26 | 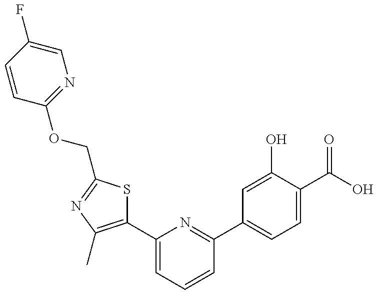 [SMILES: Cc1nc(COc2ccc(F)cn2)sc1-c1cccc(-c2ccc(C(=O)O)c(O)c2)n1] |
| 27 | 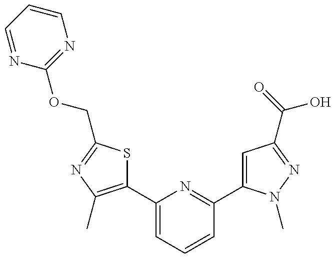 [SMILES: Cc1nc(COc2ncccn2)sc1-c1cccc(-c2cc(C(=O)O)nn2C)n1] |
| 28 | 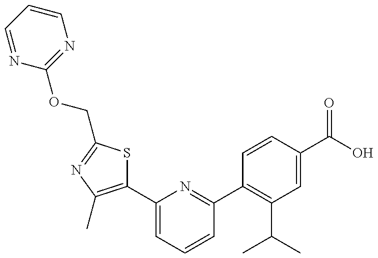 [SMILES: Cc1nc(COc2ncccn2)sc1-c1cccc(-c2ccc(C(=O)O)cc2C(C)C)n1] |
| 29 | 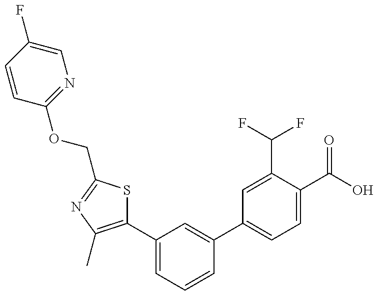 [SMILES: Cc1nc(COc2ccc(F)cn2)sc1-c1cccc(-c2ccc(C(=O)O)c(C(F)F)c2)c1] |
| 30 | 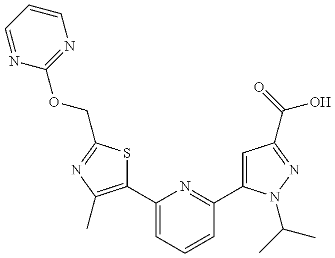 [SMILES: Cc1nc(COc2ncccn2)sc1-c1cccc(-c2cc(C(=O)O)nn2C(C)C)n1] |
| 31 | 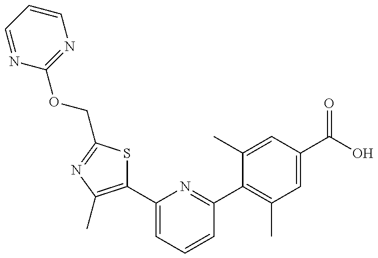 [SMILES: Cc1cc(C(=O)O)cc(C)c1-c1cccc(-c2sc(COc3ncccn3)nc2C)n1] |
| 32 | 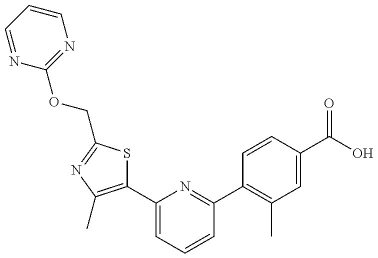 [SMILES: Cc1cc(C(=O)O)ccc1-c1cccc(-c2sc(COc3ncccn3)nc2C)n1] |
| 33 | 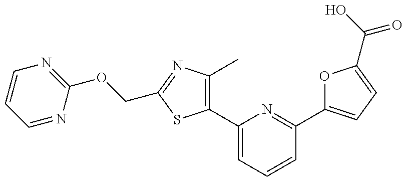 [SMILES: Cc1nc(COc2ncccn2)sc1-c1cccc(-c2ccc(C(=O)O)o2)n1] |
| 34 | 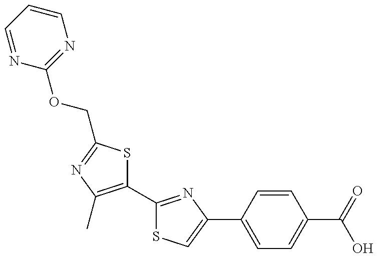 [SMILES: Cc1nc(COc2ncccn2)sc1-c1nc(-c2ccc(C(=O)O)cc2)cs1] |
| 35 | 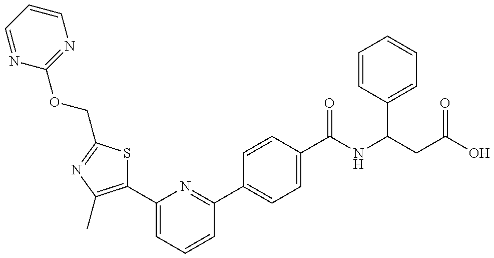 [SMILES: Cc1nc(COc2ncccn2)sc1-c1cccc(-c2ccc(C(=O)NC(CC(=O)O)c3ccccc3)cc2)n1] |
| 36 | 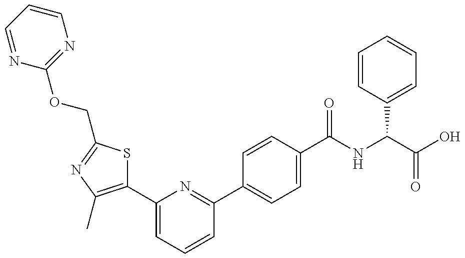 [SMILES: Cc1nc(COc2ncccn2)sc1-c1cccc(-c2ccc(C(=O)N[C@@H](C(=O)O)c3ccccc3)cc2)n1] |
| 37 | 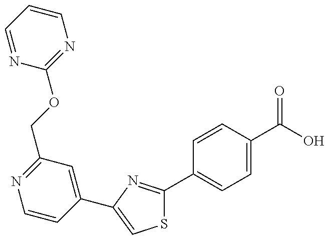 [SMILES: O=C(O)c1ccc(-c2nc(-c3ccnc(COc4ncccn4)c3)cs2)cc1] |
| 38 | 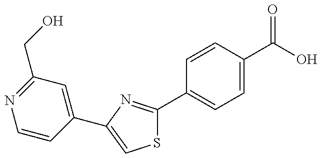 [SMILES: O=C(O)c1ccc(-c2nc(-c3ccnc(CO)c3)cs2)cc1] |
| 39 | 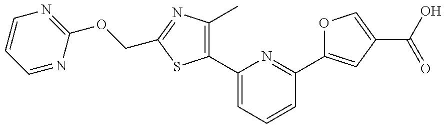 [SMILES: Cc1nc(COc2ncccn2)sc1-c1cccc(-c2cc(C(=O)O)co2)n1] |
| 40 | 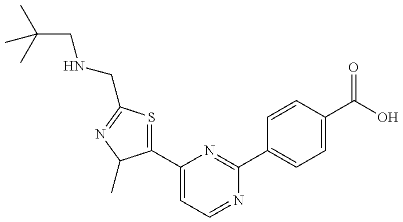 |
| 41 | 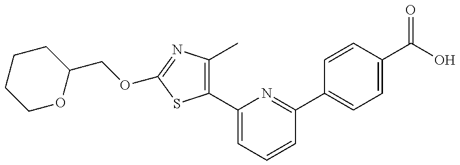 [SMILES: Cc1nc(OCC2CCCCO2)sc1-c1cccc(-c2ccc(C(=O)O)cc2)n1] |
| 42 | 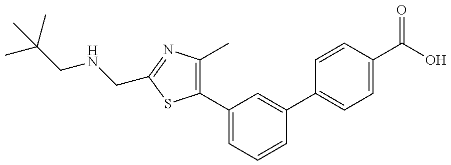 [SMILES: Cc1nc(CNCC(C)(C)C)sc1-c1cccc(-c2ccc(C(=O)O)cc2)c1] |
| 43 | 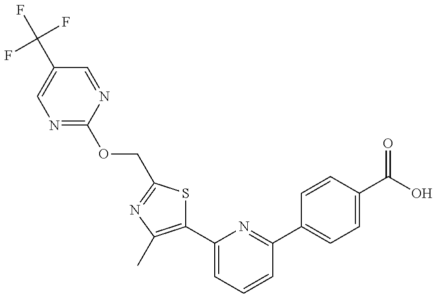 [SMILES: Cc1nc(COc2ncc(C(F)(F)F)cn2)sc1-c1cccc(-c2ccc(C(=O)O)cc2)n1] |
| 44 | 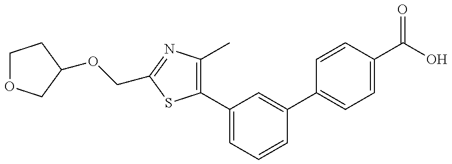 [SMILES: Cc1nc(COC2CCOC2)sc1-c1cccc(-c2ccc(C(=O)O)cc2)c1] |
| 45 | 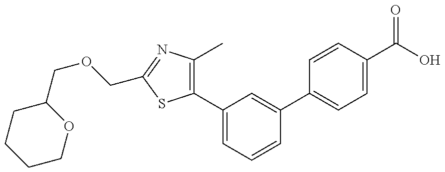 [SMILES: Cc1nc(COCC2CCCCO2)sc1-c1cccc(-c2ccc(C(=O)O)cc2)c1] |
| 46 | 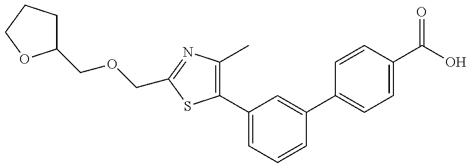 [SMILES: Cc1nc(COCC2CCCO2)sc1-c1cccc(-c2ccc(C(=O)O)cc2)c1] |
| 47 | 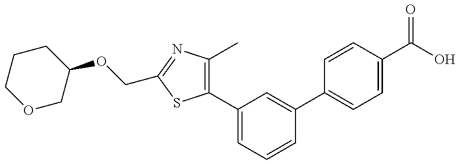 [SMILES: Cc1nc(CO[C@@H]2CCCOC2)sc1-c1cccc(-c2ccc(C(=O)O)cc2)c1] |
| 48 | 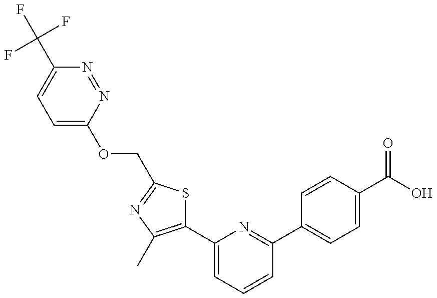 [SMILES: Cc1nc(COc2ccc(C(F)(F)F)nn2)sc1-c1cccc(-c2ccc(C(=O)O)cc2)n1] |
| 49 | 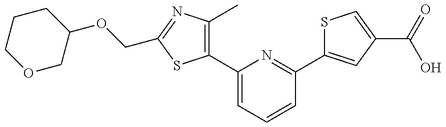 [SMILES: Cc1nc(COC2CCCOC2)sc1-c1cccc(-c2cc(C(=O)O)cs2)n1] |
| 50 | 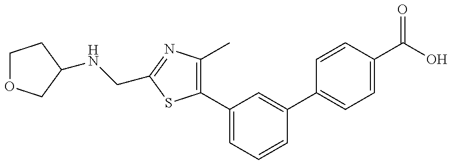 [SMILES: Cc1nc(CNC2CCOC2)sc1-c1cccc(-c2ccc(C(=O)O)cc2)c1] |
| 51 | 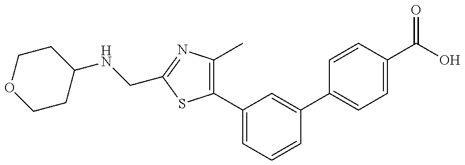 [SMILES: Cc1nc(CNC2CCOCC2)sc1-c1cccc(-c2ccc(C(=O)O)cc2)c1] |
| 52 | 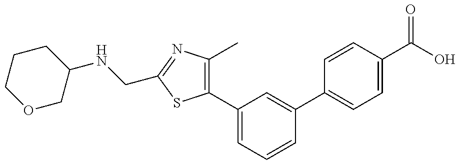 [SMILES: Cc1nc(CNC2CCCOC2)sc1-c1cccc(-c2ccc(C(=O)O)cc2)c1] |
| 53 | 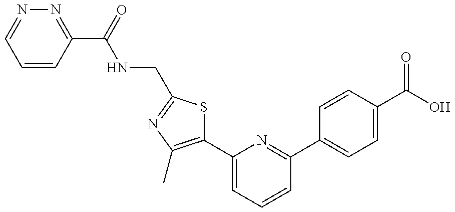 [SMILES: Cc1nc(CNC(=O)c2cccnn2)sc1-c1cccc(-c2ccc(C(=O)O)cc2)n1] |
| 54 | 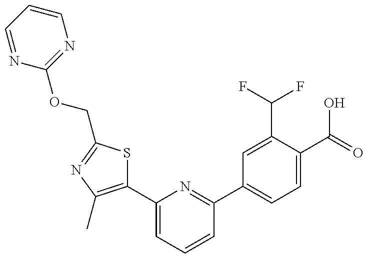 [SMILES: Cc1nc(COc2ncccn2)sc1-c1cccc(-c2ccc(C(=O)O)c(C(F)F)c2)n1] |
| 55 | 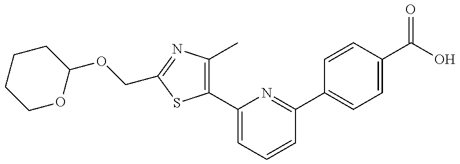 [SMILES: Cc1nc(COC2CCCCO2)sc1-c1cccc(-c2ccc(C(=O)O)cc2)n1] |
| 56 | 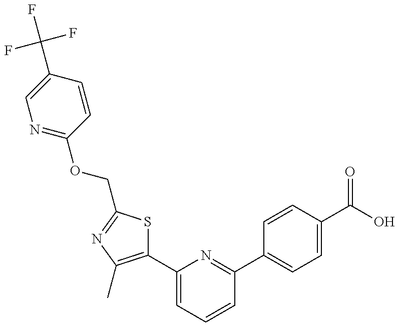 [SMILES: Cc1nc(COc2ccc(C(F)(F)F)cn2)sc1-c1cccc(-c2ccc(C(=O)O)cc2)n1] |
| 57 | 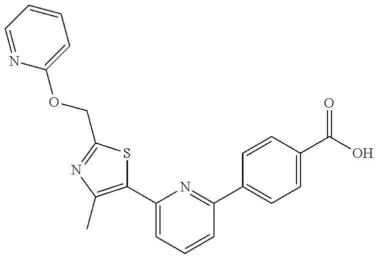 [SMILES: Cc1nc(COc2ccccn2)sc1-c1cccc(-c2ccc(C(=O)O)cc2)n1] |
| 58 | 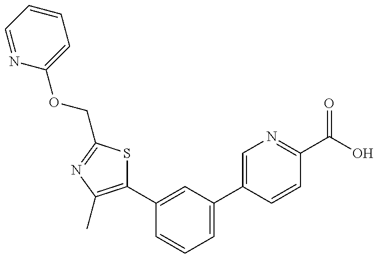 [SMILES: Cc1nc(COc2ccccn2)sc1-c1cccc(-c2ccc(C(=O)O)nc2)c1] |
| 59 | 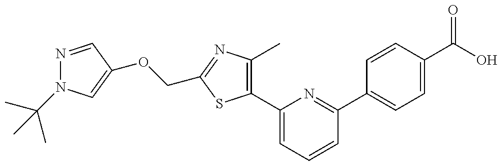 [SMILES: Cc1nc(COc2cnn(C(C)(C)C)c2)sc1-c1cccc(-c2ccc(C(=O)O)cc2)n1] |
| 60 | 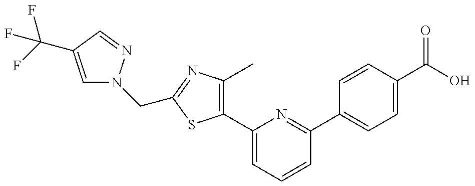 [SMILES: Cc1nc(Cn2cc(C(F)(F)F)cn2)sc1-c1cccc(-c2ccc(C(=O)O)cc2)n1] |
| 61 | 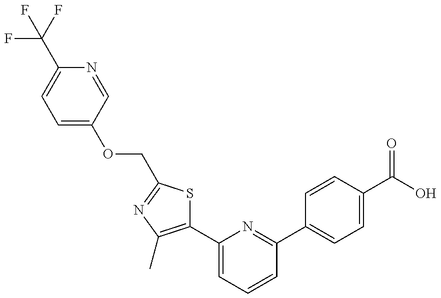 [SMILES: Cc1nc(COc2ccc(C(F)(F)F)nc2)sc1-c1cccc(-c2ccc(C(=O)O)cc2)n1] |
| 62 | 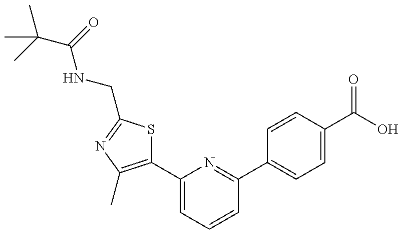 [SMILES: Cc1nc(CNC(=O)C(C)(C)C)sc1-c1cccc(-c2ccc(C(=O)O)cc2)n1] |
| 63 | 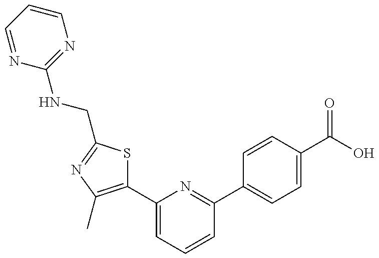 [SMILES: Cc1nc(CNc2ncccn2)sc1-c1cccc(-c2ccc(C(=O)O)cc2)n1] |
| 64 | 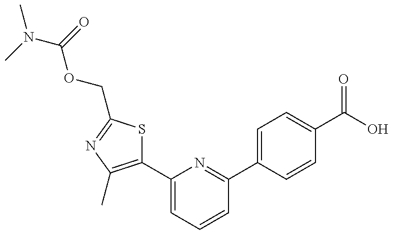 [SMILES: Cc1nc(COC(=O)N(C)C)sc1-c1cccc(-c2ccc(C(=O)O)cc2)n1] |
| 65 | 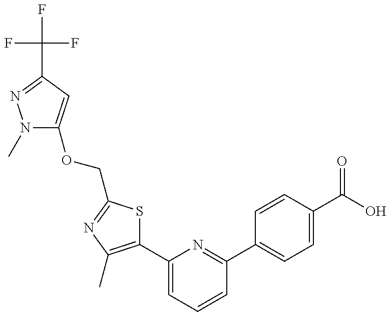 [SMILES: Cc1nc(COc2cc(C(F)(F)F)nn2C)sc1-c1cccc(-c2ccc(C(=O)O)cc2)n1] |
| 66 | 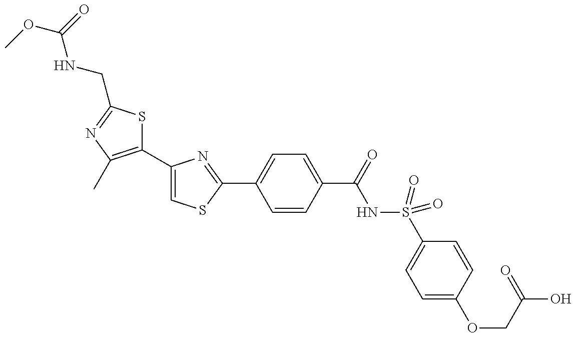 [SMILES: COC(=O)NCc1nc(C)c(-c2csc(-c3ccc(C(=O)NS(=O)(=O)c4ccc(OCC(=O)O)cc4)cc3)n2)s1] |
| 67 | 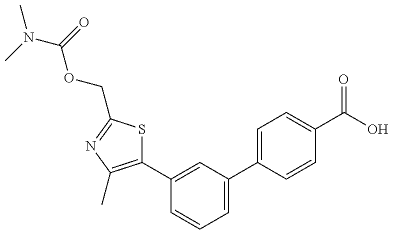 [SMILES: Cc1nc(COC(=O)N(C)C)sc1-c1cccc(-c2ccc(C(=O)O)cc2)c1] |
| 68 | 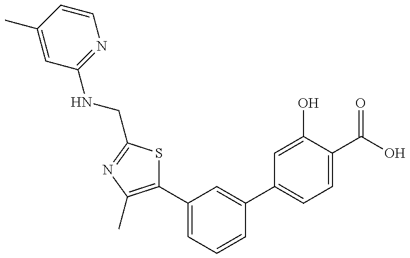 [SMILES: Cc1ccnc(NCc2nc(C)c(-c3cccc(-c4ccc(C(=O)O)c(O)c4)c3)s2)c1] |
| 69 | 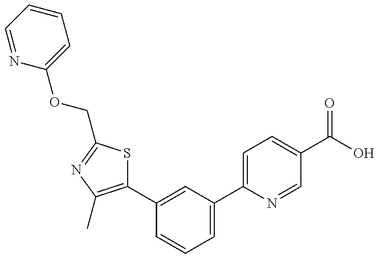 [SMILES: Cc1nc(COc2ccccn2)sc1-c1cccc(-c2ccc(C(=O)O)cn2)c1] |
| 70 | 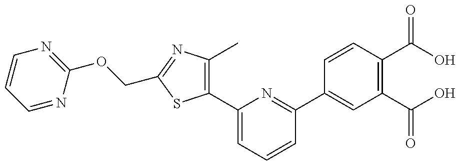 [SMILES: Cc1nc(COc2ncccn2)sc1-c1cccc(-c2ccc(C(=O)O)c(C(=O)O)c2)n1] |
| 71 | 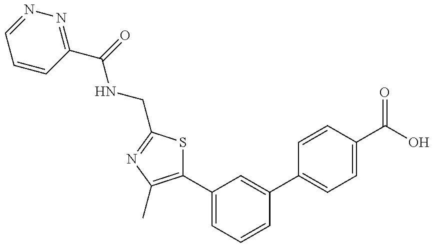 [SMILES: Cc1nc(CNC(=O)c2cccnn2)sc1-c1cccc(-c2ccc(C(=O)O)cc2)c1] |
| 72 | 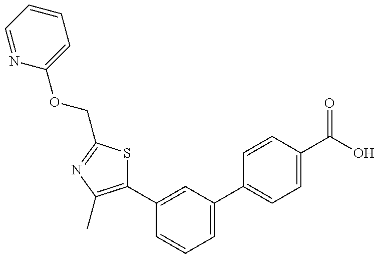 [SMILES: Cc1nc(COc2ccccn2)sc1-c1cccc(-c2ccc(C(=O)O)cc2)c1] |
| 73 | 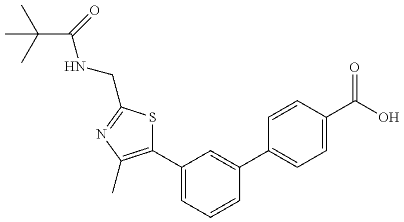 [SMILES: Cc1nc(CNC(=O)C(C)(C)C)sc1-c1cccc(-c2ccc(C(=O)O)cc2)c1] |
| 74 | 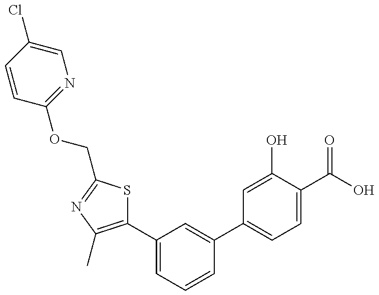 [SMILES: Cc1nc(COc2ccc(Cl)cn2)sc1-c1cccc(-c2ccc(C(=O)O)c(O)c2)c1] |
| 75 | 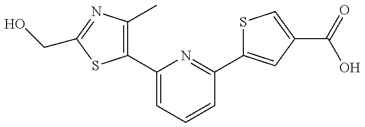 [SMILES: Cc1nc(CO)sc1-c1cccc(-c2cc(C(=O)O)cs2)n1] |
| 76 | 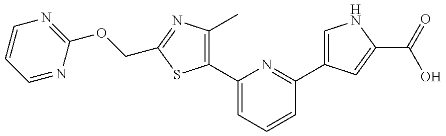 [SMILES: Cc1nc(COc2ncccn2)sc1-c1cccc(-c2c[nH]c(C(=O)O)c2)n1] |
| 77 | 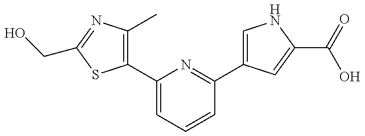 [SMILES: Cc1nc(CO)sc1-c1cccc(-c2c[nH]c(C(=O)O)c2)n1] |
| 78 | 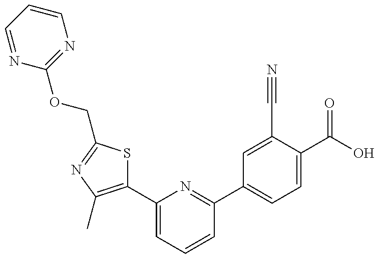 [SMILES: Cc1nc(COc2ncccn2)sc1-c1cccc(-c2ccc(C(=O)O)c(C#N)c2)n1] |
| 79 | 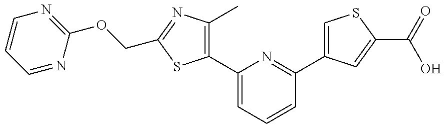 [SMILES: Cc1nc(COc2ncccn2)sc1-c1cccc(-c2csc(C(=O)O)c2)n1] |
| 80 | 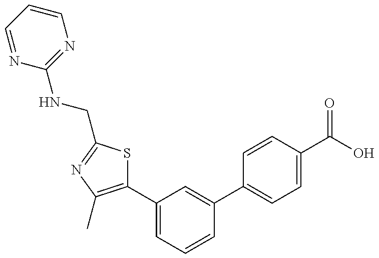 [SMILES: Cc1nc(CNc2ncccn2)sc1-c1cccc(-c2ccc(C(=O)O)cc2)c1] |
| 81 | 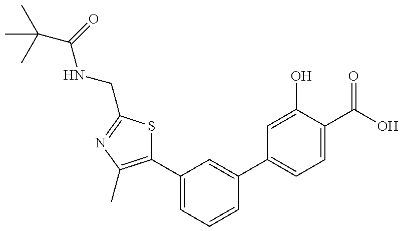 [SMILES: Cc1nc(CNC(=O)C(C)(C)C)sc1-c1cccc(-c2ccc(C(=O)O)c(O)c2)c1] |
| 82 | 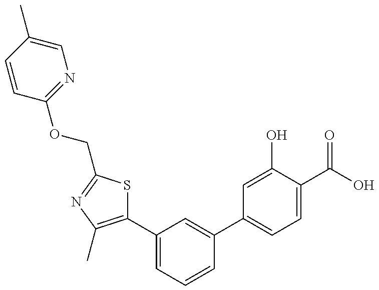 [SMILES: Cc1ccc(OCc2nc(C)c(-c3cccc(-c4ccc(C(=O)O)c(O)c4)c3)s2)nc1] |
| 83 | 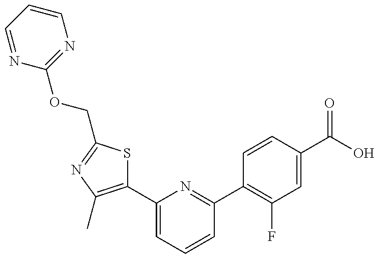 [SMILES: Cc1nc(COc2ncccn2)sc1-c1cccc(-c2ccc(C(=O)O)cc2F)n1] |
| 84 | 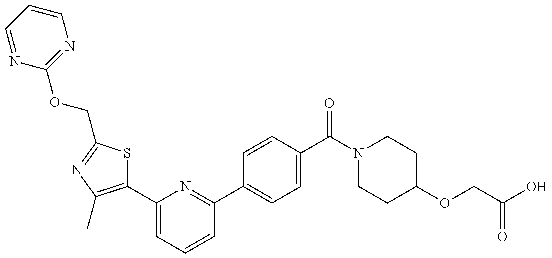 [SMILES: Cc1nc(COc2ncccn2)sc1-c1cccc(-c2ccc(C(=O)N3CCC(OCC(=O)O)CC3)cc2)n1] |
| 85 | 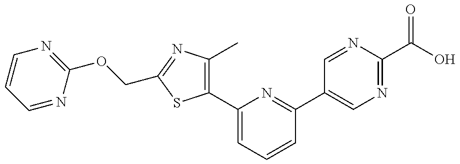 [SMILES: Cc1nc(COc2ncccn2)sc1-c1cccc(-c2cnc(C(=O)O)nc2)n1] |
| 86 | 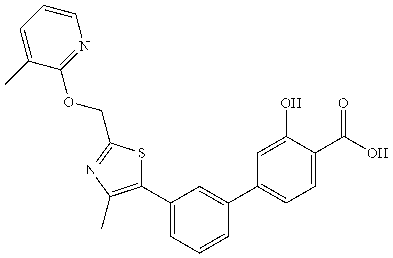 [SMILES: Cc1cccnc1OCc1nc(C)c(-c2cccc(-c3ccc(C(=O)O)c(O)c3)c2)s1] |
| 87 | 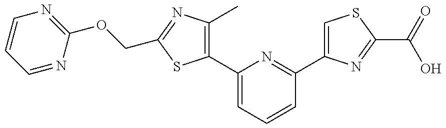 [SMILES: Cc1nc(COc2ncccn2)sc1-c1cccc(-c2csc(C(=O)O)n2)n1] |
| 88 | 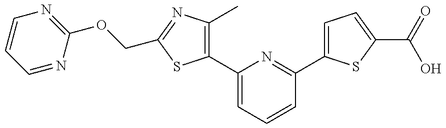 [SMILES: Cc1nc(COc2ncccn2)sc1-c1cccc(-c2ccc(C(=O)O)s2)n1] |
| 89 | 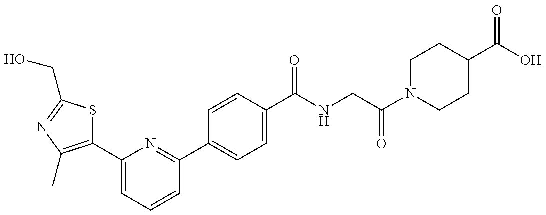 [SMILES: Cc1nc(CO)sc1-c1cccc(-c2ccc(C(=O)NCC(=O)N3CCC(C(=O)O)CC3)cc2)n1] |
| 90 | 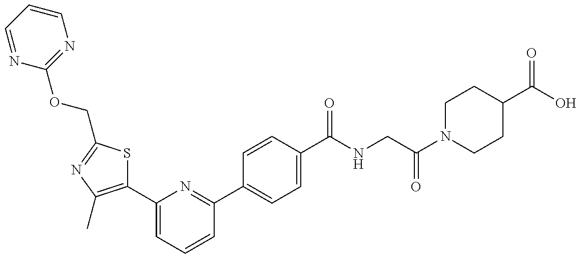 [SMILES: Cc1nc(COc2ncccn2)sc1-c1cccc(-c2ccc(C(=O)NCC(=O)N3CCC(C(=O)O)CC3)cc2)n1] |
| 91 | 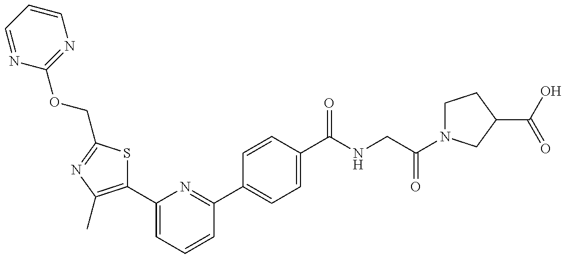 [SMILES: Cc1nc(COc2ncccn2)sc1-c1cccc(-c2ccc(C(=O)NCC(=O)N3CCC(C(=O)O)C3)cc2)n1] |
| 92 | 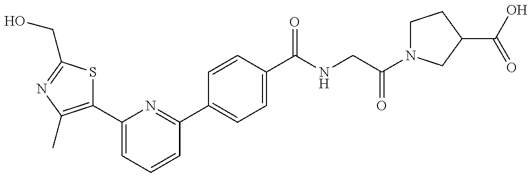 [SMILES: Cc1nc(CO)sc1-c1cccc(-c2ccc(C(=O)NCC(=O)N3CCC(C(=O)O)C3)cc2)n1] |
| 93 | 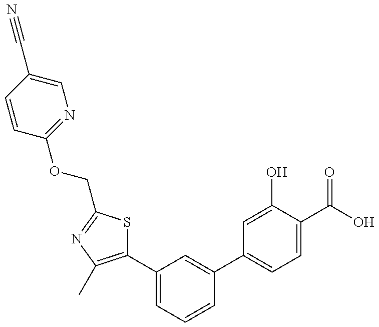 [SMILES: Cc1nc(COc2ccc(C#N)cn2)sc1-c1cccc(-c2ccc(C(=O)O)c(O)c2)c1] |
| 94 | 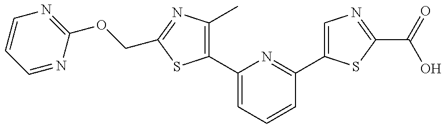 [SMILES: Cc1nc(COc2ncccn2)sc1-c1cccc(-c2cnc(C(=O)O)s2)n1] |
| 95 | 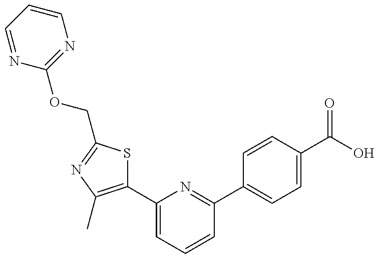 [SMILES: Cc1nc(COc2ncccn2)sc1-c1cccc(-c2ccc(C(=O)O)cc2)n1] |
| 96 | 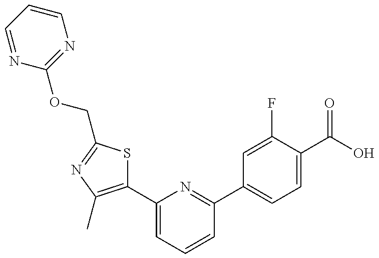 [SMILES: Cc1nc(COc2ncccn2)sc1-c1cccc(-c2ccc(C(=O)O)c(F)c2)n1] |
| 97 | 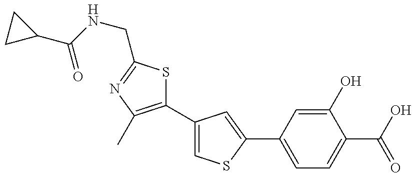 [SMILES: Cc1nc(CNC(=O)C2CC2)sc1-c1csc(-c2ccc(C(=O)O)c(O)c2)c1] |
| 98 | 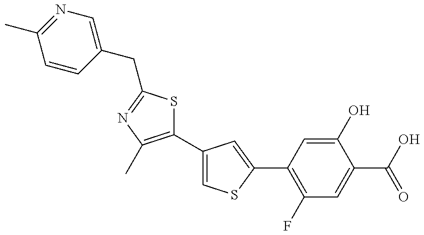 [SMILES: Cc1ccc(Cc2nc(C)c(-c3csc(-c4cc(O)c(C(=O)O)cc4F)c3)s2)cn1] |
| 99 | 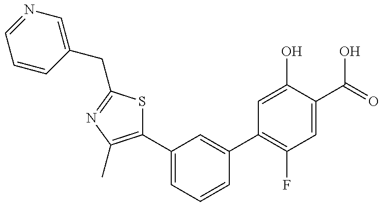 [SMILES: Cc1nc(Cc2cccnc2)sc1-c1cccc(-c2cc(O)c(C(=O)O)cc2F)c1] |
| 100 | 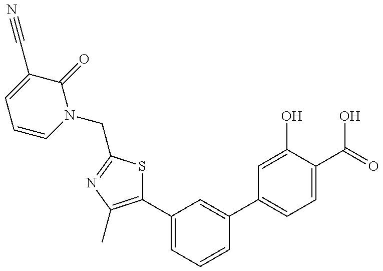 [SMILES: Cc1nc(Cn2cccc(C#N)c2=O)sc1-c1cccc(-c2ccc(C(=O)O)c(O)c2)c1] |
| 101 | 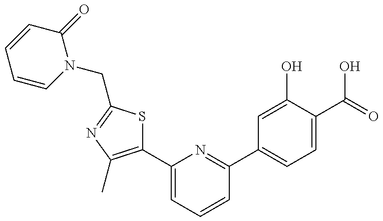 [SMILES: Cc1nc(Cn2ccccc2=O)sc1-c1cccc(-c2ccc(C(=O)O)c(O)c2)n1] |
| 102 | 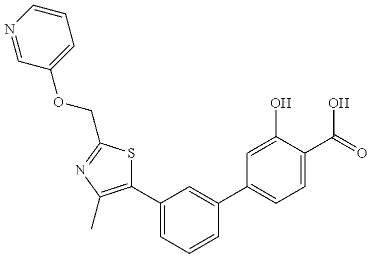 [SMILES: Cc1nc(COc2cccnc2)sc1-c1cccc(-c2ccc(C(=O)O)c(O)c2)c1] |
| 103 | 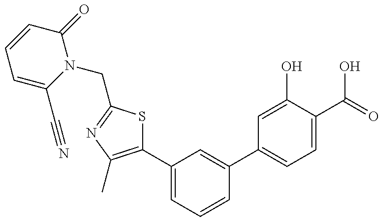 [SMILES: Cc1nc(Cn2c(C#N)cccc2=O)sc1-c1cccc(-c2ccc(C(=O)O)c(O)c2)c1] |
| 104 | 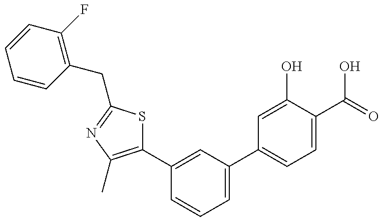 [SMILES: Cc1nc(Cc2ccccc2F)sc1-c1cccc(-c2ccc(C(=O)O)c(O)c2)c1] |
| 105 | 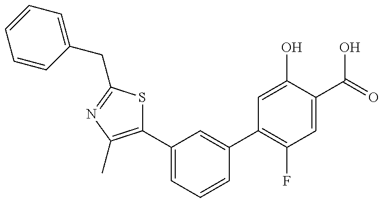 [SMILES: Cc1nc(Cc2ccccc2)sc1-c1cccc(-c2cc(O)c(C(=O)O)cc2F)c1] |
| 106 | 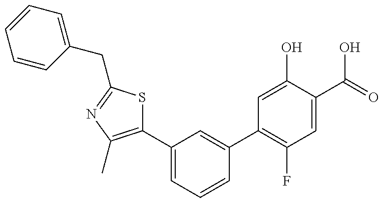 [SMILES: Cc1nc(Cc2ccccc2)sc1-c1cccc(-c2cc(O)c(C(=O)O)cc2F)c1] |
| 107 | 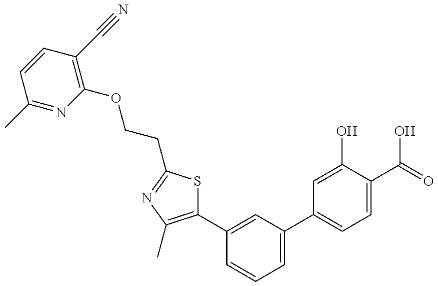 [SMILES: Cc1ccc(C#N)c(OCCc2nc(C)c(-c3cccc(-c4ccc(C(=O)O)c(O)c4)c3)s2)n1] |
| 108 | 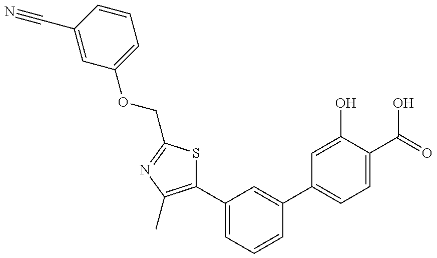 [SMILES: Cc1nc(COc2cccc(C#N)c2)sc1-c1cccc(-c2ccc(C(=O)O)c(O)c2)c1] |
| 109 | 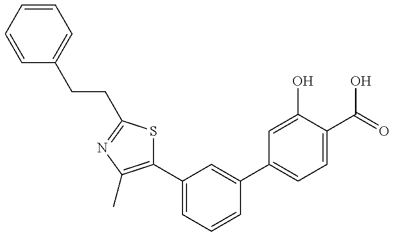 [SMILES: Cc1nc(CCc2ccccc2)sc1-c1cccc(-c2ccc(C(=O)O)c(O)c2)c1] |
| 110 | 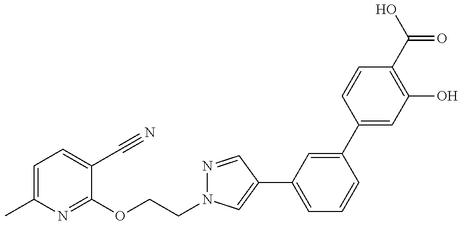 [SMILES: Cc1ccc(C#N)c(OCCn2cc(-c3cccc(-c4ccc(C(=O)O)c(O)c4)c3)cn2)n1] |
| 111 | 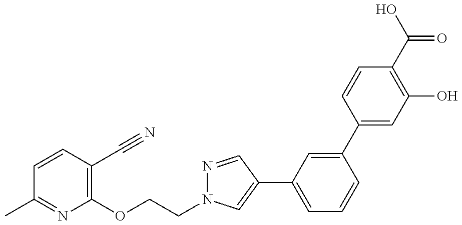 [SMILES: Cc1ccc(C#N)c(OCCn2cc(-c3cccc(-c4ccc(C(=O)O)c(O)c4)c3)cn2)n1] |
| 112 | 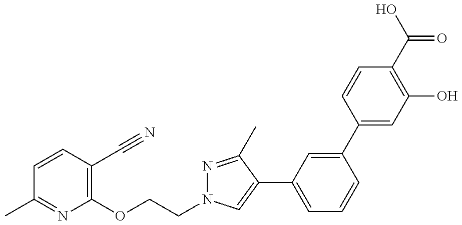 [SMILES: Cc1ccc(C#N)c(OCCn2cc(-c3cccc(-c4ccc(C(=O)O)c(O)c4)c3)c(C)n2)n1] |
| 113 | 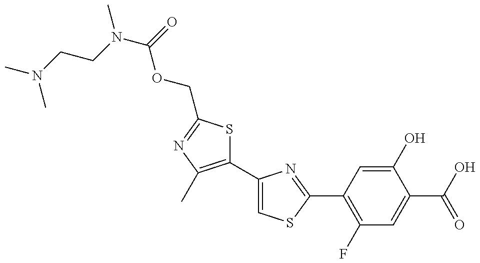 [SMILES: Cc1nc(COC(=O)N(C)CCN(C)C)sc1-c1csc(-c2cc(O)c(C(=O)O)cc2F)n1] |
| 114 | 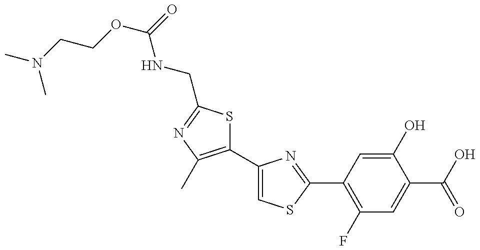 [SMILES: Cc1nc(CNC(=O)OCCN(C)C)sc1-c1csc(-c2cc(O)c(C(=O)O)cc2F)n1] |
| 115 | 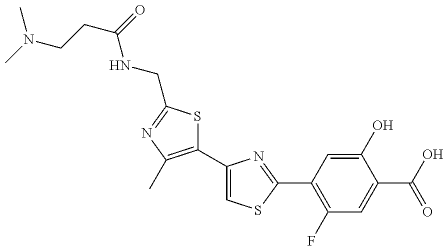 [SMILES: Cc1nc(CNC(=O)CCN(C)C)sc1-c1csc(-c2cc(O)c(C(=O)O)cc2F)n1] |
| 116 | 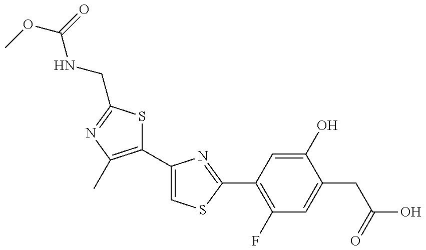 [SMILES: COC(=O)NCc1nc(C)c(-c2csc(-c3cc(O)c(CC(=O)O)cc3F)n2)s1] |
| 117 | 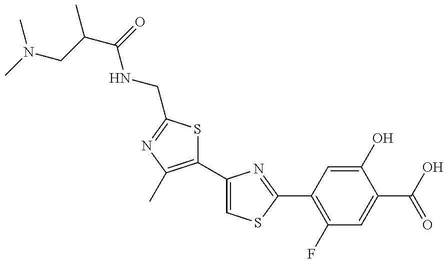 [SMILES: Cc1nc(CNC(=O)C(C)CN(C)C)sc1-c1csc(-c2cc(O)c(C(=O)O)cc2F)n1] |
| 118 | 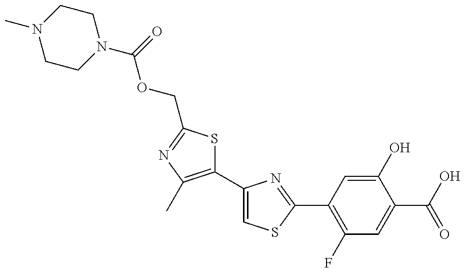 [SMILES: Cc1nc(COC(=O)N2CCN(C)CC2)sc1-c1csc(-c2cc(O)c(C(=O)O)cc2F)n1] |
| 119 | 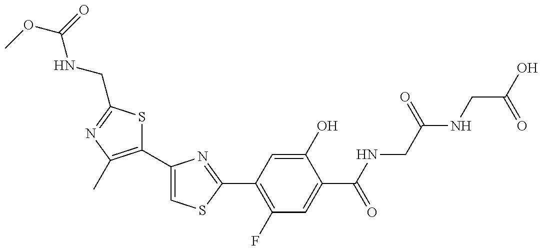 [SMILES: COC(=O)NCc1nc(C)c(-c2csc(-c3cc(O)c(C(=O)NCC(=O)NCC(=O)O)cc3F)n2)s1] |
| 120 | 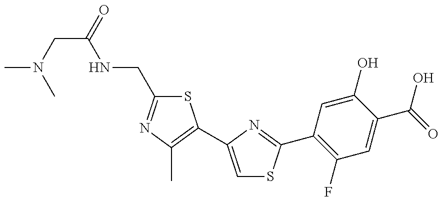 [SMILES: Cc1nc(CNC(=O)CN(C)C)sc1-c1csc(-c2cc(O)c(C(=O)O)cc2F)n1] |
| 121 | 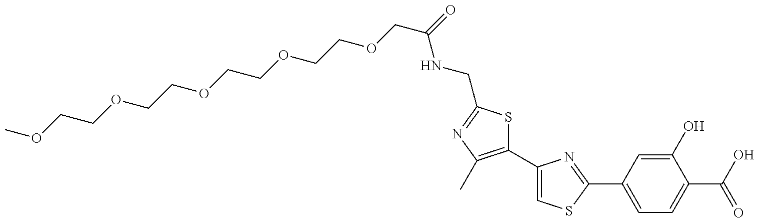 [SMILES: COCCOCCOCCOCCOCC(=O)NCc1nc(C)c(-c2csc(-c3ccc(C(=O)O)c(O)c3)n2)s1] |
| 122 | 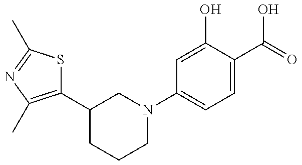 [SMILES: Cc1nc(C)c(C2CCCN(c3ccc(C(=O)O)c(O)c3)C2)s1] |
| 123 | 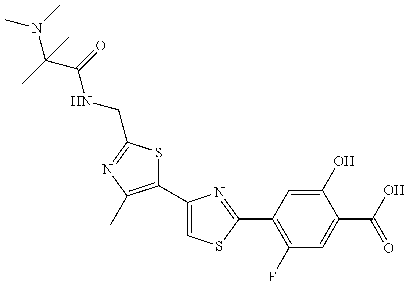 [SMILES: Cc1nc(CNC(=O)C(C)(C)N(C)C)sc1-c1csc(-c2cc(O)c(C(=O)O)cc2F)n1] |
| 124 | 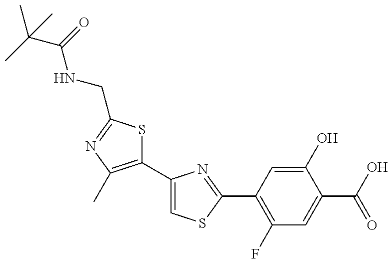 [SMILES: Cc1nc(CNC(=O)C(C)(C)C)sc1-c1csc(-c2cc(O)c(C(=O)O)cc2F)n1] |
| 125 | 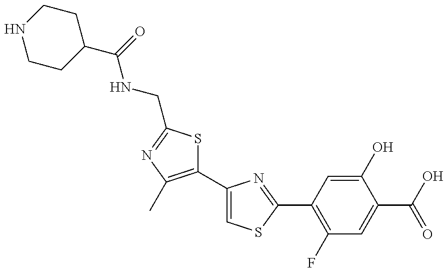 [SMILES: Cc1nc(CNC(=O)C2CCNCC2)sc1-c1csc(-c2cc(O)c(C(=O)O)cc2F)n1] |
| 126 | 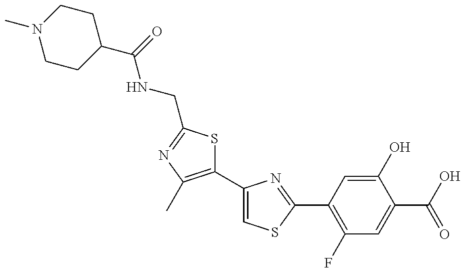 [SMILES: Cc1nc(CNC(=O)C2CCN(C)CC2)sc1-c1csc(-c2cc(O)c(C(=O)O)cc2F)n1] |
| 127 | 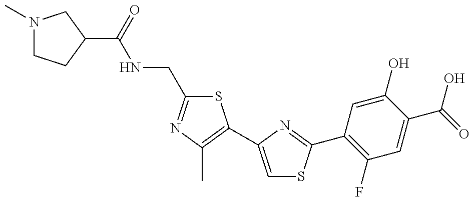 [SMILES: Cc1nc(CNC(=O)C2CCN(C)C2)sc1-c1csc(-c2cc(O)c(C(=O)O)cc2F)n1] |
| 128 | 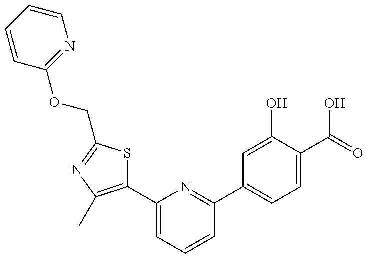 [SMILES: Cc1nc(COc2ccccn2)sc1-c1cccc(-c2ccc(C(=O)O)c(O)c2)n1] |
| 129 | 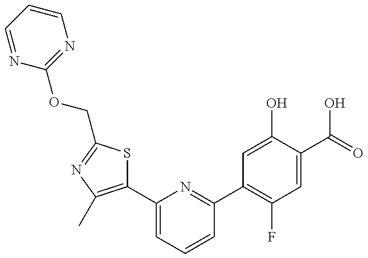 [SMILES: Cc1nc(COc2ncccn2)sc1-c1cccc(-c2cc(O)c(C(=O)O)cc2F)n1] |
| 130 | 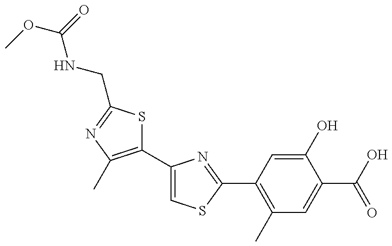 [SMILES: COC(=O)NCc1nc(C)c(-c2csc(-c3cc(O)c(C(=O)O)cc3C)n2)s1] |
| 131 | 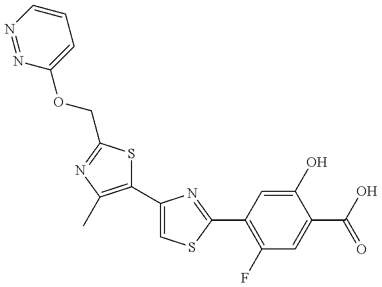 [SMILES: Cc1nc(COc2cccnn2)sc1-c1csc(-c2cc(O)c(C(=O)O)cc2F)n1] |
| 132 | 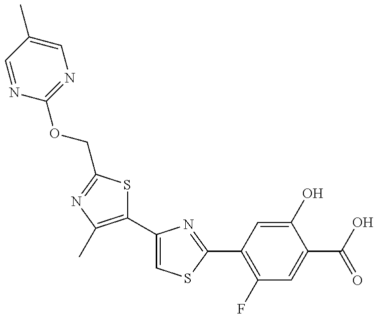 [SMILES: Cc1cnc(OCc2nc(C)c(-c3csc(-c4cc(O)c(C(=O)O)cc4F)n3)s2)nc1] |
| 133 | 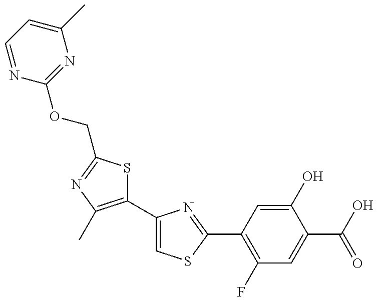 [SMILES: Cc1ccnc(OCc2nc(C)c(-c3csc(-c4cc(O)c(C(=O)O)cc4F)n3)s2)n1] |
| 134 | 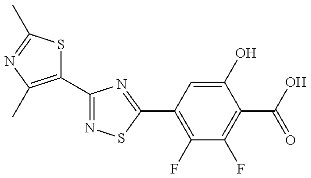 [SMILES: Cc1nc(C)c(-c2nsc(-c3cc(O)c(C(=O)O)c(F)c3F)n2)s1] |
| 135 | 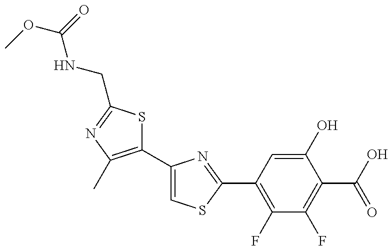 [SMILES: COC(=O)NCc1nc(C)c(-c2csc(-c3cc(O)c(C(=O)O)c(F)c3F)n2)s1] |
| 136 | 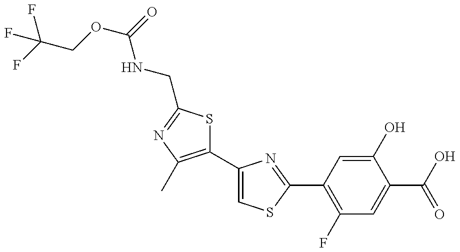 [SMILES: Cc1nc(CNC(=O)OCC(F)(F)F)sc1-c1csc(-c2cc(O)c(C(=O)O)cc2F)n1] |
| 137 | 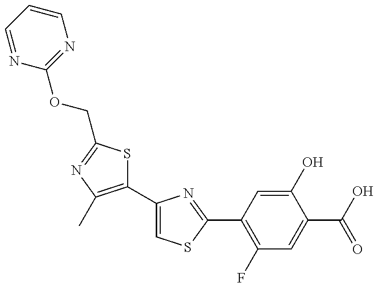 [SMILES: Cc1nc(COc2ncccn2)sc1-c1csc(-c2cc(O)c(C(=O)O)cc2F)n1] |
| 138 | 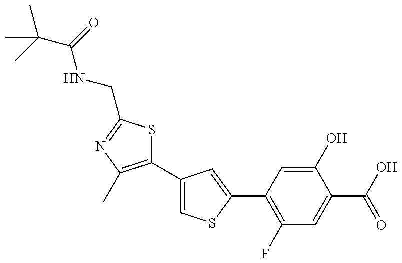 [SMILES: Cc1nc(CNC(=O)C(C)(C)C)sc1-c1csc(-c2cc(O)c(C(=O)O)cc2F)c1] |
| 139 | 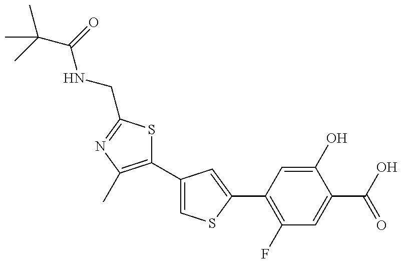 [SMILES: Cc1nc(CNC(=O)C(C)(C)C)sc1-c1csc(-c2cc(O)c(C(=O)O)cc2F)c1] |
| 140 | 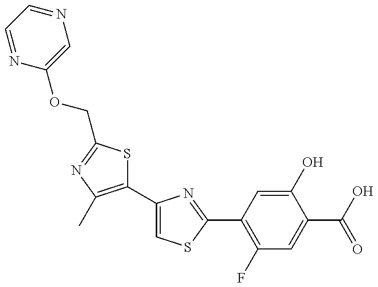 [SMILES: Cc1nc(COc2cnccn2)sc1-c1csc(-c2cc(O)c(C(=O)O)cc2F)n1] |
| 141 | 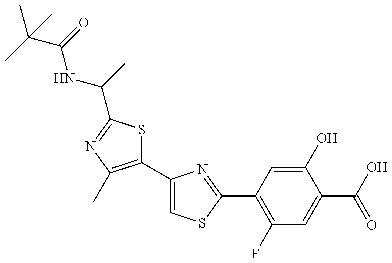 [SMILES: Cc1nc(C(C)NC(=O)C(C)(C)C)sc1-c1csc(-c2cc(O)c(C(=O)O)cc2F)n1] |
| 142 | 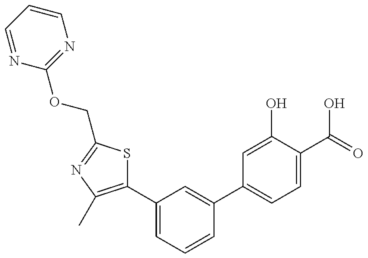 [SMILES: Cc1nc(COc2ncccn2)sc1-c1cccc(-c2ccc(C(=O)O)c(O)c2)c1] |
| 143 | 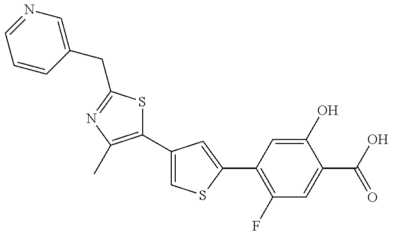 [SMILES: Cc1nc(Cc2cccnc2)sc1-c1csc(-c2cc(O)c(C(=O)O)cc2F)c1] |
| 144 | 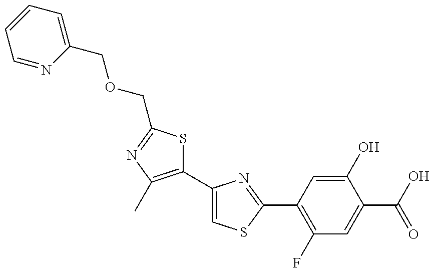 [SMILES: Cc1nc(COCc2ccccn2)sc1-c1csc(-c2cc(O)c(C(=O)O)cc2F)n1] |
| 145 | 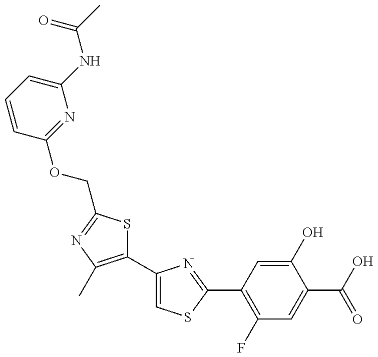 [SMILES: CC(=O)Nc1cccc(OCc2nc(C)c(-c3csc(-c4cc(O)c(C(=O)O)cc4F)n3)s2)n1] |
| 146 | 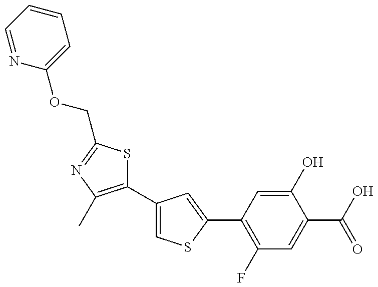 [SMILES: Cc1nc(COc2ccccn2)sc1-c1csc(-c2cc(O)c(C(=O)O)cc2F)c1] |
| 147 | 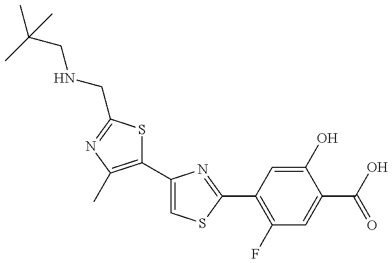 [SMILES: Cc1nc(CNCC(C)(C)C)sc1-c1csc(-c2cc(O)c(C(=O)O)cc2F)n1] |
| 148 | 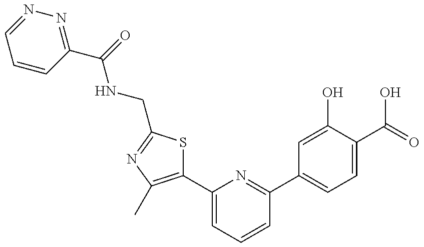 [SMILES: Cc1nc(CNC(=O)c2cccnn2)sc1-c1cccc(-c2ccc(C(=O)O)c(O)c2)n1] |
| 149 | 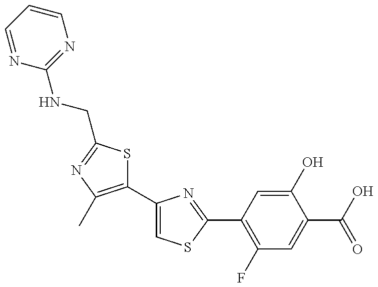 [SMILES: Cc1nc(CNc2ncccn2)sc1-c1csc(-c2cc(O)c(C(=O)O)cc2F)n1] |
| 150 | 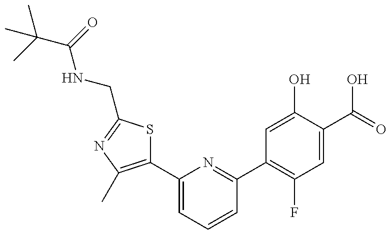 [SMILES: Cc1nc(CNC(=O)C(C)(C)C)sc1-c1cccc(-c2cc(O)c(C(=O)O)cc2F)n1] |
| 151 | 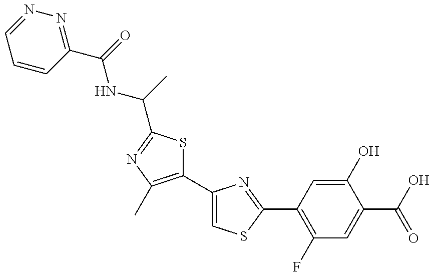 [SMILES: Cc1nc(C(C)NC(=O)c2cccnn2)sc1-c1csc(-c2cc(O)c(C(=O)O)cc2F)n1] |
| 152 | 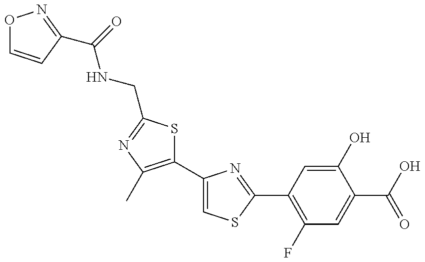 [SMILES: Cc1nc(CNC(=O)c2ccon2)sc1-c1csc(-c2cc(O)c(C(=O)O)cc2F)n1] |
| 153 | 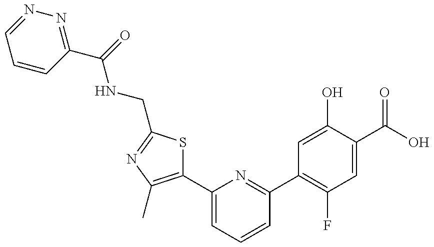 [SMILES: Cc1nc(CNC(=O)c2cccnn2)sc1-c1cccc(-c2cc(O)c(C(=O)O)cc2F)n1] |
| 154 | 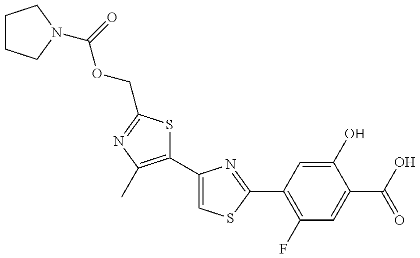 [SMILES: Cc1nc(COC(=O)N2CCCC2)sc1-c1csc(-c2cc(O)c(C(=O)O)cc2F)n1] |
| 155 | 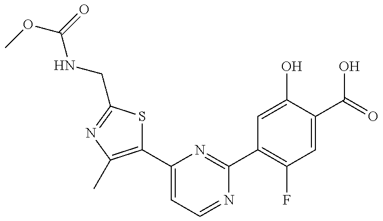 [SMILES: COC(=O)NCc1nc(C)c(-c2ccnc(-c3cc(O)c(C(=O)O)cc3F)n2)s1] |
| 156 | 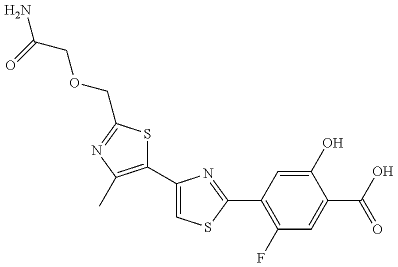 [SMILES: Cc1nc(COCC(N)=O)sc1-c1csc(-c2cc(O)c(C(=O)O)cc2F)n1] |
| 157 | 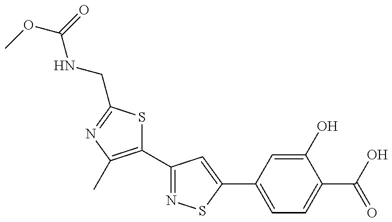 [SMILES: COC(=O)NCc1nc(C)c(-c2cc(-c3ccc(C(=O)O)c(O)c3)sn2)s1] |
| 158 | 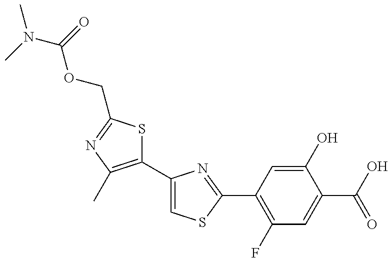 [SMILES: Cc1nc(COC(=O)N(C)C)sc1-c1csc(-c2cc(O)c(C(=O)O)cc2F)n1] |
| 159 | 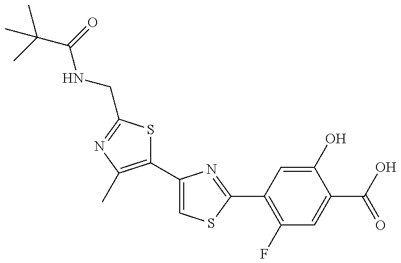 [SMILES: Cc1nc(CNC(=O)C(C)(C)C)sc1-c1csc(-c2cc(O)c(C(=O)O)cc2F)n1] |
| 160 | 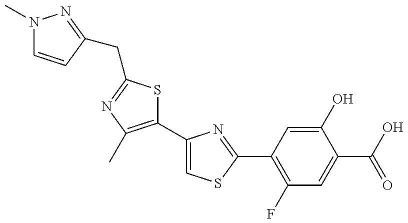 [SMILES: Cc1nc(Cc2ccn(C)n2)sc1-c1csc(-c2cc(O)c(C(=O)O)cc2F)n1] |
| 161 | 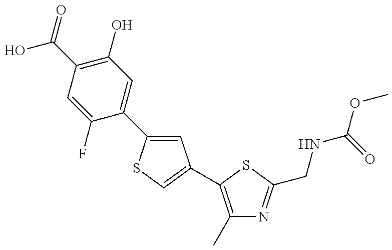 [SMILES: COC(=O)NCc1nc(C)c(-c2csc(-c3cc(O)c(C(=O)O)cc3F)c2)s1] |
| 162 | 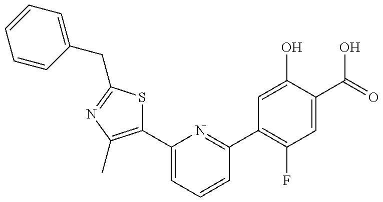 [SMILES: Cc1nc(Cc2ccccc2)sc1-c1cccc(-c2cc(O)c(C(=O)O)cc2F)n1] |
| 163 | 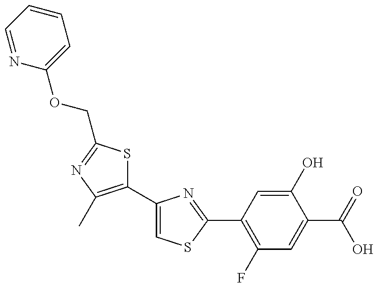 [SMILES: Cc1nc(COc2ccccn2)sc1-c1csc(-c2cc(O)c(C(=O)O)cc2F)n1] |
| 164 | 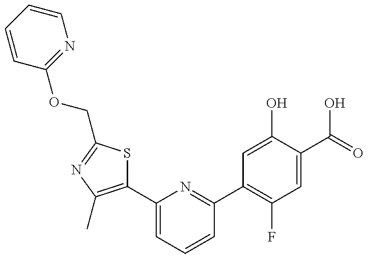 [SMILES: Cc1nc(COc2ccccn2)sc1-c1cccc(-c2cc(O)c(C(=O)O)cc2F)n1] |
| 165 | 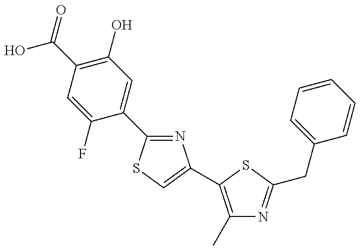 [SMILES: Cc1nc(Cc2ccccc2)sc1-c1csc(-c2cc(O)c(C(=O)O)cc2F)n1] |
| 166 | 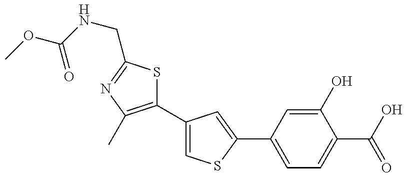 [SMILES: COC(=O)NCc1nc(C)c(-c2csc(-c3ccc(C(=O)O)c(O)c3)c2)s1] |
| 167 | 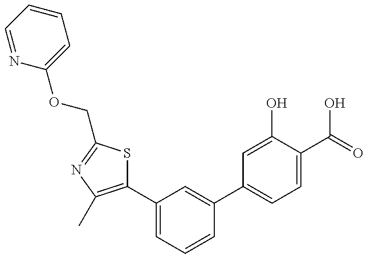 [SMILES: Cc1nc(COc2ccccn2)sc1-c1cccc(-c2ccc(C(=O)O)c(O)c2)c1] |
| 168 | 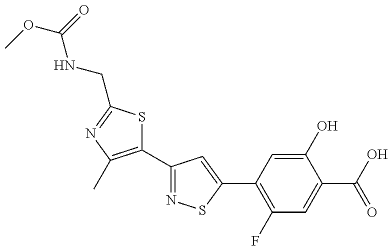 [SMILES: COC(=O)NCc1nc(C)c(-c2cc(-c3cc(O)c(C(=O)O)cc3F)sn2)s1] |
| 169 | 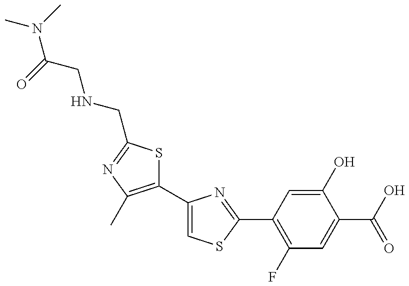 [SMILES: Cc1nc(CNCC(=O)N(C)C)sc1-c1csc(-c2cc(O)c(C(=O)O)cc2F)n1] |
| 170 | 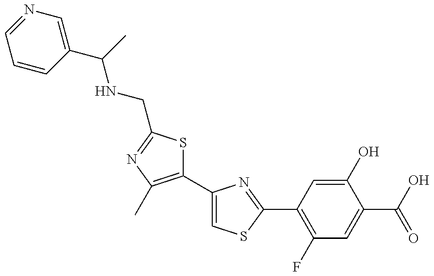 [SMILES: Cc1nc(CNC(C)c2cccnc2)sc1-c1csc(-c2cc(O)c(C(=O)O)cc2F)n1] |
| 171 | 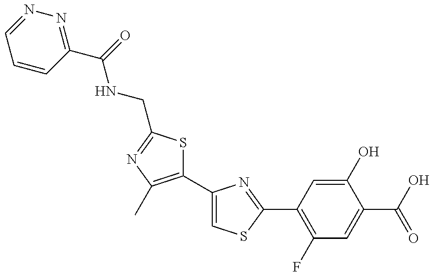 [SMILES: Cc1nc(CNC(=O)c2cccnn2)sc1-c1csc(-c2cc(O)c(C(=O)O)cc2F)n1] |
| 172 | 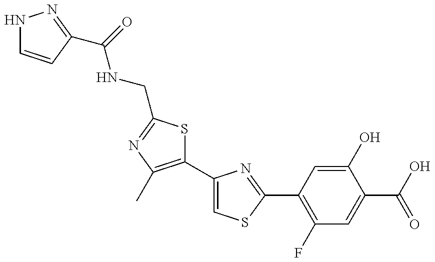 [SMILES: Cc1nc(CNC(=O)c2cc[nH]n2)sc1-c1csc(-c2cc(O)c(C(=O)O)cc2F)n1] |
| 173 | 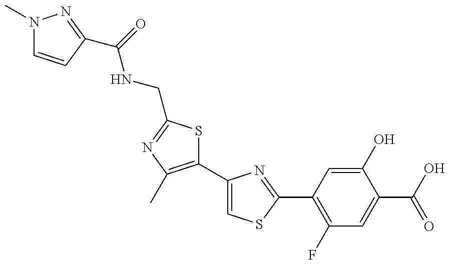 [SMILES: Cc1nc(CNC(=O)c2ccn(C)n2)sc1-c1csc(-c2cc(O)c(C(=O)O)cc2F)n1] |
| 174 | 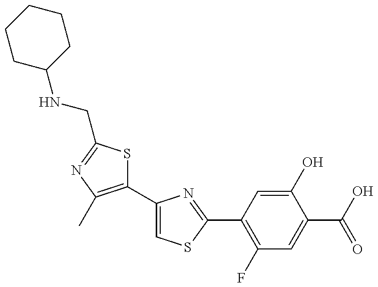 [SMILES: Cc1nc(CNC2CCCCC2)sc1-c1csc(-c2cc(O)c(C(=O)O)cc2F)n1] |
| 175 | 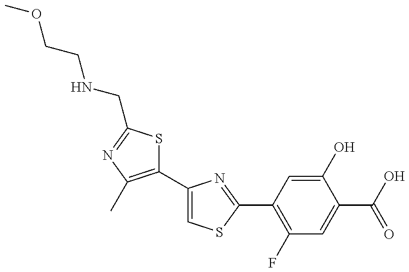 [SMILES: COCCNCc1nc(C)c(-c2csc(-c3cc(O)c(C(=O)O)cc3F)n2)s1] |
| 176 | 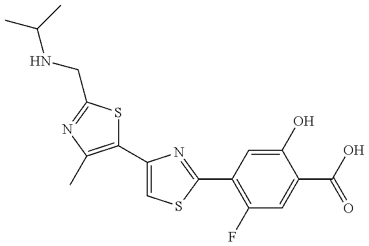 [SMILES: Cc1nc(CNC(C)C)sc1-c1csc(-c2cc(O)c(C(=O)O)cc2F)n1] |
| 177 | 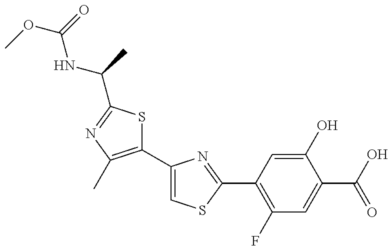 [SMILES: COC(=O)N[C@@H](C)c1nc(C)c(-c2csc(-c3cc(O)c(C(=O)O)cc3F)n2)s1] |
| 178 | 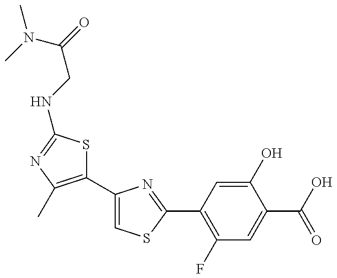 [SMILES: Cc1nc(NCC(=O)N(C)C)sc1-c1csc(-c2cc(O)c(C(=O)O)cc2F)n1] |
| 179 | 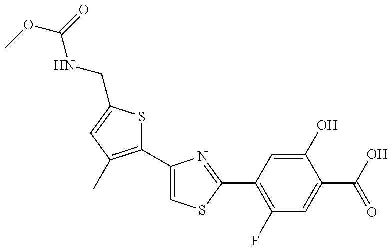 [SMILES: COC(=O)NCc1cc(C)c(-c2csc(-c3cc(O)c(C(=O)O)cc3F)n2)s1] |
| 180 | 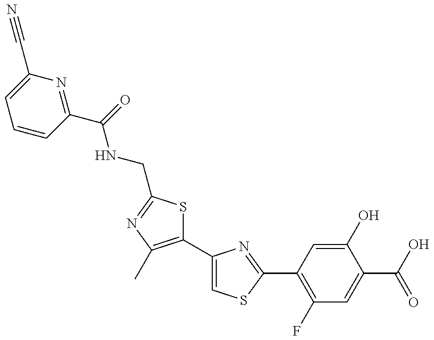 [SMILES: Cc1nc(CNC(=O)c2cccc(C#N)n2)sc1-c1csc(-c2cc(O)c(C(=O)O)cc2F)n1] |
| 181 | 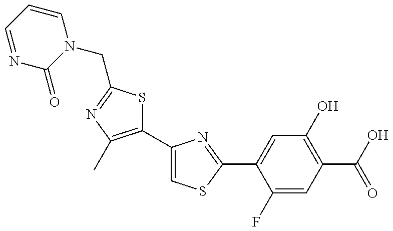 [SMILES: Cc1nc(Cn2cccnc2=O)sc1-c1csc(-c2cc(O)c(C(=O)O)cc2F)n1] |
| 182 | 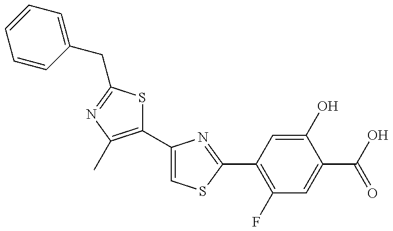 [SMILES: Cc1nc(Cc2ccccc2)sc1-c1csc(-c2cc(O)c(C(=O)O)cc2F)n1] |
| 183 | 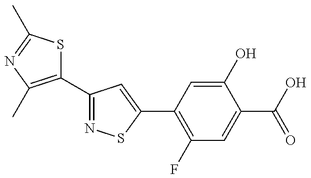 [SMILES: Cc1nc(C)c(-c2cc(-c3cc(O)c(C(=O)O)cc3F)sn2)s1] |
| 184 | 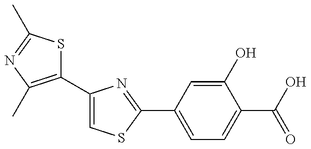 [SMILES: Cc1nc(C)c(-c2csc(-c3ccc(C(=O)O)c(O)c3)n2)s1] |
| 185 | 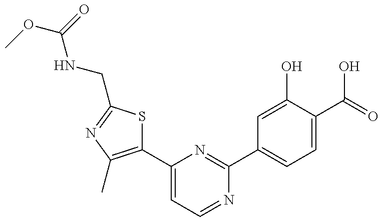 [SMILES: COC(=O)NCc1nc(C)c(-c2ccnc(-c3ccc(C(=O)O)c(O)c3)n2)s1] |
| 186 | 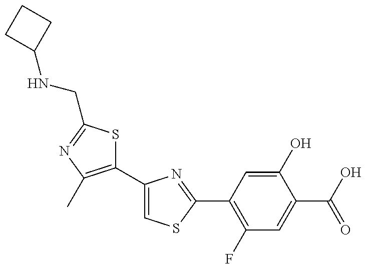 [SMILES: Cc1nc(CNC2CCC2)sc1-c1csc(-c2cc(O)c(C(=O)O)cc2F)n1] |
| 187 | 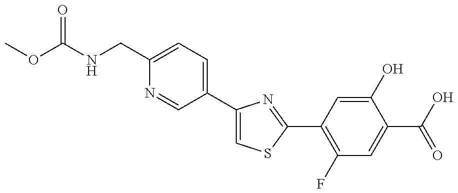 [SMILES: COC(=O)NCc1ccc(-c2csc(-c3cc(O)c(C(=O)O)cc3F)n2)cn1] |
| 188 | 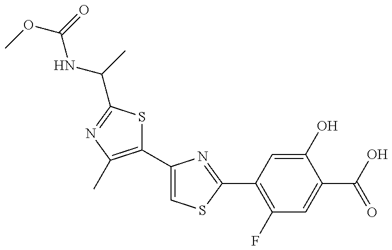 [SMILES: COC(=O)NC(C)c1nc(C)c(-c2csc(-c3cc(O)c(C(=O)O)cc3F)n2)s1] |
| 189 | 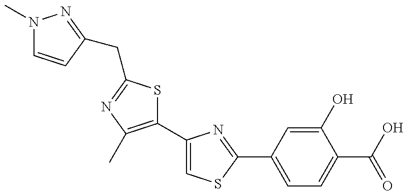 [SMILES: Cc1nc(Cc2ccn(C)n2)sc1-c1csc(-c2ccc(C(=O)O)c(O)c2)n1] |
| 190 | 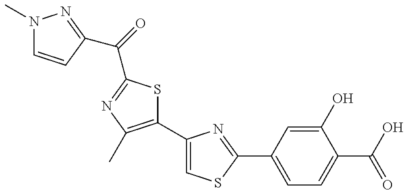 [SMILES: Cc1nc(C(=O)c2ccn(C)n2)sc1-c1csc(-c2ccc(C(=O)O)c(O)c2)n1] |
| 191 | 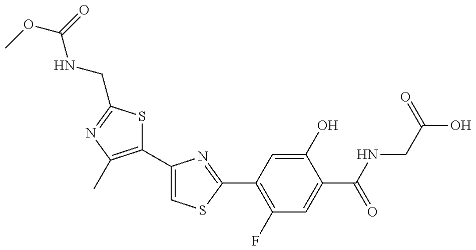 [SMILES: COC(=O)NCc1nc(C)c(-c2csc(-c3cc(O)c(C(=O)NCC(=O)O)cc3F)n2)s1] |
| 192 | 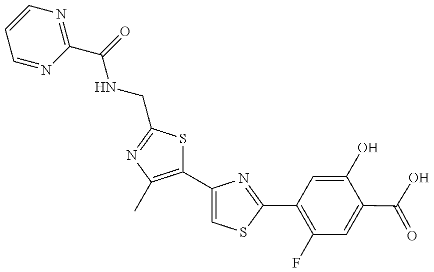 [SMILES: Cc1nc(CNC(=O)c2ncccn2)sc1-c1csc(-c2cc(O)c(C(=O)O)cc2F)n1] |
| 193 | 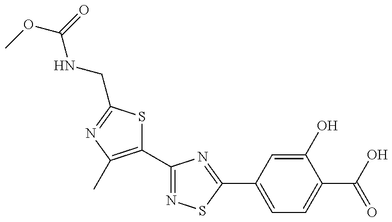 [SMILES: COC(=O)NCc1nc(C)c(-c2nsc(-c3ccc(C(=O)O)c(O)c3)n2)s1] |
| 194 | 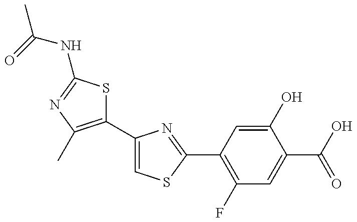 [SMILES: CC(=O)Nc1nc(C)c(-c2csc(-c3cc(O)c(C(=O)O)cc3F)n2)s1] |
| 195 | 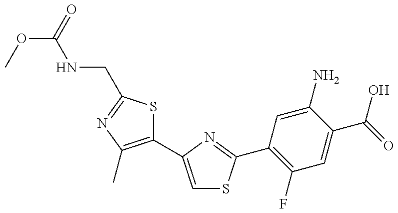 [SMILES: COC(=O)NCc1nc(C)c(-c2csc(-c3cc(N)c(C(=O)O)cc3F)n2)s1] |
| 196 | 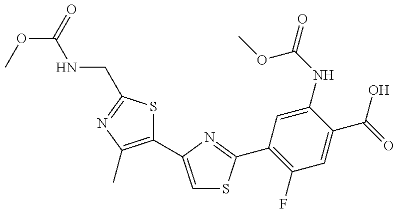 [SMILES: COC(=O)NCc1nc(C)c(-c2csc(-c3cc(NC(=O)OC)c(C(=O)O)cc3F)n2)s1] |
| 197 | 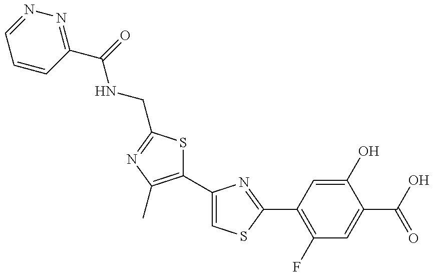 [SMILES: Cc1nc(CNC(=O)c2cccnn2)sc1-c1csc(-c2cc(O)c(C(=O)O)cc2F)n1] |
| 198 | 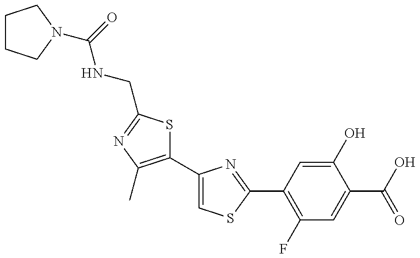 [SMILES: Cc1nc(CNC(=O)N2CCCC2)sc1-c1csc(-c2cc(O)c(C(=O)O)cc2F)n1] |
| 199 | 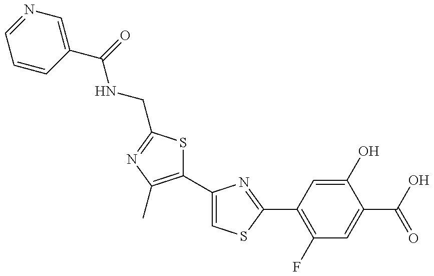 [SMILES: Cc1nc(CNC(=O)c2cccnc2)sc1-c1csc(-c2cc(O)c(C(=O)O)cc2F)n1] |
| 200 | 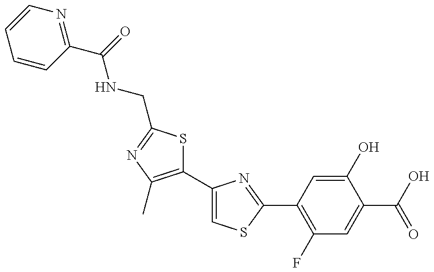 [SMILES: Cc1nc(CNC(=O)c2ccccn2)sc1-c1csc(-c2cc(O)c(C(=O)O)cc2F)n1] |
| 201 | 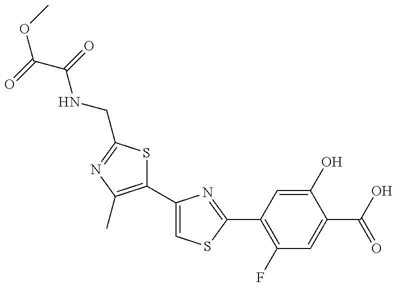 [SMILES: COC(=O)C(=O)NCc1nc(C)c(-c2csc(-c3cc(O)c(C(=O)O)cc3F)n2)s1] |
| 202 | 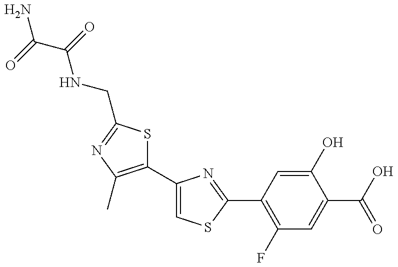 [SMILES: Cc1nc(CNC(=O)C(N)=O)sc1-c1csc(-c2cc(O)c(C(=O)O)cc2F)n1] |
| 203 | 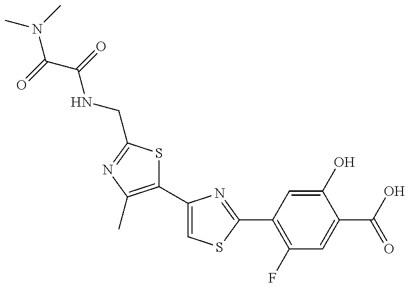 [SMILES: Cc1nc(CNC(=O)C(=O)N(C)C)sc1-c1csc(-c2cc(O)c(C(=O)O)cc2F)n1] |
| 204 | 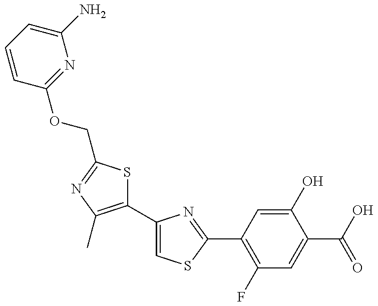 [SMILES: Cc1nc(COc2cccc(N)n2)sc1-c1csc(-c2cc(O)c(C(=O)O)cc2F)n1] |
| 205 | 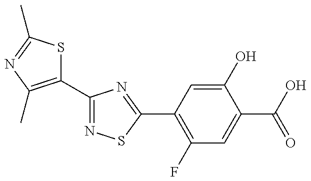 [SMILES: Cc1nc(C)c(-c2nsc(-c3cc(O)c(C(=O)O)cc3F)n2)s1] |
| 206 | 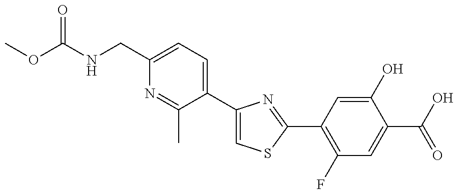 [SMILES: COC(=O)NCc1ccc(-c2csc(-c3cc(O)c(C(=O)O)cc3F)n2)c(C)n1] |
| 207 | 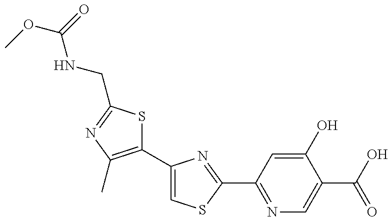 [SMILES: COC(=O)NCc1nc(C)c(-c2csc(-c3cc(O)c(C(=O)O)cn3)n2)s1] |
| 208 | 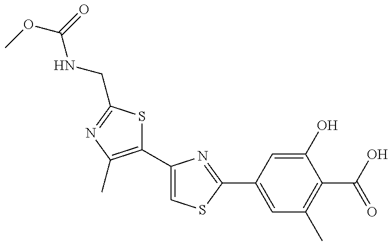 [SMILES: COC(=O)NCc1nc(C)c(-c2csc(-c3cc(C)c(C(=O)O)c(O)c3)n2)s1] |
| 209 | 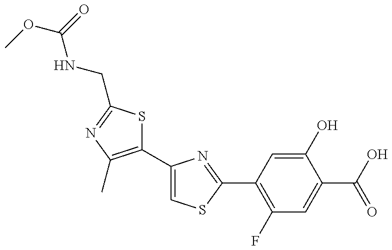 [SMILES: COC(=O)NCc1nc(C)c(-c2csc(-c3cc(O)c(C(=O)O)cc3F)n2)s1] |
| 210 | 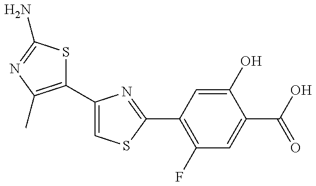 [SMILES: Cc1nc(N)sc1-c1csc(-c2cc(O)c(C(=O)O)cc2F)n1] |
| 211 | 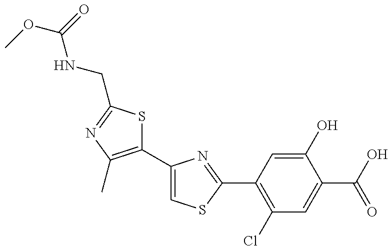 [SMILES: COC(=O)NCc1nc(C)c(-c2csc(-c3cc(O)c(C(=O)O)cc3Cl)n2)s1] |
| 212 | 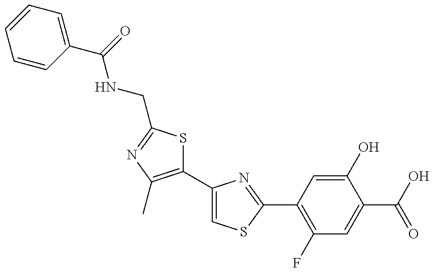 [SMILES: Cc1nc(CNC(=O)c2ccccc2)sc1-c1csc(-c2cc(O)c(C(=O)O)cc2F)n1] |
| 213 | 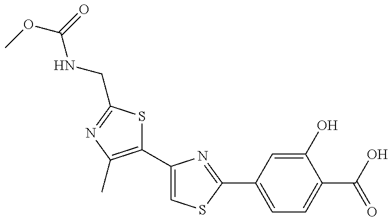 [SMILES: COC(=O)NCc1nc(C)c(-c2csc(-c3ccc(C(=O)O)c(O)c3)n2)s1] |
| 214 | 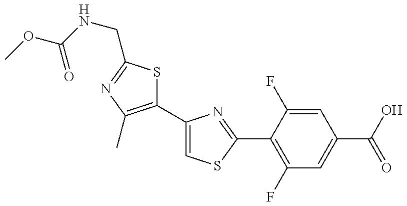 [SMILES: COC(=O)NCc1nc(C)c(-c2csc(-c3c(F)cc(C(=O)O)cc3F)n2)s1] |
| 215 | 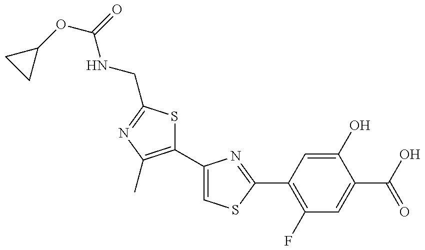 [SMILES: Cc1nc(CNC(=O)OC2CC2)sc1-c1csc(-c2cc(O)c(C(=O)O)cc2F)n1] |
| 216 | 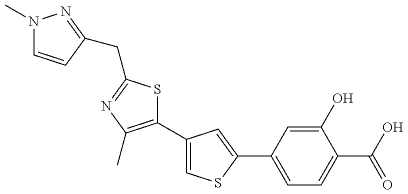 [SMILES: Cc1nc(Cc2ccn(C)n2)sc1-c1csc(-c2ccc(C(=O)O)c(O)c2)c1] |
| 217 | 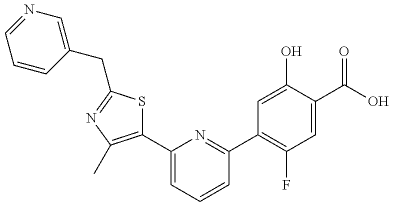 [SMILES: Cc1nc(Cc2cccnc2)sc1-c1cccc(-c2cc(O)c(C(=O)O)cc2F)n1] |
| 218 | 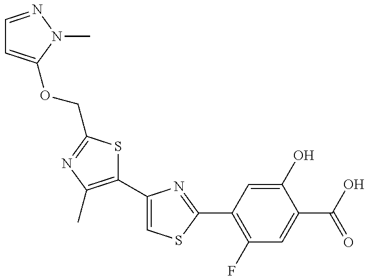 [SMILES: Cc1nc(COc2ccnn2C)sc1-c1csc(-c2cc(O)c(C(=O)O)cc2F)n1] |
| 219 | 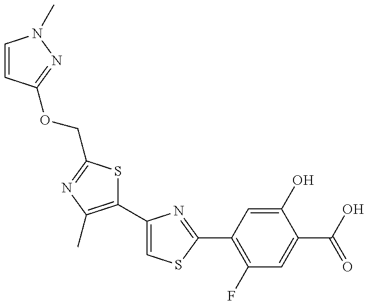 [SMILES: Cc1nc(COc2ccn(C)n2)sc1-c1csc(-c2cc(O)c(C(=O)O)cc2F)n1] |
| 220 | 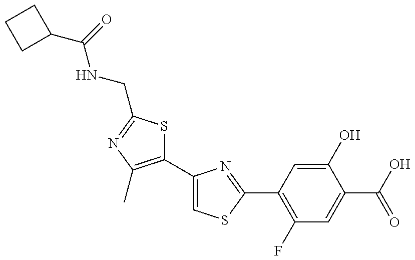 [SMILES: Cc1nc(CNC(=O)C2CCC2)sc1-c1csc(-c2cc(O)c(C(=O)O)cc2F)n1] |
| 221 | 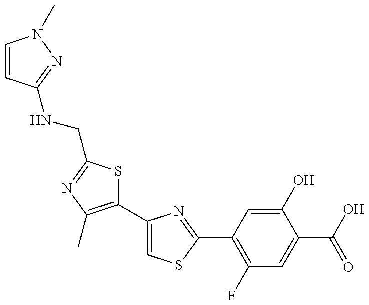 [SMILES: Cc1nc(CNc2ccn(C)n2)sc1-c1csc(-c2cc(O)c(C(=O)O)cc2F)n1] |
| 222 | 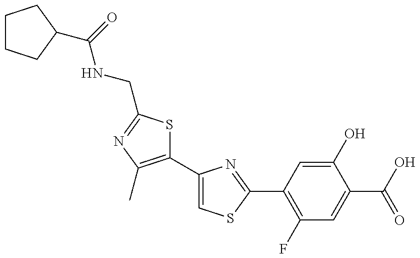 [SMILES: Cc1nc(CNC(=O)C2CCCC2)sc1-c1csc(-c2cc(O)c(C(=O)O)cc2F)n1] |
| 223 | 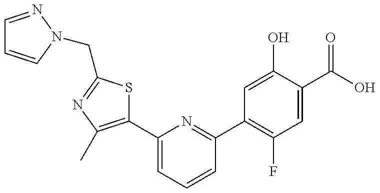 [SMILES: Cc1nc(Cn2cccn2)sc1-c1cccc(-c2cc(O)c(C(=O)O)cc2F)n1] |
| 224 | 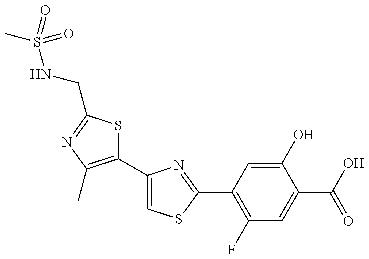 [SMILES: Cc1nc(CNS(C)(=O)=O)sc1-c1csc(-c2cc(O)c(C(=O)O)cc2F)n1] |
| 225 | 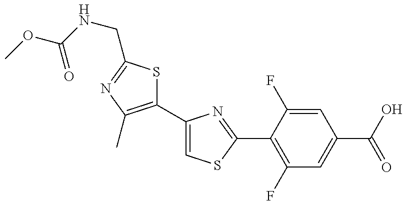 [SMILES: COC(=O)NCc1nc(C)c(-c2csc(-c3c(F)cc(C(=O)O)cc3F)n2)s1] |
| 226 | 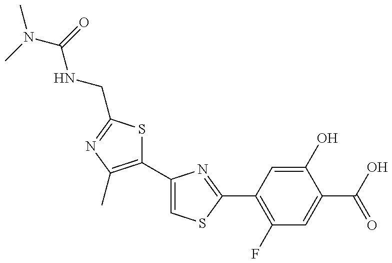 [SMILES: Cc1nc(CNC(=O)N(C)C)sc1-c1csc(-c2cc(O)c(C(=O)O)cc2F)n1] |
| 227 | 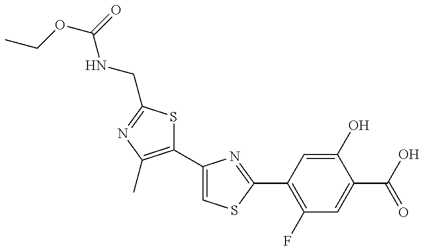 [SMILES: CCOC(=O)NCc1nc(C)c(-c2csc(-c3cc(O)c(C(=O)O)cc3F)n2)s1] |
| 228 | 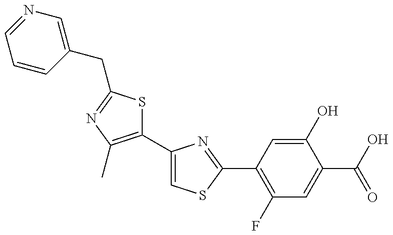 [SMILES: Cc1nc(Cc2cccnc2)sc1-c1csc(-c2cc(O)c(C(=O)O)cc2F)n1] |
| 229 | 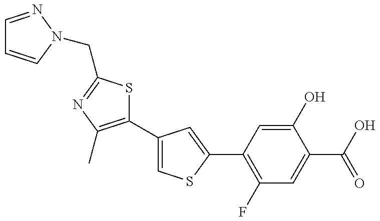 [SMILES: Cc1nc(Cn2cccn2)sc1-c1csc(-c2cc(O)c(C(=O)O)cc2F)c1] |
| 230 | 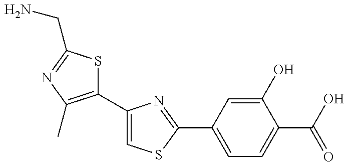 [SMILES: Cc1nc(CN)sc1-c1csc(-c2ccc(C(=O)O)c(O)c2)n1] |
| 231 | 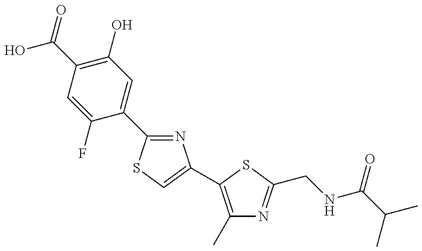 [SMILES: Cc1nc(CNC(=O)C(C)C)sc1-c1csc(-c2cc(O)c(C(=O)O)cc2F)n1] |
| 232 | 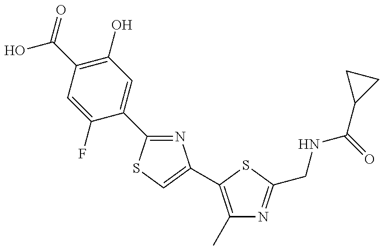 [SMILES: Cc1nc(CNC(=O)C2CC2)sc1-c1csc(-c2cc(O)c(C(=O)O)cc2F)n1] |
| 233 | 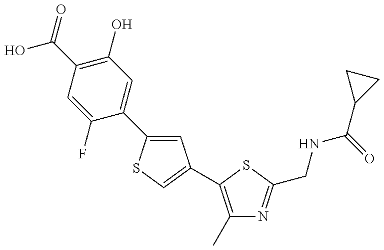 [SMILES: Cc1nc(CNC(=O)C2CC2)sc1-c1csc(-c2cc(O)c(C(=O)O)cc2F)c1] |
| 234 | 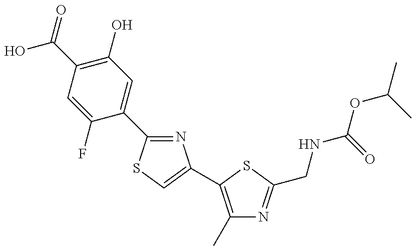 [SMILES: Cc1nc(CNC(=O)OC(C)C)sc1-c1csc(-c2cc(O)c(C(=O)O)cc2F)n1] |
| 235 | 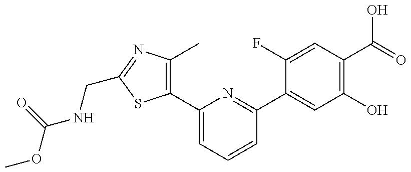 [SMILES: COC(=O)NCc1nc(C)c(-c2cccc(-c3cc(O)c(C(=O)O)cc3F)n2)s1] |
| 236 | 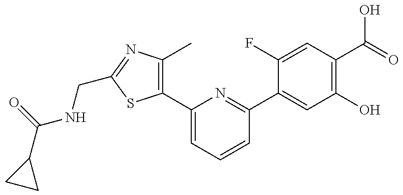 [SMILES: Cc1nc(CNC(=O)C2CC2)sc1-c1cccc(-c2cc(O)c(C(=O)O)cc2F)n1] |
| 237 | 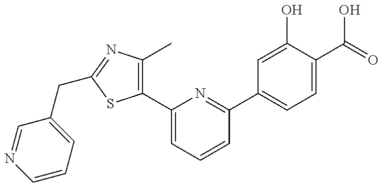 [SMILES: Cc1nc(Cc2cccnc2)sc1-c1cccc(-c2ccc(C(=O)O)c(O)c2)n1] |
| 238 | 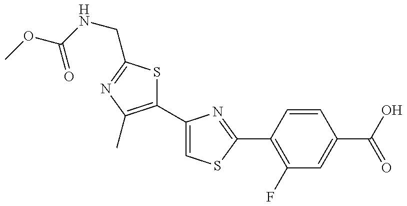 [SMILES: COC(=O)NCc1nc(C)c(-c2csc(-c3ccc(C(=O)O)cc3F)n2)s1] |
| 239 | 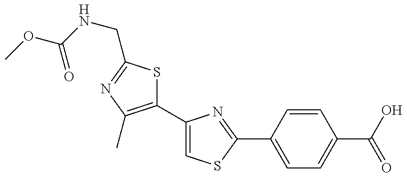 [SMILES: COC(=O)NCc1nc(C)c(-c2csc(-c3ccc(C(=O)O)cc3)n2)s1] |
| 240 | 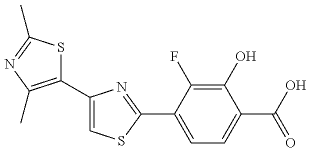 [SMILES: Cc1nc(C)c(-c2csc(-c3ccc(C(=O)O)c(O)c3F)n2)s1] |
| 241 | 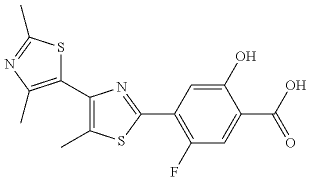 [SMILES: Cc1nc(C)c(-c2nc(-c3cc(O)c(C(=O)O)cc3F)sc2C)s1] |
| 242 | 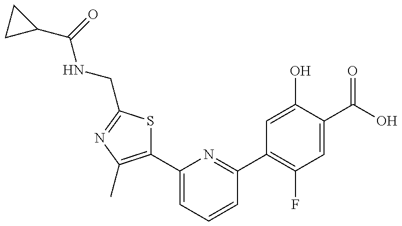 [SMILES: Cc1nc(CNC(=O)C2CC2)sc1-c1cccc(-c2cc(O)c(C(=O)O)cc2F)n1] |
| 243 | 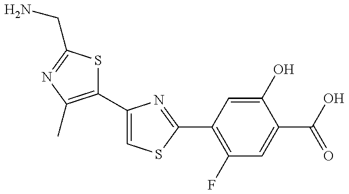 [SMILES: Cc1nc(CN)sc1-c1csc(-c2cc(O)c(C(=O)O)cc2F)n1] |
| 244 | 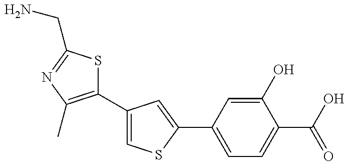 [SMILES: Cc1nc(CN)sc1-c1csc(-c2ccc(C(=O)O)c(O)c2)c1] |
| 245 | 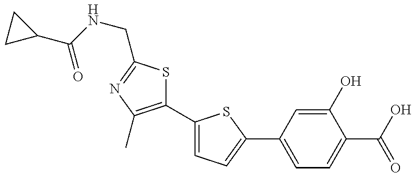 [SMILES: Cc1nc(CNC(=O)C2CC2)sc1-c1ccc(-c2ccc(C(=O)O)c(O)c2)s1] |
| 246 | 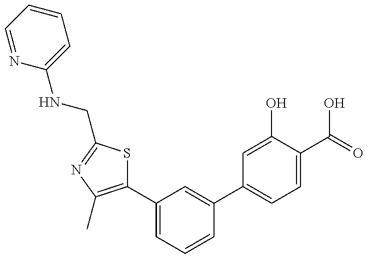 [SMILES: Cc1nc(CNc2ccccn2)sc1-c1cccc(-c2ccc(C(=O)O)c(O)c2)c1] |
| 247 | 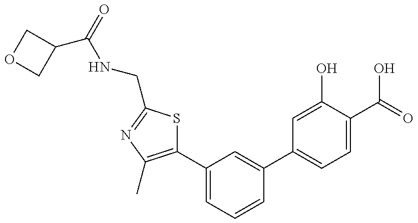 [SMILES: Cc1nc(CNC(=O)C2COC2)sc1-c1cccc(-c2ccc(C(=O)O)c(O)c2)c1] |
| 248 | 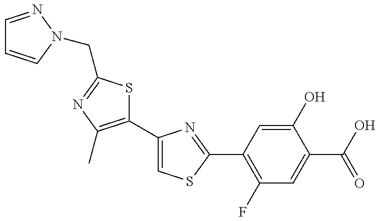 [SMILES: Cc1nc(Cn2cccn2)sc1-c1csc(-c2cc(O)c(C(=O)O)cc2F)n1] |
| 249 | 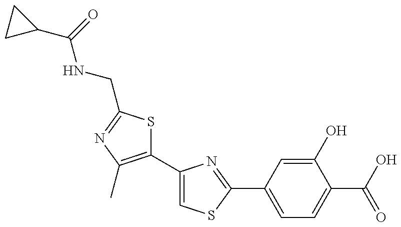 [SMILES: Cc1nc(CNC(=O)C2CC2)sc1-c1csc(-c2ccc(C(=O)O)c(O)c2)n1] |
| 250 | 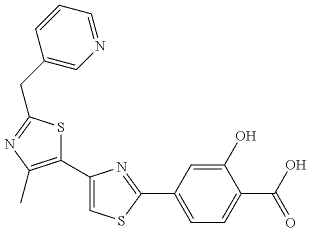 [SMILES: Cc1nc(Cc2cccnc2)sc1-c1csc(-c2ccc(C(=O)O)c(O)c2)n1] |
| 251 | 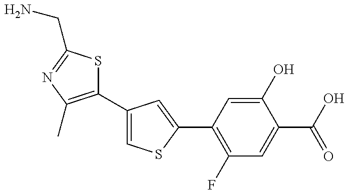 [SMILES: Cc1nc(CN)sc1-c1csc(-c2cc(O)c(C(=O)O)cc2F)c1] |
| 252 | 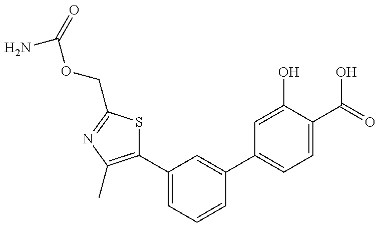 [SMILES: Cc1nc(COC(N)=O)sc1-c1cccc(-c2ccc(C(=O)O)c(O)c2)c1] |
| 253 | 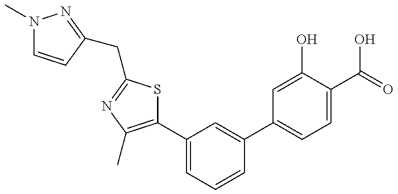 [SMILES: Cc1nc(Cc2ccn(C)n2)sc1-c1cccc(-c2ccc(C(=O)O)c(O)c2)c1] |
| 254 | 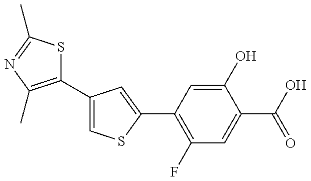 [SMILES: Cc1nc(C)c(-c2csc(-c3cc(O)c(C(=O)O)cc3F)c2)s1] |
| 255 | 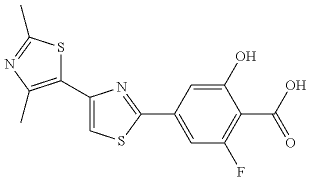 [SMILES: Cc1nc(C)c(-c2csc(-c3cc(O)c(C(=O)O)c(F)c3)n2)s1] |
| 256 | 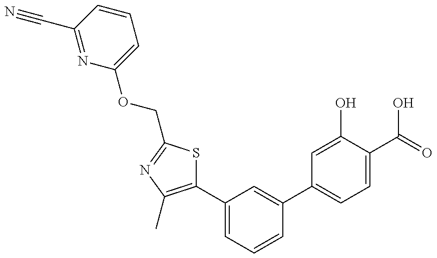 [SMILES: Cc1nc(COc2cccc(C#N)n2)sc1-c1cccc(-c2ccc(C(=O)O)c(O)c2)c1] |
| 257 | 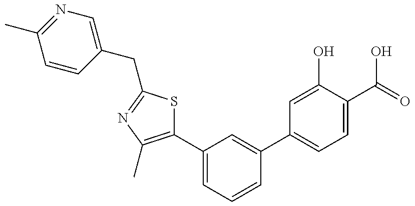 [SMILES: Cc1ccc(Cc2nc(C)c(-c3cccc(-c4ccc(C(=O)O)c(O)c4)c3)s2)cn1] |
| 258 | 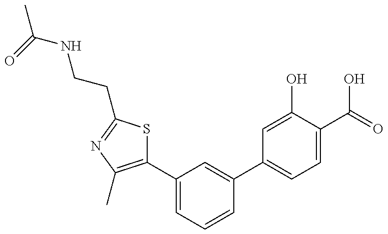 [SMILES: CC(=O)NCCc1nc(C)c(-c2cccc(-c3ccc(C(=O)O)c(O)c3)c2)s1] |
| 259 | 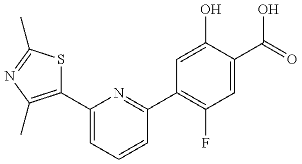 [SMILES: Cc1nc(C)c(-c2cccc(-c3cc(O)c(C(=O)O)cc3F)n2)s1] |
| 260 | 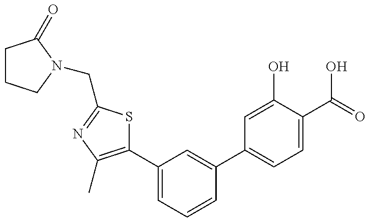 [SMILES: Cc1nc(CN2CCCC2=O)sc1-c1cccc(-c2ccc(C(=O)O)c(O)c2)c1] |
| 261 | 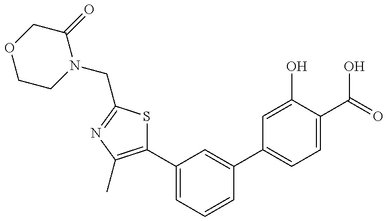 [SMILES: Cc1nc(CN2CCOCC2=O)sc1-c1cccc(-c2ccc(C(=O)O)c(O)c2)c1] |
| 262 | 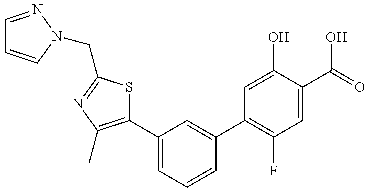 [SMILES: Cc1nc(Cn2cccn2)sc1-c1cccc(-c2cc(O)c(C(=O)O)cc2F)c1] |
| 263 | 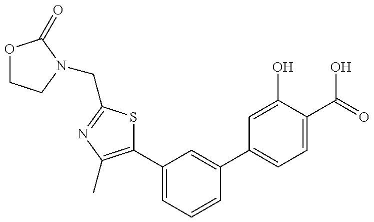 [SMILES: Cc1nc(CN2CCOC2=O)sc1-c1cccc(-c2ccc(C(=O)O)c(O)c2)c1] |
| 264 | 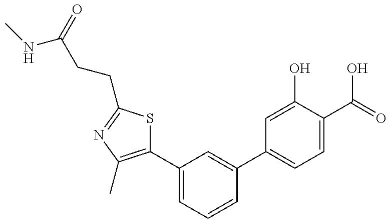 [SMILES: CNC(=O)CCc1nc(C)c(-c2cccc(-c3ccc(C(=O)O)c(O)c3)c2)s1] |
| 265 | 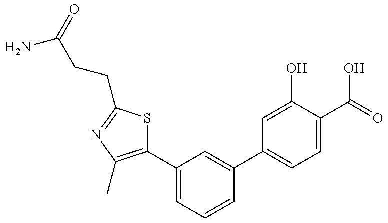 [SMILES: Cc1nc(CCC(N)=O)sc1-c1cccc(-c2ccc(C(=O)O)c(O)c2)c1] |
| 266 | 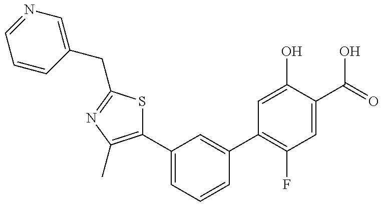 [SMILES: Cc1nc(Cc2cccnc2)sc1-c1cccc(-c2cc(O)c(C(=O)O)cc2F)c1] |
| 267 | 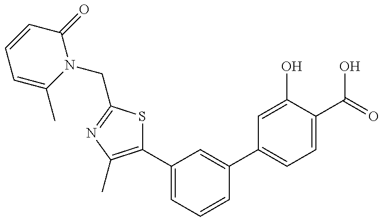 [SMILES: Cc1nc(Cn2c(C)cccc2=O)sc1-c1cccc(-c2ccc(C(=O)O)c(O)c2)c1] |
| 268 | 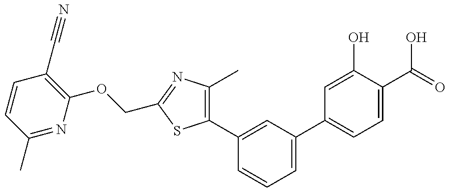 [SMILES: Cc1ccc(C#N)c(OCc2nc(C)c(-c3cccc(-c4ccc(C(=O)O)c(O)c4)c3)s2)n1] |
| 269 | 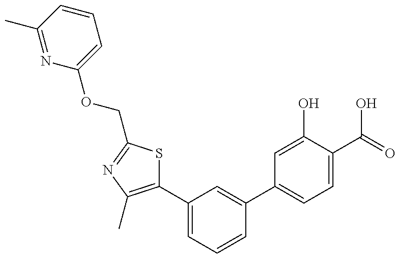 [SMILES: Cc1cccc(OCc2nc(C)c(-c3cccc(-c4ccc(C(=O)O)c(O)c4)c3)s2)n1] |
| 270 | 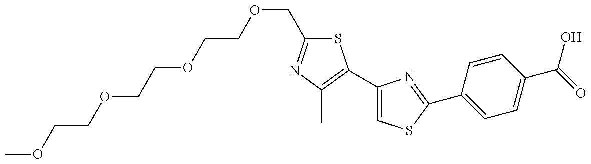 [SMILES: COCCOCCOCCOCc1nc(C)c(-c2csc(-c3ccc(C(=O)O)cc3)n2)s1] |
| 271 | 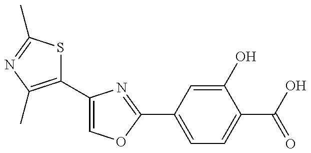 [SMILES: Cc1nc(C)c(-c2coc(-c3ccc(C(=O)O)c(O)c3)n2)s1] |
| 272 | 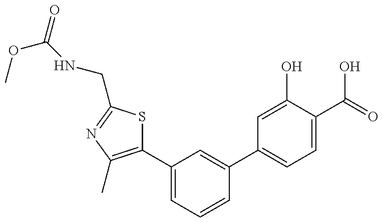 [SMILES: COC(=O)NCc1nc(C)c(-c2cccc(-c3ccc(C(=O)O)c(O)c3)c2)s1] |
| 273 | 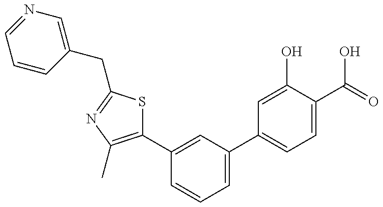 [SMILES: Cc1nc(Cc2cccnc2)sc1-c1cccc(-c2ccc(C(=O)O)c(O)c2)c1] |
| 274 | 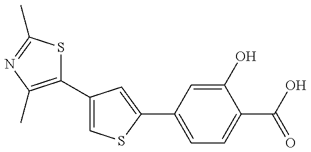 [SMILES: Cc1nc(C)c(-c2csc(-c3ccc(C(=O)O)c(O)c3)c2)s1] |
| 275 | 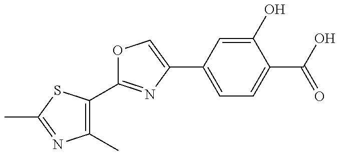 [SMILES: Cc1nc(C)c(-c2nc(-c3ccc(C(=O)O)c(O)c3)co2)s1] |
| 276 | 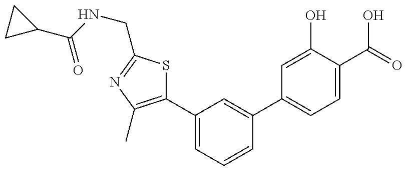 [SMILES: Cc1nc(CNC(=O)C2CC2)sc1-c1cccc(-c2ccc(C(=O)O)c(O)c2)c1] |
| 277 | 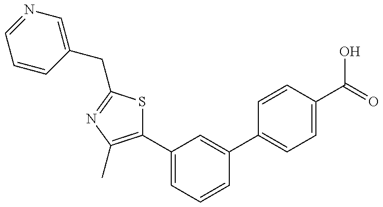 [SMILES: Cc1nc(Cc2cccnc2)sc1-c1cccc(-c2ccc(C(=O)O)cc2)c1] |
| 278 | 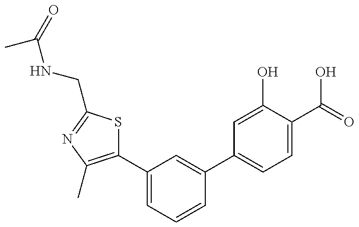 [SMILES: CC(=O)NCc1nc(C)c(-c2cccc(-c3ccc(C(=O)O)c(O)c3)c2)s1] |
| 279 | 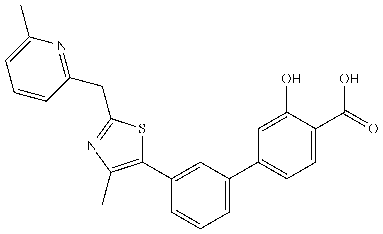 [SMILES: Cc1cccc(Cc2nc(C)c(-c3cccc(-c4ccc(C(=O)O)c(O)c4)c3)s2)n1] |
| 280 | 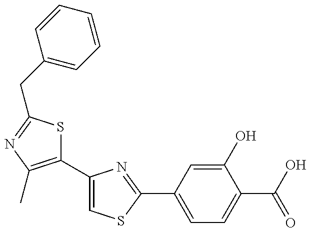 [SMILES: Cc1nc(Cc2ccccc2)sc1-c1csc(-c2ccc(C(=O)O)c(O)c2)n1] |
| 281 | 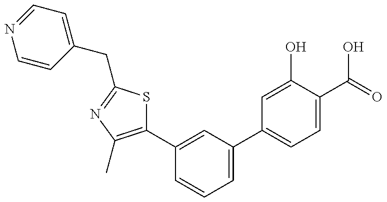 [SMILES: Cc1nc(Cc2ccncc2)sc1-c1cccc(-c2ccc(C(=O)O)c(O)c2)c1] |
| 282 | 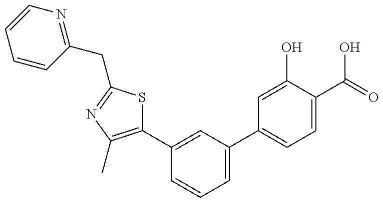 [SMILES: Cc1nc(Cc2ccccn2)sc1-c1cccc(-c2ccc(C(=O)O)c(O)c2)c1] |
| 283 | 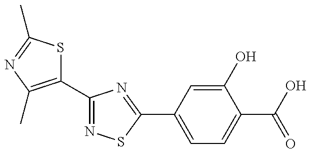 [SMILES: Cc1nc(C)c(-c2nsc(-c3ccc(C(=O)O)c(O)c3)n2)s1] |
| 284 | 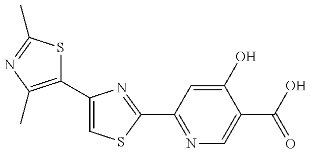 [SMILES: Cc1nc(C)c(-c2csc(-c3cc(O)c(C(=O)O)cn3)n2)s1] |
| 285 | 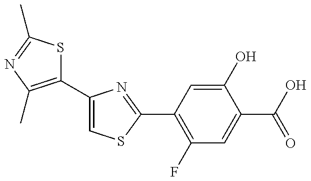 [SMILES: Cc1nc(C)c(-c2csc(-c3cc(O)c(C(=O)O)cc3F)n2)s1] |
| 286 | 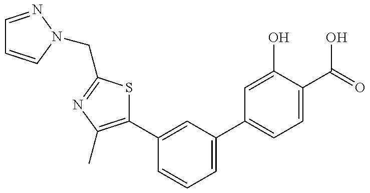 [SMILES: Cc1nc(Cn2cccn2)sc1-c1cccc(-c2ccc(C(=O)O)c(O)c2)c1] |
| 287 | 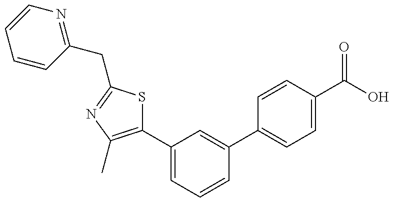 [SMILES: Cc1nc(Cc2ccccn2)sc1-c1cccc(-c2ccc(C(=O)O)cc2)c1] |
| 288 | 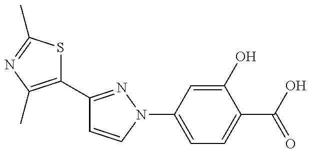 [SMILES: Cc1nc(C)c(-c2ccn(-c3ccc(C(=O)O)c(O)c3)n2)s1] |
| 289 | 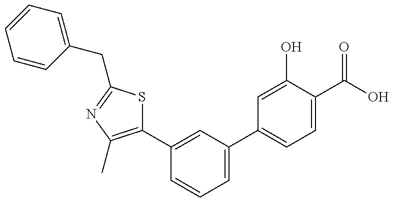 [SMILES: Cc1nc(Cc2ccccc2)sc1-c1cccc(-c2ccc(C(=O)O)c(O)c2)c1] |
| 290 | 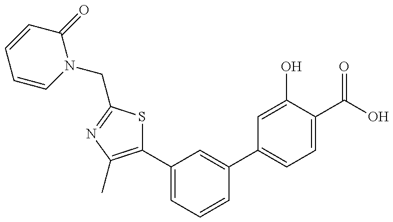 [SMILES: Cc1nc(Cn2ccccc2=O)sc1-c1cccc(-c2ccc(C(=O)O)c(O)c2)c1] |
| 291 | 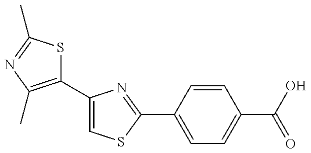 [SMILES: Cc1nc(C)c(-c2csc(-c3ccc(C(=O)O)cc3)n2)s1] |
| 292 | 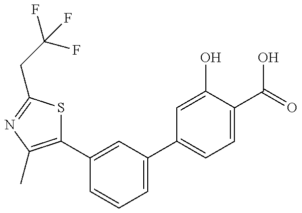 [SMILES: Cc1nc(CC(F)(F)F)sc1-c1cccc(-c2ccc(C(=O)O)c(O)c2)c1] |
| 293 | 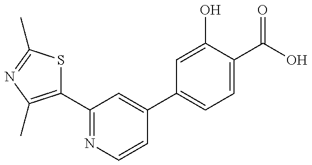 [SMILES: Cc1nc(C)c(-c2cc(-c3ccc(C(=O)O)c(O)c3)ccn2)s1] |
| 294 | 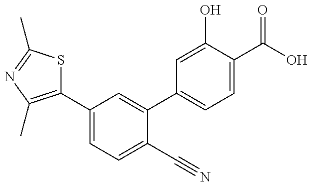 [SMILES: Cc1nc(C)c(-c2ccc(C#N)c(-c3ccc(C(=O)O)c(O)c3)c2)s1] |
| 295 | 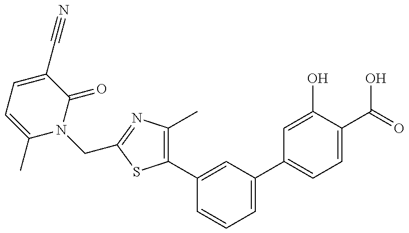 [SMILES: Cc1nc(Cn2c(C)ccc(C#N)c2=O)sc1-c1cccc(-c2ccc(C(=O)O)c(O)c2)c1] |
| 296 | 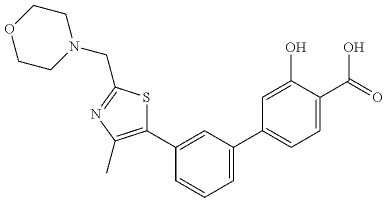 [SMILES: Cc1nc(CN2CCOCC2)sc1-c1cccc(-c2ccc(C(=O)O)c(O)c2)c1] |
| 297 | 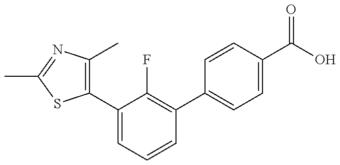 [SMILES: Cc1nc(C)c(-c2cccc(-c3ccc(C(=O)O)cc3)c2F)s1] |
| 298 | 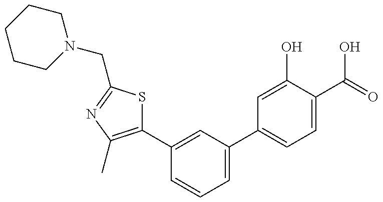 [SMILES: Cc1nc(CN2CCCCC2)sc1-c1cccc(-c2ccc(C(=O)O)c(O)c2)c1] |
| 299 | 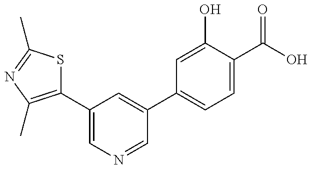 [SMILES: Cc1nc(C)c(-c2cncc(-c3ccc(C(=O)O)c(O)c3)c2)s1] |
| 300 | 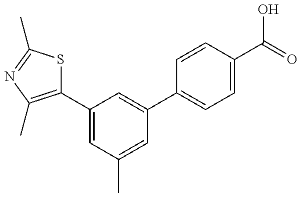 [SMILES: Cc1cc(-c2ccc(C(=O)O)cc2)cc(-c2sc(C)nc2C)c1] |
| 301 | 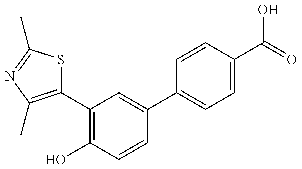 [SMILES: Cc1nc(C)c(-c2cc(-c3ccc(C(=O)O)cc3)ccc2O)s1] |
| 302 | 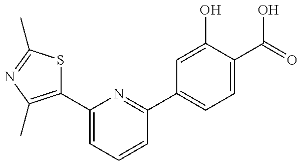 [SMILES: Cc1nc(C)c(-c2cccc(-c3ccc(C(=O)O)c(O)c3)n2)s1] |
| 303 | 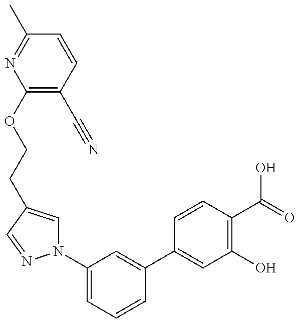 [SMILES: Cc1ccc(C#N)c(OCCc2cnn(-c3cccc(-c4ccc(C(=O)O)c(O)c4)c3)c2)n1] |
| 304 | 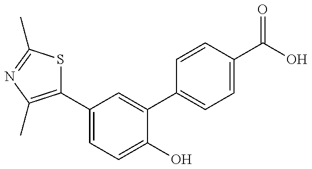 [SMILES: Cc1nc(C)c(-c2ccc(O)c(-c3ccc(C(=O)O)cc3)c2)s1] |
| 305 | 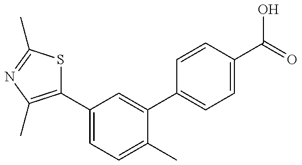 [SMILES: Cc1nc(C)c(-c2ccc(C)c(-c3ccc(C(=O)O)cc3)c2)s1] |
| 306 | 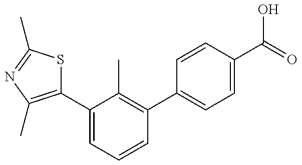 [SMILES: Cc1nc(C)c(-c2cccc(-c3ccc(C(=O)O)cc3)c2C)s1] |
| 307 | 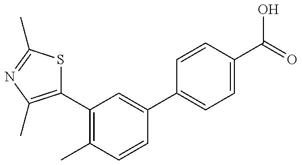 [SMILES: Cc1nc(C)c(-c2cc(-c3ccc(C(=O)O)cc3)ccc2C)s1] |
| 308 | 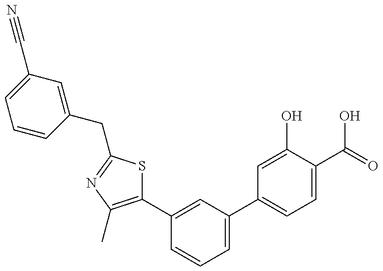 [SMILES: Cc1nc(Cc2cccc(C#N)c2)sc1-c1cccc(-c2ccc(C(=O)O)c(O)c2)c1] |
| 309 | 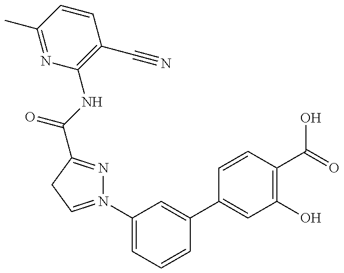 |
| 310 | 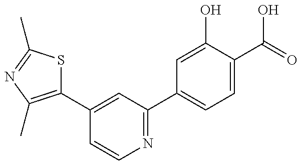 [SMILES: Cc1nc(C)c(-c2ccnc(-c3ccc(C(=O)O)c(O)c3)c2)s1] |
| 311 | 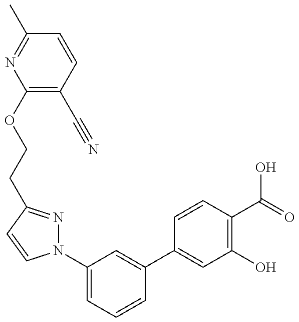 [SMILES: Cc1ccc(C#N)c(OCCc2ccn(-c3cccc(-c4ccc(C(=O)O)c(O)c4)c3)n2)n1] |
| 312 | 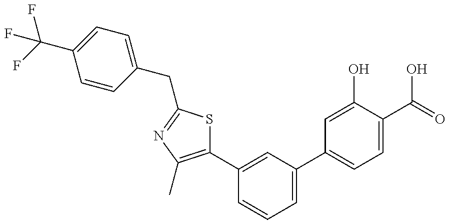 [SMILES: Cc1nc(Cc2ccc(C(F)(F)F)cc2)sc1-c1cccc(-c2ccc(C(=O)O)c(O)c2)c1] |
| 313 | 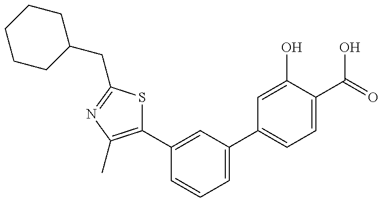 [SMILES: Cc1nc(CC2CCCCC2)sc1-c1cccc(-c2ccc(C(=O)O)c(O)c2)c1] |
| 314 | 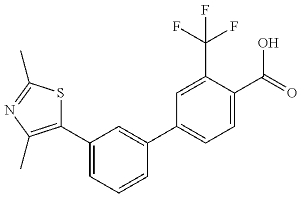 [SMILES: Cc1nc(C)c(-c2cccc(-c3ccc(C(=O)O)c(C(F)(F)F)c3)c2)s1] |
| 315 | 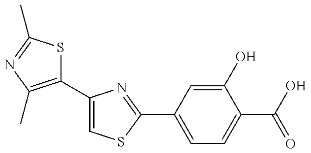 [SMILES: Cc1nc(C)c(-c2csc(-c3ccc(C(=O)O)c(O)c3)n2)s1] |
| 316 | 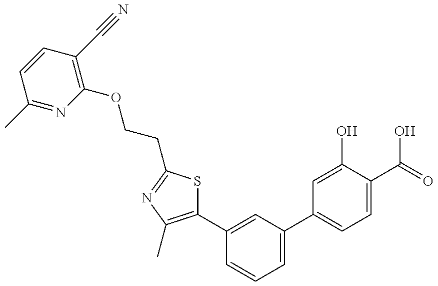 [SMILES: Cc1ccc(C#N)c(OCCc2nc(C)c(-c3cccc(-c4ccc(C(=O)O)c(O)c4)c3)s2)n1] |
| 317 | 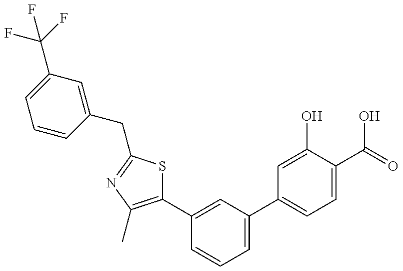 [SMILES: Cc1nc(Cc2cccc(C(F)(F)F)c2)sc1-c1cccc(-c2ccc(C(=O)O)c(O)c2)c1] |
| 318 | 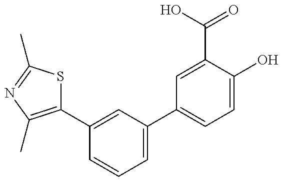 [SMILES: Cc1nc(C)c(-c2cccc(-c3ccc(O)c(C(=O)O)c3)c2)s1] |
| 319 | 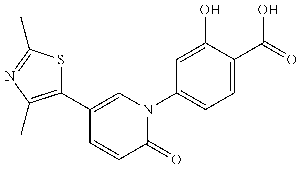 [SMILES: Cc1nc(C)c(-c2ccc(=O)n(-c3ccc(C(=O)O)c(O)c3)c2)s1] |
| 320 | 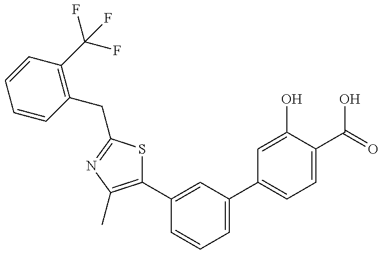 [SMILES: Cc1nc(Cc2ccccc2C(F)(F)F)sc1-c1cccc(-c2ccc(C(=O)O)c(O)c2)c1] |
| 321 | 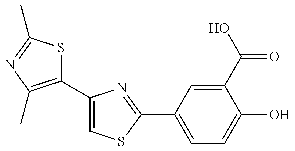 [SMILES: Cc1nc(C)c(-c2csc(-c3ccc(O)c(C(=O)O)c3)n2)s1] |
| 322 | 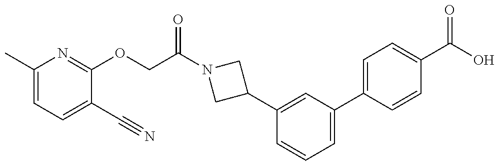 [SMILES: Cc1ccc(C#N)c(OCC(=O)N2CC(c3cccc(-c4ccc(C(=O)O)cc4)c3)C2)n1] |
| 323 | 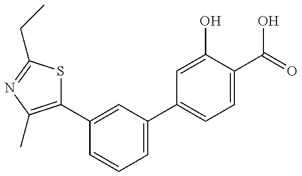 [SMILES: CCc1nc(C)c(-c2cccc(-c3ccc(C(=O)O)c(O)c3)c2)s1] |
| 324 | 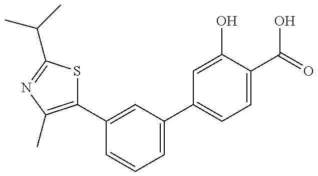 [SMILES: Cc1nc(C(C)C)sc1-c1cccc(-c2ccc(C(=O)O)c(O)c2)c1] |
| 325 | 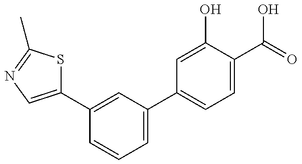 [SMILES: Cc1ncc(-c2cccc(-c3ccc(C(=O)O)c(O)c3)c2)s1] |
| 326 | 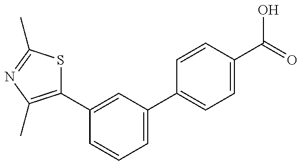 [SMILES: Cc1nc(C)c(-c2cccc(-c3ccc(C(=O)O)cc3)c2)s1] |
| 327 | 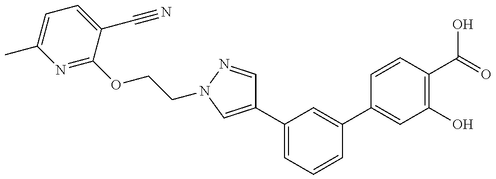 [SMILES: Cc1ccc(C#N)c(OCCn2cc(-c3cccc(-c4ccc(C(=O)O)c(O)c4)c3)cn2)n1] |
| 328 | 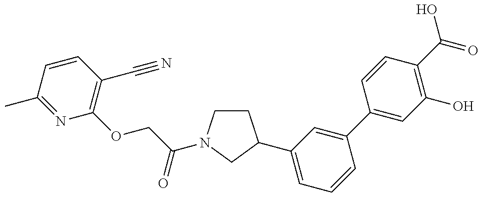 [SMILES: Cc1ccc(C#N)c(OCC(=O)N2CCC(c3cccc(-c4ccc(C(=O)O)c(O)c4)c3)C2)n1] |
| 329 | 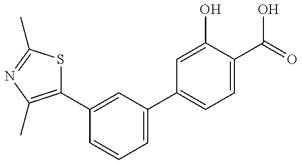 [SMILES: Cc1nc(C)c(-c2cccc(-c3ccc(C(=O)O)c(O)c3)c2)s1] |
| 330 | 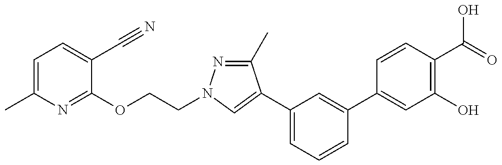 [SMILES: Cc1ccc(C#N)c(OCCn2cc(-c3cccc(-c4ccc(C(=O)O)c(O)c4)c3)c(C)n2)n1] |
| 331 | 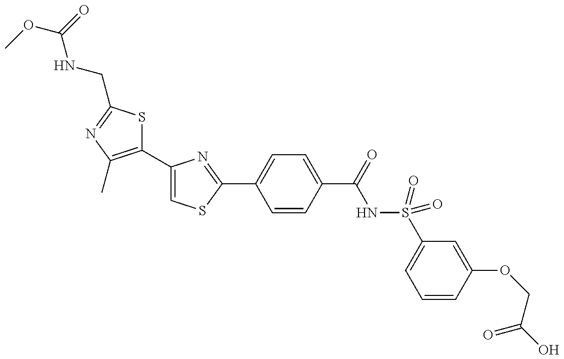 [SMILES: COC(=O)NCc1nc(C)c(-c2csc(-c3ccc(C(=O)NS(=O)(=O)c4cccc(OCC(=O)O)c4)cc3)n2)s1] |
| 332 | 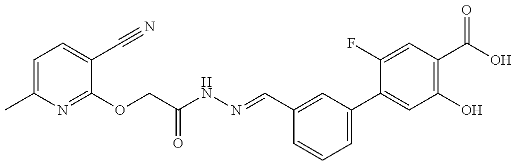 [SMILES: Cc1ccc(C#N)c(OCC(=O)N/N=C/c2cccc(-c3cc(O)c(C(=O)O)cc3F)c2)n1] |
| 333 | 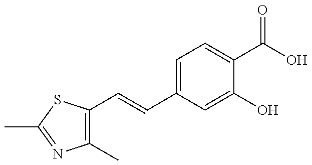 [SMILES: Cc1nc(C)c(/C=C/c2ccc(C(=O)O)c(O)c2)s1] |
| 334 | 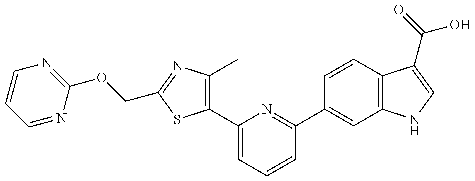 [SMILES: Cc1nc(COc2ncccn2)sc1-c1cccc(-c2ccc3c(C(=O)O)c[nH]c3c2)n1] |
| 335 | 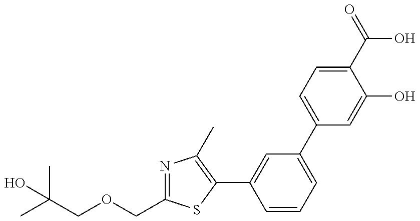 [SMILES: Cc1nc(COCC(C)(C)O)sc1-c1cccc(-c2ccc(C(=O)O)c(O)c2)c1] |
| 336 | 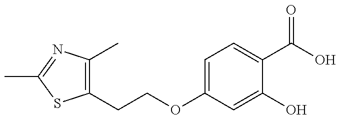 [SMILES: Cc1nc(C)c(CCOc2ccc(C(=O)O)c(O)c2)s1] |
| 337 | 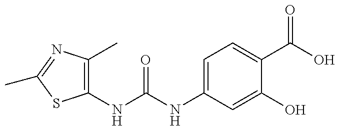 [SMILES: Cc1nc(C)c(NC(=O)Nc2ccc(C(=O)O)c(O)c2)s1] |
| 338 | 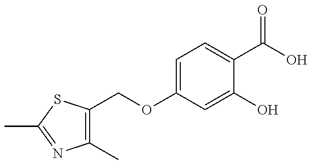 [SMILES: Cc1nc(C)c(COc2ccc(C(=O)O)c(O)c2)s1] |
| 339 | 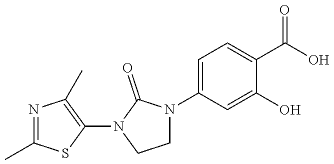 [SMILES: Cc1nc(C)c(N2CCN(c3ccc(C(=O)O)c(O)c3)C2=O)s1] |
| 340 | 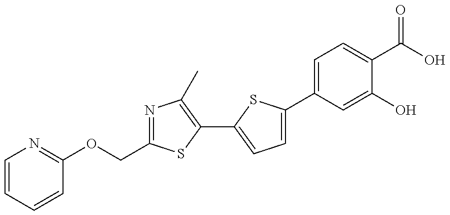 [SMILES: Cc1nc(COc2ccccn2)sc1-c1ccc(-c2ccc(C(=O)O)c(O)c2)s1] |
| 341 | 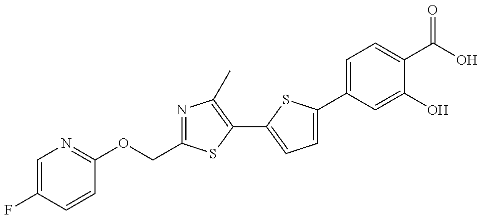 [SMILES: Cc1nc(COc2ccc(F)cn2)sc1-c1ccc(-c2ccc(C(=O)O)c(O)c2)s1] |
| 342 | 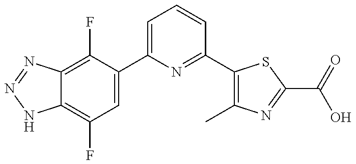 [SMILES: Cc1nc(C(=O)O)sc1-c1cccc(-c2cc(F)c3[nH]nnc3c2F)n1] |

or a pharmaceutically acceptable salt thereof.

### Methods of Synthesis

Compounds of Formula (I) or any variation thereof may be prepared according to the general reactions shown in Schemes 1, 2, and 3.

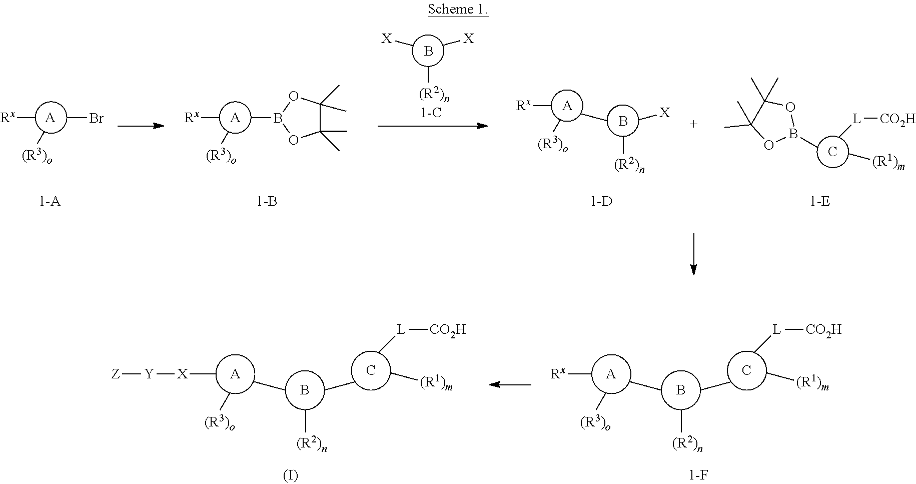

Scheme 1 shows an exemplary route for preparing compounds of Formula (I), wherein R^{X} can be a group useful for conversion to a desired Z—Y—X group (such as shown in the non-limiting examples, Schemes 2 and 3) and the remaining variables are as described herein. In some embodiments, compound 1-A can be derivatized to form compound 1-B, which can subsequently be coupled (e.g. under Suzuki coupling conditions) with an appropriate dihalide, compound 1-C(wherein X is Cl or Br), to form compound 1-D. Compound 1-F can be achieved from compound 1-D with an appropriate boronic acid ester, compound 1-E, and metal catalyst. R^{X} can subsequently be derivatized to form the desired Z—Y—X group according to methods known in the art. In some embodiments, ring A, B, and C can be connected in any order (e.g., ring A and ring B, followed by ring C as shown in Scheme 1; or ring B and ring C, followed by ring A) according to similar methods as described herein.

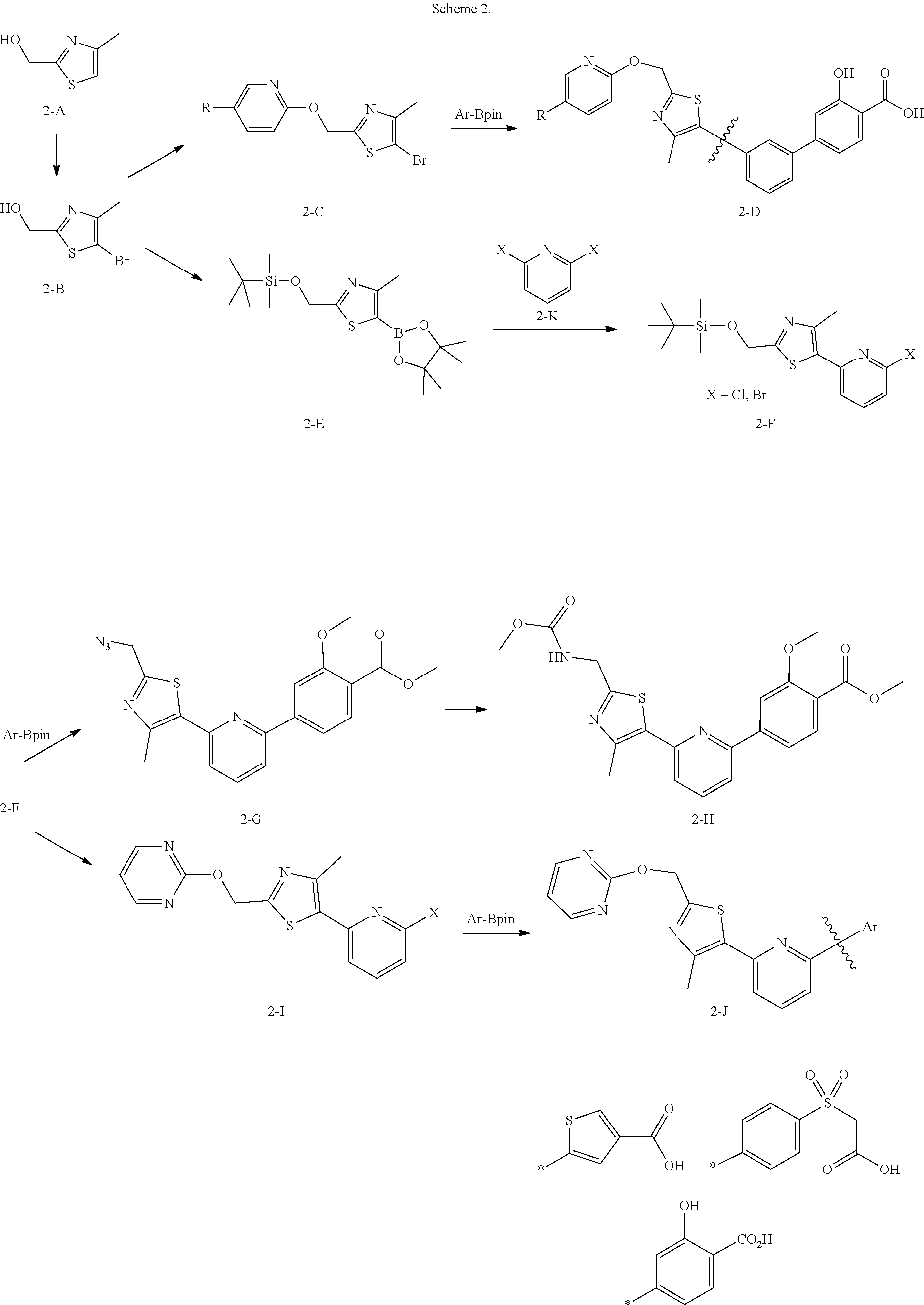

Scheme 2 shows several routes for preparing compounds of Formula (I) wherein Ring B is a phenylene group or a heteroarylene group such as pyridinylene. In some embodiments, compound 2-B of Scheme 2 is commercially available or may be obtained by brominating compound 2-A of Scheme 2. In some embodiments, compound 2-C of Scheme 2 (wherein R corresponds to R¹⁰ of a compound of Formula (I) as described herein) may be obtained via standard coupling conditions, such as standard Mitsunobu conditions (e.g., DEAD and PPH₃). Compound 2-D (i.e. an exemplary compound of Formula (I)) can be achieved via Suzuki coupling conditions (e.g. with the appropriate boronic ester and metal catalyst, such as a palladium catalyst). In some embodiments, compound 2-B can be derivatized to form compound 2-E, which can subsequently be coupled (e.g. under Suzuki coupling conditions) with an appropriate pyridinyl dihalide, compound 2-K (wherein X is Cl or Br), to form compound 2-F. Compound 2-G can be achieved with an appropriate boronic acid ester and metal catalyst and introduction of the azido group under standard conditions; standard coupling conditions subsequently achieves compound 2-H. In some embodiments, compound 2-F can obtained via, for example, standard Mitsunobu conditions. Coupling conditions, such as Suzuki conditions, can then be used to achieve compound 2-J (wherein Ar of compound I-J can be the substituents as shown in Scheme 2).

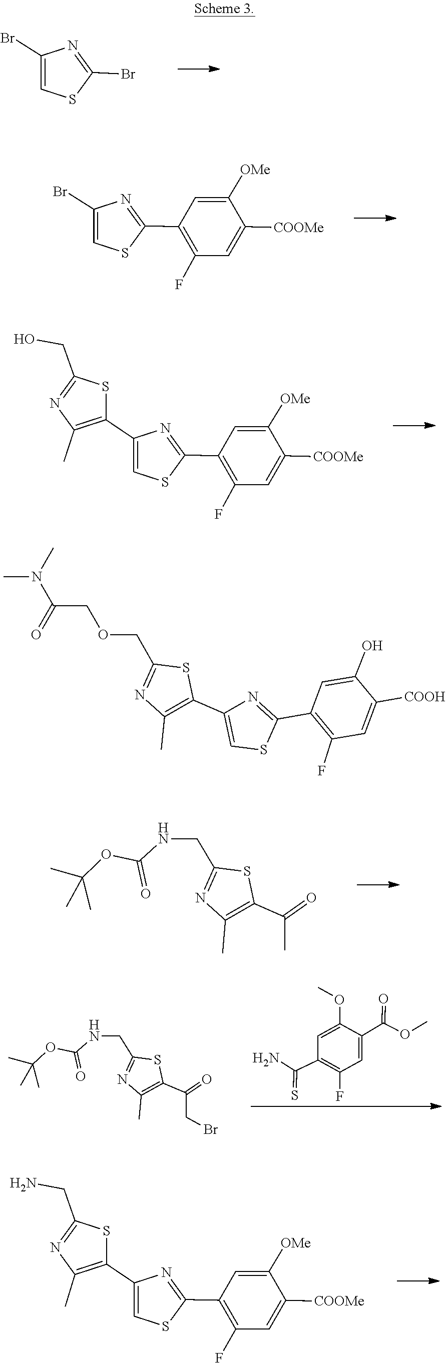

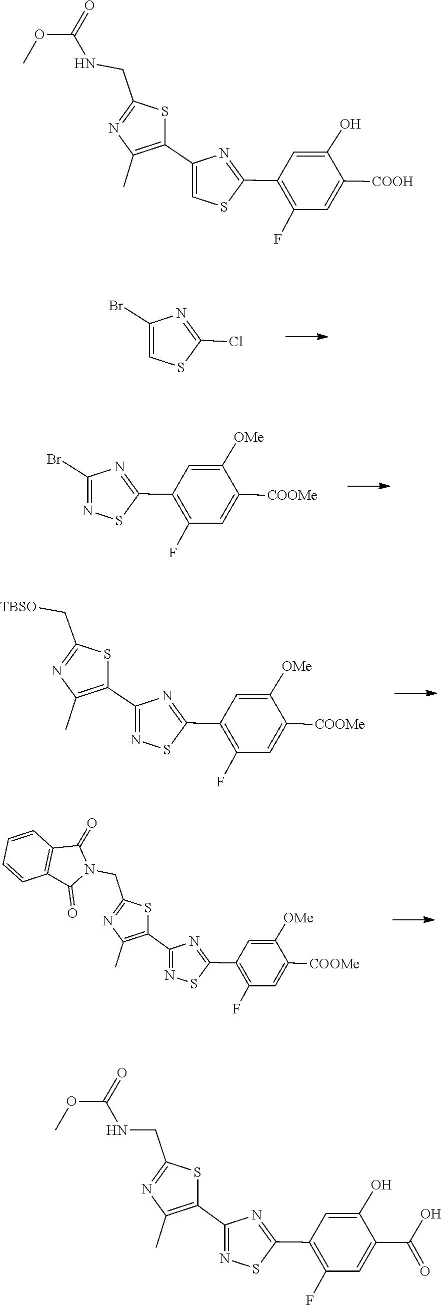 [SMILES: COC(=O)NCc1nc(C)c(-c2csc(-c3cc(O)c(C(=O)O)cc3F)n2)s1 ; Clc1nc(Br)cs1 ; COC(=O)c1cc(F)c(-c2nc(Br)ns2)cc1OC ; COC(=O)c1cc(F)c(-c2nc(-c3sc(CO[Si](C)(C)C(C)(C)C)nc3C)ns2)cc1OC ; COC(=O)c1cc(F)c(-c2nc(-c3sc(CN4C(=O)c5ccccc5C4=O)nc3C)ns2)cc1OC ; COC(=O)NCc1nc(C)c(-c2nsc(-c3cc(O)c(C(=O)O)cc3F)n2)s1]

Scheme 3 shows several routes for preparing compounds of Formula (I) wherein Ring B is a heteroarylene group such as thiazolylene and thiadiazolylene as described in the Examples.

It is understood that the synthetic processes disclosed herein may be modified to arrive at various compounds of the present disclosure by selection of appropriate reagents and starting materials. In some embodiments, the synthetic processes disclosed herein may be modified to arrive at compounds of Formula (I) having other substituents as those shown in Scheme 1, Scheme 2, and Scheme 3 by selection of appropriate reagents and starting materials.

All compounds of Formula (I) or any variation thereof as described herein which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

### Pharmaceutical Compositions and Formulations

In another aspect, provided herein are pharmaceutical compositions of any of the compounds detailed herein. Thus, the present disclosure includes pharmaceutical compositions comprising a compound disclosed herein, or a pharmaceutically acceptable salt thereof, and a pharmaceutically acceptable carrier or excipient. Pharmaceutical compositions according to the disclosure may take a form suitable for oral, buccal, parenteral, nasal, topical, or rectal administration, or a form suitable for administration by inhalation. Pharmaceutical compositions of the present disclosure comprise a compound of Formula (I), or a pharmaceutically acceptable salt thereof, and a pharmaceutically acceptable carrier, diluent, or excipient.

A compound described herein can be used in the preparation of a composition, such as a pharmaceutical composition, by combining the compound as an active ingredient with a pharmaceutically acceptable excipient. Some examples of materials which can serve as pharmaceutically acceptable excipients include: sugars, such as lactose, glucose and sucrose; starches, such as corn starch and potato starch; cellulose and its derivatives, such as sodium carboxymethyl cellulose, ethyl cellulose and cellulose acetate; surfactants, such as polysorbate 80 (i.e., Tween 80); powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Pharmaceutical formulations may be prepared by known pharmaceutical methods. Suitable formulations can be found in, for example, Remington: The Science and Practice of Pharmacy, Lippincott Williams & Wilkins, 21^{st} ed. (2005), which is incorporated herein by reference.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol and the like; and metal chelating agents, such as citric acid, ethylenediamine tetraacetic acid (EDTA), sorbitol, tartaric acid, phosphoric acid and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, this amount will range from about 1% to about 99% of active ingredient, or from about 5% to about 70%, or from about 10% to about 30%.

In certain embodiments, a formulation of the present disclosure comprises an excipient selected from the group consisting of cyclodextrins, liposomes, micelle forming agents, e.g., bile acids and polymeric carriers, e.g., polyesters and polyanhydrides; and a compound of the present disclosure. In certain embodiments, an aforementioned formulation renders orally bioavailable a compound of the present disclosure.

Formulations of the disclosure suitable for oral administration may be in the form of capsules, cachets, pills, tablets, lozenges (using a flavored basis, usually sucrose and acacia or tragacanth), powders, granules or as a solution or a suspension in an aqueous or non-aqueous liquid or as an oil-in-water or water-in-oil liquid emulsion or as an elixir or syrup or as pastilles (using an inert base, such as gelatin and glycerin or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules and the like), the active ingredient is mixed with one or more pharmaceutically-acceptable carriers, such as sodium citrate or dicalcium phosphate and/or any of the following: fillers or extenders, such as starches, lactose, sucrose, glucose, mannitol and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates and sodium carbonate; solution retarding agents, such as paraffin; absorption accelerators, such as quaternary ammonium compounds; wetting agents, such as, for example, cetyl alcohol, glycerol monostearate and non-ionic surfactants; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter or by incorporating sterilizing agents in the form of sterile solid compositions that can be dissolved in sterile water or some other sterile injectable medium immediately before use. These compositions may also optionally contain opacifying agents and may be of a composition that they release the active ingredient(s) only or, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, and fatty acid esters of sorbitan and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth and mixtures thereof.

Formulations of the pharmaceutical compositions of the disclosure for rectal or vaginal administration may be presented as a suppository, which may be prepared by mixing one or more compounds of the disclosure with one or more suitable nonirritating excipients or carriers comprising, for example, cocoa butter, polyethylene glycol, a suppository wax or a salicylate and which is solid at room temperature, but liquid at body temperature and, therefore, will melt in the rectum or vaginal cavity and release the active compound.

Dosage forms for the topical or transdermal administration of a compound of this disclosure include powders, sprays, ointments, pastes, creams, lotions, gels, solutions, patches and inhalants. The active compound may be mixed under sterile conditions with a pharmaceutically-acceptable carrier and with any preservatives, buffers or propellants which may be required.

The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound of this disclosure, excipients such as lactose, talc, silicic acid, aluminum hydroxide, calcium silicates, and polyamide powder or mixtures of these substances. Sprays can additionally contain customary propellants, such as chlorofluorohydrocarbons and volatile unsubstituted hydrocarbons, such as butane and propane.

Pharmaceutical compositions of this disclosure suitable for parenteral administration comprise one or more compounds of the disclosure in combination with one or more pharmaceutically-acceptable sterile isotonic aqueous or nonaqueous solutions, dispersions, suspensions or emulsions or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the disclosure include water, ethanol, polyols (such as glycerol, propylene glycol, polyethylene glycol and the like) and suitable mixtures thereof, vegetable oils, such as olive oil and injectable organic esters, such as ethyl oleate. Proper fluidity can be maintained, for example, by the use of coating materials, such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenyl sorbic acid and the like. It may also be desirable to include isotonic agents, such as sugars, sodium chloride and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

### Methods of Use/Treatments

Compounds and compositions detailed herein, such as a pharmaceutical composition containing a compound of any formula provided herein, or a pharmaceutically acceptable salt thereof, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In one aspect, provided herein is a method of modulating bis-phosphoglycerate mutase (BPGM) comprising contacting either an effective amount of a compound described herein, or a pharmaceutically acceptable salt thereof, or an effective amount of a pharmaceutical composition provided herein, with the BPGM. In certain embodiments, the compounds of the present disclosure are allosteric modulators of BPGM that affect both the synthase and phosphatase functions of the enzyme.

In another aspect, provided herein is a method of treating sickle cell disease in a subject in need thereof, comprising administering to the subject a therapeutically effective amount of a compound described here, or a pharmaceutically acceptable salt thereof, or a therapeutically effective amount of a pharmaceutical composition described herein. In some embodiments, the sickle cell disease is HbSS, also referred to as sickle cell anemia. In some embodiments, the sickle cell disease is HbSC. In some embodiments, the sickle cell disease is HbS beta thalassemia. In some embodiments, the sickle cell disease is HbSD, HbSE, or HbSO.

In some embodiments, the subject is a mammal. In some embodiments, the subject is a primate, dog, cat, rabbit, or rodent. In some embodiments, the subject is a primate. In some embodiments, the subject is a human. In some embodiments, the human is at least about or is about any of 18, 21, 30, 50, 60, 65, 70, 80, or 85 years old. In some embodiments, the human is a child. In some embodiments, the human is less than about or about any of 21, 18, 15, 10, 5, 4, 3, 2, or 1 years old.

### Dosing and Method of Administration

The phrases “parenteral administration” and “administered parenterally” as used herein mean modes of administration other than enteral and topical administration, usually by injection and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present disclosure, or the pharmaceutical compositions of the present disclosure, are formulated into pharmaceutically acceptable dosage forms by conventional methods known to those of skill in the art.

Actual dosage levels of the active ingredients in the pharmaceutical compositions of this disclosure may be varied so as to obtain an amount of the active ingredient that is effective to achieve the desired therapeutic response for a particular patient, composition and mode of administration, without being toxic to the patient.

The selected dosage level will depend upon a variety of factors including the activity of the particular compound of the present disclosure employed or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated and like factors well known in the medical arts. A daily, weekly or monthly dosage (or other time interval) can be used.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required to achieve the desired therapeutic effect and then gradually increasing the dosage until the desired effect is achieved.

In general, a suitable daily dose of a compound of the disclosure will be that amount of the compound that is the lowest dose effective to produce a therapeutic effect (e.g., inhibit necrosis). Such an effective dose will generally depend upon the factors described above. Generally, doses of the compounds of this disclosure for a patient, when used for the indicated effects, will range from about 0.0001 to about 100 mg per kg of body weight per day. In some embodiments, the daily dosage will range from 0.001 to 50 mg of compound per kg of body weight, and in some embodiments, from 0.01 to 10 mg of compound per kg of body weight.

If desired, the effective daily dose of the active compound may be administered as two, three, four, five, six or more sub-doses administered separately at appropriate intervals throughout the day, optionally, in unit dosage forms.

In certain embodiments, the present disclosure relates to compounds for modulating BPGM, wherein the compounds are represented by Formula (I). In certain embodiments, the compounds of the present disclosure are allosteric modulators of BPGM that affect both the synthase and phosphatase functions of the enzyme. In any event, the compounds of the present disclosure, in some embodiments, exert their effect on modulating BPGM at a concentration less than about 50 micromolar, or at a concentration less than about 10 micromolar or at a concentration less than 1 micromolar.

When the compounds of the present disclosure are administered as pharmaceuticals, to humans and animals, they can be given per se or as a pharmaceutical composition containing, for example, 0.1% to 99.5% (or, 0.5% to 90%) of active ingredient in combination with a pharmaceutically acceptable carrier.

The compounds of the present application or the compositions thereof may be administered once, twice, three or four times daily, using any suitable mode described above. Also, administration or treatment with the compounds may be continued for a number of days; for example, commonly treatment would continue for at least 7 days, 14 days or 28 days, for one cycle of treatment. Treatment cycles are well known and are frequently alternated with resting periods of about 1 to 28 days, commonly about 7 days or about 14 days, between cycles. The treatment cycles, in certain embodiments, may also be continuous.

When administered orally, the total daily dosage for a human subject may be between 1 mg and 1,000 mg, between about 1,000-2,000 mg/day, between about 10-500 mg/day, between about 50-300 mg/day, between about 75-200 mg/day or between about 100-150 mg/day.

The daily dosage may also be described as a total amount of a compound described herein administered per dose or per day. Daily dosage of a compound may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

In certain embodiments, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week or once per week.

In certain embodiments, a compound or pharmaceutical preparation is administered orally. In certain embodiments, the compound or pharmaceutical preparation is administered intravenously. Alternative routes of administration include sublingual, intramuscular and transdermal administrations.

The preparations of the present disclosure may be given orally, parenterally, topically or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. In certain embodiments, the administration is oral.

### Kits/Articles of Manufacture0

Also provided herein are kits that include a compound of the disclosure, or a pharmaceutically acceptable salt thereof, and suitable packaging. In certain embodiments, a kit further includes instructions for use. In one aspect, a kit includes a compound of the disclosure, or a pharmaceutically acceptable salt thereof, and a label and/or instructions for use of the compounds in the treatment of the indications, including the diseases or conditions, described herein.

Provided herein are also articles of manufacture that include a compound described herein, or a pharmaceutically acceptable salt thereof, in a suitable container. The container may be a vial, jar, ampoule, preloaded syringe and intravenous bag.

The kit can also contain instructions for using the compounds according to the disclosure. The kit can be compartmentalized to receive the containers in close confinement. As used herein, a kit such as a compartmentalized kit includes any kit in which compounds or agents are contained in separate containers. Illustrative examples of such containers include, but are not limited to, small glass containers, plastic containers or strips of plastic or paper. In some embodiments, the types of containers allow the skilled worker to efficiently transfer reagents from one compartment to another compartment such that the samples and reagents are not cross-contaminated and the agents or solutions of each container can be added in a quantitative fashion from one compartment to another. Such containers include, but are not limited to, a container that will accept a compound or combination of compounds and/or other agents of the disclosure. One or more compounds or agents can be provided as a powder (e.g. lyophilized powder) or precipitate. Such compound(s) can be resuspended prior to administration in a solution that may be provided as part of the kit or separately available. A kit can contain compounds or agents in other forms such as liquids, gels, solids, as described herein. Different compounds and/or agents may be provided in different forms in a single kit.

### Examples

The examples and preparations provided below further illustrate and exemplify the compounds of the present disclosure and methods for testing such compounds. It is to be understood that the scope of the present disclosure is not limited in any way by the scope of the following examples.

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be performed by modifications apparent to those skilled in the art, for example by appropriately protecting interfering groups, by utilizing other suitable reagents known in the art other than those described, or by making routine modification of reaction conditions, reagents, and starting materials. Alternatively, other reactions disclosed herein or known in the art will be recognized as having applicability for preparing other compounds of the present disclosure.

The following abbreviations may be relevant for the application.

### Abbreviations

ACN: acetonitrile
DCM: dichloromethane
DEAD: diethyl azodicarboxylate
DIEA: N,N-diisopropylethylamine
DMF: N,N-dimethylformamide
DMSO: dimethylsulfoxide
ES-MS: electrospray ionization mass spectrometry
EtOAc: ethyl acetate
EtOH: ethanol
h: hour(s)
HATU: (1-[bis(dimethylamino)methylene]-1H-1,2,3-triazolo[4,5-b]pyridinium 3-oxide
hexafluorophosphate
HPLC: high performance liquid chromatography
PrOH: isopropanol
MeOH: methanol
MsCl: methanesulfonyl chloride
NMR: nuclear magnetic resonance
Pd(dppf)Cl₂: [1,1′-bis(diphenylphosphino)ferrocene]dichloropalladium(II)
Pd(OAc)₂: palladium(II) acetate
PE: petroleum ether
PPh₃: triphenylphosphine
rt or RT: room temperature
THF: tetrahydrofuran
TBAF: tetra-n-butylammonium fluoride
TBAI: tetrabutylammonium iodide
TLC: thin layer chromatography

### Synthetic Examples

### Example S1. Preparation of 4-[3-[2-[(5-fluoro-2-pyridyl)oxymethyl]-4-methyl-thiazol-5-yl]phenyl]-2-hydroxy-benzoic Acid (Compound 1)

### Step 1: Synthesis of 5-bromo-2-[(5-fluoro-2-pyridyl)oxymethyl]-4-methyl-thiazole (1a)

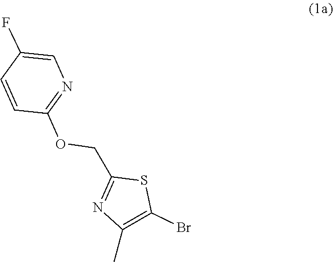 [SMILES: Cc1nc(COc2ccc(F)cn2)sc1Br]

To a solution of (5-bromo-4-methyl-thiazol-2-yl)methanol (1 g, 4.33 mmol, 1 eq) and 5-fluoropyridin-2-ol (978.30 mg, 8.65 mmol, 2 eq) in THF (20 mL) were added PPh₃ (2.27 g, 8.65 mmol, 2 eq) and DEAD (1.51 g, 8.65 mmol, 1.57 mL, 2 eq) at 0° C. After 12 h at 25° C., the mixture was concentrated under reduced pressure, diluted with water (30 mL), and extracted with EtOAc (30 mL×3). The combined organic layers were washed with brine (30 mL), dried over anhydrous Na₂SO₄, filtered and concentrated to give the residue, which was purified with flash silica gel chromatography (ISCO®; 20 g SepaFlash® Silica Flash Column, Eluent of 0˜5% Ethyl acetate/Petroleum ether gradient @ 30 mL/min) to provide 5-bromo-2-[(5-fluoro-2-pyridyl)oxymethyl]-4-methyl-thiazole (1a) (300 mg, 890.65 umol, 20.6% yield, 90% purity) as a solid. ¹H NMR (400 MHz, CDCl₃) δ ppm 8.02 (d, J=2.8 Hz, 1H), 7.41-7.37 (m, 1H), 6.82 (dd, J=3.6, 9.2 Hz, 1H), 5.53 (s, 2H), 2.41 (s, 3H).

Step 2: Synthesis of 4-[3-[2-[(5-fluoro-2-pyridyl)oxymethyl]-4-methyl-thiazol-5-yl]phenyl]-2-hydroxy-benzoic acid (1). To a solution of 5-bromo-2-[(5-fluoro-2-pyridyl)oxymethyl]-4-methyl-thiazole (1a) (150 mg, 445.32 μmol, 1 eq) in dioxane (2 mL) and H₂O (0.2 mL) were added 2-hydroxy-4-[3-(4,4,5,5-tetramethyl-1,3,2-dioxaborolan-2-yl)phenyl]benzoic acid (196.94 mg, 578.92 μmol, 1.3 eq), Pd(dppf)Cl₂ (32.58 mg, 44.53 μmol, 0.1 eq), and Cs₂CO₃ (435.29 mg, 1.34 mmol, 3 eq). After 3 h at 80° C. under N₂ atmosphere, the mixture was concentrated, diluted with water (30 mL), and extracted with EtOAc (30 mL×3). The combined organic layer was washed with brine (30 mL), dried over anhydrous Na₂SO₄, filtered, and concentrated under reduced pressure to give a residue, which was purified with reversed-phase HPLC (0.1% NH₃·H₂O) and then further purified with prep-HPLC (column: YMC-Actus Triart C18 150*30 mm*5 um; mobile phase: [water (0.05% ammonia hydroxide v/v)-ACN]; B %: 30%-50%, 10 min) to provide 4-[3-[2-[(5-fluoro-2-pyridyl)oxymethyl]-4-methyl-thiazol-5-yl]phenyl]-2-hydroxy-benzoic acid (1) (31.4 mg, 71.70 μmol, 16.1% yield, 99.66% purity) as a solid. ¹H NMR (400 MHz, DMSO-d₆) δ ppm 8.22 (d, J=3.2 Hz, 1H), 7.80-7.71 (m, 2H), 7.66-7.62 (m, 2H), 7.52 (t, J=8.0 Hz, 1H), 7.46-7.42 (m, 1H), 7.03 (dd, J=3.6, 9.0 Hz, 1H), 6.93-6.90 (m, 2H), 5.59 (s, 2H), 2.47 (s, 3H); HPLC (Purity: 99.66%); ES-MS m/z 437.2 [M+H]+.

### Example S2. Preparation of 2-[4-[6-[4-methyl-2-(pyrimidin-2-yloxymethyl)thiazol-5-yl]-2-pyridyl]phenyl]sulfonylacetic Acid (Compound 2)

### Step 1: Synthesis of 4-bromobenzenesulfonohydrazide (2a)

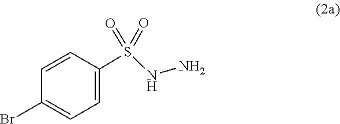 [SMILES: NNS(=O)(=O)c1ccc(Br)cc1]

To a mixture of 4-bromobenzenesulfonyl chloride (1 g, 3.91 mmol, 1 eq) in THF (20 mL) was added hydrazine hydrate (599.28 mg, 10.18 mmol, 85% purity, 2.6 eq) at 5° C. After 30 min at 5° C., the mixture was partitioned between EtOAc (30 mL) and water (30 mL). The organic layer was separated, washed with brine (20 mL×3), dried over Na₂SO₄, filtered, and concentrated to give 4-bromobenzenesulfonohydrazide (2a) (1 g, 3.58 mmol, 91.6% yield, 90% purity) as a solid. ¹H NMR (400 MHz, DMSO-d₆) δ ppm 8.49 (br s, 1H), 7.83 (d, J=8.4 Hz, 2H), 7.72 (d, J=8.4 Hz, 2H), 4.20 (d, J=3.2 Hz, 2H).

### Step 2: Synthesis of ethyl 2-(4-bromophenyl)sulfonylacetate (2b)

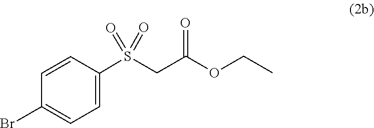 [SMILES: CCOC(=O)CS(=O)(=O)c1ccc(Br)cc1]

To a mixture of 4-bromobenzenesulfonohydrazide (2a) (800.00 mg, 2.87 mmol, 1 eq) and ethyl 2-diazoacetate (654.35 mg, 5.73 mmol, 2 eq) in isopropanol (5 mL) was added TBAI (211.82 mg, 573.48 mol, 0.2 eq). After 12 h at 80° C., the mixture was concentrated under reduced pressure to give the residue, which was purified with flash silica gel chromatography (ISCO®; 40 g SepaFlash® Silica Flash Column, Eluent of 0˜20% Ethyl acetate/Petroleum ether gradient @ 35 mL/min) to provide ethyl 2-(4-bromophenyl)sulfonylacetate (2b) (0.8 g, 2.34 mmol, 81.8% yield, 90% purity) as a solid. ¹H NMR (400 MHz, DMSO-d₆) δ ppm 7.93-7.90 (m, 2H), 7.87-7.85 (m, 2H), 4.74-4.61 (m, 2H), 4.10-4.04 (m, 2H), 1.08 (t, J=7.2 Hz, 3H).

### Step 3: Synthesis of ethyl 2-[4-(4,4,5,5-tetramethyl-1,3,2-dioxaborolan-2-yl)phenyl]sulfonylacetate (2c)

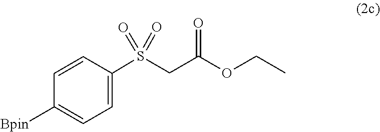

A mixture of ethyl 2-(4-bromophenyl)sulfonylacetate (2b) (0.6 g, 1.76 mmol, 1 eq), Pd(dppf)Cl₂ (64.32 mg, 87.90 μmol, 0.05 eq), KOAc (431.33 mg, 4.40 mmol, 2.5 eq), 4,4,5,5-tetramethyl-2-(4,4,5,5-tetramethyl-1,3,2-dioxaborolan-2-yl)-1,3,2-dioxaborolane (892.86 mg, 3.52 mmol, 2 eq) in dioxane (20 mL) was degassed and purged with N₂ for 3 times, and then the mixture was stirred at 85° C. for 12 h. The mixture was concentrated and then water (20 mL) was added. The mixture was extracted with EtOAc (20 mL×3). The combined organic layers were washed with brine (20 mL), dried over Na₂SO₄, filtered and concentrated to afford the residue, which was purified with flash silica gel chromatography (ISCO®; 40 g SepaFlash® Silica Flash Column, Eluent of 0˜20% Ethyl acetate/Petroleum ether gradient @ 35 mL/min) to give ethyl 2-[4-(4,4,5,5-tetramethyl-1,3,2-dioxaborolan-2-yl)phenyl]sulfonylacetate (2c) (0.6 g, 1.52 mmol, 86.7% yield, 90% purity) as a solid. ¹H NMR (400 MHz, DMSO-d₆) δ ppm 7.96-7.91 (m, 4H), 4.75-4.55 (m, 2H), 4.05-4.01 (m, 2H), 1.33 (s, 12H), 1.07-1.04 (m, 3H).

### Step 4: Synthesis of ethyl 2-[4-[6-[4-methyl-2-(pyrimidin-2-yloxymethyl)thiazol-5-yl]-2-pyridyl]phenyl]sulfonylacetate (2d)

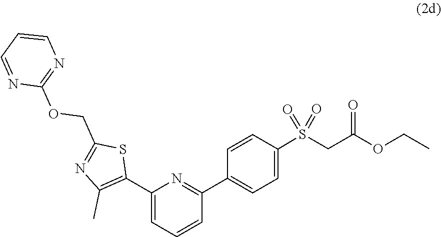 [SMILES: CCOC(=O)CS(=O)(=O)c1ccc(-c2cccc(-c3sc(COc4ncccn4)nc3C)n2)cc1]

A mixture of 5-(6-chloro-2-pyridyl)-4-methyl-2-(pyrimidin-2-yloxymethyl)thiazole (100 mg, 282.33 mol, 1 eq), Pd(dppf)Cl₂ (20.66 mg, 28.23 μmol, 0.1 eq), Cs₂CO₃ (229.97 mg, 705.81 μmol, 2.5 eq), ethyl 2-[4-(4,4,5,5-tetramethyl-1,3,2-dioxaborolan-2-yl)phenyl]sulfonylacetate (2c) (122.23 mg, 310.56 mol, 1.1 eq) in dioxane (12 mL) was degassed and purged with N₂ for 3 times, and then the mixture was stirred at 80° C. for 1 h. The mixture was concentrated to give the residue, which was purified with flash silica gel chromatography (ISCO®; 20 g SepaFlash® Silica Flash Column, Eluent of 0˜10% MeOH/DCM gradient @ 35 mL/min) to afford ethyl 2-[4-[6-[4-methyl-2-(pyrimidin-2-yloxymethyl)thiazol-5-yl]-2-pyridyl]phenyl]sulfonylacetate (2d) (100 mg, 176.27 μmol, 62.4% yield, 90% purity) as a solid. ¹H NMR (400 MHz, DMSO-d₆) δ ppm 8.70 (d, J=4.8 Hz, 2H), 8.37 (s, 1H), 8.13-8.02 (m, 4H), 7.60-7.52 (m, 2H), 7.25 (t, J=4.8 Hz, 1H), 5.68 (s, 2H), 4.75-4.47 (m, 2H), 4.08-4.02 (m, 2H), 2.72 (s, 3H), 1.09 (s, 3H).

Step 5: Synthesis of 2-[4-[6-[4-methyl-2-(pyrimidin-2-yloxymethyl)thiazol-5-yl]-2-pyridyl]phenyl]sulfonylacetic acid (2). A mixture of ethyl 2-[4-[6-[4-methyl-2-(pyrimidin-2-yloxymethyl)thiazol-5-yl]-2-pyridyl]phenyl]sulfonylacetate (2d) (90 mg, 158.64 μmol, 1 eq), LiOH·H₂O (33.28 mg, 793.21 μmol, 5 eq) in THF (4 mL) and H₂O (1 mL) was stirred at 20° C. for 2 h. The mixture was concentrated, diluted with water (5 mL), and filtered after its pH was adjusted into 6-7 with 2 N HCl. The filtrate was concentrated to give the residue, which was purified with prep-HPLC (column: YMC-Actus Triart C18 150*30 mm*7 um; mobile phase: [water (0.05% ammonia hydroxide v/v)-ACN]; B %: 13%-53%, 10 min) to provide 2-[4-[6-[4-methyl-2-(pyrimidin-2-yloxymethyl)thiazol-5-yl]-2-pyridyl]phenyl]sulfonylacetic acid (2) (30.8 mg, 62.66 μmol, 39.5% yield, 98.16% purity) as a solid. ¹H NMR (400 MHz, DMSO-d₆) δ ppm 8.70 (d, J=4.8 Hz, 2H), 8.30 (d, J=8.4 Hz, 2H), 8.10-8.01 (m, 4H), 7.81-7.73 (m, 1H), 7.25 (t, J=4.8 Hz, 1H), 5.68 (s, 2H), 4.11 (br. s, 2H), 2.72 (s, 3H); HPLC (98.16% purity); ES-MS m/z 483.1 [M+H]+.

### Example S3. Preparation of 5-(6-(4-methyl-2-((pyrimidin-2-yloxy)methyl)thiazol-5-yl)pyridin-2-yl)thiophene-3-carboxylic Acid (Compound 3)

### Step 1: Synthesis of methyl 5-(6-(4-methyl-2-((pyrimidin-2-yloxy)methyl)thiazol-5-yl)pyridin-2-yl)thiophene-3-carboxylate (3a)

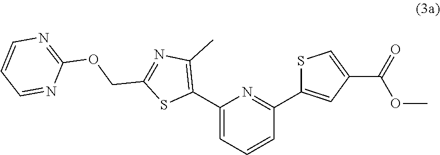 [SMILES: COC(=O)c1csc(-c2cccc(-c3sc(COc4ncccn4)nc3C)n2)c1]

To a solution of 5-(6-bromopyridin-2-yl)-4-methyl-2-((pyrimidin-2-yloxy)methyl)thiazole (80 mg, 220.24 μmol) in 1,4-dioxane (8 ml, 93.52 mmol) were added (4-(methoxycarbonyl)thiophen-2-yl)boronic acid (81.93 mg, 440.49 μmol) and Cs₂CO₃ (143.52 mg, 440.49 μmol), after 15 minutes under N₂, followed by addition of PdCl₂(dppf)-CH₂Cl₂ adduct (8.99 mg, 11.01 μmol). After 3 h at 80° C., the mixture was diluted with EtOAc (40 ml) and water (20 ml). The organic layer was washed brine (10 ml), dried over Na₂SO₄, filtered, and concentrated to give a residue, which was purified by chromatography using 30-100% EtOAc in heptane as eluent to give methyl 5-(6-(4-methyl-2-((pyrimidin-2-yloxy)methyl)thiazol-5-yl)pyridin-2-yl)thiophene-3-carboxylate (3a) as a solid (0.046 g, 49%). M+H (425 m/z). ¹H NMR (400 MHz, Chloroform-d) δ 8.58 (d, J=4.8 Hz, 2H), 8.15 (d, J=1.2 Hz, 1H), 7.97 (d, J=1.3 Hz, 1H), 7.75 (t, J=7.9 Hz, 1H), 7.57 (dd, J=7.9, 0.8 Hz, 1H), 7.42 (dd, J=7.8, 0.8 Hz, 1H), 7.02 (t, J=4.8 Hz, 1H), 5.71 (s, 2H), 3.90 (s, 3H), 2.79 (s, 3H) ppm.

Step 2: Synthesis of 5-(6-(4-methyl-2-((pyrimidin-2-yloxy)methyl)thiazol-5-yl)pyridin-2-yl)thiophene-3-carboxylic acid (3). To a solution of methyl 5-(6-(4-methyl-2-((pyrimidin-2-yloxy)methyl)thiazol-5-yl)pyridin-2-yl)thiophene-3-carboxylate (3a) (45 mg, 106.01 μmol) in MeOH (3 ml, 74.15 mmol), THF (3 ml, 36.61 mmol) and H₂O (3 ml, 166.53 mmol) was added LiOH (12.69 mg, 530.04 μmol). After 3 days at room temperature, the mixture was concentrated to give a residue, which was purified by chromatography using 20-100% ACN in water as eluent to give 5-(6-(4-methyl-2-((pyrimidin-2-yloxy)methyl)thiazol-5-yl)pyridin-2-yl)thiophene-3-carboxylic acid (3) as a solid (0.012 g, 28%). M+H (411 m/z). ¹H NMR (400 MHz, DMSO-d6) δ 8.70 (d, J=4.9 Hz, 2H), 8.34 (s, 1H), 8.13 (s, 1H), 7.95 (d, J=10.1 Hz, 2H), 7.62 (d, J=7.4 Hz, 1H), 7.25 (t, J=5.0 Hz, 1H), 5.67 (s, 2H), 2.71 (s, 3H) ppm.

### Example S4. Preparation of 2-hydroxy-4-[3-[4-methyl-2-(2-pyridyloxymethyl)thiazol-5-yl]phenyl]benzoic Acid (Compound 4)

### Step 1: Synthesis of 5-bromo-4-methyl-2-(2-pyridyloxymethyl)thiazole (4a)

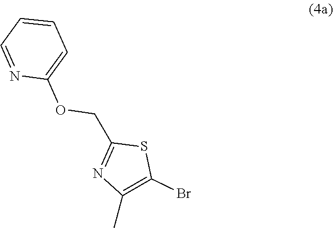

To a solution of (5-bromo-4-methyl-thiazol-2-yl)methanol (10 g, 43.25 mmol, 1 eq) and pyridin-2-ol (8.23 g, 86.51 mmol, 2 eq) in THF (100 mL) were added DEAD (11.30 g, 64.88 mmol, 11.79 mL, 1.5 eq) and PPh₃ (17.02 g, 64.88 mmol, 1.5 eq). After 1 h at 25° C., the mixture was concentrated to give the residue, which was purified with flash silica gel chromatography (ISCO®; 80 g SepaFlash® Silica Flash Column, Eluent of 0˜10% Ethylacetate/Petroleum ether gradient @ 40 mL/min) to give 5-bromo-4-methyl-2-(2-pyridyloxymethyl)thiazole (4a) (3 g, 9.47 mmol, 21.89% yield, 90% purity) as a solid. ¹H NMR (400 MHz, CDCl₃) δ ppm 8.19 (dd, J=1.2, 5.2 Hz, 1H), 7.65-7.61 (m, 1H), 6.96-6.92 (m, 1H), 6.84 (d, J=8.4 Hz, 1H), 5.52 (s, 2H), 2.41 (s, 3H).

Step 2: Synthesis of 2-hydroxy-4-[3-[4-methyl-2-(2-pyridyloxymethyl)thiazol-5-yl]phenyl]benzoic acid (4). A mixture of 5-bromo-4-methyl-2-(2-pyridyloxymethyl)thiazole (4a) (1.5 g, 4.73 mmol, 1 eq), 2-hydroxy-4-[3-(4,4,5,5-tetramethyl-1,3,2-dioxaborolan-2-yl)phenyl]benzoic acid (2.42 g, 7.10 mmol, 1.5 eq), Cs₂CO₃ (3.08 g, 9.47 mmol, 2 eq), and Pd(dppf)Cl₂ (346.40 mg, 473.42 μmol, 0.1 eq) in 1,4-dioxane (7.5 mL) and H₂O (7.5 mL) was stirred at 85° C. for 1 h under N₂. The mixture was concentrated and filtered to give the crude product, which was purified with prep-HPLC (column: Boston Prime C18 150*30 mm*5 um; mobile phase: [water(0.04% NH₃H₂O+10 mM NH₄HCO₃)-ACN]; B %: 30%-60%, 8 min) to afford 2-hydroxy-4-[3-[4-methyl-2-(2-pyridyloxymethyl)thiazol-5-yl]phenyl]benzoic acid (4) (420 mg, 994.44 μmol, 21.0% yield, 99.08% purity) as a solid. ¹H NMR (400 MHz, CD₃OD) δ ppm 8.17 (d, J=3.6 Hz, 1H), 7.90 (d, J=8.4 Hz, 1H), 7.75-7.68 (m, 1H), 7.67-7.61 (m, 2H), 7.51 (t, J=7.6 Hz, 1H), 7.46-7.40 (m, 1H), 7.07-7.02 (m, 2H), 7.02-6.97 (m, 1H), 6.90 (d, J=8.4 Hz, 1H), 5.62 (s, 2H), 2.50 (s, 3H).

### Example S5. Preparation of 5-fluoro-2-hydroxy-4-[4-[2-[(methoxycarbonylamino)methyl]-4-methyl-thiazol-5-yl]thiazol-2-yl]benzoic Acid (Compound 5)

### Step 1: Synthesis of 1-[2-(aminomethyl)-4-methyl-thiazol-5-yl]-2-bromo-ethanone (5a)

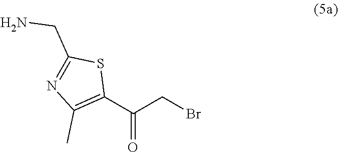

To a solution of tert-butyl N-[(5-acetyl-4-methyl-thiazol-2-yl)methyl]carbamate (4 g, 13.17 mmol, 1 eq) in CH₃COOH (40 mL) was added HBr (4.74 g, 26.34 mmol, 3.18 mL, 45% purity, 2 eq). After 30 min at 60° C., Br₂ (2.53 g, 15.80 mmol, 814.61 μL, 1.2 eq) was added. After 1 h at 60° C., the mixture was filtered and washed with DCM (50 mL) to give 1-[2-(aminomethyl)-4-methyl-thiazol-5-yl]-2-bromo-ethanone (5a) (3 g, crude) as a solid.

### Step 2: Synthesis of methyl 4-[4-[2-(aminomethyl)-4-methyl-thiazol-5-yl]thiazol-2-yl]-5-fluoro-2-methoxy-benzoate (5b)

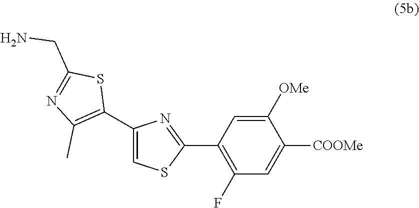 [SMILES: COC(=O)c1cc(F)c(-c2nc(-c3sc(CN)nc3C)cs2)cc1OC]

To a solution of 1-[2-(aminomethyl)-4-methyl-thiazol-5-yl]-2-bromo-ethanone (5a) (2.03 g, 4.07 mmol, 1.1 eq) in MeOH (10 mL) was added methyl 4-carbamothioyl-5-fluoro-2-methoxy-benzoate (1 g, 3.70 mmol, 1 eq). After 30 min at 80° C., the mixture was concentrated and the residue was washed with EtOAc (20 mL×3) to give methyl 4-[4-[2-(aminomethyl)-4-methyl-thiazol-5-yl]thiazol-2-yl]-5-fluoro-2-methoxy-benzoate 5b (1 g, 2.29 mmol, 61.8% yield, 90% purity) as a solid. ¹H NMR (400 MHz, CDCl₃) δ ppm 7.99 (d, J=6.0 Hz, 1H), 7.70 (d, J=10.8 Hz, 1H), 7.48 (s, 1H), 4.19 (s, 2H), 4.03 (s, 3H), 3.94 (s, 3H), 2.70 (s, 3H).

### Step 3: Synthesis of methyl 4-[4-[2-(aminomethyl)-4-methyl-thiazol-5-yl]thiazol-2-yl]-5-fluoro-2-hydroxy-benzoate (5c)

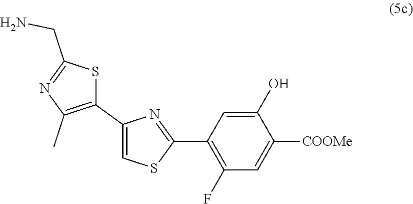 [SMILES: COC(=O)c1cc(F)c(-c2nc(-c3sc(CN)nc3C)cs2)cc1O]

To a solution of methyl 4-[4-[2-(aminomethyl)-4-methyl-thiazol-5-yl]thiazol-2-yl]-5-fluoro-2-methoxy-benzoate (5b) (950 mg, 2.17 mmol, 1 eq) in DCM (10 mL) was added BBr₃ (4.36 g, 17.38 mmol, 1.68 mL, 8 eq). After 3 min at −78° C., the mixture was filtered and washed with DCM (20 mL×3). Then the filtrate was adjusted into pH=9 with saturated NaHCO₃ solution and extracted with DCM/MeOH=10/1 (100 mL×2). The combined organic layers were washed with brine, dried over Na₂SO₄, filtered and concentrated to give the residue, which was triturated with EtOAc (20 mL) and filtered to afford methyl 4-[4-[2-(aminomethyl)-4-methyl-thiazol-5-yl]thiazol-2-yl]-5-fluoro-2-hydroxy-benzoate (5c) (900 mg, 2.13 mmol, 98.2% yield, 90% purity) as a solid. ¹H NMR (400 MHz, CDCl₃) δ ppm 10.53 (br s, 1H), 8.00 (d, J=6.0 Hz, 1H), 7.68 (d, J=11.2 Hz, 1H), 7.48 (s, 1H), 4.18 (s, 2H), 4.00 (s, 3H), 2.70 (s, 3H).

### Step 4: Synthesis of 4-[4-[2-(aminomethyl)-4-methyl-thiazol-5-yl]thiazol-2-yl]-5-fluoro-2-hydroxy-benzoic Acid (5d)

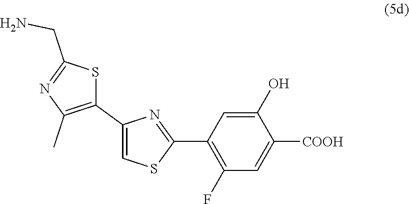 [SMILES: Cc1nc(CN)sc1-c1csc(-c2cc(O)c(C(=O)O)cc2F)n1]

To a solution of methyl 4-[4-[2-(aminomethyl)-4-methyl-thiazol-5-yl]thiazol-2-yl]-5-fluoro-2-hydroxy-benzoate (5c) (250 mg, 593.00 μmol, 1 eq) in THF (3 mL) and H₂O (3 mL) was added LiOH·H₂O (199.07 mg, 4.74 mmol, 8 eq). After 12 h at 50° C., the mixture was adjusted into pH=5 with 1 M HCl and filtered to give 4-[4-[2-(aminomethyl)-4-methyl-thiazol-5-yl]thiazol-2-yl]-5-fluoro-2-hydroxy-benzoic acid (5d) (200 mg, 492.61 μmol, 83.1% yield, 90% purity) as a solid. ¹H NMR (400 MHz, DMSO-d₆) δ ppm 7.93 (s, 1H), 7.50 (d, J=11.6 Hz, 1H), 7.40 (d, J=6.0 Hz, 1H), 4.03 (s, 2H), 2.58 (s, 3H).

Step 5: Synthesis of 5-fluoro-2-hydroxy-4-[4-[2-[(methoxycarbonylamino)methyl]-4-methyl-thiazol-5-yl]thiazol-2-yl]benzoic acid (5). To a solution of 4-[4-[2-(aminomethyl)-4-methyl-thiazol-5-yl]thiazol-2-yl]-5-fluoro-2-hydroxy-benzoic acid (5d) (200 mg, 492.61 mol, 1 eq) in DMF (5 mL) were added DIEA (76.40 mg, 591.13 mol, 102.96 μL, 1.2 eq) and methyl chloroformate (46.55 mg, 492.61 mol, 38.16 μL, 1 eq). After 2 h at 25° C., the mixture was filtered and the filtrate was concentrated to give the residue, which was purified with prep-HPLC (basic condition, Boston Prime C18 150*30 mm 5 um, water (0.04% NH₃H₂O+10 mM NH₄HCO₃)-ACN, B %10%-40%, 8 min) to give 5-fluoro-2-hydroxy-4-[4-[2-[(methoxycarbonylamino)methyl]-4-methyl-thiazol-5-yl]thiazol-2-yl]benzoic acid (5) (30 mg, 68.91 μmol, 14% yield, 97.26% purity) as a solid. ¹H NMR (400 MHz, DMSO-d₆) δ ppm 8.09-8.06 (m, 1H), 8.05 (s, 1H), 7.65 (d, J=11.2 Hz, 1H), 7.62 (d, J=6.0 Hz, 1H), 4.40 (d, J=6.0 Hz, 2H), 3.57 (s, 3H), 2.58 (s, 3H); HPLC (Purity: 97.26%); ES-MS m/z 424.1 [M+H]*.

### Example S6. Preparation of 2-hydroxy-4-[6-[4-methyl-2-(pyrimidin-2-yloxymethyl)thiazol-5-yl]-2-pyridyl]benzoic Acid (Compound 6)

### Step 1: Synthesis of tert-butyl-[[5-(6-chloro-2-pyridyl)-4-methyl-thiazol-2-yl]methoxy]-dimethyl-silane (6a)

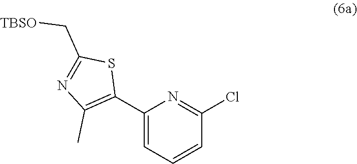 [SMILES: Cc1nc(CO[Si](C)(C)C(C)(C)C)sc1-c1cccc(Cl)n1]

To a mixture solution of 2,6-dichloropyridine (2.34 g, 15.84 mmol, 1.3 eq) in dioxane (120 mL) and H₂O (12 mL) were added tert-butyl-dimethyl-[[4-methyl-5-(4,4,5,5-tetramethyl-1,3,2-dioxaborolan-2-yl)thiazol-2-yl]methoxy]silane (5 g, 12.18 mmol, 1 eq), Cs₂CO₃ (11.91 g, 36.55 mmol, 3 eq) and Pd(dppf)Cl₂ (534.81 mg, 730.91 μmol, 0.06 eq). After 2 h at 90° C., the mixture was diluted with water (100 mL) and extracted with EtOAc (80 mL×3). The combined organic layer was washed with brine (100 mL), dried over anhydrous Na₂SO₄, filtered, and concentrated in vacuo to afford the residue, which was purified with flash silica gel chromatography (ISCO®; 20 g SepaFlash® Silica Flash Column, Eluent of 0-10% Ethyl acetate/Petroleum ether gradient @ 35 mL/min) to give tert-butyl-[[5-(6-chloro-2-pyridyl)-4-methyl-thiazol-2-yl]methoxy]-dimethyl-silane (6a) (1.5 g, 3.80 mmol, 31.2% yield, 90% purity) as an oil. ¹H NMR (400 MHz, CDCl₃) δ ppm 7.68 (t, J=8.0 Hz, 1H), 7.44 (d, J=8.0 Hz, 1H), 7.21 (d, J=8.0 Hz, 1H), 4.93 (s, 2H), 2.67 (s, 3H), 0.97 (s, 9H), 0.15 (s, 6H).

### Step 2: Synthesis of [5-(6-chloro-2-pyridyl)-4-methyl-thiazol-2-yl]methanol (6b)

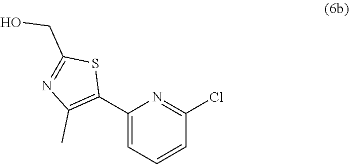

To a mixture solution of tert-butyl-[[5-(6-chloro-2-pyridyl)-4-methyl-thiazol-2-yl]methoxy]-dimethyl-silane (6a) (1.5 g, 3.80 mmol, 1 eq) in THF (15 mL) was added TBAF (1 M in THF, 4.56 mL, 1.2 eq). After 1 h at 25° C., the mixture was concentrated in vacuo to afford the residue, which was purified with flash silica gel chromatography (ISCO®; 12 g SepaFlash® Silica Flash Column, Eluent of 100% Ethyl acetate ether gradient @ 30 mL/min) to give [5-(6-chloro-2-pyridyl)-4-methyl-thiazol-2-yl]methanol (6b) (1.0 g, 3.74 mmol, 98.3% yield, 90% purity) as a solid. ¹H NMR (400 MHz, CDCl₃) δ ppm 7.70 (t, J=8.0 Hz, 1H), 7.45 (d, J=8.0 Hz, 1H), 7.23 (d, J=8.0 Hz, 1H), 4.93 (s, 2H), 2.69 (s, 3H).

### Step 3: Synthesis of 5-(6-chloropyridin-2-yl)-4-methyl-2-((pyrimidin-2-yloxy)methyl)thiazole (6c)

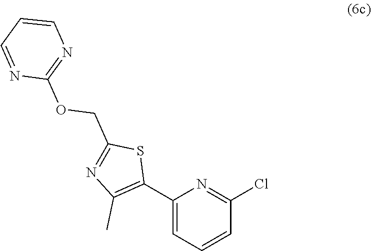 [SMILES: Cc1nc(COc2ncccn2)sc1-c1cccc(Cl)n1]

To a solution of [5-(6-chloro-2-pyridyl)-4-methyl-thiazol-2-yl]methanol 6b (100 mg, 373.90 μmol, 1 eq) in DMF (2 mL) was added NaH (16.45 mg, 411.29 μmol, 60% purity, 1.1 eq), after 15 min at 0° C., followed by addition of 2-chloropyrimidine (128.47 mg, 1.12 mmol, 3 eq). After 45 min at 25° C. the mixture was quenched with water (10 mL) and extracted with ethyl acetate (15 mL×2). The combined organic layer was washed with brine (20 mL), dried over anhydrous Na₂SO₄, filtered, and concentrated in vacuo to afford the residue, which was purified with flash silica gel chromatography (ISCO®; 4 g SepaFlash® Silica Flash Column, Eluent of 0˜10% MeOH/DCM ether gradient @18 mL/min) to give 5-(6-chloro-2-pyridyl)-4-methyl-2-(pyrimidin-2-yloxymethyl)thiazole 6c (85 mg, 239.98 μmol, 64.2% yield, 90% purity) as a solid. ¹H NMR (400 MHz, DMSO-d₆) δ ppm 8.69 (d, J=4.8 Hz, 2H), 7.92-7.98 (m, 1H), 7.72 (d, J=8.0 Hz, 1H), 7.46 (d, J=8.0 Hz, 1H), 7.24 (t, J=4.8 Hz, 1H), 5.66 (s, 2H), 2.64 (s, 3H).

Step 4: Synthesis of 2-hydroxy-4-[6-[4-methyl-2-(pyrimidin-2-yloxymethyl)thiazol-5-yl]-2-pyridyl]benzoic acid (6). To a mixture solution of 5-(6-chloro-2-pyridyl)-4-methyl-2-(pyrimidin-2-yloxymethyl)thiazole 6c (80 mg, 225.86 μmol, 1 eq) in dioxane (2 mL) and H₂O (0.2 mL) were added 2-hydroxy-4-(4,4,5,5-tetramethyl-1,3,2-dioxaborolan-2-yl)benzoic acid (86.15 mg, 293.62 μmol, 1.3 eq), Cs₂CO₃ (220.77 mg, 677.58 μmol, 3 eq), and Pd(dppf)Cl₂ (16.53 mg, 22.59 μmol, 0.1 eq). After 3 h at 90° C., the mixture was filtered and concentrated to give the residue which was purified with prep-HPLC (column: Boston Prime C18 150*30 mm*5 um; mobile phase: [water(0.04% NH₃H₂O+10 mM NH₄HCO₃)-ACN]; B %: 15%-45%, 8 min) to give 2-hydroxy-4-[6-[4-methyl-2-(pyrimidin-2-yloxymethyl)thiazol-5-yl]-2-pyridyl]benzoic acid 6 (40.4 mg, 95.05 μmol, 42.1% yield, 98.9% purity) as a solid. ¹H NMR (400 MHz, DMSO-d₆) δ ppm 8.70 (d, J=4.8 Hz, 2H), 7.96-8.04 (m, 2H), 7.90 (d, J=7.6 Hz, 1H), 7.73 (d, J=7.2 Hz, 1H), 7.61-7.66 (m, 2H), 7.24 (t, J=4.8 Hz, 1H), 5.67 (s, 2H), 2.71 (s, 3H); HPLC (Purity: 98.92%); ES-MS m/z 421.1 [M+H]*.

### Example S7. Preparation of 4-[4-[2-[[2-(dimethylamino)-2-oxo-ethoxy]methyl]-4-methyl-thiazol-5-yl]thiazol-2-yl]-5-fluoro-2-hydroxy-benzoic Acid (Compound 7)

### Step 1: Synthesis of methyl 4-(4-bromothiazol-2-yl)-5-fluoro-2-methoxybenzoate (7a)

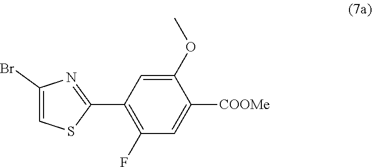 [SMILES: COC(=O)c1cc(F)c(-c2nc(Br)cs2)cc1OC]

To a solution of methyl 5-fluoro-2-methoxy-4-(4,4,5,5-tetramethyl-1,3,2-dioxaborolan-2-yl)benzoate (217.00 mg, 699.72 μmol, 1 eq) and 2,4-dibromothiazole (220.97 mg, 909.63 μmol, 488.21 μL, 1.3 eq) in 1,4-dioxane (5 mL) and water (0.5 mL) were added Pd(dppf)Cl₂ (25.60 mg, 34.99 μmol, 0.05 eq) and K₂CO₃ (193.42 mg, 1.40 mmol, 2 eq) under N₂ atmosphere. After 12 h at 80° C., under N₂ atmosphere, the mixture was concentrated, diluted with aqueous HCl (1 M) to adjust pH=6-7, and extracted with EtOAc (30 mL×3). The combined organic layers were dried over Na₂SO₄, filtered, and concentrated to give the residue, which was purified with flash silica gel chromatography (ISCO®; 12 g SepaFlash® Silica Flash Column, Eluent of 0˜30% Ethyl acetate/Petroleum ether gradient @ 30 mL/min) to give methyl 4-(4-bromothiazol-2-yl)-5-fluoro-2-methoxy-benzoate 7a (200 mg, 519.97 μmol, 74.3% yield, 90% purity) as a solid. ¹H NMR (400 MHz, CDCl3) δ ppm 7.89 (d, J=6.0 Hz, 1H), 7.69 (d, J=11.6 Hz, 2H), 7.43 (s, 1H), 4.03 (s, 3H), 4.00 (brs, 3H).

### Step 2: Synthesis of methyl 4-(2′-(hydroxymethyl)-4′-methyl-[4,5′-bithiazol]-2-yl)-2-methoxybenzoate (7b)

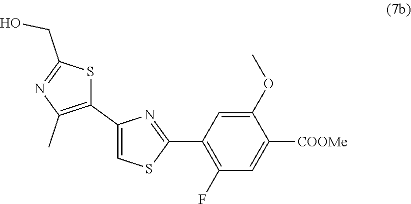 [SMILES: COC(=O)c1cc(F)c(-c2nc(-c3sc(CO)nc3C)cs2)cc1OC]

To a solution of Pd(OAc)₂ (62.69 mg, 279.21 mmol, 0.1 eq) in toluene (3 mL) was added bis(1-adamantyl)-butyl-phosphane (200.22 mg, 558.42 mmol, 0.2 eq). After stirring for 30 minutes under N₂ at 25° C., the solution was added to a solution of (5-bromo-4-methyl-thiazol-2-yl)methoxy-tert-butyl-dimethyl-silane (1.0 g, 2.79 mmol, 1.0 eq), methyl 4-(4-bromothiazol-2-yl)-2-methoxy-benzoate 7a (1.32 g, 3.63 mmol, 1.3 eq), 4,4,5,5-tetramethyl-2-(4,4,5,5-tetramethyl-1,3,2-dioxaborolan-2-yl)-1,3,2-dioxaborolane (921.73 mg, 3.63 mmol, 1.3 eq), and CsF (848.26 mg, 5.58 mmol, 205.89 mL, 2.0 eq) in MeOH (20 mL) at 50° C. under N₂. After 12 h at 80° C., the mixture was concentrated, diluted with H₂O (10 mL), and extracted with EtOAc (20 mL×3). The combined organic layers were washed with brine (15 mL×3), dried over Na₂SO₄, filtered, and concentrated under reduced pressure to give a residue, which was purified with flash silica gel chromatography (ISCO®; 24 g SepaFlash® Silica Flash Column, Eluent of 0˜40% Ethylacetate/Petroleum ether gradient @ 50 mL/min) to give methyl 4-[4-[2-(hydroxymethyl)-4-methyl-thiazol-5-yl]thiazol-2-yl]-2-methoxy-benzoate 7b (200 mg, 478.15 mmol, 17.13% yield, 90% purity) as an oil. ¹H NMR (400 MHz, CD₃OD) δ ppm 7.87 (d, J=8.4 Hz, 1H), 7.74 (s, 2H), 7.62 (dd, J=1.2, 8.0 Hz, 1H), 4.83 (s, 2H), 4.00 (s, 3H), 3.89 (s, 3H), 2.65 (s, 3H).

### Step 3: Synthesis of methyl 5-fluoro-2-hydroxy-4-(2′-(hydroxymethyl)-4′-methyl-[4,5′-bithiazol]-2-yl) benzoate (7c)

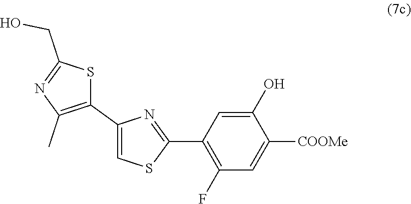 [SMILES: COC(=O)c1cc(F)c(-c2nc(-c3sc(CO)nc3C)cs2)cc1O]

To a solution of methyl 5-fluoro-4-[4-[2-(hydroxymethyl)-4-methyl-thiazol-5-yl]thiazol-2-yl]-2-methoxy-benzoate 7b (300 mg, 684.52 μmol, 1 eq) in DCM (10 mL) was added dropwise BBr₃ (857.43 mg, 3.42 mmol, 329.78 μL, 5 eq) at −78° C. under N₂. The mixture was stirred at −78° C. for 1.5 h. TLC (Ethyl acetate) indicated some of starting material was remained. The mixture was quenched with saturated NaHCO₃ solution (10 mL) at 0° C. and extracted with DCM (25 mL×4). The combined organic layers were dried over anhydrous sodium sulfate, filtered and concentrated under reduced pressure to give methyl 5-fluoro-2-hydroxy-4-[4-[2-(hydroxymethyl)-4-methyl-thiazol-5-yl]thiazol-2-yl]benzoate 7c (250 mg, 328.59 μmol, 48.0% yield, 50% purity) as a solid which was used in next step without further purification. ¹H NMR (400 MHz, CDCl₃) δ ppm 10.55 (s, 1H), 8.00 (d, J=6.0 Hz, 1H), 7.70 (d, J=11.2 Hz, 1H), 7.51 (s, 1H), 4.96 (s, 2H), 3.94 (s, 3H), 2.72 (s, 3H); ES-MS m/z 380.7 [M+H]+.

### Step 4: Synthesis of methyl 4-(2′-((2-ethoxy-2-oxoethoxy)methyl)-4′-methyl-[4,5′-bithiazol]-2-yl)-5-fluoro-2-hydroxybenzoate (7d)

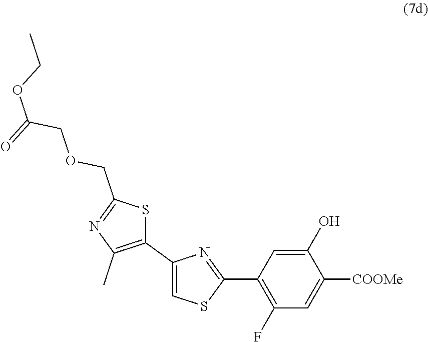 [SMILES: CCOC(=O)COCc1nc(C)c(-c2csc(-c3cc(O)c(C(=O)OC)cc3F)n2)s1]

To a solution of methyl 5-fluoro-2-hydroxy-4-[4-[2-(hydroxymethyl)-4-methyl-thiazol-5-yl] thiazol-2-yl]benzoate 7c (230 mg, 302.30 μmol, 1 eq) in DMF (13 mL) was added NaH (60.45 mg, 1.51 mmol, 60% purity in mineral, 5 eq) in portions at 0° C., after 30 min at 0° C., followed by addition of ethyl 2-bromoacetate (55.53 mg, 332.53 μmol, 36.78 μL, 1.1 eq). After 1 h at 0° C., the mixture was quenched with saturated NH₄Cl solution (15 mL) at 0° C. and extracted with ethyl acetate (30 mL×3). The combined organic layers were washed with H₂O (15 mL×2) and brine (15 mL), dried over anhydrous sodium sulfate, filtered and concentrated under reduced pressure. The residue was purified with flash silica gel chromatography (ISCO®; 12 g SepaFlash® Silica Flash Column, Eluent of 0˜100% Ethyl acetate/Petroleum ether gradient @ 35 mL/min) and then further purified with prep-TLC (SiO₂, petroleum ether/ethyl acetate=1/1) to give methyl 4-[4-[2-[(2-ethoxy-2-oxo-ethoxy)methyl]-4-methyl-thiazol-5-yl]thiazol-2-yl]-5-fluoro-2-hydroxy-benzoate 7d (45 mg, 77.17 μmol, 25.5% yield, 80% purity) as a solid. ¹H NMR (400 MHz, CDCl₃) δ ppm 10.54 (s, 1H), 7.99 (d, J=6.0 Hz, 1H), 7.71-7.67 (m, 1H), 7.51 (s, 1H), 4.92 (s, 2H), 4.29-4.23 (m, 4H), 4.03 (s, 3H), 2.71 (s, 3H), 1.31 (t, J=7.2 Hz, 3H); ES-MS m/z 467.1 [M+H]*.

### Step 5: Synthesis of 4-(2′-((carboxymethoxy)methyl)-4′-methyl-[4,5′-bithiazol]-2-yl)-5-fluoro-2-hydroxybenzoic Acid (7e)

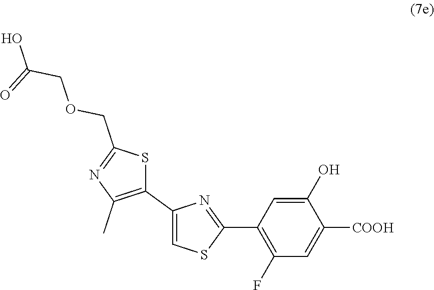 [SMILES: Cc1nc(COCC(=O)O)sc1-c1csc(-c2cc(O)c(C(=O)O)cc2F)n1]

A mixture of methyl 4-[4-[2-[(2-ethoxy-2-oxo-ethoxy)methyl]-4-methyl-thiazol-5-yl]thiazol-2-yl]-5-fluoro-2-hydroxy-benzoate 7d (45 mg, 77.17 μmol, 1 eq) and LiOH·H₂O (16.19 mg, 385.85 μmol, 5 eq) in THF (2 mL) and H₂O (0.4 mL) was stirred at 20° C. for 12 h. The mixture was concentrated under reduced pressure to give the residue, which was acidified to pH=5 with 1 N HCl and extracted with DCM:ⁱ-PrOH (8:1, 30 mL x 5). The combined organic layers were dried over anhydrous sodium sulfate, filtered and concentrated under reduced pressure to give 4-[4-[2-(carboxymethoxymethyl)-4-methyl-thiazol-5-yl]thiazol-2-yl]-5-fluoro-2-hydroxy-benzoic acid 7e (35 mg, 69.27 μmol, 89.8% yield, 84% purity) as a solid which was used in next step without further purification. ¹H NMR (400 MHz, CD₃OD) δ ppm 7.98 (s, 1H), 7.87 (d, J=6.4 Hz, 1H), 7.77 (d, J=11.2 Hz, 1H), 4.97 (s, 2H), 4.33 (s, 2H), 2.73 (s, 3H); ES-MS m/z 425.0 [M+H]+.

Step 6: Synthesis of 4-(2′-((2-(dimethylamino)-2-oxoethoxy)methyl)-4′-methyl-[4,5′-bithiazol]-2-yl)-5-fluoro-2-hydroxybenzoic acid (7). A mixture of 4-[4-[2-(carboxymethoxymethyl)-4-methyl-thiazol-5-yl]thiazol-2-yl]-5-fluoro-2-hydroxy-benzoic acid 7e (35 mg, 69.27 μmol, 1 eq), N-methylmethanamine hydrochloride (11.30 mg, 138.54 μmol, 2 eq), HATU (39.51 mg, 103.91 μmol, 1.5 eq), and Et₃N (35.05 mg, 346.35 μmol, 48.21 μL, 5 eq) in DMF (2 mL) was stirred at 20° C. for 12 h. The mixture was filtered and concentrated to give the residue, which was purified with prep-HPLC (column: Phenomenex Gemini-NX 150*30 mm*5 um; mobile phase: [water (0.05% ammonia hydroxide v/v)-ACN]; B %: 0%-39%, 10 min) to give 4-[4-[2-[[2-(dimethylamino)-2-oxo-ethoxy]methyl]-4-methyl-thiazol-5-yl]thiazol-2-yl]-5-fluoro-2-hydroxy-benzoic acid 7 (5.8 mg, 12.82 μmol, 18.5% yield, 99.76% purity) as a solid. ¹H NMR (400 MHz, DMSO-d) 6 ppm 8.07 (s, 1H), 7.62 (d, J=11.6 Hz, 1H), 7.56 (d, J=6.0 Hz, 1H), 4.81 (s, 2H), 4.37 (s, 2H), 2.92 (s, 3H), 2.85 (s, 3H), 2.64 (s, 3H); HPLC (99.76% purity); ES-MS m/z 452.1 [M+H]*.

### Example S8. Preparation of 5-fluoro-2-hydroxy-4-[3-[2-[(methoxycarbonylamino)methyl]-4-methyl-thiazol-5-yl]-1,2,4-thiadiazol-5-yl]benzoic Acid (Compound 8)

### Step 1: Synthesis of methyl 4-(3-bromo-1,2,4-thiadiazol-5-yl)-5-fluoro-2-methoxy-benzoate (8a)

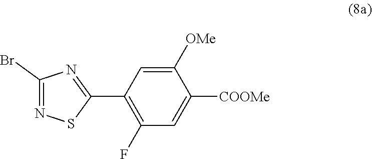 [SMILES: COC(=O)c1cc(F)c(-c2nc(Br)ns2)cc1OC]

To a solution of methyl 5-fluoro-2-methoxy-4-(4,4,5,5-tetramethyl-1,3,2-dioxaborolan-2-yl)benzoate (1.5 g, 4.35 mmol, 1 eq) and 3-bromo-5-chloro-1,2,4-thiadiazole (955.08 mg, 4.79 mmol, 1.1 eq) in 1,4-dioxane (30 mL) and H₂O (4 mL) were added Pd(dppf)Cl₂ (318.52 mg, 435.31 umol, 0.1 eq) and Cs₂CO₃ (4.25 g, 13.06 mmol, 3 eq). After 4 h at 85° C., the mixture was partitioned between EtOAc (35 mL) and H₂O (35 mL) and extracted with EtOAc (35 mL×3). The extracts were dried over with Na₂SO₄, filtered, and concentrated to give a residue, which was purified with flash silica gel chromatography (ISCO®; 40 g SepaFlash® Silica Flash Column, Eluent of 0˜15% Ethyl acetate/Petroleum ether gradient @ 35 mL/min) to give methyl 4-(3-bromo-1,2,4-thiadiazol-5-yl)-5-fluoro-2-methoxy-benzoate 8a (1 g, 2.59 mmol, 59.55% yield, 90% purity) as a solid. ¹H NMR (400 MHz, CDCl₃) δ ppm 7.87 (d, J=5.2 Hz, 1H), 7.70 (d, J=10.2 Hz, 1H), 4.02 (s, 3H), 3.94 (s, 3H).

### Step 2: Synthesis of methyl 4-[3-[2-[[tert-butyl(dimethyl)silyl]oxymethyl]-4-methyl-thiazol-5-yl]-1,2,4-thiadiazol-5-yl]-5-fluoro-2-methoxy-benzoate (8b)

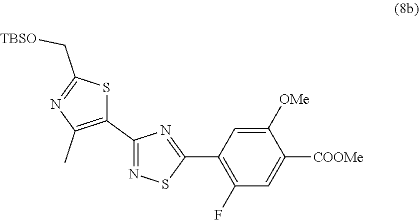 [SMILES: COC(=O)c1cc(F)c(-c2nc(-c3sc(CO[Si](C)(C)C(C)(C)C)nc3C)ns2)cc1OC]

A solution of methyl 4-(3-bromo-1,2,4-thiadiazol-5-yl)-5-fluoro-2-methoxy-benzoate 8a (1 g, 2.59 mmol, 1 eq), tert-butyl-dimethyl-[[4-methyl-5-(4,4,5,5-tetramethyl-1,3,2-dioxaborolan-2-yl)thiazol-2-yl]methoxy]silane (1.17 g, 2.85 mmol, 1.1 eq) and Cs₂CO₃ (1.27 g, 3.89 mmol, 1.5 eq) in dioxane (8 mL) and water (2 mL) was purged with nitrogen for 3 times before addition of Pd(dppf)Cl₂ (189.69 mg, 259.25 umol, 0.1 eq). After 1 h at 100° C. under microwave (1 bar), TLC (PE/EtOAc=5/1) showed the reaction was completed. The reaction was concentrated to give the residue, which was purified with flash silica gel chromatography (ISCO®; 12 g SepaFlash® Silica Flash Column, Eluent of 0˜15% Ethyl acetate/Petroleum ether gradient @ 35 mL/min) to give methyl 4-[3-[2-[[tert-butyl(dimethyl)silyl]oxymethyl]-4-methyl-thiazol-5-yl]-1,2,4-thiadiazol-5-yl]-5-fluoro-2-methoxy-benzoate 8b (0.47 g, 829.92 umol, 32.01% yield, 90% purity) as a solid. ¹H NMR (400 MHz, CDCl₃) δ ppm 7.98 (d, J=5.2 Hz, 1H), 7.74 (d, J=10.4, 1H), 4.98 (s, 2H), 4.05 (s, 3H), 3.95 (s, 3H), 2.92 (s, 3H), 0.99 (s, 9H), 0.18 (s, 6H).

### Step 3: Synthesis of methyl 5-fluoro-4-[3-[2-(hydroxymethyl)-4-methyl-thiazol-5-yl]-1,2,4-thiadiazol-5-yl]-2-methoxy-benzoate (8c)

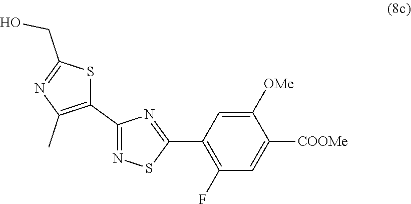 [SMILES: COC(=O)c1cc(F)c(-c2nc(-c3sc(CO)nc3C)ns2)cc1OC]

To a solution of methyl 4-[3-[2-[[tert-butyl(dimethyl)silyl]oxymethyl]-4-methyl-thiazol-5-yl]-1,2,4-thiadiazol-5-yl]-5-fluoro-2-methoxy-benzoate 8b (0.47 g, 829.92 umol, 1 eq) in THF (10 mL) was added TBAF (1 M, 995.90 uL, 1.2 eq). After 0.5 h at 25° C., the mixture was concentrated to give the residue, which was purified by flash silica gel chromatography (ISCO®; 12 g SepaFlash® Silica Flash Column, Eluent of 0˜90% Ethyl acetate/Petroleum ether gradient @ 35 mL/min) to give methyl 5-fluoro-4-[3-[2-(hydroxymethyl)-4-methyl-thiazol-5-yl]-1,2,4-thiadiazol-5-yl]-2-methoxy-benzoate 8c (0.2 g, 455.20 umol, 54.85% yield, 90% purity) as a solid. ¹H NMR (400 MHz, CDCl₃) δ ppm 7.95 (d, J=5.2 Hz, 1H), 7.73 (d, J=10.4, 1H), 4.98 (d, J=6.0, 2H), 4.04 (s, 3H), 3.95 (s, 3H), 2.92 (s, 3H).

### Step 4: Synthesis of methyl 4-[3-[2-(chloromethyl)-4-methyl-thiazol-5-yl]-1,2,4-thiadiazol-5-yl]-5-fluoro-2-methoxy-benzoate (8d)

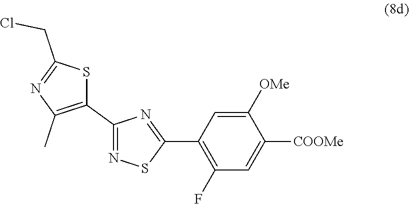 [SMILES: COC(=O)c1cc(F)c(-c2nc(-c3sc(CCl)nc3C)ns2)cc1OC]

To a solution of methyl 5-fluoro-4-[3-[2-(hydroxymethyl)-4-methyl-thiazol-5-yl]-1,2,4-thiadiazol-5-yl]-2-methoxy-benzoate 8c (0.2 g, 455.20 umol, 1 eq) in DCM (2 mL) was added SOCl₂ (81.23 mg, 682.80 umol, 49.53 uL, 1.5 eq). After 0.5 h at 25° C., TLC (PE/EtOAc=1/1, Rf=0.4) showed the reaction was completed. The reaction was concentrated to give methyl 4-[3-[2-(chloromethyl)-4-methyl-thiazol-5-yl]-1,2,4-thiadiazol-5-yl]-5-fluoro-2-methoxy-benzoate 8d (150 mg, 326.19 umol, 71.66% yield, 90% purity) as a solid. ¹H NMR (400 MHz, DMSO) δ ppm 7.89 (d, J=5.2 Hz, 1H), 7.85 (d, J=10.4, 1H), 5.13 (s, 2H), 3.95 (s, 3H), 3.86 (s, 3H), 2.86 (s, 3H).

### Step 5: Synthesis of methyl 4-[3-[2-[(1,3-dioxo-3a,7a-dihydroisoindol-2-yl)methyl]-4-methyl-thiazol-5-yl]-1,2,4-thiadiazol-5-yl]-5-fluoro-2-methoxy-benzoate (8e)

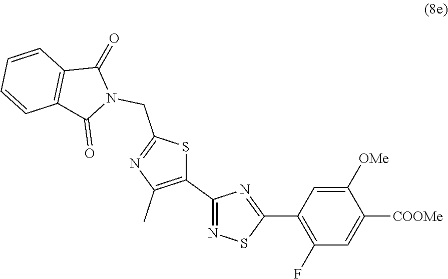 [SMILES: COC(=O)c1cc(F)c(-c2nc(-c3sc(CN4C(=O)c5ccccc5C4=O)nc3C)ns2)cc1OC]

A solution of methyl 4-[3-[2-(chloromethyl)-4-methyl-thiazol-5-yl]-1,2,4-thiadiazol-5-yl]-5-fluoro-2-methoxy-benzoate 8d (150 mg, 326.19 umol, 1 eq) and (1,3-dioxoisoindolin-2-yl)potassium (241.67 mg, 1.30 mmol, 4 eq) in DMF (5 mL) was stirred at 100° C. for 0.5 h. After cooling to rt, the precipitated solid was collected by filtration and dried to give methyl 4-[3-[2-[(1,3-dioxo-3a,7a-dihydroisoindol-2-yl)methyl]-4-methyl-thiazol-5-yl]-1,2,4-thiadiazol-5-yl]-5-fluoro-2-methoxy-benzoate 8e (150 mg, 256.38 umol, 78.60% yield, 90% purity) as a solid. ¹H NMR (400 MHz, CDCl₃) δ ppm 7.94-7.96 (m, 3H), 7.79-7.81 (m, 2H) 7.74 (d, J=10.4, 1H), 5.21 (s, 2H), 4.05 (s, 3H), 3.96 (s, 3H), 2.92 (s, 3H).

### Step 6: Synthesis of methyl 4-[3-[2-[(1,3-dioxoisoindolin-2-yl)methyl]-4-methyl-thiazol-5-yl]-1,2,4-thiadiazol-5-yl]-5-fluoro-2-hydroxy-benzoate (8f)

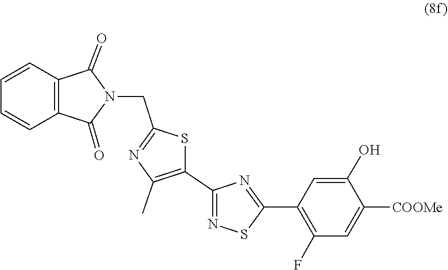 [SMILES: COC(=O)c1cc(F)c(-c2nc(-c3sc(CN4C(=O)c5ccccc5C4=O)nc3C)ns2)cc1O]

To a solution of methyl 4-[3-[2-[(1,3-dioxoisoindolin-2-yl)methyl]-4-methyl-thiazol-5-yl]-1,2,4-thiadiazol-5-yl]-5-fluoro-2-methoxy-benzoate 8e (150 mg, 257.37 umol, 1 eq) in DCM (10 mL) was added BBr₃ (644.76 mg, 2.57 mmol, 247.99 uL, 10 eq) at −70° C. After 0.5 h at 70° C., the mixture was cooled, quenched with MeOH (3 mL) at −70° C., and concentrated to give methyl 4-[3-[2-[(1,3-dioxoisoindolin-2-yl)methyl]-4-methyl-thiazol-5-yl]-1,2,4-thiadiazol-5-yl]-5-fluoro-2-hydroxy-benzoate 8f (250 mg, 244.85 umol, 95.14% yield, 50% purity) as a solid. ¹H NMR (400 MHz, DMSO) δ ppm 7.55-7.98 (m, 6H), 5.13 (s, 2H), 3.88 (s, 3H), 2.81 (s, 3H).

### Step 7: Synthesis of 4-[3-[2-[[(2-carboxybenzoyl)amino]methyl]-4-methyl-thiazol-5-yl]-1,2,4-thiadiazol-5-yl]-5-fluoro-2-hydroxy-benzoic acid (8g)

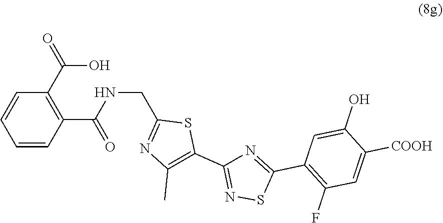 [SMILES: Cc1nc(CNC(=O)c2ccccc2C(=O)O)sc1-c1nsc(-c2cc(O)c(C(=O)O)cc2F)n1]

To a solution of methyl 4-[3-[2-[(1,3-dioxoisoindolin-2-yl)methyl]-4-methyl-thiazol-5-yl]-1,2,4-thiadiazol-5-yl]-5-fluoro-2-hydroxy-benzoate 8f (0.25 g, 244.85 umol, 1 eq) in THF (2 mL) and H₂O (1 mL) was added LiOH (58.64 mg, 2.45 mmol, 367.39 uL, 10 eq). After 0.5 h at 60° C., the mixture was concentrated, diluted with water, and acidified to pH-1 by 3N HCl to afford the solid which was filtered to give 4-[3-[2-[[(2-carboxybenzoyl)amino]methyl]-4-methyl-thiazol-5-yl]-1,2,4-thiadiazol-5-yl]-5-fluoro-2-hydroxy-benzoic acid 8g (120 mg, 198.25 umol, 80.97% yield, 85% purity) as a solid. ¹H NMR (400 MHz, DMSO) δ ppm 9.32 (t, J=6.0 Hz, 1H), 7.77-7.83 (m, 3H), 7.53-7.65 (m, 4H) 4.69 (d, J=6.0 Hz, 2H), 2.89 (s, 3H).

### Step 8: Synthesis of methyl 4-[4-[6-(aminomethyl)-3-pyridyl]thiazol-2-yl]-5-fluoro-2-methoxy-benzoate (8h)

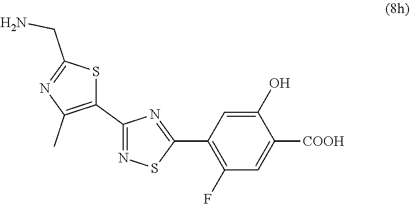 [SMILES: Cc1nc(CN)sc1-c1nsc(-c2cc(O)c(C(=O)O)cc2F)n1]

To a solution of 4-[3-[2-[[(2-carboxybenzoyl)amino]methyl]-4-methyl-thiazol-5-yl]-1,2,4-thiadiazol-5-yl]-5-fluoro-2-hydroxy-benzoic acid 8g (0.12 g, 198.25 umol, 1 eq) in EtOH (2 mL) was added N₂H₄·H₂O (202.54 mg, 3.96 mmol, 196.64 uL, 98% purity, 20.0 eq). After 12 h at 90° C., the mixture was concentrated to the residue, which was purified by prep-HPLC (basic condition column YMC-Actus Triart C18 150*30 mm*7 um, Condition water (0.05% ammonia hydroxide v/v)-ACN, Begin B 17 End B 57, Gradient Time(min) 11, 100% B Hold Time (min) 2, FlowRate (ml/min) 30, Injections 3) to give 4-[3-[2-(aminomethyl)-4-methyl-thiazol-5-yl]-1,2,4-thiadiazol-5-yl]-5-fluoro-2-hydroxy-benzoic acid 8h (60 mg, 147.38 umol, 74.34% yield, 90% purity) as a solid. ¹H NMR (400 MHz, DMSO) δ ppm 7.63 (d, J=10.8 Hz, 1H), 7.49-7.51 (m, 1H), 4.16 (d, J=6.8 Hz, 2H), 2.85 (s, 3H).

Step 9: Synthesis of 5-fluoro-2-hydroxy-4-[3-[2-[(methoxycarbonylamino)methyl]-4-methyl-thiazol-5-yl]-1,2,4-thiadiazol-5-yl]benzoic acid (8). To a solution of 4-[3-[2-(aminomethyl)-4-methyl-thiazol-5-yl]-1,2,4-thiadiazol-5-yl]-5-fluoro-2-hydroxy-benzoic acid 8h (60 mg, 147.38 umol, 1 eq) and DIEA (22.86 mg, 176.86 umol, 30.81 uL, 1.2 eq) in DMF (1 mL) and water (0.1 mL) was added methyl carbonochloridate (15.32 mg, 162.12 umol, 12.56 uL, 1.1 eq). After 0.5 h at 15° C., the mixture was filtered and the filtrate was concentrated to give the residue which was purified by prep-HPLC (Base condition; column: Welch Xtimate C18 150*25 mm*5 um; mobile phase: water (0.05% ammonia hydroxide v/v)-ACN; B %: 5%-45%, 11 min) to give 5-fluoro-2-hydroxy-4-[3-[2-[(methoxycarbonylamino)methyl]-4-methyl-thiazol-5-yl]-1,2,4-thiadiazol-5-yl]benzoic acid 8 (36.4 mg, 84.82 umol, 57.55% yield, 98.9% purity) as a solid. ¹H NMR (400 MHz, DMSO) δ ppm 8.15 (brs, 1H), 7.66 (d, J=11.2 Hz, 1H), 7.55 (d, J=5.6 Hz, 1H), 4.47 (d, J=6.0 Hz, 2H), 3.62 (s, 3H), 2.83 (s, 3H).

### Example S9. Preparation of 2-hydroxy-4-[6-[2-[(methoxycarbonylamino)methyl]-4-methyl-thiazol-5-yl]-2-pyridyl]benzoic Acid (Compound 9)

### Step 1: Synthesis of methyl 4-[6-[2-(azidomethyl)-4-methyl-thiazol-5-yl]-2-pyridyl]-2-methoxy-benzoate (9a)

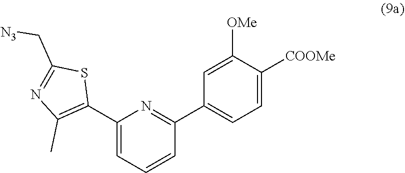 [SMILES: COC(=O)c1ccc(-c2cccc(-c3sc(CN=[N+]=[N-])nc3C)n2)cc1OC]

To a mixture of methyl 4-[6-[2-(hydroxymethyl)-4-methyl-thiazol-5-yl]-2-pyridyl]-2-methoxy-benzoate (300 mg, 631.71 μmol, 1 eq) and DIEA (408.21 mg, 3.16 mmol, 550.15 μL, 5 eq) in DCM (5 mL) was added MsCl (290 mg, 2.53 mmol, 195.95 μL, 4.01 eq). After 0.5 h at 0° C., NaN₃ (200 mg, 3.08 mmol, 4.87 eq) in DMF (5 mL) was added. After 2 h at 20° C., the mixture was partitioned between DCM (50 mL) and water (30 mL), and extracted with DCM (30 mL×3). The combined extracts were washed with brine (50 mL), dried over Na₂SO₄, filtered and concentrated to give methyl 4-[6-[2-(azidomethyl)-4-methyl-thiazol-5-yl]-2-pyridyl]-2-methoxy-benzoate 9a (280 mg, 566.47 μmol, 89.7% yield, 80% purity) as a solid. ¹H NMR (400 MHz, DMSO-d₆) δ ppm 8.09-8.01 (m, 2H), 7.88 (s, 1H), 7.82-7.73 (m, 3H), 4.84 (s, 2H), 3.96 (s, 3H), 3.83 (s, 3H), 2.73 (s, 3H).

### Step 2: Synthesis of methyl 4-[6-[2-(aminomethyl)-4-methyl-thiazol-5-yl]-2-pyridyl]-2-methoxy-benzoate (9b)

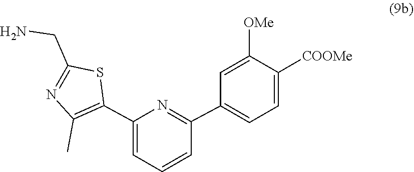 [SMILES: COC(=O)c1ccc(-c2cccc(-c3sc(CN)nc3C)n2)cc1OC]

To a solution of methyl 4-[6-[2-(azidomethyl)-4-methyl-thiazol-5-yl]-2-pyridyl]-2-methoxy-benzoate 9a (280 mg, 566.47 μmol, 1 eq) in THF (4 mL) and H₂O (1 mL) was added PPh₃ (193.15 mg, 736.41 μmol, 1.3 eq). After 12 h at 25° C., the mixture was concentrated, adjusted to pH=3-4 with 10% HCl, and then extracted with EtOAc (30 mL). The separated aqueous layer was basified with NaOH (10%) into pH=8-9 and extracted with DCM (30 mL×3). The combined organic layers were dried over Na₂SO₄, filtered and concentrated to give methyl 4-[6-[2-(aminomethyl)-4-methyl-thiazol-5-yl]-2-pyridyl]-2-methoxy-benzoate 9b (144 mg, 311.83 μmol, 55.1% yield, 80% purity) as a solid. ¹H NMR (400 MHz, DMSO-d₆) δ ppm 7.98 (s, 1H), 7.94-7.90 (m, 1H), 7.86-7.79 (m, 2H), 7.76 (s, 1H), 7.54 (d, J=3.6 Hz, 1H), 3.91 (s, 2H), 3.81-3.75 (m, 6H), 2.64 (s, 3H).

### Step 3: Synthesis of 4-[6-[2-(aminomethyl)-4-methyl-thiazol-5-yl]-2-pyridyl]-2-hydroxy-benzoic Acid (9c)

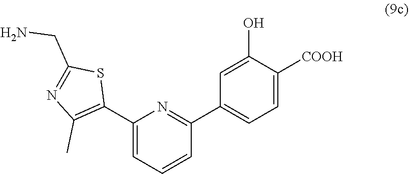 [SMILES: Cc1nc(CN)sc1-c1cccc(-c2ccc(C(=O)O)c(O)c2)n1]

To a solution of methyl 4-[6-[2-(aminomethyl)-4-methyl-thiazol-5-yl]-2-pyridyl]-2-methoxy-benzoate 9b (144 mg, 311.83 μmol, 1 eq) in DCM (2 mL) was added BBr₃ (234.36 mg, 935.48 μmol, 90.14 uL, 3 eq). After 0.5 h at −20° C., the mixture was quenched with MeOH (10 mL) and concentrated to give the residue which was treated with LiOH·H₂O (39.26 mg, 935.48 μmol, 3 eq) in THF (2 mL) and H₂O (0.5 mL). After 48 h at 25° C., the mixture was concentrated, filtered, and washed with DCM:MeOH=10:1 (25 mL×2). The filtrate was concentrated to give 4-[6-[2-(aminomethyl)-4-methyl-thiazol-5-yl]-2-pyridyl]-2-hydroxy-benzoic acid 9c (120 mg, 281.21 μmol, 90.18% yield, 80% purity) as a solid. ¹H NMR (400 MHz, DMSO-d₆) δ ppm 8.03-7.97 (m, 1H), 7.95-7.92 (m, 1H), 7.81 (s, 1H), 7.73 (d, J=7.8 Hz, 1H), 7.52 (s, 1H), 7.46 (br d, J=8.0 Hz, 1H), 4.53 (br s, 1H), 4.45 (br s, 2H), 2.73 (s, 3H).

### Step 4: Synthesis of 2-hydroxy-4-[6-[2-[(methoxycarbonylamino)methyl]-4-methyl-thiazol-5-yl]-2-pyridyl]benzoic Acid (9)

To a solution of 4-[6-[2-(aminomethyl)-4-methyl-thiazol-5-yl]-2-pyridyl]-2-hydroxy-benzoic acid 9c (100 mg, 234.34 μmol, 1 eq) in DMF (4 mL) was added DIEA (90.86 mg, 703.02 μmol, 122.45 L, 3 eq) and methyl chloroformate (26.57 mg, 281.21 μmol, 21.78 μL, 1.2 eq). After 0.5 h at 25° C., the mixture was filtered to give the residue which was purified with prep-HPLC (column: Xtimate C18 10μ 250 mm*50 mm; mobile phase: [water(0.04% NH₃H₂O+10 mM NH₄HCO₃)-ACN]; B %: 5%-35%, 8 min) to give 2-hydroxy-4-[6-[2-[(methoxycarbonylamino)methyl]-4-methyl-thiazol-5-yl]-2-pyridyl]benzoic acid 9 (21.4 mg, 52.10 μmol, 22.23% yield, 97.25% purity) as a solid. ¹H NMR (400 MHz, CD₃OD) δ ppm 8.00 (d, J=8.0 Hz, 1H), 7.95-7.89 (m, 1H), 7.81 (d, J=7.8 Hz, 1H), 7.63 (d, J=7.8 Hz, 1H), 7.60 (s, 1H), 7.55 (br d, J=8.3 Hz, 1H), 4.58 (s, 2H), 3.74 (s, 3H), 2.73 (s, 3H); HPLC (Purity: 97.25%); ES-MS m/z 400.1 [M+H]*.

### Example S10. Preparation of 4-[4-[2-(acetamidomethyl)-4-methyl-thiazol-5-yl]thiazol-2-yl]-5-fluoro-2-hydroxy-benzoic Acid (Compound 10)

### Step 1: Synthesis of tert-butyl N-[(5-acetyl-4-methyl-thiazol-2-yl)methyl]carbamate (10a)

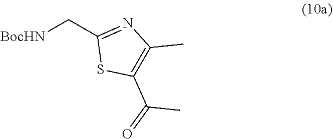 [SMILES: CC(=O)c1sc(CNC(=O)OC(C)(C)C)nc1C]

A mixture of tert-butyl N-(2-amino-2-thioxo-ethyl)carbamate (4.8 g, 25.23 mmol, 1 eq) 3-chloropentane-2,4-dione (3.39 g, 25.23 mmol, 3.00 mL, 1 eq) in EtOH (50 mL) was stirred at 80° C. for 1 h. The mixture was concentrated and diluted with water, then adjusted pH=9 with NaHCO₃ solution and extracted with EtOAc (100 mL×3). The combined organic layers were washed with brine (30 mL), dried over Na₂SO₄, filtered and concentrated under reduced pressure to give a residue which was purified with flash silica gel chromatography (ISCO®; 40 g SepaFlash® Silica Flash Column, Eluent of 0˜30% Ethyl acetate/Petroleum ether gradient @ 40 mL/min) to give tert-butyl N-[(5-acetyl-4-methyl-thiazol-2-yl)methyl]carbamate 10a (3.9 g, 12.98 mmol, 51.46% yield, 90% purity) as a solid. ¹H NMR (400 MHz, CDCl₃) δ ppm 5.26 (br s, 1H), 4.55 (br d, J=5.6 Hz, 2H), 2.68 (s, 3H), 2.50 (s, 3H), 1.46 (s, 9H).

### Step 2: Synthesis of tert-butyl N-[[5-(2-bromoacetyl)-4-methyl-thiazol-2-yl]methyl]carbamate (10b)

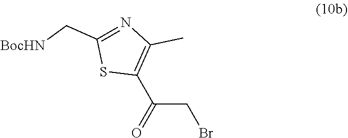 [SMILES: Cc1nc(CNC(=O)OC(C)(C)C)sc1C(=O)CBr]

To a solution of tert-butyl N-[(5-acetyl-4-methyl-thiazol-2-yl)methyl]carbamate 10a (2 g, 6.66 mmol, 1 eq) in THF (4 mL) was added trimethyl phenyl ammonium tribromide (3.75 g, 9.99 mmol, 1.5 eq). The mixture was stirred at 25° C. for 12 h. The reaction mixture was partitioned between H₂O (20 mL) and EtOAc (60 mL), then extracted with EtOAc (60 mL×2). The organic phase was separated, washed with brine (50 mL), dried over Na₂SO₄, filtered and concentrated under reduced pressure to give a residue which was purified with flash silica gel chromatography (ISCO®; 24 g SepaFlash® Silica Flash Column, Eluent of 0˜20% Ethylacetate/Petroleum ether gradient @ 35 mL/min) to give tert-butyl N-[[5-(2-bromoacetyl)-4-methyl-thiazol-2-yl]methyl]carbamate 10b (1.5 g, 3.87 mmol, 58.1% yield, 90% purity) as an oil. ¹H NMR (400 MHz, CDCl₃) δ ppm 5.26 (br s, 1H), 4.60 (br d, J=6.4 Hz, 2H), 4.24 (s, 2H), 2.74 (s, 3H), 1.49 (s, 9H).

### Step 3: Synthesis of methyl4-[4-[2-(aminomethyl)-4-methyl-thiazol-5-yl]thiazol-2-yl]-5-fluoro-2-methoxy-benzoate (10c)

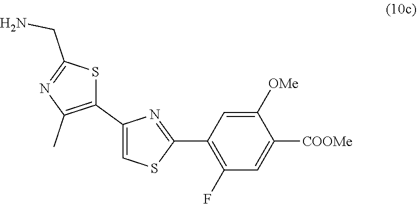 [SMILES: COC(=O)c1cc(F)c(-c2nc(-c3sc(CN)nc3C)cs2)cc1OC]

To a solution of tert-butyl N-[[5-(2-bromoacetyl)-4-methyl-thiazol-2-yl]methyl]carbamate 10b (1.2 g, 2.06 mmol, 1 eq) in EtOH (12 mL) was added methyl 4-carbamothioyl-5-fluoro-2-methoxy-benzoate (501.49 mg, 1.86 mmol, 0.9 eq). The mixture was stirred at 80° C. for 1 h and concentrated to give the residue which was purified with flash silica gel chromatography (ISCO®; 20 g SepaFlash® Silica Flash Column, Eluent of 0˜10% Methanol/Dichloromethane gradient @ 35 mL/min) to give methyl 4-[4-[2-(aminomethyl)-4-methyl-thiazol-5-yl]thiazol-2-yl]-5-fluoro-2-methoxy-benzoate 10c (700 mg, 1.60 mmol, 77.7% yield, 90% purity) as a solid. ¹H NMR (400 MHz, CD₃OD) δ ppm 7.99 (s, 1H), 7.87 (d, J=6.0 Hz, 1H), 7.69 (d, J=11.2 Hz, 1H), 3.96 (s, 2H), 3.91 (s, 3H), 3.57 (s, 3H), 2.59 (s, 3H).

### Step 4: Synthesis of methyl4-[4-[2-(aminomethyl)-4-methyl-thiazol-5-yl]thiazol-2-yl]-5-fluoro-2-hydroxy-benzoate (10d)

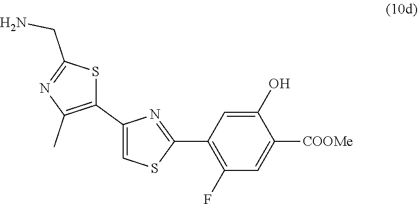 [SMILES: COC(=O)c1cc(F)c(-c2nc(-c3sc(CN)nc3C)cs2)cc1O]

To a solution of methyl 4-[4-[2-(aminomethyl)-4-methyl-thiazol-5-yl]thiazol-2-yl]-5-fluoro-2-methoxy-benzoate 10c (700 mg, 1.60 mmol, 1 eq) in CH₂Cl₂(10 mL) was added BBr₃ (3.21 g, 12.81 mmol, 1.23 mL, 8 eq). The mixture was stirred at −78° C. for 0.5 h. The mixture was quenched with saturated NaHCO₃ solution, adjusted into pH=5, and then extracted with DCM/MeOH=10/1 (100 mL×2). The combined organic layers were washed with brine, dried over Na₂SO₄, filtered and concentrated to give methyl 4-[4-[2-(aminomethyl)-4-methyl-thiazol-5-yl]thiazol-2-yl]-5-fluoro-2-hydroxy-benzoate 10d (600 mg, 1.42 mmol, 88.9% yield, 90% purity) as a solid. ¹H NMR (400 MHz, DMSO-d₆) δ ppm 8.05 (s, 1H), 7.82 (d, J=6.4 Hz, 1H), 7.71 (d, J=11.6 Hz, 1H), 3.96 (s, 2H), 3.88 (s, 3H), 2.59 (s, 3H).

### Step 5: Synthesis of 4-[4-[2-(aminomethyl)-4-methyl-thiazol-5-yl]thiazol-2-yl]-5-fluoro-2-hydroxy-benzoic Acid (10e)

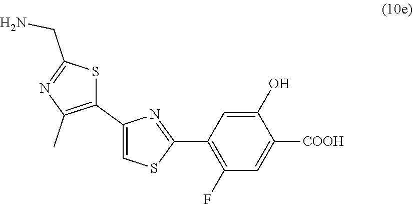 [SMILES: Cc1nc(CN)sc1-c1csc(-c2cc(O)c(C(=O)O)cc2F)n1]

To a solution of methyl 4-[4-[2-(aminomethyl)-4-methyl-thiazol-5-yl]thiazol-2-yl]-5-fluoro-2-hydroxy-benzoate 10d (600 mg, 1.42 mmol, 1 eq) in THF (5 mL) H₂O (5 mL) was added LiOH·H₂O (477.78 mg, 11.39 mmol, 8 eq). The mixture was stirred at 25° C. for 12 h. The mixture was adjusted into pH=5 with 1 M HCl and filtered to give the residue. 100 mg of the residue was purified by prep-HPLC (basic condition, Boston Prime C18 150*30 mm 5 um, water(0.04% NH₃H₂O+10 mM NH₄HCO₃)-ACN, B %: 10%-40%, 8 min) to give 4-[4-[2-(aminomethyl)-4-methyl-thiazol-5-yl]thiazol-2-yl]-5-fluoro-2-hydroxy-benzoic acid 10e (53.2 mg, 145.59 μmol, 10.23% yield, 100% purity) as a solid. ¹H NMR (400 MHz, DMSO-d₆) δ ppm 8.31 (br s, 2H), 8.08 (s, 1H), 7.54 (d, J=11.6 Hz, 1H), 7.43 (d, J=6.0 Hz, 1H), 4.41 (s, 2H), 2.67 (s, 3H); HPLC (Purity: 100.00%); ES-MS m/z 366.1 [M−H]—.

Step 6: Synthesis of 4-[4-[2-(acetamidomethyl)-4-methyl-thiazol-5-yl]thiazol-2-yl]-5-fluoro-2-hydroxy-benzoic acid (10). To a solution of 4-[4-[2-(aminomethyl)-4-methyl-thiazol-5-yl]thiazol-2-yl]-5-fluoro-2-hydroxy-benzoic acid 10e (100 mg, 246.30 μmol, 1 eq) in DMF (3 mL) was added DIEA (63.67 mg, 492.61 μmol, 85.80 μL, 2 eq), followed by acetyl chloride (25.13 mg, 320.20 mol, 22.85 μL, 1.3 eq) at 0° C. After 2h at 25° C., the mixture was filtered and the filtrate was purified with prep-HPLC (basic condition, Boston Prime C18 150*30 mm 5 um, water (0.04% NH₃H₂O+10 mM NH₄HCO₃)-ACN, B %: 10%-40%, 8 min) to give 4-[4-[2-(acetamidomethyl)-4-methyl-thiazol-5-yl]thiazol-2-yl]-5-fluoro-2-hydroxy-benzoic acid 10 (50.4 mg, 123.70 μmol, 50.22% yield, 100% purity) as a solid. ¹H NMR (400 MHz, DMSO-d₆) δ ppm 8.87-8.66 (m, 1H), 8.02 (s, 1H), 7.60 (d, J=11.6 Hz, 1H), 7.53 (d, J=6.0 Hz, 1H), 4.49 (d, J=6.0 Hz, 2H), 2.61 (s, 3H), 1.93 (s, 3H); HPLC (Purity: 100.00%); ES-MS m/z 408.1 [M+H]*.

Example 511. Compounds 11-331 were prepared in accordance with the procedures described in Examples S1-S10 using suitable reagents and modifications as would be known by a person of skill in the art.

Compounds described herein may be prepared according to the methods and examples as described herein and were characterized using standard techniques known in the art, such as ¹H NMR and mass spectra.

### Biological Examples

### Example B1. BPGM Synthase Assay

Materials

The following materials were used in this assay: deionized water; Bisphosphoglycerate mutase (BPGM; Standard Buffer: 50 mM Tris, 0.01% Tween 20, pH 7.4); BPGM Reaction Buffer (50 mM Tris, 0.01% Tween 20, 3.23 mM KH₂PO₄, pH 7.4); dithiothreitol (DTT; Stock solution: 100 mM in Standard Buffer); 384-Well Black Assay Plate (Corning, P/N 3575); DMSO; BPGM Stock Solution (500 nM in BPGM Standard Buffer); Glyceraldehyde 3-phosphate dehydrogenase (GAPDH; Stock Solution: 40 U/mL in 20 mM Tris-HCL Buffer with 20% Glycerol, 1 mM EDTA, 1 mM DTT); Glyceraldehyde 3-phosphate (GAP; Stock Solution: 20 mM in BPGM Standard Buffer); 3-PG Stock Solution (500 μM in BPGM Standard Buffer); Nicotinamide adenine dinucleotide (NAD; Stock Solution: 50 mM in BPGM Standard Buffer); Koningic Acid (1 mM Stock in DMSO); Resazurin Stock Solution (10 mM in DMSO); Diaphorase Stock Solution (40 U/mL in BPGM Standard Buffer); DTT Reaction Buffer (1 mM DTT in BPGM Reaction Buffer); Enzyme Solution (5 nM BPGM, 0.4 U/mL GAPDH in DTT Reaction Buffer); GAPDH Solution (0.4 U/mL GAPDH in DTT Reaction Buffer); Substrate Solution (2 mM NAD, 20 μM 3PG, 800 μM GAP in DTT Reaction Buffer); and Resazurin Solution (5 U/mL Diaphorase, 500 μM Resazurin in BPGM Standard Buffer).

Procedures

To the assay plate was added Enzyme Solution (20 uL) to columns 2-22 and 24 of the assay plate. To columns 1 and 23 was added GAPDH Solution (20 uL). A separate compound plate was prepared at the desired concentrations and all wells were normalized with 5% DMSO. The compound plate was incubated for 30 minutes at RT. Next, the substrate solution (20 μL) was dispensed to all reaction wells and the plate was incubated for 60 minutes at RT. Koningic acid (1 μL) was added and the plate was incubated for 10 minutes at RT. During this time period, the NADH fluorescence was read at ex: 360/em: 460. Next, Resazurin Solution (10 μL) was added to all reaction wells and the plate was incubated for 30 minutes at RT. The resorufin fluorescence was then read at ex:544/em:590.

The results of the BPGM synthase assay for select exemplary compounds are shown in Table 3.

TABLE 3
hBPGM Synthase IC₅₀ Values for Exemplary Compounds.

| Compound No. | hBPGM Synthase IC₅₀ (uM) |
|---|---|
| 1 | 1.5 |
| 2 | 0.146 |
| 3 | 0.26 |
| 4 | 0.59 |
| 5 | 0.063 |
| 6 | 0.07 |
| 7 | 0.06 |
| 8 | 0.095 |
| 9 | 0.317 |
| 10 | 0.095 |

### Example B2. BPGM Phosphatase Assay

Materials

The following materials were used in this assay: Tris buffer (50 mM Tris, 0.01% Tween 20, pH 7.4); cyclohexylammonium salt of 2,3-BPG (stock solution of 500 mM in ultrapure distilled water; human Bisphosphoglycerate mutase (hBPGM; stock solution of 110 μM in Standard Buffer); 3-Phosphoglyceric acid (3-PG; stock solution of 100 mM in ultrapure distilled water); 384-Well Polystyrene Plates (Fisherbrand™, Catalog No.: 12-566-625); BIOMOL GREEN REAGENT (Enzo life sciences, BML-AK111-0250); and 1 mM DMSO stock of exemplary compounds.

Procedures

A 3-PG enzyme mix solution (final enzyme concentration 62.5 nM, 3-PG concentration at 50 M, 14.5 uL) was added to onto a 384-well clear plate. Solutions of the test compounds were then added to the plate and the DMSO content was normalized across the plate. The plate was incubated at RT for 15 min. Next, the substrate 2,3-BPG (5 μL) was added to all the wells, and the plate was sealed and incubated for 24 hr at RT. After incubation, BIOMOL green dye (40 μL) was added to all the wells, the plate was shaken to mix, and the plate was incubated at RT for 20-30 min. Absorbance measurements were recorded at 620 nm.

Results

The results of the BPGM phosphatase assay for select exemplary compounds are shown in Table 4.

TABLE 4
hBPGM Phosphatase EC₅₀ Values for Exemplary Compounds.

| Compound No. | hBPGM Phosphatase EC₅₀ (nM) |
|---|---|
| 3 | 0.32 |
| 5 | 0.068 |
| 6 | 0.11 |
| 7 | 0.06 |
| 8 | 0.06 |
| 9 | 0.46 |
| 10 | 0.175 |

### Example B3. Human Cell-Based Assay

Materials

The following materials were used in this assay: DMSO; Hemox buffer (30 mM TES, 135 mM NaCl, 5 mM KCl, pH 7.4); Inosine (100 mM in hemox buffer); Glucose (1 M in water); human red blood cells; LC/MS Optima grade methanol; LC/MS Optima grade water; and Incubation buffer (30 mM TES, 135 mM NaCl, 5 mM KCl, 50 mM inosine, 20 mM glucose pH 7.4).

Procedures

Test compounds were loaded onto a 96-well plate (final concentration of DMSO was 1%). 10 mL whole blood was resuspended with an equivalent amount of 2× incubation buffer to make 25% het & 50% plasma solution. The final solution contains 10 mM glucose and 25 mM inosine. The blood suspension (100 uL) was added to the wells of the plate. The plate was covered with an adhesive foil cover and plastic lid, and incubated at 37° C. for 24 h. The samples were extracted through an SPE column and the metabolites (2,3-BPG, ATP) were analyzed by LC/MS.

Results

The results of the cell-based assay for select exemplary compounds are shown in Table 5.

TABLE 5
hRBC EC₅₀ Values for Exemplary Compounds.

| Compound No. | hRBC EC₅₀ (uM) |
|---|---|
| 2 | >100.000 |
| 4 | 23.501 |

Additional results from the hBPGM synthase and hBPGM phosphate assays as described herein are shown in Table 6.

TABLE 6
Additional Assay Results

| | huBPGM: phosphatase | huBPGM: synthase |
|---|---|---|
| Compound No. | Enzyme EC50 (M) | Enzyme IC50 (M) |
| 1 | | 1.00E−06 |
| 2 | | 1.50E−07 |
| 3 | 3.20E−07 | 1.80E−07 |
| 4 | 1.80E−07 | 7.10E−07 |
| 5 | 5.70E−08 | 5.90E−08 |
| 6 | 1.10E−07 | 6.90E−08 |
| 7 | 6.00E−08 | 5.90E−08 |
| 8 | 6.00E−08 | 9.40E−08 |
| 9 | 4.60E−07 | 3.20E−07 |
| 10 | 1.60E−07 | 9.10E−08 |
| 17 | | 8.90E−07 |
| 18 | | 6.50E−07 |
| 19 | | 8.50E−07 |
| 20 | | 2.10E−06 |
| 21 | | 2.90E−07 |
| 22 | | 2.90E−07 |
| 23 | | 1.70E−05 |
| 24 | | >2.5E−05 |
| 25 | | >2.5E−05 |
| 26 | | 4.30E−07 |
| 27 | | 7.40E−06 |
| 28 | | >2.5E−05 |
| 29 | | 1.80E−06 |
| 30 | | 1.50E−05 |
| 31 | | >2.5E−05 |
| 33 | | 1.10E−05 |
| 34 | | 3.40E−07 |
| 35 | | 2.60E−06 |
| 36 | | 2.70E−06 |
| 37 | | 7.60E−06 |
| 38 | | >2.5E−05 |
| 39 | | 1.80E−05 |
| 40 | | >2.5E−05 |
| 41 | | 1.90E−05 |
| 42 | | >2.5E−05 |
| 43 | | 2.30E−05 |
| 44 | | 1.90E−06 |
| 45 | | 1.40E−06 |
| 46 | | 1.20E−06 |
| 47 | | 9.80E−07 |
| 48 | | 1.90E−06 |
| 49 | 5.20E−07 | 3.50E−07 |
| 50 | >2.5E−05 | >2.5E−05 |
| 51 | 4.30E−06 | 2.10E−05 |
| 52 | >2.5E−05 | 1.60E−05 |
| 54 | | 1.80E−07 |
| 55 | | 3.80E−07 |
| 56 | | 3.30E−06 |
| 57 | | >2.5E−05 |
| 58 | | >2.5E−05 |
| 59 | | 9.70E−07 |
| 60 | | 7.60E−07 |
| 61 | | 1.30E−06 |
| 62 | | 1.80E−06 |
| 63 | >2.5E−05 | 6.30E−07 |
| 64 | | 9.10E−07 |
| 65 | >2.5E−05 | 2.60E−06 |
| 66 | 1.10E−07 | 6.10E−08 |
| 67 | 5.50E−07 | 8.70E−07 |
| 68 | 6.00E−08 | 1.40E−06 |
| 69 | | 5.50E−06 |
| 70 | | 1.10E−05 |
| 71 | >2.5E−05 | 5.60E−07 |
| 72 | >2.5E−05 | 4.80E−07 |
| 73 | >2.5E−05 | 2.20E−06 |
| 74 | 1.10E−06 | 1.80E−06 |
| 75 | >2.5E−05 | 4.60E−06 |
| 76 | >2.5E−05 | 3.50E−06 |
| 77 | | >2.5E−05 |
| 78 | 7.00E−08 | 1.80E−07 |
| 79 | 1.10E−07 | 5.50E−07 |
| 80 | 1.70E−06 | 2.20E−07 |
| 81 | 1.10E−06 | 4.50E−07 |
| 82 | 2.10E−07 | 1.10E−06 |
| 83 | 1.40E−06 | 3.60E−07 |
| 84 | | >2.5E−05 |
| 85 | | >2.5E−05 |
| 86 | 2.00E−07 | 1.50E−06 |
| 87 | 8.70E−06 | 3.40E−06 |
| 88 | 1.20E−05 | 1.00E−06 |
| 89 | | >2.5E−05 |
| 90 | | >2.5E−05 |
| 91 | | >2.5E−05 |
| 92 | | >2.5E−05 |
| 93 | 2.70E−07 | 3.30E−06 |
| 94 | >2.5E−05 | 1.10E−05 |
| 95 | 2.10E−06 | 2.80E−07 |
| 96 | 1.00E−08 | 1.30E−06 |
| 97 | 1.60E−07 | 1.00E−07 |
| 98 | 1.00E−07 | 1.70E−07 |
| 99 | 2.80E−07 | 4.00E−07 |
| 100 | 4.20E−07 | 5.60E−07 |
| 101 | 7.30E−06 | 7.60E−07 |
| 102 | 3.60E−07 | 9.00E−07 |
| 103 | 1.30E−07 | 9.50E−07 |
| 104 | | 1.50E−06 |
| 105 | 2.90E−07 | 1.60E−06 |
| 107 | | 1.90E−06 |
| 108 | 5.00E−07 | 3.20E−06 |
| 109 | | 8.10E−06 |
| 110 | | 8.80E−06 |
| 111 | | 1.50E−05 |
| 112 | | 3.50E−05 |
| 113 | 1.70E−07 | 1.30E−07 |
| 114 | 1.70E−07 | 1.50E−07 |
| 115 | 1.80E−07 | 6.70E−07 |
| 116 | >2.5E−05 | 6.90E−06 |
| 117 | 8.70E−06 | 1.70E−07 |
| 118 | 1.30E−07 | 1.80E−07 |
| 119 | 1.80E−06 | 3.40E−07 |
| 120 | >2.5E−05 | 5.00E−07 |
| 121 | 3.20E−06 | 1.80E−06 |
| 122 | | >2.5E−04 |
| 123 | 9.70E−06 | 5.30E−07 |
| 124 | 5.00E−08 | 4.90E−08 |
| 125 | 3.40E−07 | 4.10E−07 |
| 126 | 2.40E−07 | 4.60E−07 |
| 127 | 4.20E−07 | 4.90E−07 |
| 128 | 7.90E−08 | 1.90E−07 |
| 129 | 4.00E−08 | 4.00E−08 |
| 130 | 4.00E−08 | 7.00E−08 |
| 131 | 4.00E−08 | 4.20E−08 |
| 132 | 5.00E−08 | 4.50E−08 |
| 133 | 8.00E−08 | 5.00E−08 |
| 134 | 3.40E−07 | 1.00E−06 |
| 135 | 7.00E−08 | 4.00E−08 |
| 136 | 9.00E−08 | 3.10E−07 |
| 137 | 2.00E−08 | 2.80E−08 |
| 139 | 9.00E−08 | 8.40E−08 |
| 140 | 3.00E−08 | 9.50E−08 |
| 141 | 7.00E−08 | 1.30E−07 |
| 142 | 1.30E−07 | 1.90E−07 |
| 143 | 8.00E−08 | 2.00E−07 |
| 144 | | 2.00E−07 |
| 145 | 5.00E−08 | 2.50E−07 |
| 146 | 5.00E−08 | 4.60E−07 |
| 147 | 5.00E−08 | 6.10E−07 |
| 148 | 2.80E−07 | 2.50E−07 |
| 149 | 7.00E−08 | 3.00E−08 |
| 150 | | 5.00E−07 |
| 151 | 7.00E−08 | 6.50E−08 |
| 152 | 4.00E−08 | 6.70E−08 |
| 153 | 7.00E−08 | 9.90E−08 |
| 154 | 4.00E−08 | 1.70E−07 |
| 155 | >2.5E−05 | 3.10E−07 |
| 156 | 1.50E−06 | 3.50E−07 |
| 157 | | 8.00E−07 |
| 158 | >2.5E−05 | 4.40E−08 |
| 160 | 1.30E−07 | 6.50E−08 |
| 161 | 7.90E−08 | 5.90E−08 |
| 162 | 6.00E−08 | 1.40E−07 |
| 163 | 6.00E−08 | 1.70E−07 |
| 164 | 7.00E−08 | 1.70E−07 |
| 165 | 4.00E−08 | 2.40E−07 |
| 166 | 7.00E−08 | 2.50E−07 |
| 168 | 5.30E−07 | 3.70E−07 |
| 169 | 1.10E−07 | 4.80E−07 |
| 170 | | 1.20E−06 |
| 171 | 3.00E−08 | 2.80E−08 |
| 172 | 4.00E−08 | 4.00E−08 |
| 173 | 1.00E−07 | 4.90E−08 |
| 174 | 2.30E−07 | 5.30E−07 |
| 175 | 3.20E−07 | 5.20E−07 |
| 176 | | 8.90E−07 |
| 177 | 1.60E−07 | 5.90E−08 |
| 178 | 3.10E−06 | 2.00E−07 |
| 179 | | 6.00E−07 |
| 180 | 2.00E−08 | 2.80E−08 |
| 181 | 8.00E−08 | 2.10E−07 |
| 182 | 5.00E−08 | 5.60E−07 |
| 183 | 2.80E−07 | 6.60E−07 |
| 184 | 8.00E−08 | 1.50E−06 |
| 185 | 3.00E−08 | 1.60E−07 |
| 186 | 3.00E−08 | 3.80E−07 |
| 187 | | 1.70E−06 |
| 188 | 1.60E−07 | 5.50E−08 |
| 189 | 3.30E−07 | 1.20E−07 |
| 190 | 6.10E−07 | 3.60E−07 |
| 191 | 5.70E−07 | 7.80E−07 |
| 192 | 1.20E−07 | 4.90E−08 |
| 193 | 3.60E−07 | 1.70E−07 |
| 194 | 1.40E−07 | 2.50E−07 |
| 195 | | 1.20E−06 |
| 196 | | 5.60E−06 |
| 197 | 2.20E−07 | 2.40E−08 |
| 198 | 1.90E−07 | 5.00E−08 |
| 199 | 3.20E−07 | 6.70E−08 |
| 200 | 2.70E−07 | 7.30E−08 |
| 201 | 6.40E−07 | 1.10E−07 |
| 202 | 1.50E−07 | 1.20E−07 |
| 203 | 9.80E−07 | 2.10E−07 |
| 204 | 2.50E−07 | 2.70E−07 |
| 205 | | 1.10E−06 |
| 206 | | 2.00E−06 |
| 207 | 3.40E−07 | 2.80E−07 |
| 208 | 4.20E−07 | 3.60E−07 |
| 209 | 4.70E−07 | 2.50E−07 |
| 210 | 8.30E−07 | 9.30E−07 |
| 211 | 1.90E−07 | 1.00E−07 |
| 212 | 1.00E−07 | 1.60E−07 |
| 213 | 1.40E−07 | 1.30E−07 |
| 215 | 5.50E−08 | 9.50E−08 |
| 216 | 2.00E−07 | 1.90E−07 |
| 217 | 1.20E−07 | 1.10E−07 |
| 218 | 1.70E−07 | 1.30E−07 |
| 219 | 6.50E−08 | 1.20E−07 |
| 220 | 6.30E−08 | 5.20E−08 |
| 221 | 3.50E−08 | 9.00E−08 |
| 222 | 1.30E−07 | 1.10E−07 |
| 223 | 1.90E−07 | 1.50E−07 |
| 224 | 3.00E−07 | 2.60E−07 |
| 225 | 7.30E−06 | 8.30E−07 |
| 226 | 1.10E−07 | 5.60E−08 |
| 227 | 9.00E−08 | 7.60E−08 |
| 228 | 5.50E−08 | 8.70E−08 |
| 229 | 3.00E−08 | 1.90E−07 |
| 230 | >2.5E−05 | 4.40E−06 |
| 231 | 1.70E−07 | 5.20E−08 |
| 232 | 9.30E−08 | 6.00E−08 |
| 233 | 1.10E−07 | 7.80E−08 |
| 234 | 8.00E−08 | 8.40E−08 |
| 235 | 1.40E−07 | 1.10E−07 |
| 236 | 3.60E−07 | 1.30E−07 |
| 237 | 1.70E−07 | 1.40E−07 |
| 238 | >2.5E−05 | 1.30E−06 |
| 239 | >2.5E−05 | 7.30E−07 |
| 240 | 2.50E−06 | 2.30E−06 |
| 241 | | 2.10E−05 |
| 243 | 9.90E−07 | 8.30E−07 |
| 244 | >2.5E−05 | 8.40E−07 |
| 245 | 8.20E−07 | 6.90E−07 |
| 246 | 1.10E−07 | 8.20E−07 |
| 247 | 3.90E−06 | 1.00E−06 |
| 248 | 1.10E−07 | 1.10E−07 |
| 249 | 2.10E−07 | 1.80E−07 |
| 250 | 2.90E−07 | 2.50E−07 |
| 251 | 4.00E−07 | 2.70E−07 |
| 252 | 1.00E−06 | 1.00E−06 |
| 253 | 1.50E−07 | 1.90E−07 |
| 254 | 2.20E−07 | 2.90E−07 |
| 255 | 1.80E−06 | 7.60E−07 |
| 256 | 7.00E−08 | 8.80E−07 |
| 257 | 1.60E−07 | 3.60E−07 |
| 258 | | 1.10E−05 |
| 259 | >2.5E−05 | 5.70E−07 |
| 260 | 5.60E−06 | 1.40E−06 |
| 261 | | 2.00E−05 |
| 262 | 5.10E−07 | 5.30E−07 |
| 263 | 1.40E−06 | 1.30E−06 |
| 264 | | 7.00E−06 |
| 265 | | 7.80E−06 |
| 267 | 3.50E−07 | 5.20E−07 |
| 268 | | 1.20E−06 |
| 269 | 2.70E−07 | 2.00E−06 |
| 270 | >2.5E−05 | 4.60E−06 |
| 271 | | 8.00E−06 |
| 272 | 5.20E−07 | 2.90E−07 |
| 273 | 1.70E−07 | 3.90E−07 |
| 274 | 3.90E−07 | 4.30E−07 |
| 275 | >2.5E−05 | 5.00E−06 |
| 276 | 5.90E−07 | 4.80E−07 |
| 277 | 1.10E−06 | 8.60E−07 |
| 278 | 2.10E−06 | 9.00E−07 |
| 279 | 6.90E−06 | 2.60E−06 |
| 280 | 1.40E−07 | 3.30E−07 |
| 281 | 1.20E−07 | 7.30E−07 |
| 282 | 1.10E−07 | 7.50E−07 |
| 283 | 6.50E−06 | 1.30E−06 |
| 284 | 2.90E−06 | 2.80E−06 |
| 285 | 4.00E−07 | 3.20E−07 |
| 286 | 3.10E−06 | 8.10E−07 |
| 287 | | 9.10E−07 |
| 288 | | 3.80E−06 |
| 289 | 1.30E−07 | 1.20E−06 |
| 290 | | 2.20E−06 |
| 291 | | 3.30E−06 |
| 292 | | 5.80E−06 |
| 293 | | 9.30E−06 |
| 294 | | 4.40E−05 |
| 295 | | 6.00E−07 |
| 296 | | 3.70E−06 |
| 297 | | 2.80E−05 |
| 298 | | 5.00E−06 |
| 299 | | 2.50E−05 |
| 300 | | 1.60E−04 |
| 301 | | >2.5E−04 |
| 302 | | 1.20E−06 |
| 303 | | 1.70E−05 |
| 304 | | 5.40E−05 |
| 305 | | 8.20E−05 |
| 306 | | >2.5E−04 |
| 307 | | >2.5E−04 |
| 308 | | 1.00E−06 |
| 309 | | 1.80E−06 |
| 310 | | 2.00E−06 |
| 311 | | 2.10E−05 |
| 312 | | 6.60E−06 |
| 313 | | 9.10E−06 |
| 314 | | 1.00E−04 |
| 315 | | 1.40E−06 |
| 317 | | 3.00E−06 |
| 318 | | 4.80E−06 |
| 319 | | >2.5E−04 |
| 320 | | 7.00E−06 |
| 321 | | 7.30E−06 |
| 322 | | >2.5E−04 |
| 323 | | 1.60E−06 |
| 324 | | 2.30E−06 |
| 325 | | 3.50E−06 |
| 326 | | 6.40E−06 |
| 328 | | 4.50E−05 |
| 329 | | 2.30E−06 |
| 330 | | 6.60E−06 |
| 331 | | >2.5E−05 |
| 332 | | 4.50E−07 |
| 333 | | 1.10E−05 |
| 334 | | 1.70E−06 |
| 335 | | 3.10E−07 |
| 336 | | >2.5E−05 |
| 337 | | >2.5E−05 |
| 338 | | >2.5E−05 |
| 339 | | >2.5E−05 |
| 340 | | 3.60E−06 |
| 341 | | 4.10E−06 |
| 342 | | 1.90E−06 |

Although the foregoing embodiments has been described in some detail by way of illustration and example for purposes of clarity of understanding, the descriptions and examples should not be construed as limiting the scope of the disclosure. The disclosures of all patent and scientific literature cited herein are expressly incorporated herein in their entirety by reference.

1. A compound of Formula (I): 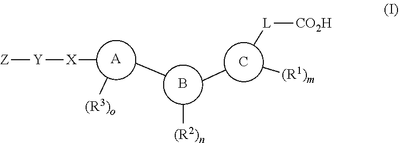
or a pharmaceutically acceptable salt thereof, wherein:
Ring A is phenylene, a 5- to 6-membered heteroarylene, or a 4- to 6-membered heterocyclylene, wherein the heteroarylene and heterocyclylene contain 1-3 heteroatoms selected from N and S;
Ring B is phenylene, a 5- to 6-membered heteroarylene, or a 4- to 6-membered heterocyclylene, wherein the heteroarylene and heterocyclylene contain 1-3 heteroatoms selected from N, O, and S;
Ring C is phenylene or a 5- to 6-membered heteroarylene, wherein the heteroarylene contains 1-3 heteroatoms selected from N, O, and S;
each R¹ is independently —OH, halo, C₁-C₆ alkyl, C₁-C₆ haloalkyl, —CN, —CO₂H, —NR⁵R⁶, or —N(H)CO₂(C₁-C₆ alkyl);
each R² is independently C₁-C₆ alkyl, C₁-C₆ haloalkyl, —CN, halo, —OH, oxo, or phenyl optionally substituted with 1-3 halo or —OH groups;
each R³ is independently C₁-C₆ alkyl or C₁-C₆ haloalkyl;
m is 0-4;
n is 0-4;
o is 0-4;
L is a bond, —OCR⁵R⁶—, —CR⁵R⁶—, —C(O)N(H)CR⁵R⁶CH₂—, —C(O)N(H)CR⁵R⁶—, —(C(O)N(H)CH₂)_{q}—, —C(O)N(H)SO₂(C₆H₄)OCH₂—, —C(O)-(5- to 6-membered heterocyclylene)-OCR⁵CR⁶—, —C(O)N(H)CR⁵R⁶C(O)-(5- to 6-membered heterocyclylene)-, or —S(O)₂CR⁵R⁶—, wherein the heterocyclylene contains 1-3 heteroatoms selected from N and 0;
each R⁵ and R⁶ is independently H, C₁-C₆ alkyl, C₁-C₆ haloalkyl, or phenyl;
q is 1 or 2;
X is —CR⁷R⁸—, —C(O)—, —N(H)—, or a bond;
Y is —O—, —N(H)—, —CR⁷R⁸—, —OCR⁷R⁸—, or a bond;
each R⁷ and R⁸ is independently H or C₁-C₆ alkyl;
Z is Z¹ or Z²;
Z¹ is H, halo, C₁-C₆ alkyl, C₁-C₆ haloalkyl, —C(O)(C₁-C₆ alkyl), —C(O)NR⁵R⁶, —CH₂C(O)NR⁵R⁶, —CO₂(C₁-C₆ alkyl), —CO₂(C₁-C₆ haloalkyl), —C(O)(C₁-C₆ alkyl), —C(O)(C₁-C₆ haloalkyl), —SO₂(C₁-C₆ alkyl), —C(O)(C₁-C₆ alkylene)-NR⁵R⁶, —CO₂(C₁-C₆ alkylene)-NR⁵R⁶, —N(H)C(O)(C₁-C₆ alkyl), —C(O)NR⁹(C₁-C₆ alkylene)-NR⁵R⁶, —(C₁-C₆ alkylene)-OR⁹, —C(O)C(O)O(C₁-C₆ alkyl), —C(O)C(O)—NR⁵R⁶, —(CH₂CH₂O)ᵣ(C₁-C₆ alkyl), or —C(O)CH₂(OCH₂CH₂)ᵣO(C₁-C₆ alkyl),
wherein C₁-C₆ alkylene is optionally substituted with 1-6 halo, C₁-C₆ alkyl, or C₁-C₆ haloalkyl;
R⁹ is H or C₁-C₆ alkyl;
r is 1-4;
Z² is phenyl, —C(O)(phenyl), 5- to 6-membered heteroaryl, —C(O)-(5- to 6-membered heteroaryl), —CR⁵R⁶-(5- to 6-membered heteroaryl), 4- to 6-membered heterocyclyl,
—CR⁵R⁶-(4- to 6-membered heterocyclyl), —C(O)-(4- to 6-membered heterocyclyl), C₃-C₆ cycloalkyl, —C(O)(C₃-C₆ cycloalkyl), —CO₂(C₃-C₆ cycloalkyl), or —CR⁵R⁶—(C₃-C₆ cycloalkyl),
wherein the heteroaryl and heterocyclyl contain 1-3 heteroatoms selected from N and O, and
wherein the phenyl, heteroaryl, and heterocyclyl are optionally substituted by 1-5 R¹⁰; and
each R¹⁰ is independently halo, —OH, C₁-C₆ alkyl, C₁-C₆ haloalkyl, —CN, oxo, —NR⁵R⁶, or —N(H)C(O)(C₁-C₆ alkyl).

2. A compound of Formula (I-a): 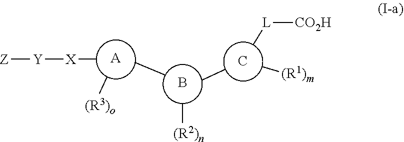
or a pharmaceutically acceptable salt thereof, wherein:
Ring A is thiazolylene;
Ring B is phenylene, a 5- to 6-membered heteroarylene, or a 4- to 6-membered heterocyclylene, wherein the heteroarylene and heterocyclylene contain 1-3 heteroatoms selected from N, O, and S;
Ring C is phenylene or a 5- to 6-membered heteroarylene, wherein the heteroarylene contains 1-3 heteroatoms selected from N, O, and S;
each R¹ is independently —OH, halo, C₁-C₆ alkyl, C₁-C₆ haloalkyl, —CN, —CO₂H, —NR⁵R⁶, or —N(H)CO₂(C₁-C₆ alkyl);
each R² is independently C₁-C₆ alkyl, C₁-C₆ haloalkyl, —CN, halo, —OH, oxo, or phenyl optionally substituted with 1-3 halo or —OH groups;
each R³ is independently C₁-C₆ alkyl or C₁-C₆ haloalkyl;
m is 0-4;
n is 0-4;
o is 0-4;
L is a bond, —OCR⁵R⁶—, —CR⁵R⁶—, —C(O)N(H)CR⁵R⁶CH₂—, —C(O)N(H)CR⁵R⁶—, —C(O)N(H)SO₂(C₆H₄)OCH₂—, —(C(O)N(H)CH₂)_{q}—, —C(O)-(5- to 6-membered heterocyclylene)-OCR⁵CR⁶—, —C(O)N(H)CR⁵R⁶C(O)-(5- to 6-membered heterocyclylene)-, or —S(O)₂CR⁵R⁶—, wherein the heterocyclylene contains 1-3 heteroatoms selected from N and 0;
each R⁵ and R⁶ is independently H, C₁-C₆ alkyl, C₁-C₆ haloalkyl, or phenyl;
q is 1 or 2;
X is —CR⁷R⁸—;
Y is —O—, —N(H)—, —CR⁷R⁸—, —OCR⁷R⁸—, or a bond;
each R⁷ and R⁸ is independently H or C₁-C₆ alkyl;
Z is Z¹ or Z²;
Z¹ is H, halo, C₁-C₆ alkyl, C₁-C₆ haloalkyl, —C(O)(C₁-C₆ alkyl), —C(O)NR⁵R⁶, —CH₂C(O)NR⁵R⁶, —CO₂(C₁-C₆ alkyl), —CO₂(C₁-C₆ haloalkyl), —C(O)(C₁-C₆ alkyl), —C(O)(C₁-C₆ haloalkyl), —SO₂(C₁-C₆ alkyl), —C(O)(C₁-C₆ alkylene)-NR⁵R⁶, —CO₂(C₁-C₆ alkylene)-NR⁵R⁶, —N(H)C(O)(C₁-C₆ alkyl), —C(O)NR⁹(C₁-C₆ alkylene)-NR⁵R⁶, —(C₁-C₆ alkylene)-OR⁹, —C(O)C(O)O(C₁-C₆ alkyl), —C(O)C(O)—NR⁵R⁶, —(CH₂CH₂O)ᵣ(C₁-C₆ alkyl), or —C(O)CH₂(OCH₂CH₂)ᵣO(C₁-C₆ alkyl),
wherein C₁-C₆ alkylene is optionally substituted with 1-6 halo, C₁-C₆ alkyl, or C₁-C₆ haloalkyl;
R⁹ is H or C₁-C₆ alkyl;
r is 1-4;
Z² is phenyl, —C(O)(phenyl), 5- to 6-membered heteroaryl, —C(O)-(5- to 6-membered heteroaryl), —CR⁵R⁶-(5- to 6-membered heteroaryl), 4- to 6-membered heterocyclyl, —CR⁵R⁶-(4- to 6-membered heterocyclyl), —C(O)-(4- to 6-membered heterocyclyl), C₃-C₆ cycloalkyl, —C(O)(C₃-C₆ cycloalkyl), —CO₂(C₃-C₆ cycloalkyl), or —CR⁵R⁶—(C₃-C₆ cycloalkyl),
wherein the heteroaryl and heterocyclyl contain 1-3 heteroatoms selected from N and O, and
wherein the phenyl, heteroaryl, and heterocyclyl are optionally substituted by 1-5 R¹⁰; and
each R¹⁰ is independently halo, —OH, C₁-C₆ alkyl, C₁-C₆ haloalkyl, —CN, oxo, —NR⁵R⁶, or —N(H)C(O)(C₁-C₆ alkyl).

3. A compound of Formula (I-al): 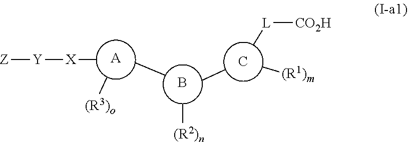
or a pharmaceutically acceptable salt thereof, wherein:
Ring A is thiazolylene;
Ring B is phenylene, a 5- to 6-membered heteroarylene, or a 4- to 6-membered heterocyclylene, wherein the heteroarylene and heterocyclylene contain 1-3 heteroatoms selected from N, O, and S;
Ring C is phenylene or a 5- to 6-membered heteroarylene, wherein the heteroarylene contains 1-3 heteroatoms selected from N, O, and S;
each R¹ is independently —OH, halo, C₁-C₆ alkyl, C₁-C₆ haloalkyl, —CN, —CO₂H, —NR⁵R⁶, or —N(H)CO₂(C₁-C₆ alkyl);
each R² is independently C₁-C₆ alkyl, C₁-C₆ haloalkyl, —CN, halo, —OH, oxo, or phenyl optionally substituted with 1-3 halo or —OH groups;
each R³ is independently C₁-C₆ alkyl or C₁-C₆ haloalkyl;
m is 0-4;
n is 0-4;
o is 0-4;
L is a bond, —OCR⁵R⁶—, —CR⁵R⁶—, —C(O)N(H)CR⁵R⁶CH₂—, —C(O)N(H)CR⁵R⁶—, —C(O)N(H)SO₂(C₆H₄)OCH₂—, —(C(O)N(H)CH₂)_{q}—, —C(O)-(5- to 6-membered heterocyclylene)-OCR⁵CR⁶—, —C(O)N(H)CR⁵R⁶C(O)-(5- to 6-membered heterocyclylene)-, or —S(O)₂CR⁵R⁶—, wherein the heterocyclylene contains 1-3 heteroatoms selected from N and 0;
each R⁵ and R⁶ is independently H, C₁-C₆ alkyl, C₁-C₆ haloalkyl, or phenyl;
q is 1 or 2;
X is —CR⁷R⁸—;
Y is —O—, —N(H)—, —CR⁷R⁸—, or —OCR⁷R⁸—;
each R⁷ and R⁸ is independently H or C₁-C₆ alkyl;
Z is Z²;
Z² is phenyl, —C(O)(phenyl), 5- to 6-membered heteroaryl, —C(O)-(5- to 6-membered heteroaryl), —CR⁵R⁶-(5- to 6-membered heteroaryl), 4- to 6-membered heterocyclyl, —CR⁵R⁶-(4- to 6-membered heterocyclyl), —C(O)-(4- to 6-membered heterocyclyl), C₃-C₆ cycloalkyl, —C(O)(C₃-C₆ cycloalkyl), —CO₂(C₃-C₆ cycloalkyl), or —CR⁵R⁶—(C₃-C₆ cycloalkyl),
wherein the heteroaryl and heterocyclyl contain 1-3 heteroatoms selected from N and O, and
wherein the phenyl, heteroaryl, and heterocyclyl are optionally substituted by 1-5 R¹⁰; and
each R¹⁰ is independently halo, —OH, C₁-C₆ alkyl, C₁-C₆ haloalkyl, —CN, oxo, —NR⁵R⁶, or —N(H)C(O)(C₁-C₆ alkyl).

4. The compound of any one of claims 1-3, or a pharmaceutically acceptable salt thereof, wherein Ring C is phenylene, pyrazolylene, furanylene, thienylene, pyridinylene, pyrrolylene, pyrimidinylene, or thiazolylene.

5. The compound of any one of claims 1-4, or a pharmaceutically acceptable salt thereof, wherein L is a bond.

6. The compound of any one of claims 1-4, or a pharmaceutically acceptable salt thereof, wherein L is —OCR⁵R⁶—, —CR⁵R⁶—, —C(O)N(H)CR⁵R⁶CH₂—, —C(O)N(H)CR⁵R⁶—, —C(O)N(H)SO₂(C₆H₄)OCH₂—, —SO₂CR⁵R⁶—, —C(O)-(5- to 6-membered heterocyclylene)-OCR⁵CR⁶—, —C(O)N(H)CR⁵R⁶C(O)-(5- to 6-membered heterocyclylene)-, or —(C(O)N(H)CH₂)_{q}—.

7. The compound of claim 6, or a pharmaceutically acceptable salt thereof, wherein R⁵ and R⁶ are each H.

8. The compound of claim 6, or a pharmaceutically acceptable salt thereof, wherein R⁵ is H; and R⁶ is phenyl.

9. The compound of any one of claims 1-4, or a pharmaceutically acceptable salt thereof, wherein:
-L-CO₂H is —CO₂H, —CH₂CO₂H, —OCH₂CO₂H, —C(O)N(H)CH(C₆H₅)CH₂CO₂H, —C(O)N(H)CH(C₆H₅)CO₂H, —SO₂CH₂CO₂H, —C(O)N(H)CH₂C(O)N(H)CH₂CO₂H, —C(O)N(H)CH₂CO₂H, —C(O)N(H)SO₂(C₆H₄)OCH₂CO₂H,

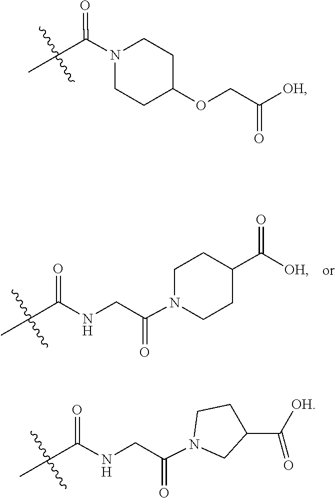 [SMILES: *C(=O)N1CCC(OCC(=O)O)CC1 ; *C(=O)NCC(=O)N1CCC(C(=O)O)CC1 ; *C(=O)NCC(=O)N1CCC(C(=O)O)C1]

10. The compound of any one of claims 1-9, or a pharmaceutically acceptable salt thereof, wherein:
each R¹ is independently halo, —OH, C₁-C₃ haloalkyl, C₁-C₃ alkyl, —CO₂H, —CN, —NH₂, or —N(H)CO₂(C₁-C₃ alkyl).

11. The compound of claim 10, or a pharmaceutically acceptable salt thereof, wherein:
each R¹ is independently F, Cl, —OH, —CHF₂, —CF₃, isopropyl, —CH₃, —CO₂H, —CN, —NH₂, or —N(H)CO₂CH₃.

12. The compound of any one of claims 1-10, or a pharmaceutically acceptable salt thereof, wherein m is 0, 1, 2, or 3.

13. The compound of any one of claims 1-12, or a pharmaceutically acceptable salt thereof, wherein:
Ring B is pyrazolylene, thienylene, phenylene, pyridinylene, triazolylene, pyrimidinylene, thiazolylene, piperidinylene, thiadiazolylene, isothiazolylene, oxadiazolylene, oxazolylene, or 2H-pyridinylene.

14. The compound of any one of claims 1-13, or a pharmaceutically acceptable salt thereof, wherein each R² is independently C₁-C₃ alkyl, halo, phenyl substituted with 1-2 OH groups, —CN, —OH, or oxo.

15. The compound of claim 14, or a pharmaceutically acceptable salt thereof, wherein each R² is independently —CH₃, 2-hydroxyphenyl, —CN, F, —OH, or oxo.

16. The compound of any one of claims 1-13, or a pharmaceutically acceptable salt thereof, wherein n is 0 or 1.

17. The compound of claim 1, or a pharmaceutically acceptable salt thereof, wherein: Ring A is thiazolylene, piperidinylene, pyridinylene, pyrazolylene, phenylene, azetidinylene, or pyrrolidinylene.

18. The compound of any one of claims 1-17, or a pharmaceutically acceptable salt thereof, wherein each R³ is independently C₁-C₃ alkyl.

19. The compound of claim 18, or a pharmaceutically acceptable salt thereof, wherein each R³ is independently —CH₃ or isopropyl.

20. The compound of claim 1, or a pharmaceutically acceptable salt thereof, wherein X is —CR⁷R⁸—.

21. The compound of any one of claims 1-20, or a pharmaceutically acceptable salt thereof, wherein R⁷ and R⁸ are independently H or —CH₃.

22. The compound of claim 21, or a pharmaceutically acceptable salt thereof, wherein R⁷ and R⁸ are each H.

23. The compound of claim 19, or a pharmaceutically acceptable salt thereof, wherein R⁷ is H; and R⁸ is —CH₃.

24. The compound of claim 1, or a pharmaceutically acceptable salt thereof, wherein X is —C(O)—, —N(H)—, or a bond.

25. The compound of any one of claims 1-24, or a pharmaceutically acceptable salt thereof, wherein Y is —O—.

26. The compound of any one of claims 1-24, or a pharmaceutically acceptable salt thereof, wherein Y is a bond, —N(H)—, —OCR⁷R⁸—, or —CR⁷R⁸—.

27. The compound of claim 26, or a pharmaceutically acceptable salt thereof, wherein R⁷ and R⁸ are each H.

28. The compound of any one of claims 1-2, or a pharmaceutically acceptable salt thereof, wherein Z is Z¹.

29. The compound of claim 28, or a pharmaceutically acceptable salt thereof, wherein:
Z¹ is H, halo, C₁-C₃ alkyl, C₁-C₃ haloalkyl, —C(O)(C₁-C₃ alkyl), —C(O)NR⁵R⁶, —CH₂C(O)NR⁵R⁶, —CO₂(C₁-C₃ alkyl), —CO₂(C₁-C₃ haloalkyl), —C(O)(C₁-C₃ alkyl), —C(O)(C₁-C₃ haloalkyl), —SO₂(C₁-C₃ alkyl), —C(O)(C₁-C₃ alkylene)-NR⁵R⁶, —CO₂(C₁-C₃ alkylene)-NR⁵R⁶, —N(H)C(O)(C₁-C₃ alkyl), —(C₁-C₃ alkylene)-OR⁹, —C(O)NR⁹(C₁-C₃ alkylene)-NR⁵R⁶, —C(O)C(O)O(C₁-C₆ alkyl), —C(O)C(O)—NR⁵R⁶, —(CH₂CH₂O)ᵣ(C₁-C₃ alkyl), or —C(O)CH₂(OCH₂CH₂)ᵣO(C₁-C₃ alkyl),
wherein C₁-C₃ alkylene is optionally substituted with 1-2 halo, C₁-C₃ alkyl, or C₁-C₃ haloalkyl;
R⁵ and R⁶ are independently H or C₁-C₃ alkyl; and
R⁹ is H or C₁-C₃ alkyl.

30. The compound of claim 29, or a pharmaceutically acceptable salt thereof, wherein:
Z¹ is —C(O)N(CH₃)₂, —C(O)N(H)CH₃, —C(O)NH₂, H, —CH₂C(CH₃)₃, —C(O)C(CH₃)₃, —C(O)N(CH₃)CH₂CH₂N(CH₃)₂, —C(O)OCH₂CH₂N(CH₃)₂, —C(O)CH₂CH₂N(CH₃)₂, —C(O)OCH₃, —C(O)CH(CH₃)CH₂N(CH₃)₂, —C(O)CH₂N(CH₃)₂, —C(O)CH₂(OCH₂CH₂)₄OCH₃, —C(O)C(CH₃)₂N(CH₃)₂, —C(O)OCH₂CF₃, —CH₂C(O)NH₂, —CH₂C(O)N(CH₃)₂, —CH₂CH₂OCH₃, isopropyl, —C(O)CH₃, —C(O)CO₂CH₃, —C(O)C(O)NH₂, —C(O)C(O)N(CH₃)₂, —SO₂CH₃, —CO₂CH₂CH₃, —CO(isopropyl), —CO₂(isopropyl), F, —N(H)C(O)CH₃, —(CH₂CH₂O)₃CH₃, or —CF₃.

31. The compound of any one of claims 1 and 3, or a pharmaceutically acceptable salt thereof, wherein Z is Z².

32. The compound of claim 31, or a pharmaceutically acceptable salt thereof, wherein:
Z² is phenyl, —C(O)(phenyl), 5- to 6-membered heteroaryl, —C(O)-(5- to 6-membered heteroaryl), —CR⁵R⁶-(5- to 6-membered heteroaryl), 4- to 6-membered heterocyclyl, —CR⁵R⁶-(4- to 6-membered heterocyclyl), —C(O)-(4- to 6-membered heterocyclyl), C₃-C₆ cycloalkyl, —C(O)(C₃-C₆ cycloalkyl), —CO₂(C₃-C₆ cycloalkyl), or —CR⁵R⁶—(C₃-C₆ cycloalkyl),
wherein the heteroaryl and heterocyclyl contain 1-3 heteroatoms selected from N and O, and
wherein the phenyl, heteroaryl, and heterocyclyl are optionally substituted by 1-5 R¹⁰; and
R⁵ and R⁶ are independently H or C₁-C₃ alkyl.

33. The compound of claim 32, or a pharmaceutically acceptable salt thereof, wherein:
Z² is pyridinyl, pyrimidinyl, pyrrolidinyl, tetrahydropyranyl, tetrahydrofuranyl, pyridazinyl, —C(O)(pyradazinyl), pyrazolyl, —C(O)(cyclopropyl), —CO₂(cyclopropyl), dihydropyridinyl, dihydropyrimidinyl, phenyl, —C(O)(phenyl), —C(O)(piperazinyl), —C(O)(piperidinyl), —C(O)(pyrrolidinyl), —CH₂(pyridinyl), —C(O)(isoxazolyl), —CH(CH₃)(pyridinyl), —C(O)(pyrazolyl), cyclohexyl, cyclobutyl, —C(O)(pyridinyl), —C(O)(pyrimidinyl), —C(O)(cyclopentyl), —C(O)(oxetanyl), morpholinyl, oxazolidinyl, or piperidinyl,
wherein the phenyl, heteroaryl, and heterocyclyl are optionally substituted by 1-3 R¹⁰.

34. The compound of any one of claims 1, 3, and 31-33, or a pharmaceutically acceptable salt thereof, wherein:
each R¹⁰ is independently halo, —OH, C₁-C₃ haloalkyl, C₁-C₃ alkyl, —CN, oxo, —NH₂, or —N(H)C(O)(C₁-C₃ alkyl).

35. The compound of claim 34, or a pharmaceutically acceptable salt thereof, wherein:
each R¹⁰ is independently F, Cl, —OH, —CF₃, isopropyl, —CH₃, —CN, oxo, —NH₂, or —NHC(O)CH₃.

36. The compound of claim 1, or a pharmaceutically acceptable salt thereof, wherein:
Z—Y—X— is

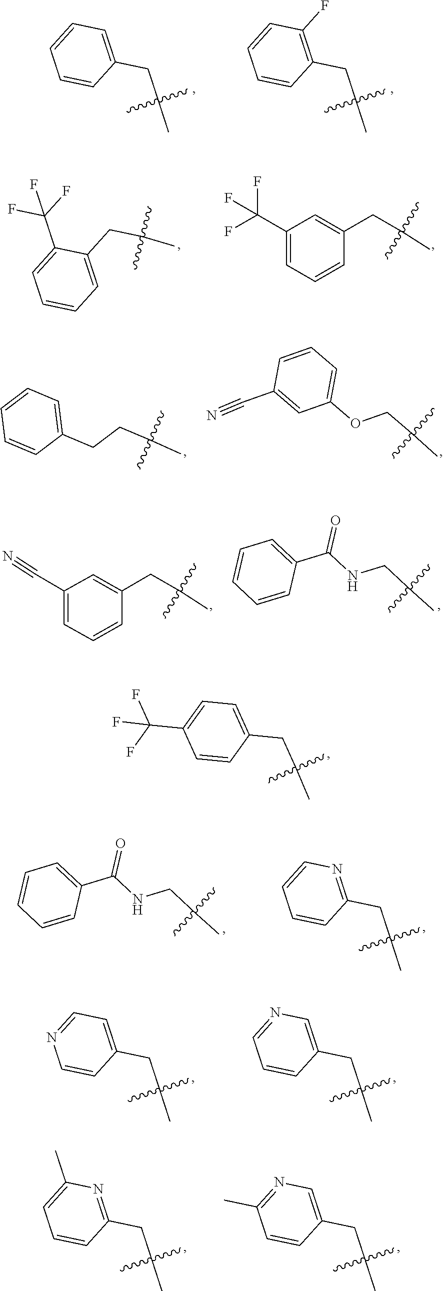

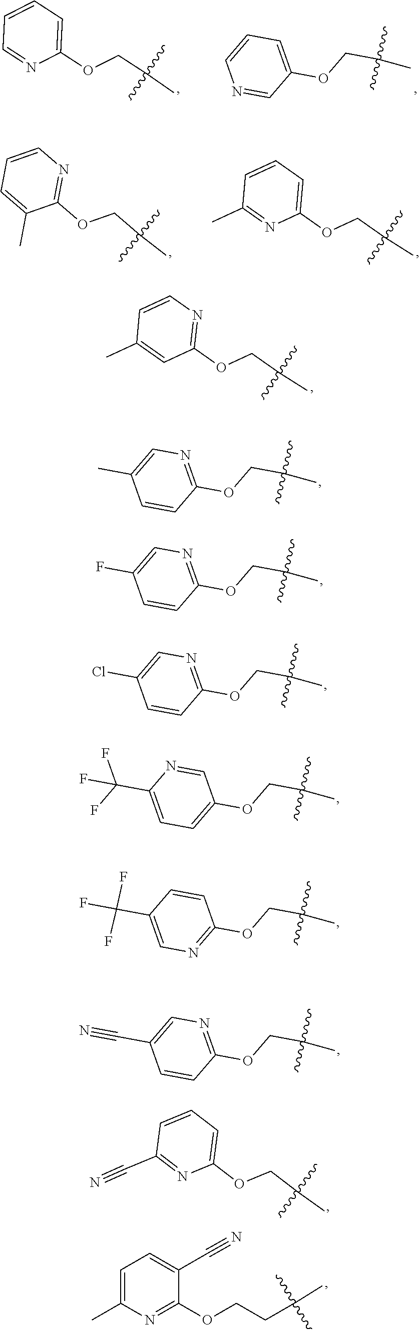

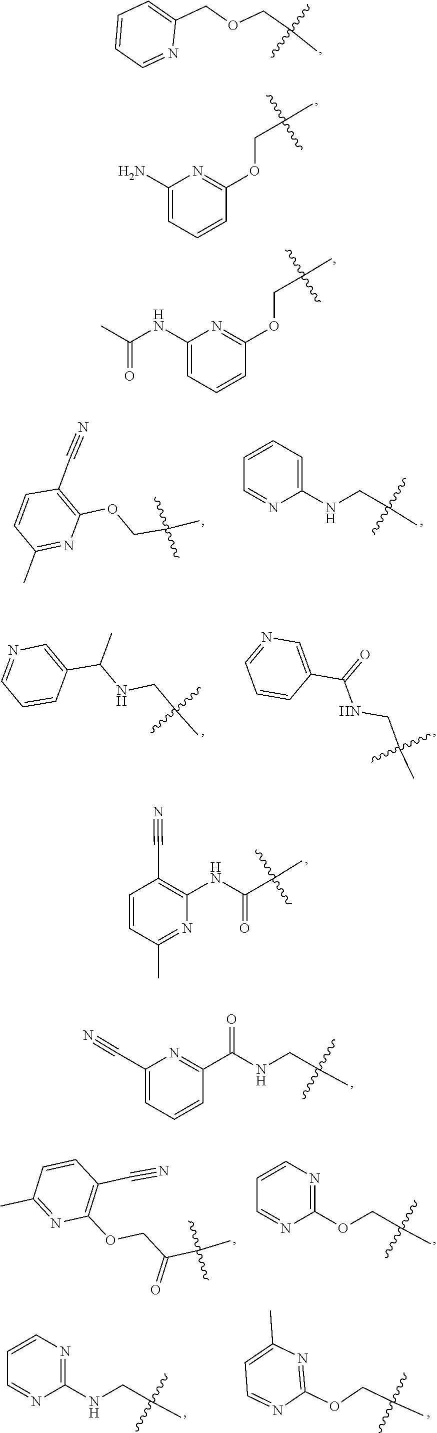

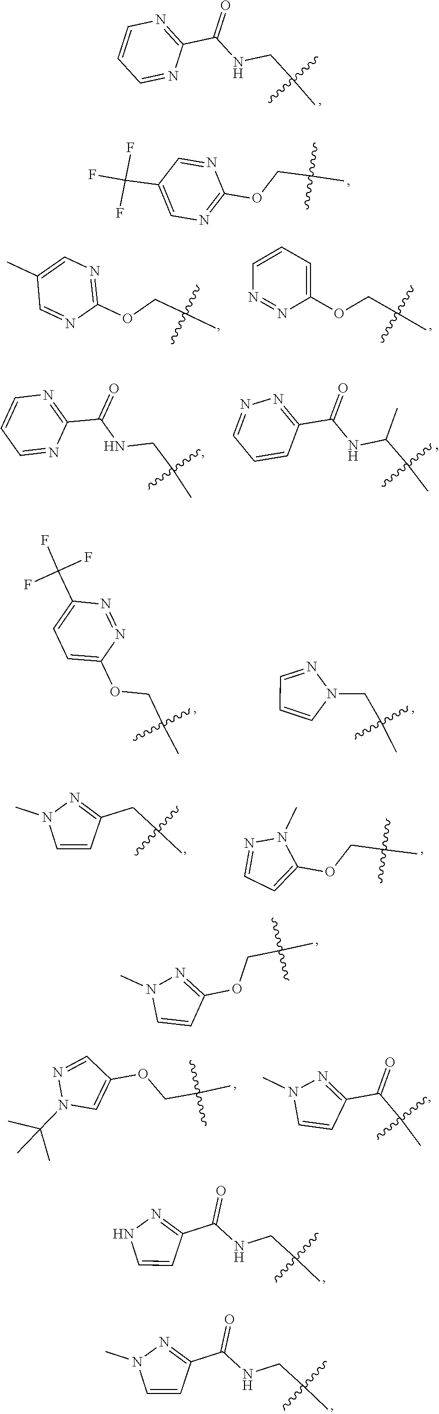

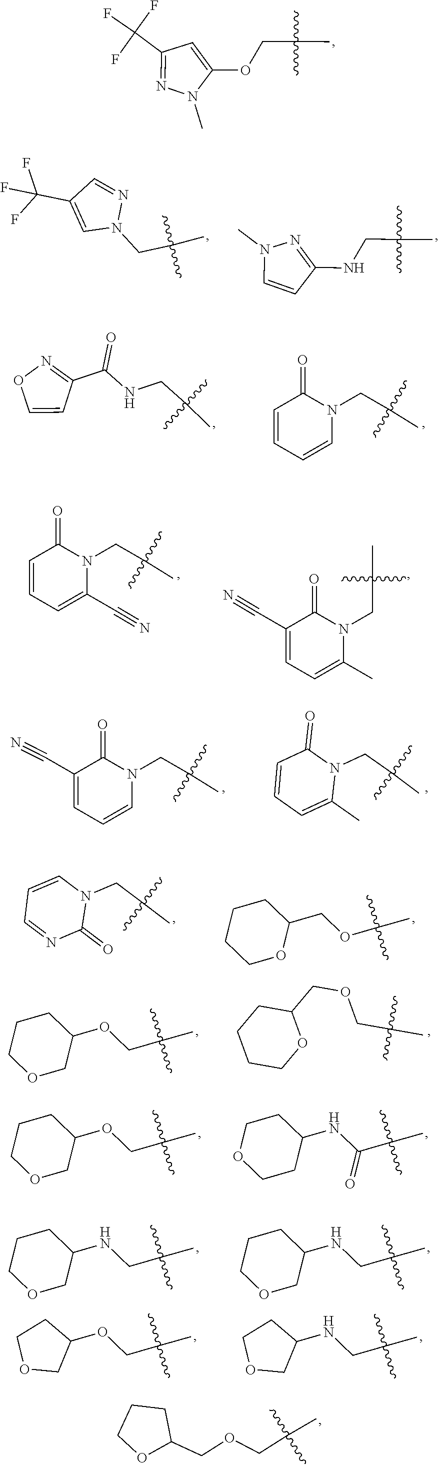

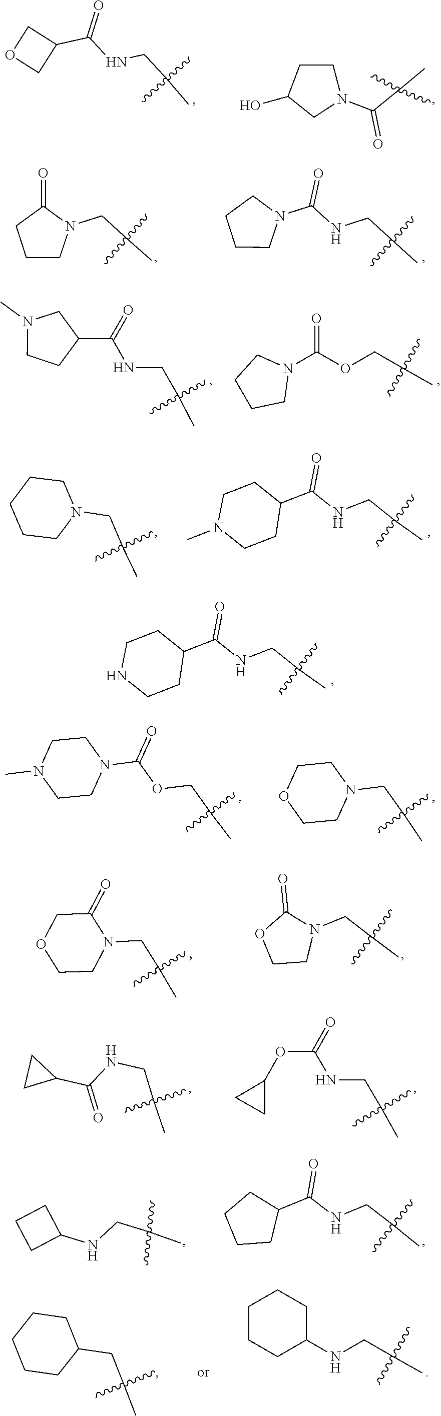

37. The compound of claim 1, or a pharmaceutically acceptable salt thereof, wherein:
Z—Y—X— is

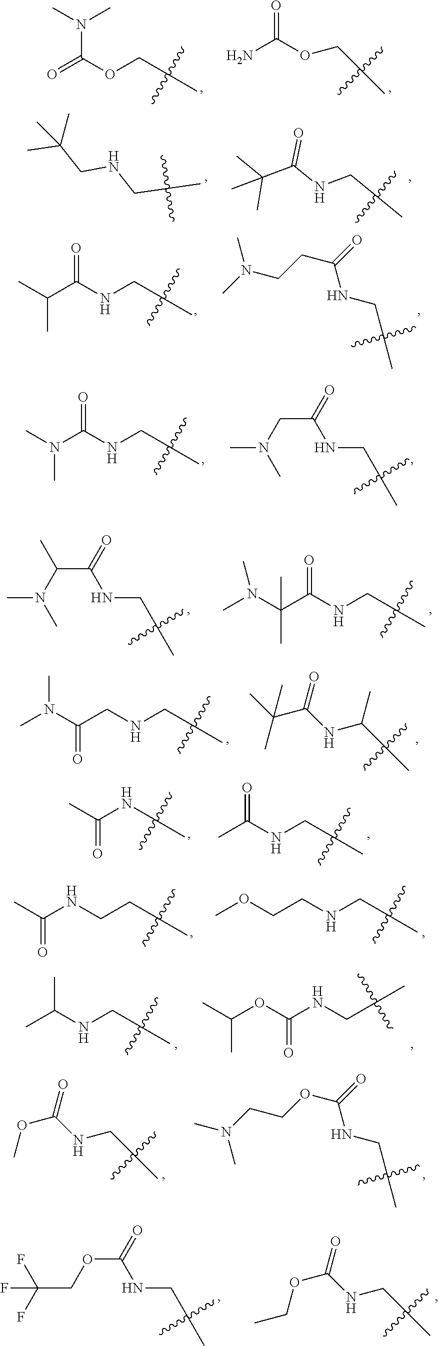

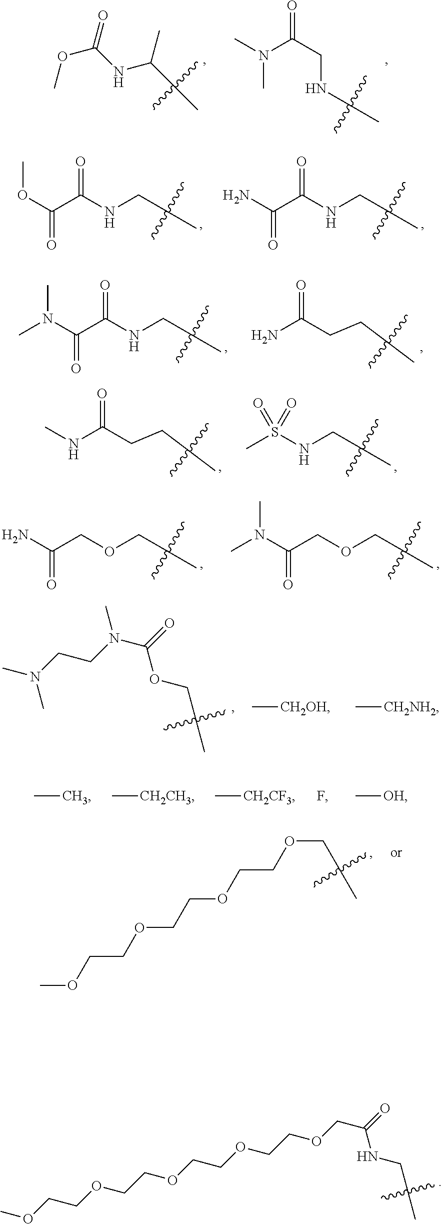

38. A compound selected from the compounds in Table 1, or a pharmaceutically acceptable salt thereof.

39. A compound selected from the compounds in Table 2, or a pharmaceutically acceptable salt thereof.

40. A pharmaceutical composition comprising the compound of any one of claims 1-39, or a pharmaceutically acceptable salt thereof, and at least one pharmaceutically acceptable excipient.

41. A method of modulating bis-phosphoglycerate mutase (BPGM) comprising contacting an effective amount of the compound of any one of claims 1-39, or a pharmaceutically acceptable salt thereof, or the pharmaceutical composition of claim 40, with the BPGM.

42. A method of treating sickle cell disease in a subject in need thereof, comprising administering to the subject a therapeutically effective amount of the compound of any one of claims 1-39, or a pharmaceutically acceptable salt thereof, or the pharmaceutical composition of claim 40.
